(19) United States
(12) Patent Application Publication
(10) Pub. No.: US 2025/0134891 A1
(43) Pub. Date: May 1, 2025

(54) USE OF SOS1 INHIBITORS WITH MTOR INHIBITORS TO TREAT CANCERS

(71) Applicant: Revolution Medicines, Inc., Redwood City, CA (US)

(72) Inventors: Bianca Jennifer LEE, Redwood City, CA (US); Grace J. LEE, San Francisco, CA (US); David Church MONTGOMERY, San Mateo, CA (US); Elsa QUINTANA, Belmont, CA (US); Mallika SINGH, San Francisco, CA (US); Jacqueline SMITH, Redwood City, CA (US); David E. WILDES, San Francisco, CA (US); Yu Chi YANG, San Mateo, CA (US)

(21) Appl. No.: 18/559,932

(22) PCT Filed: May 11, 2022
(86) PCT No.: PCT/US2022/028711

§ 371 Date: Nov. 9, 2023

Related U.S. Application Data
(60) Provisional application No. 63/187,768, filed on May 12, 2021.

(51) Int. Cl. A61K 31/519; A61K 31/5377; A61K 31/5386; A61K 31/55; A61K 31/675; A61P 35/00

(52) U.S. Cl. CPC A61K 31/519; A61K 31/5377; A61K 31/5386; A61K 31/55; A61K 31/675; A61P 35/00

(57) ABSTRACT

The present disclosure relates to combinations of inhibitors of SOS1 and inhibitors of mTOR useful in the treatment of diseases or disorders.

### CROSS REFERENCE TO RELATED APPLICATION

The present disclosure claims the benefit of priority to U.S. Provisional Application Ser. No. 63/187,768, which was filed May 12, 2021, the disclosure of which is hereby incorporated by reference as if set forth in its entirety.

### FIELD OF THE DISCLOSURE

The present disclosure relates to combinations of inhibitors of SOS1 and inhibitors of mTOR useful in the treatment of diseases or disorders.

### BACKGROUND OF THE DISCLOSURE

It has been well established in literature that RAS proteins (KRAS, HRAS and NRAS) play an essential role in various human cancers and are therefore appropriate targets for anticancer therapy. Indeed, mutations in RAS proteins account for approximately 30% of all human cancers in the United States, many of which are fatal. Dysregulation of RAS proteins by activating mutations, overexpression or upstream activation is common in human tumors, and activating mutations in RAS are frequently found in human cancer. For example, activating mutations at codon 12 in RAS proteins function by inhibiting both GTPase-activating protein (GAP)-dependent and intrinsic hydrolysis rates of GTP, significantly skewing the population of RAS mutant proteins to the “on” (GTP-bound) state (RAS(ON)), leading to oncogenic MAPK signaling. Notably, RAS exhibits a picomolar affinity for GTP, enabling RAS to be activated even in the presence of low concentrations of this nucleotide. Mutations at codons 13 (e.g., G13D) and 61 (e.g., Q61K) of RAS are also responsible for oncogenic activity in some cancers.

Despite extensive drug discovery efforts against RAS during the last several decades, a drug directly targeting RAS is still not approved. Additional efforts are needed to uncover additional medicines for cancers driven by the various RAS aberrations and mutations.

SHP2 is a non-receptor protein tyrosine phosphatase encoded by the PTPN11 gene that contributes to multiple cellular functions including proliferation, differentiation, cell cycle maintenance, and migration. SHP2 is involved in signaling through the RAS-mitogen-activated protein kinase (MAPK), the JAK-STAT pathway, and/or the phosphoinositol 3-kinase-AKT pathway.

SHP2 has two N-terminal Src homology 2 domains (N—SH2 and C—SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N—SH2 and PTP domains. Stimulation by, for example, cytokines or growth factors acting through RTKs leads to exposure of the catalytic site resulting in enzymatic activation of SHP2.

Allosteric SHP2 inhibitors show reduced potency against clinically-relevant SHP2 mutants when the mutant SHP2 is in an activated state. See, e.g., Pádua et al., Nat Commun 9:4507 (2018); LaRochelle et al., Nat Commun 9:4508 (2018). Accordingly, a need exists for treating a disease or disorder associated with cells containing a mutant SHP2.

### BRIEF SUMMARY

In some aspects, the present disclosure is directed to a method of treating a subject having a disease or disorder, the method comprising administering to a subject in need of such treatment an SOS1 inhibitor as disclosed herein, or a pharmaceutically acceptable salt, solvate, isomer, stereoisomer, prodrug, or tautomer thereof, and further comprises administering to the subject a therapeutically effective amount of a bi-steric mTOR inhibitor, or a pharmaceutically acceptable salt, solvate, isomer, stereoisomer, prodrug, or tautomer thereof.

### BRIEF DESCRIPTION OF THE DRAWINGS

FIG. 1A is an illustration of the RAS-MAPK pathway. SHP2 activates SOS1, which, in turn, activates RAS, leading to signaling that drives cell growth and survival.

FIG. 1B is an illustration of the PI3K/mTOR pathway. mTOR kinase forms the core of two distinct complexes, mTORC1 and mTORC2, which support the growth and proliferation of normal and tumor cells. In response to nutrients and growth factors, mTORC1 regulates mRNA translation and cell size by multiple mechanisms, including phosphorylation of ribosomal S6 kinase (S6K) and eukaryotic initiation factor 4E-binding proteins (4EBPs), whereas mTORC2 regulates cell metabolism, proliferation, and survival by phosphorylating AGC family kinases such as AKT. Activated mTORC1 activates protein synthesis by phosphorylating 4EBP proteins and relieving their inhibition of cap-dependent translation.

FIG. 2A is a graph depicting activating mutations in SHP2, which reduce sensitivity to inhibition with a SHP2 allosteric inhibitor, RMC-4550. As shown in the graph, cellular sensitivity correlates with energetic magnitude (ΔGₒₚ) of SHP2 activating mutations. Lower ΔGₒₚ indicates stronger activation. FIG. 2B is a table showing the IC₅₀ concentrations of RMC-4550 sufficient to inhibit SHP2 proteins with activating mutations. As shown in the table, strongly activating mutations, such as G503V, require high concentrations of allosteric SHP2 inhibitor to achieve a response.

FIG. 3 is a graph depicting the in vitro combination effect of a SOS1 inhibitor and an mTORC1 inhibitor observed in Pan02 cells. The effect of the mTORC1 inhibitor as a single agent is shown with black circles. The effect of the mTORC1 inhibitor in combination with 100 nM of the SOS1 inhibitor is shown with grey triangles. An increase in apparent potency (left-ward shift of curve) in the combination is an indication of a positive interaction between the compounds. These data were obtained according to the method of Example 1.

FIG. 4 is a graph depicting the in vitro combination effect of a SOS1 inhibitor and an mTORC1 inhibitor observed in SW837 cells. The effect of the mTORC1 inhibitor as a single agent is shown with black circles. The effect of the mTORC1 inhibitor in combination with 1000 nM (1 μM) of the SOS1 inhibitor is shown with grey triangles. An increase in apparent potency (left-ward shift of curve) in the combination is an indication of a positive interaction between the compounds. These data were obtained according to the method of Example 2.

FIG. 5 is a graph depicting the in vitro combination effect of a SOS1 inhibitor and an mTORC1 inhibitor observed in MIA PaCa-2 cells. The effect of the mTORC1 inhibitor as a single agent is shown with black circles. The effect of the mTORC1 inhibitor in combination with 10 μM of the SOS1 inhibitor is shown with grey triangles. An increase in apparent potency (left-ward shift of curve) in the combination is an indication of a positive interaction between the compounds. These data were obtained according to the method of Example 3.

FIG. 6 is a graph depicting the in vitro combination effect of a SOS1 inhibitor and an mTORC1 inhibitor observed in AsPc-1 cells. The effect of the mTORC1 inhibitor as a single agent is shown with black circles. The effect of the mTORC1 inhibitor in combination with 10 μM of the SOS1 inhibitor is shown with grey triangles. An increase in apparent potency (left-ward shift of curve) in the combination is an indication of a positive interaction between the compounds. These data were obtained according to the method of Example 4.

FIG. 7 is a graph depicting the in vitro combination effect of a SOS1 inhibitor and an mTORC1 inhibitor observed in HCT-15 cells. The effect of the mTORC1 inhibitor as a single agent is shown with black circles. The effect of the mTORC1 inhibitor in combination with 3.3 μM of the SOS1 inhibitor is shown with grey triangles. An increase in apparent potency (left-ward shift of curve) in the combination is an indication of a positive interaction between the compounds. These data were obtained according to the method of Example 5.

FIG. 8 is a graph depicting the in vitro combination effect of a SOS1 inhibitor and an mTORC1 inhibitor observed in NCI-H1355 cells. The effect of the mTORC1 inhibitor as a single agent is shown with black circles. The effect of the mTORC1 inhibitor in combination with 316 nM of the SOS1 inhibitor is shown with grey triangles. An increase in apparent potency (left-ward shift of curve) in the combination is an indication of a positive interaction between the compounds. These data were obtained according to the method of Example 6.

FIG. 9A, FIG. 9B and FIG. 9C depict the in vivo combination effect of a SOS1 inhibitor (SOS1-(C)) and an mTORC1 inhibitor (RM-006, also known as RMC-6272) in a murine pancreatic ductal adenocarcinoma PAN02 PTPN11^{G5031} syngeneic model. These data were obtained according to the method of Example 7.

FIG. 10A and FIG. 10B depict the in vivo combinatorial effects of a SOS1 inhibitor (SOS1-(C)) and an mTORC1 inhibitor (RM-006, also known as RMC-6272) in a human model of PTPN11-mutant glioblastoma LN229 PTPN11^{A472S/WT}. These data were obtained according to the method of Example 8.

### DETAILED DESCRIPTION OF THE DISCLOSURE

The details of the present disclosure are set forth in the accompanying description below. Although methods and materials similar or equivalent to those described herein can be used in the practice or testing of the present disclosure, illustrative methods and materials are now described. Other features, objects, and advantages of the present disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which the present disclosure belongs. All patents and publications cited in this specification are incorporated herein by reference in their entireties.

### Terms

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise. The use of the term “or” is used to mean “and/or” unless explicitly indicated to refer to alternatives only or the alternative are mutually exclusive, although the disclosure supports a definition that refers to only alternatives and “and/or.”

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

The term “isomer,” as used herein, means any tautomer, stereoisomer, atropisomer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present disclosure include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present disclosure described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present disclosure may be utilized in any such form, including in any solid form. In some embodiments, compounds described or depicted herein may be provided or utilized in hydrate or solvate form.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

Pharmaceutically acceptable salts of compounds disclosed herein are contemplated by the present invention. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

A “therapeutic agent” is any substance, e.g., a compound or composition, capable of treating a disease or disorder. In some embodiments, therapeutic agents that are useful in connection with the present disclosure include RAS inhibitors and cancer chemotherapeutics. Many such therapeutic agents are known in the art and are disclosed herein.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

A “therapeutic regimen” refers to a dosing regimen whose administration across a relevant population is correlated with a desired or beneficial therapeutic outcome.

The disclosure also includes pharmaceutical compositions comprising an effective amount of a disclosed compound and a pharmaceutically acceptable carrier. The term “carrier”, as used in this disclosure, encompasses excipients and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Excipients include, but are not limited to: butylated optionally substituted hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

The term “treatment” (also “treat” or “treating”), in its broadest sense, refers to any administration of a substance (e.g., a compound of the present disclosure) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

The terms “inhibiting” and “reducing,” or any variation of these terms, includes any measurable or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity (e.g., SOS1:Ras-family protein binding activity) compared to normal.

The term “binding” is used in this disclosure to mean association (e.g., non-covalent or covalent, hydrogen bonds, van der Waals interaction, hydrophobic interactions, magnetism, and combinations thereof, between or among two or more entities. “Direct” binding involves physical contact between entities or moieties; indirect binding involves physical interaction by way of physical contact with one or more intermediate entities. Binding between two or more entities can typically be assessed in any of a variety of contexts, including where interacting entities or moieties are studied in isolation or in the context of more complex systems (e.g., while covalently or otherwise associated with a carrier entity or in a biological system or cell).

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal or vitreal.

As used herein, the term “dosage form” refers to a physically discrete unit of a compound (e.g., a compound of the present disclosure) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present disclosure) has a recommended dosing regimen, which may involve one or more doses. In some embodiments, a dosing regimen includes a plurality of doses each of which are separated from one another by a time period of the same length; in some embodiments, a dosing regimen includes a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen includes a first dose in a first dose amount, followed by one or more additional doses in a second dose amount different from the first dose amount. In some embodiments, a dosing regimen includes a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

The term “disorder” is used in this disclosure to mean, and is used interchangeably with, the terms disease, condition, or illness, unless otherwise indicated.

A “patient” or “subject” is a mammal, e.g., a human, mouse, rat, guinea pig, dog, cat, horse, cow, pig, or non-human primate, such as a monkey, chimpanzee, baboon or rhesus.

The term “sample” or “biological sample,” as used herein, refers to a sample obtained from a subject, e.g., a human subject or a patient, which may be tested for a particular molecule, for example wild type. Samples may include, but are not limited to, biopsies, tissues, cells, buccal swab sample, body fluids, including blood, serum, plasma, urine, saliva, cerebral spinal fluid, tears, pleural fluid and the like.

As used herein, the term “inhibitor” refers to a compound that prevents a biomolecule, (e.g., a protein, nucleic acid) from completing or initiating a reaction. An inhibitor can inhibit a reaction by competitive, uncompetitive, or non-competitive means, for example. With respect to its binding mechanism, an inhibitor may be an irreversible inhibitor or a reversible inhibitor. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein, e.g., that is involved in signal transduction, therapeutic agents, pharmaceutical compositions, drugs, and combinations of these. In some embodiments, the inhibitor is a small molecule, e.g., a low molecular weight organic compound, e.g., an organic compound having a molecular weight (MW) of less than 1200 Daltons (Da). In some embodiments, the MW is less than 1100 Da. In some embodiments, the MW is less than 1000 Da. In some embodiments, the MW is less than 900 Da. In some embodiments, the range of the MW of the small molecule is between 800 Da and 1200 Da. Some inhibitors of the present invention, such as bi-steric mTORC inhibitors, have a greater molecular weight than traditional small molecules. In some embodiments, a bi-steric mTOR inhibitor has a MW of 2100 Da or less. In some embodiments, a bi-steric mTOR inhibitor has a MW of 2000 Da or less. In some embodiments, a bi-steric mTOR inhibitor has a MW of 1900 Da or less. In some embodiments, a bi-steric mTOR inhibitor has a MW of 1800 Da or less. In some embodiments, the range of the MW of a bi-steric mTOR inhibitor is between 1600 and 2100 Da. Inhibitors include cyclic and acyclic compounds. Inhibitors include natural products, derivatives, and analogs thereof. In some embodiments, the inhibitor can be nucleic acid molecules including, but not limited to, siRNA that reduce the amount of functional protein in a cell. Accordingly, compounds said to be “capable of inhibiting” a particular protein, e.g., mTORC1, RAS or SOS1, comprise any such inhibitor.

The term “wild-type” refers to an entity having a structure or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

The term “mutation” as used herein indicates any modification of a nucleic acid and/or polypeptide which results in an altered nucleic acid or polypeptide. The term “mutation” may include, for example, point mutations, deletions of a single or multiple residues in a polynucleotide, or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications and/or chromosomal breaks or translocations.

The term “SHP2” means “Src Homology 2 domain-containing protein tyrosine phosphatase 2” and is also known as SH-PTP2, SH-PTP3, Syp, PTPID, PTP2C, SAP-2 or PTPN11. SHP2 is a non-receptor protein tyrosine phosphatase encoded by the PTPN11 gene that contributes to multiple cellular functions including proliferation, differentiation, cell cycle maintenance and migration. SHP2 is involved in signaling through the RAS-mitogen-activated protein kinase (MAPK), the JAK-STAT and/or the phosphoinositol 3-kinase-AKT pathways. SHP2 has two N-terminal Src homology 2 domains (N—SH2 and C—SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N—SH2 and PTP domains. Stimulation by, for example, cytokines or growth factors acting through RTKs leads to exposure of the catalytic site resulting in enzymatic activation of SHP2. SHP2 can exist in wild-type and mutant forms.

The term “allosteric SHP2 inhibitor” means an agent (e.g., a small-molecule compound (e.g., less than 750 Da)) capable of inhibiting SHP2 through binding to SHP2 at a site other than the active site of the enzyme.

The term “inhibitor-resistant mutation” when used in reference to a SHP2 mutation, means a SHP2 mutation that renders a SHP2 polypeptide refractory or resistant to inhibition with a SHP2 inhibitor. Thus, in some embodiments, an inhibitor-resistant mutation in a SHP2 polypeptide decreases the inhibitory effect that a SHP2 inhibitor has on the SHP2 polypeptide as compared to the effect the inhibitor has on a similar SHP2 polypeptide differing only in the absence of the inhibitor-resistant mutation. Such activity may be measured using any suitable activity assay known in the art or disclosed herein. In some embodiments, an inhibitor-resistant mutation in a SHP2 polypeptide abolishes all detectable inhibitory effects that a SHP2 inhibitor has on the activity of the SHP2 polypeptide, wherein the inhibitor has detectable inhibitory efficacy on a similar SHP2 polypeptide differing only in the absence of the inhibitor-resistant mutation. Such inhibitor-resistant mutations include, without limitation, mutations that destabilize the auto-inhibited conformation of SHP2. An inhibitor-resistant mutation may be an allosteric inhibitor-resistant mutation.

As used herein, the term “activating SHP2 mutation” or “activated mutation of SHP2” or similar refers to a mutation of SHP2 that destabilizes the auto-inhibited conformation of SHP2, as measured by the free energy of opening (ΔGₒₚ) of the mutation. Wild-type SHP2 has a ΔGₒₚ of 2.8 kcal/mol. Values of ΔGₒₚ below 2.8 in mutant SHP2 indicate activation, with lower values indicating stronger activation. A weakly activating SHP2 mutant is defined as one with a ΔGₒₚ not more than 1.5 kcal/mol below wild type SHP2. A moderately activating SHP2 mutant has a ΔGₒₚ between 1.5 kcal/mol and 2.24 kcal/mol below wild-type, and a strongly activating SHP2 mutation has a ΔGₒₚ more than 2.24 kcal/mol below wild type. Methods of measuring ΔGₒₚ are provided in Example 4.

The term “allosteric inhibitor-resistant mutation” when used in reference to a SHP2 mutation, means a SHP2 mutation that renders a SHP2 polypeptide refractory or resistant to inhibition with a SHP2 allosteric inhibitor. Thus, in some embodiments, an allosteric inhibitor-resistant mutation in a SHP2 polypeptide decreases the inhibitory effect that a SHP2 allosteric inhibitor has on the SHP2 polypeptide as compared to the effect the inhibitor has on a similar SHP2 polypeptide differing only in the absence of the allosteric inhibitor-resistant mutation. Such activity may be measured using any suitable activity assay known in the art or disclosed herein. In some embodiments, an allosteric inhibitor-resistant mutation in a SHP2 polypeptide abolishes all detectable inhibitory effects that a SHP2 allosteric inhibitor has on the activity of the SHP2 polypeptide, wherein the inhibitor has detectable inhibitory efficacy on a similar SHP2 polypeptide differing only in the absence of the allosteric inhibitor-resistant mutation. Such allosteric inhibitor-resistant mutations include, without limitation, mutations that destabilize the auto-inhibited conformation of SHP2. In some embodiments, the allosteric inhibitor-resistant mutation is a SHP2 mutation occurring at one or more of the following positions, including any combination thereof: G60, D61, E69, A72, T73, E76, S189, L262, F285, N308, T468, P491, G503, Q506, T507, S502, T253, or Q257. In some embodiments, the allosteric inhibitor-resistant mutation is a SHP2 mutation selected from any one of G60V, D61G, D61V, E69K, A72S, A72T, A72V, T73I, E76A, E76G, E76K, E76Q, S189A, L262R, F285S, N308D, T468M, P491S, G503V, Q506P, T507K, S502P, T253M/Q257L, and any combination thereof.

The term “SOS” (e.g., a “SOS mutation”) refers to SOS genes, which are known in the art to include RAS guanine nucleotide exchange factor proteins that are activated by receptor tyrosine kinases to promote GTP loading of RAS and signaling. The term SOS includes all SOS homologs that promotes the exchange of Ras-bound GDP by GTP. In particular embodiments, SOS refers specifically to “son of sevenless homolog 1” (“SOS1”). SOS1 is critically involved in the activation of RAS-family protein signaling in cancer via mechanisms other than mutations in RAS-family proteins. SOS1 interacts with the adaptor protein Grb2 and the resulting SOS1/Grb2 complex binds to activated/phosphorylated Receptor Tyrosine Kinases (e.g., EGFR, ErbB2, ErbB3, ErbB4, PDGFR-A/B, FGFR 1/2/3, IGF1 R, INSR, ALK, ROS, TrkA, TrkB, TrkC, RET, c-MET, VEGFR1/2/3, AXL) (Pierre et al., Biochem. Pharmacol., 2011, 82 (9): 1049-56). SOS1 is also recruited to other phosphorylated cell surface receptors such as the T cell Receptor (TCR), B cell Receptor (BCR) and monocyte colony-stimulating factor receptor (Salojin et al., J. Biol. Chem. 2000, 275 (8): 5966-75). This localization of SOS1 to the plasma membrane, proximal to RAS-family proteins, enables SOS1 to promote RAS-family protein activation. SOS1-activation of RAS-family proteins can also be mediated by the interaction of SOS1/Grb2 with the BCR-ABL oncoprotein commonly found in chronic myelogenous leukemia (Kardinal et al., 2001, Blood, 98:1773-81; Sini et al., Nat. Cell Biol., 2004, 6 (3): 268-74). SOS1 is also a GEF for the activation of the GTPases RAC1 (Ras-related C3 botulinum toxin substrate 1) (Innocenti et al., J. Cell Biol., 2002, 156 (1): 125-36). RAC1, like RAS-family proteins, is implicated in the pathogenesis of a variety of human cancers and other diseases (Bid et al., Mol. Cancer Ther. 2013, 12 (10): 1925-34). Son of sevenless 2 (SOS2), a homolog of SOS1 in mammalian cells, also acts as a GEF for the activation of RAS-family proteins (Pierre et al., Biochem. Pharmacol., 2011, 82 (9): 1049-56; Buday et al., Biochim. Biophys. Acta., 2008, 1786 (2): 178-87). Published data from mouse knockout models suggests a redundant role for SOS1 and SOS2 in homeostasis in the adult mouse. Whilst germline knockout of SOS1 in mice results in lethality during mid-embryonic gestation (Qian et al., EMBO J., 2000, 19 (4): 642-54), systemic conditional SOS1 knockout adult mice are viable (Baltanas et al., Mol. Cell. Biol., 2013, 33 (22): 4562-78). SOS2 gene targeting did not result in any overt phenotype in mice (Esteban et al., Mol. Cell. Biol., 2000, 20 (17): 6410-3). In contrast, double SOS1 and SOS2 knockout leads to rapid lethality in adult mice (Baltanas et al., Mol. Cell. Biol., 2013, 33 (22): 4562-78). These published data suggest that selective targeting of individual SOS isoforms (e.g., selective SOS1 targeting) may be adequately tolerated to achieve a therapeutic index between SOS1/RAS-family protein driven cancers (or other SOS1/RAS-family protein pathologies) and normal cells and tissues. Selective pharmacological inhibition of the binding of the catalytic site of SOS1 to RAS-family proteins is expected to prevent SOS1-mediated activation of RAS-family proteins to the GTP-bound form. Such SOS1 inhibitor compounds are be expected to consequently inhibit signaling in cells downstream of RAS-family proteins (e.g., ERK phosphorylation). In cancer cells associated with dependence on RAS-family proteins (e.g., KRAS mutant cancer cell lines), SOS1 inhibitor compounds are be expected to deliver anti-cancer efficacy (e.g., inhibition of proliferation, survival, metastasis, etc.). High potency towards inhibition of SOS1:RAS-family protein binding (nanomolar level IC50 values) and ERK phosphorylation in cells (nanomolar level IC50 values) are desirable characteristics for a SOS1 inhibitor compound. Furthermore, a desirable characteristic of a SOS1 inhibitor compound would be the selective inhibition of SOS1 over SOS2. This conclusion is based on the viable phenotype of SOS1 knockout mice and lethality of SOS1/SOS2 double knockout mice, as described above.

As used herein, a “SOS1 inhibitor” refers to any agent, (e.g., a small molecule (e.g., less than 750 Da)) capable of inhibiting SOS1. SOS1 inhibitors can include selective SOS1 inhibitors and inhibitors that also inhibit other proteins. In some embodiments, SOS1 inhibitors may also inhibit SOS2, with a selectivity ratio less than 10-fold for inhibition of SOS1 relative to SOS2. In some embodiments, SOS1 inhibitors will selectively inhibit SOS1, with a selectivity ratio greater of at least about 10-fold, such as greater than at least about 30-fold, for inhibition of SOS1 relative to SOS2.

The terms “RAS pathway” and “RAS/MAPK pathway” are used interchangeably herein to refer to a signal transduction cascade downstream of various cell surface growth factor receptors in which activation of RAS (and its various isoforms and alleotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

The term “mTOR” refers to the protein “mechanistic target of rapamycin (serine/threonine kinase)” or “mammalian target of rapamycin.” The term “mTOR” may include both the wild-type form of the nucleotide sequences or proteins as well as any mutants thereof. In some embodiments, “mTOR” is wild-type mTOR. In some embodiments, “mTOR” is one or more mutant forms. The term “mTOR” XYZ may refer to a nucleotide sequence or protein of a mutant mTOR wherein the Y numbered amino acid of mTOR that normally has an X amino acid in the wildtype, instead has a Z amino acid in the mutant. In embodiments, an mTOR is the human mTOR.

The term “mTORC1” refers to the protein complex including mTOR and Raptor (regulatory-associated protein of mTOR). mTORC1 may also include MLST8 (mammalian lethal with SEC 13 protein 8), PRAS40, and/or DEPTOR. mTORC1 may function as a nutrient/energy/redox sensor and regulator of protein synthesis. The term “mTORC1 pathway” or “mTORC1 signal transduction pathway” may refer to a cellular pathway including mTORC1. An mTORC1 pathway includes the pathway components upstream and downstream from mTORC1. An mTORC1 pathway is a signaling pathway that is modulated by modulation of mTORC1 activity. In embodiments, an mTORC1 pathway is a signaling pathway that is modulated by modulation of mTORC1 activity but not by modulation of mTORC2 activity. In embodiments, an mTORC1 pathway is a signaling pathway that is modulated to a greater extent by modulation of mTORC1 activity than by modulation of mTORC2 activity.

The term “mTORC2” refers to the protein complex including mTOR and RICTOR (rapamycin-insensitive companion of mTOR). mTORC2 may also include GBL, mSIN1 (mammalian stress-activated protein kinase interacting protein 1), Protor 1/2, DEPTOR, TTI1, and/or TEL2. mTORC2 may regulate cellular metabolism and the cytoskeleton. The term “mTORC2 pathway” or “mTORC2 signal transduction pathway” may refer to a cellular pathway including mTORC2. An mTORC2 pathway includes the pathway components upstream and downstream from mTORC2. An mTORC2 pathway is a signaling pathway that is modulated by modulation of mTORC2 activity. In embodiments, an mTORC2 pathway is a signaling pathway that is modulated by modulation of mTORC2 activity but not by modulation of mTORC1 activity. In embodiments, an mTORC2 pathway is a signaling pathway that is modulated to a greater extent by modulation of mTORC2 activity than by modulation of mTORC1 activity.

The term “mTOR inhibitor” is used to refer to inhibitors of the mammalian target of rapamycin (mTOR), which is a serine/threonine-specific protein kinase that belongs to the family of phosphatidylinositol-3 kinase (PI3K) related kinases (PIKKs). mTOR regulates cellular metabolism, growth, and proliferation by forming and signaling through two protein complexes, mTORC1 and mTORC2. The most established mTOR inhibitors are so-called rapalogs (rapamycin and its analogs), which have shown tumor responses in clinical trials against various tumor types.

The terms “bi-steric mTOR inhibitor” and “bi-steric inhibitor of mTOR” are used interchangeably in this disclosure to refer to two pharmacophores in a single compound. One pharmacophore binds to the well-known FRB (FKBP12-rapamycin binding) site on mTORC1 and the other binds to the mTOR kinase active site. As a result of these two binding interactions, such compounds exhibit two biologically useful features: (1) selectivity for mTORC1 over mTORC2, which is characteristic of the natural compound rapamycin, and (2) deep inhibition of mTORC1, which is characteristic of known active site inhibitors. These properties enable selective inhibition of phosphorylation of mTORC1 substrates, including 4EBP1. In some embodiments, a bi-steric mTOR inhibitor has a molecular weight of between 1600 and 2100 Da and exhibits selective (>10-fold) inhibition of mTORC1 over mTORC2.

Reference to a “subtype” of a cell (e.g., a KRAS^{G12C} subtype, a KRAS^{G12S} subtype, a KRAS^{G12D} subtype, a KRAS^{G12V} subtype) means that the cell contains a gene mutation encoding a change in the protein of the type indicated. For example, a cell classified as a “KRAS^{G12C} subtype” contains at least one KRAS allele that encodes an amino acid substitution of cysteine for glycine at position 12 (^{G12C}); and, similarly, other cells of a particular subtype (e.g., KRAS^{G12D}, KRAS^{G12S} and KRAS^{G12V} subtypes) contain at least one allele with the indicated mutation (e.g., a KRAS^{G12D} mutation, a KRAS^{G12S} mutation or a KRAS^{G12V} mutation, respectively). Unless otherwise noted, all amino acid position substitutions referenced herein (such as, e.g., “^{G12C}” in KRAS^{G12C}) correspond to substitutions in the human version of the referenced protein, i.e., KRAS^{G12C} refers to a G→C substitution in position 12 of human KRAS.

The term “monotherapy” refers to a method of treatment comprising administering to a subject a single therapeutic agent, optionally as a pharmaceutical composition. For example, a monotherapy may comprise administration of a pharmaceutical composition comprising a therapeutic agent and one or more pharmaceutically acceptable carrier, excipient, diluent, and/or surfactant. The therapeutic agent may be administered in an effective amount. The therapeutic agent may be administered in a therapeutically effective amount.

The term “combination therapy” refers to a method of treatment comprising administering to a subject at least two therapeutic agents, optionally as one or more pharmaceutical compositions. For example, a combination therapy may comprise administration of a single pharmaceutical composition comprising at least two therapeutic agents and one or more pharmaceutically acceptable carrier, excipient, diluent, and/or surfactant. A combination therapy may comprise administration of two or more pharmaceutical compositions, each composition comprising one or more therapeutic agent and one or more pharmaceutically acceptable carrier, excipient, diluent, and/or surfactant. In various embodiments, at least one of the therapeutic agents is a SOS1 inhibitor. In various embodiments, at least one of the therapeutic agents is a RAS inhibitor. The two agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due an additive or synergistic effect of combining the two or more therapeutics.

### Methods of Treatment

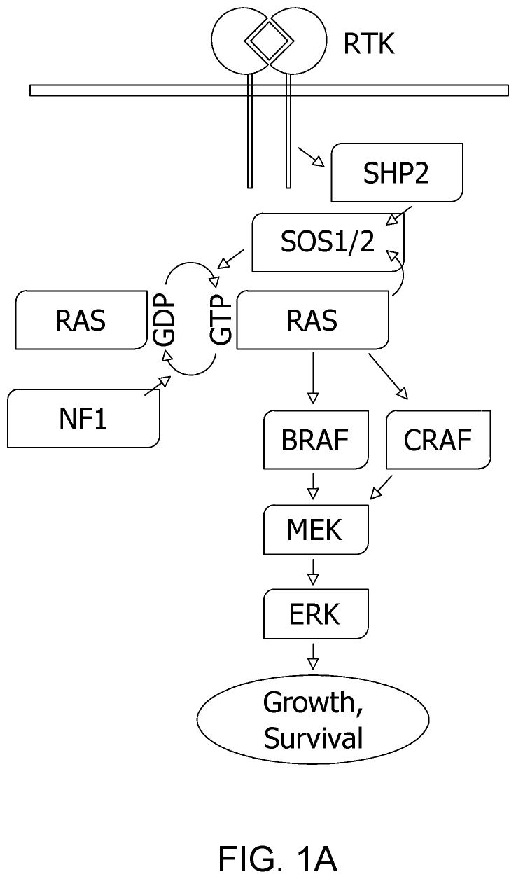

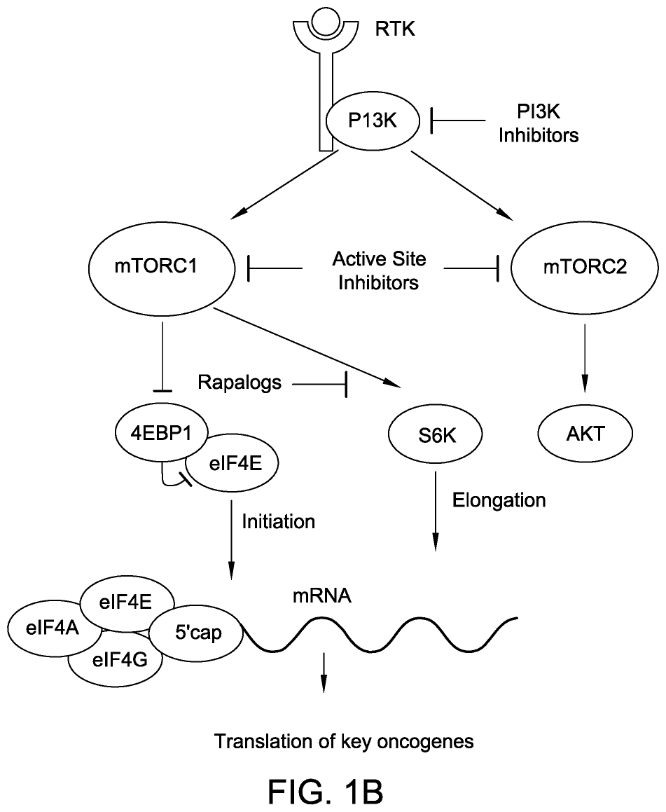

Disruption of the RAS/MAPK signaling pathway (see FIG. 1A) is a common driver of abnormal growth and proliferation in many types of cancer and has also been implicated in developmental diseases such as Noonan Syndrome. Oncogenic hyperactivation of this pathway can occur through alterations in the levels of active GTP-bound RAS and inactive GDP-bound RAS, such as mutations resulting in disruption of RAS guanine nucleotide exchange factors (GEFs) and GTPase-activating proteins (GAPs). SHP2 is a non-receptor protein tyrosine phosphatase encoded by the PTPN11 gene that functions upstream of RAS. SHP2, e.g., wild-type SHP2, can regulate RAS signaling through activation of SOS1, a GEF that converts inactive RAS-GDP to RAS-GTP. The development of inhibitors targeting either SHP2, SOS1, or RAS is an emerging and attractive approach toward treatment of RAS-driven cancers, and several such candidates are currently undergoing clinical trials.

The mammalian target of rapamycin (mTOR) is a serine-threonine kinase related to the lipid kinases of the phosphoinositide 3-kinase (PI3K) family. mTOR exists in two complexes, mTORC1 and mTORC2, which are differentially regulated, have distinct substrate specificities, and are differentially sensitive to rapamycin. mTORC1 integrates signals from growth factor receptors with cellular nutritional status and controls the level of cap-dependent mRNA translation by modulating the activity of key translational components such as the cap-binding protein and oncogene eIF4E. See FIG. 1B. Hyperactivation of the PI3K/mTOR pathway occurs frequently in human cancer, via mutation or deletion of different components.

Various inhibitors of mTOR exist and have differential specificity for the two mTOR complexes. The most established mTOR inhibitors are so-called rapalogs (rapamycin and its analogs), which have shown tumor responses in clinical trials against various tumor types. However, despite clear biological rationale, PI3K/mTOR pathway inhibitors have been largely unsuccessful in “all-comers” clinical trials, attributed to the lack of biomarker-guided patient stratification. A class of selective mTORC1 inhibitors, termed ‘bi-steric’, which comprise a rapamycin-like core moiety covalently linked to an mTOR active-site inhibitor, has recently been developed. Bi-steric mTORC1 inhibitors exhibit potent and selective (>10-fold) inhibition of mTORC1 over mTORC2, durably suppress S6K and 4EBP1 phosphorylation, and induce growth suppression and apoptosis in multiple cancer cell lines. These inhibitors provide the mTORC1 selectivity of rapalogs and potently inhibit translation initiation by the 4EBP1-eIF4E axis while sparing mTORC2. In various embodiments, any one or more of these bi-steric mTOR inhibitors may be utilized in any of methods disclosed herein.

The present invention is directed to a method of treating a subject having a disease or disorder, the method comprising administering to a subject in need of such treatment an SOS1 inhibitor as disclosed herein, or a pharmaceutically acceptable salt, solvate, isomer, stereoisomer, prodrug, or tautomer thereof, and further comprises administering to the subject a therapeutically effective amount of an mTOR inhibitor, or a pharmaceutically acceptable salt, solvate, isomer, stereoisomer, prodrug, or tautomer thereof. In some embodiments, the SOS1 inhibitor targets a disease or disorder mediated by a mutant SHP2 protein. In some embodiments, the disease or disorder is a RAS protein-related disease or disorder. In some embodiments, the RAS protein-related disease or disorder is associated with a RAS protein mutation.

According to embodiments of the present invention, the method comprises administering to the subject a therapeutically effective amount of SOS1 inhibitor as disclosed herein, or a pharmaceutically acceptable salt, solvate, isomer, stereoisomer, prodrug, or tautomer thereof, and further comprises administering to the subject a therapeutically effective amount of an mTOR inhibitor, or a pharmaceutically acceptable salt, solvate, isomer, stereoisomer, prodrug, or tautomer thereof, and a combination thereof in the treatment of a tumor or cancer. In some embodiments, the disease or disorder is selected from the group consisting of tumors of hematopoietic and lymphoid system; a myeloproliferative syndrome; a myelodysplastic syndromes; leukemia; acute myeloid leukemia; juvenile myelomonocytic leukemia; esophageal cancer; breast cancer; lung cancer; colon cancer; gastric cancer; neuroblastoma; bladder cancer; prostate cancer; glioblastoma; urothelial carcinoma; uterine carcinoma; adenoid and ovarian serous cystadenocarcinoma; paraganglioma; pheochromocytoma; pancreatic cancer; adrenocortical carcinoma; stomach adenocarcinoma; sarcoma; rhabdomyosarcoma; lymphoma; head and neck cancer; skin cancer; peritoneum cancer; intestinal cancer (e.g., small and/or large intestinal cancer); thyroid cancer; endometrial cancer; cancer of the biliary tract; soft tissue cancer; ovarian cancer; central nervous system cancer (e.g., primary CNS lymphoma); stomach cancer; pituitary cancer; genital tract cancer; urinary tract cancer; salivary gland cancer; cervical cancer; liver cancer; eye cancer; cancer of the adrenal gland; cancer of autonomic ganglia; cancer of the upper aerodigestive tract; bone cancer; testicular cancer; pleura cancer; kidney cancer; penis cancer; parathyroid cancer; cancer of the meninges; vulvar cancer; and melanoma. In some embodiments, the disease or disorder is selected from brain glioblastoma (GBM), lung adenocarcinoma, colon adenocarcinoma (CRC), bone marrow leukemia, acute myelocytic leukemia (AML), breast carcinoma (NOS), unknown primary melanoma, non-small cell lung carcinoma (NOS), skin melanoma, breast invasive ductal carcinoma (IDC), lung squamous cell carcinoma (SCC), unknown primary adenocarcinoma, bone marrow multiple myeloma, gastroesophageal junction adenocarcinoma, bone marrow myelodysplastic syndrome (MDS), prostate acinar adenocarcinoma, bladder urothelial (transitional cell) carcinoma, uterus endometrial adenocarcinoma (NOS), bone marrow leukemia B cell acute (B-ALL), stomach adenocarcinoma (NOS), and unknown primary carcinoma (NOS). In some embodiments, the disease or disorder is selected from brain glioblastoma (GBM), lung adenocarcinoma, colon adenocarcinoma (CRC), bone marrow leukemia non-lymphocytic acute myelocytic (AML) and breast carcinoma (NOS). In some embodiments, the disease or disorder is selected from pancreatic cancer, colorectal cancer, non-small cell lung carcinoma and glioblastoma. In some embodiments, the disease or disorder is selected from endometrial cancer, uterine cancer, breast cancer, small-cell lung cancer, non-small cell lung carcinoma (e.g., harboring a KRAS mutation (e.g., KRASG12C), a STK11 mutation or a KEAP1 mutation, or a combination thereof), squamous cell lung carcinoma, cervical cancer, glioblastoma, colorectal cancer, melanoma, head and neck cancer (e.g., squamous cell cancer of the head and neck (HNSCC)), ovarian cancer, prostate cancer, FGFR mutant cancer, hepatocellular carcinoma, hepatoblastoma, glioma, rhabdomyosarcoma, bladder cancer, hematological cancer, pancreatic cancer, renal cancer, neuro-endocrine cancer, thyroid gland adenocarcinoma, a cancer characterized by increased mTORC1 activity, a cancer with an mTORC1 pathway mutation (e.g., STK11, PIK3CA, PTEN, mTOR, KEAP1, TSC1, or TSC2), a cancer having a MYC amplification (e.g., MYC, MYCL, or MYCN), a cancer characterized by decreased 4EBP1 activity, and a cancer with a PTEN mutation.

### SHP2 and SHP2 Mutations

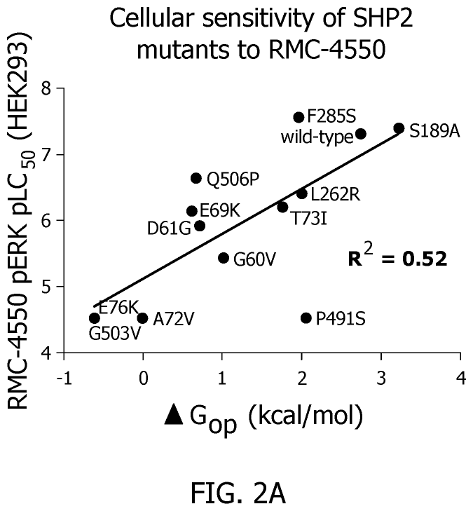

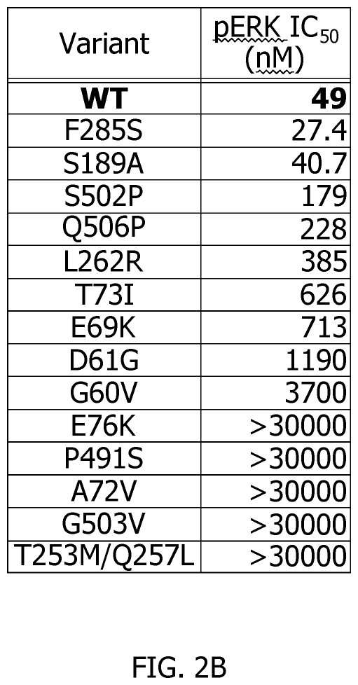

Potent and selective allosteric SHP2 inhibitors, such as RMC-4550, have proven effective in vitro and in vivo across a wide range of histotypes and genotypes, at disrupting RAS-MAPK signaling, suppressing cell proliferation, and inducing tumor growth inhibition. Allosteric SHP2 inhibitors are effective in preclinical models of cancers driven by the RAS/MAPK signaling pathway, in part because they block activation of the RAS GEFs SOS1 and SOS2. See FIG. 1A, which illustrates the RAS-MAPK pathway. However, SHP2 inhibitors, including allosteric SHP2 inhibitors, have previously been demonstrated to exhibit significantly reduced efficacy against a spectrum of specific clinically relevant mutations in SHP2 that induce an activated form of the SHP2 protein. Mutations can occur in SHP2 that, to varying degrees, activate signaling and reduce sensitivity to allosteric inhibitors. These mutations may arise during tumor development as drivers or be acquired as resistance mutations in response to treatment with allosteric SHP2 inhibitors. See FIGS. 2A and 2B, which depict several activating mutations in SHP2 proteins. In addition, patients treated with SHP2 inhibitors, including allosteric SHP2 inhibitors, may develop tumors with somatic SHP2 mutations, inducing pathway reactivation and drug resistance. Therefore, there exists an unmet medical need for alternative treatments for cancers associated with activating mutations of SHP2 protein.

Potent SOS1 inhibitors, including selective SOS1 inhibitors, have also been developed and have more recently shown preclinical promise as an alternative treatment for RAS/MAPK pathway-driven cancers. The first clinical trial involving a SOS1 inhibitor has recently begun. SOS1 inhibitors, which act directly downstream of SHP2, would represent an attractive alternative treatment in this regard if they maintain or show improved efficacy in the context of activated mutant SHP2. The present disclosure provides evidence in support of this concept, showing that in contrast to SHP2 inhibitors, e.g., allosteric SHP2 inhibitors, SOS1 inhibition is efficacious in vitro and in vivo in cells or tissue with activating SHP2 mutations, even when SHP2 allosteric inhibitors are not effective.

Unexpectedly, genetic knockdown of SOS1 appears to induce a greater reduction in pathway activity in cells with activated SHP2, indicating that cancer cells harboring activating SHP2 mutations are uniquely dependent on SOS1. Experimental results showed that all cells are sensitive to simultaneous knockdown of SOS1 and SOS2, but unexpectedly, a trend is seen of greater effect of SOS1 knockdown in cells with more strongly activating SHP2 mutations. In contrast, SOS2 knockdown has only a small and similar effect in all cell lines. This suggests that strongly activating SHP2 mutations increase dependence on SOS1, and activating SHP2 mutations may therefore increase sensitivity to SOS1 inhibitors such as BI-3406, BI-1701963, and Compound SOS1-(A). This observation provides the basis for a therapeutic strategy whereby cancer patients with mutations in SHP2, e.g., activating SHP2 mutations, either present initially or arising in response to therapy with SHP2 inhibitors, e.g., allosteric SHP2 inhibitors, may be effectively treated with SOS1 inhibitors.

According to some embodiments of the present invention, a method is provided for treating a subject having a SHP2 mutation, e.g., a SHP2 mutation that induces an activated form of SHP2. These mutations act by destabilizing an auto-inhibited conformation of SHP2. Different activating mutations destabilize this conformation to different degrees, which can be expressed quantitatively as the free energy of opening (ΔGₒₚ) of the mutation. Wild-type SHP2 has a ΔGₒₚ of 2.8 kcal/mol. Values of ΔGₒₚ below 2.8 in mutant SHP2 indicate activation, with lower values indicating stronger activation. A weakly activating mutant is defined as one with a ΔGₒₚ not more than 1.5 kcal/mol below wild type SHP2. A moderately activating mutant has a ΔGₒₚ between 1.5 kcal/mol and 2.24 kcal/mol below wild-type. A strongly activating mutation has a ΔGₒₚ more than 2.24 kcal/mol below wild type. See FIG. 2A, which is a graph correlating the RMC-4550 PERK IC₅₀ as a function of ΔGₒₚ. FIG. 2B is a table showing the pERK IC₅₀ values for RMC-4550 in a variety of activating mutations of SHP2 protein. Clinically relevant mutations may include, without limitation, a SHP2 mutation occurring at one or more of the following positions, including any combination thereof: G60, D61, E69, A72, T73, E76, S189, L262, F285, N308, T468, P491, G503, Q506, T507, S502, T253 or Q257. In some embodiments, a mutation is one or more of the following: G60V, D61G, D61V, E69K, A72S, A72T, A72V, T73I, E76A, E76G, E76K, E76Q, S189A, L262R, F285S, N308D, T468M, P491S, G503V, Q506P, T507K, S502P, T253M/Q257L, and any combination thereof. The following Table 1 summarizes the model parameters for SHP2 and mutants.

TABLE 1
Summary of model parameters for SHP2 and mutants

| | ΔGₒₚ | ΔG_{peptide} | ΔGᵢ | Aₒₚ |
|---|---|---|---|---|
| Variant | (kcal/mol) | (kcal/mol) | (kcal/mol) | (mRFU/s/nM) |
| wild-type | 2.74 | −10.78 | −11.87 | 35 |
| G60V | 1.01 | −11.48 | −10.38 | 31.77 |
| D61G | 0.72 | −10.55 | −11.94 | 34.51 |
| D61V | 0.37 | −11.39 | −11.23 | 33.39 |
| E69K | 0.61 | −11.54 | −11.59 | 30.25 |
| A72S | 2.03 | −11.07 | −11.56 | 28.24 |
| A72T | 0.31 | −10.72 | −11.18 | 33.21 |
| A72V | −0.01 | −11.05 | −10.62 | 31.84 |
| T73I | 1.76 | −11.69 | −11.56 | 38.4 |
| E76A | 0.41 | −11.26 | −11.51 | 32.1 |
| E76G | 0.53 | −11.14 | −11.45 | 35.64 |
| E76K | −0.60 | −10.91 | −10.92 | 29.51 |
| E76Q | 1.24 | −11.33 | −11.91 | 30.83 |
| S189A | 3.22 | −10.52 | −11.95 | 35 |
| L262R | 2.00 | −10.94 | −11.85 | 37.72 |
| F285S | 1.97 | −10.77 | −11.94 | 19.81 |
| N308D | 1.89 | −11.12 | −11.96 | 34.42 |
| T468M | 3.63 | −10.52 | −11.11 | 5 |
| P491S | 2.06 | −11.13 | −9.13 | 35.67 |
| G503V | −0.62 | −10.57 | −9.31 | 53.97 |
| Q506P | 0.67 | −10.38 | −11.19 | 2.679 |
| T507K | 1.97 | −10.53 | −11.23 | 30.3 |

As shown in FIG. 2A and Table 1, G503V, a mutation resulting in the lowest free energy of opening (ΔGₒₚ), is a particularly activating mutation of SHP2. This is further shown, for example, by the pERK IC₅₀ value of >30,000 nM for G503V in FIG. 2B.

In some embodiments, the SHP2 mutation is expressed in a subject after a course of treatment with a SHIP2 inhibitor. In some embodiments, the SHIP2 mutation is expressed in a subject after a course of treatment with an allosteric SHP2 inhibitor. In some embodiments, the SHP2 mutation is expressed in a subject after a course of treatment with an active site SHP2 inhibitor. In some embodiments, the SHIP2 mutation is expressed in a subject after a course of treatment with an allosteric SHP2 inhibitor. The SHP2 inhibitor, e.g., allosteric SHP2 inhibitor, is generally an inhibitor of wild-type SHP2 protein. SHP2 inhibitors, e.g., allosteric SHP2 inhibitors, may be selected from among those disclosed, without limitation, in WO 2022063190, WO 2022043685, WO 2022042331, WO 2022033430, WO 2022033430, WO 2022017444, WO 2022007869, WO 2021259077, WO 2021249449, WO 2021249057, WO 2021244659, WO 2021218755, WO 2021281752, WO 2021197542, WO 2021176072, WO 2021149817, WO 2021148010, WO 2021147879, WO 2021143823, WO 2021143701, WO 2021143680, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, US20210085677, US 10858359, US 10934302, US 10954243, US 10988466, US 11001561, US 11033547, US 11034705, US 11044675, CN 114213417, CN 114163457, CN 113896710, CN 113248521, CN 113248449, CN 113135924, CN 113024508, CN 112920131, CN 112823796, CN 112402385, CN 111848599, CN 111704611, CN 111265529, and CN 108113848, or a pharmaceutically acceptable salt, solvate, isomer (e.g., stereoisomer), prodrug, or tautomer thereof, each of which is incorporated herein by reference. A non-limiting list of exemplary such allosteric SHP2 inhibitors include ERAS-601, BBP-398, RLY-1971, JAB-3068, JAB-3312, TNO155, SHP099, SH3809, PF-07284892, RMC-4550, and RMC-4630. A non-limiting list of exemplary active-site SHP2 inhibitors include NSC-87877, IIB-08, 11a-1, and GS-493. In some embodiments, a course of treatment with these SHP2 inhibitors, including allosteric SHP2 inhibitors, active site SHP2 inhibitors, or other such inhibitors of wild type SHP2, may induce a mutation, e.g., an activating mutation, in SHP2. In some embodiments, the SHP2 mutation confers resistance to the SHP2 inhibitor. Alternatively, the resistance to the SHP2 inhibitor may occur due to a natural mutation in the SHP2 protein. In any event, the mutation may induce pathway reactivation and drug resistance to the SHP2 inhibitor.

Accordingly, the present invention is directed to a method of treating a subject having a disease or disorder associated with cells having a mutation in SHP2 protein. In some embodiments, the mutation is an activating SHP2 mutation. According to embodiments of the present invention, the method comprises administering to the subject a therapeutically effective amount of a SOS1 inhibitor. In some embodiments, SOS1 inhibitors may also inhibit SOS2, i.e., the method comprises administering to the subject a therapeutically effective amount of a dual SOS1/SOS2 inhibitor. In some embodiments, such a SOS1 inhibitor is characterized by a selectivity ratio less than 10-fold for inhibition of SOS1 relative to SOS2. In some embodiments, the method comprises administering to the subject a therapeutically effective amount of a selective SOS1 inhibitor. In some embodiments, such a SOS1 inhibitor is characterized by a selectivity ratio greater of at least about 10-fold, such as greater than at least about 30-fold, for inhibition of SOS1 relative to SOS2.

### RAS and RAS Mutations

In some embodiments, the method of the present invention targets a RAS-protein-related disease or disorder. In some embodiments, the method of the present invention targets a RAS-protein-related disease or disorder characterized by a RAS protein having a mutation. In some embodiments, the disease is characterized by a KRAS mutant, a NRAS mutant, or an HRAS mutant. In certain embodiments, RAS mutant is selected from:
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, Y96D, or G13V, and combinations thereof;
(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, or A59T, and combinations thereof;
or a combination of any of the foregoing (e.g., both K-Ras G12C and K-Ras G13C). In some embodiments, the cancer comprises a Ras mutation selected from the group consisting of G12C, G13C, G12A, G12D, G13D, G12S, G13S, G12V and G13V. In some embodiments, the cancer comprises at least two Ras mutations selected from the group consisting of G12C, G13C, G12A, G12D, G13D, G12S, G13S, G12V and G13V. Mutations at these positions may result in RAS-driven tumors.

Methods of detecting Ras mutations are known in the art. Such means include, but are not limited to direct sequencing, and utilization of a high-sensitivity diagnostic assay (with CE-IVD mark), e.g., as described in Domagala, et al., Pol J Pathol 3:145-164 (2012), incorporated herein by reference in its entirety, including TheraScreen PCR; AmoyDx; PNAClamp; RealQuality; EntroGen; LightMix; StripAssay; Hybcell plexA; Devyser; Surveyor; Cobas; and TheraScreen Pyro. See, also, e.g., WO 2020/106640.

Also provided is a method of inhibiting a Ras protein in a cell, the method comprising contacting the cell with an effective amount of a compound of the present invention, or a pharmaceutically acceptable salt thereof. A method of inhibiting RAF-Ras binding, the method comprising contacting the cell with an effective amount of a compound of the present invention, or a pharmaceutically acceptable salt thereof, is also provided. The cell may be a cancer cell. The cancer cell may be of any type of cancer described herein. The cell may be in vivo or in vitro.

mTOR

The mammalian target of rapamycin (mTOR) is a serine-threonine kinase related to the lipid kinases of the phosphoinositide 3-kinase (PI3K) family. mTOR exists in two complexes, mTORC1 and mTORC2, which are differentially regulated, have distinct substrate specificities, and are differentially sensitive to rapamycin. mTORC1 integrates signals from growth factor receptors with cellular nutritional status and controls the level of cap-dependent mRNA translation by modulating the activity of key translational components such as the cap-binding protein and oncogene eIF4E. See FIG. 1B.

mTOR signaling has been deciphered in increasing detail. The differing pharmacology of inhibitors of mTOR has been particularly informative. The first reported inhibitor of mTOR, Rapamycin is now understood to be an incomplete inhibitor of mTORC1. Rapamycin is a selective mTORC1 inhibitor through the binding to the FK506 Rapamycin Binding (FRB) domain of mTOR kinase with the aid of FK506 binding protein 12 (FKBP12). The FRB domain of mTOR is accessible in the mTORC1 complex, but less so in the mTORC2 complex. Interestingly, the potency of inhibitory activities against downstream substrates of mTORC1 by the treatment of Rapamycin is known to be diverse among the mTORC1 substrates. For example, Rapamycin strongly inhibits phosphorylation of the mTORC1 substrate S6K and, indirectly, phosphorylation of the downstream ribosomal protein S6 which control ribosomal biogenesis. On the other hand, Rapamycin shows only partial inhibitory activity against phosphorylation of 4E-BP1, a major regulator of eIF4E which controls the initiation of cap-dependent translation. As a result, more complete inhibitors of mTORC1 signaling are of interest.

In some embodiments, the method of the present invention comprises administering to the subject a therapeutically effective amount of a bi-steric mTOR inhibitor, or a pharmaceutically acceptable salt, solvate, isomer, stereoisomer, prodrug, or tautomer thereof.

### SOS1 Inhibitors

In some embodiments, the SOS1 inhibitor is selected from those disclosed in WO 2018/115380, WO 2018/172250, WO 2019/122129, WO 2019/201848, and WO 2021/074227, or a pharmaceutically acceptable salt, solvate, isomer (e.g., stereoisomer), prodrug, or tautomer thereof, the disclosures of each of which are hereby incorporated by reference as if set forth in their entirety. In some embodiments, the SOS1 inhibitor is selected from those disclosed in priority document U.S. Provisional Application Ser. No. 63/187,768, or a pharmaceutically acceptable salt, solvate, isomer (e.g., stereoisomer), prodrug, or tautomer thereof, the disclosure of which is hereby incorporated by reference as if set forth in its entirety. In some embodiments, the SOS1 inhibitor is selected from those disclosed in International Application No. PCT/US2021/063685, or a pharmaceutically acceptable salt, solvate, isomer (e.g., stereoisomer), prodrug, or tautomer thereof, the disclosure of which is hereby incorporated by reference as if set forth in its entirety. In some embodiments, the SOS1 inhibitor is selected from those disclosed in WO 2021/092115, or a pharmaceutically acceptable salt, solvate, isomer (e.g., stereoisomer), prodrug, or tautomer thereof, the disclosure of which is hereby incorporated by reference as if set forth in its entirety. In some embodiments, the SOS1 inhibitor is selected from those disclosed in WO 2020/180768, or a pharmaceutically acceptable salt, solvate, isomer (e.g., stereoisomer), prodrug, or tautomer thereof, the disclosure of which is hereby incorporated by reference as if set forth in its entirety. In some embodiments, the SOS1 inhibitor is selected from those disclosed in WO 2020/180770, or a pharmaceutically acceptable salt, solvate, isomer (e.g., stereoisomer), prodrug, or tautomer thereof, the disclosure of which is hereby incorporated by reference as if set forth in its entirety.

In some embodiments, the SOS1 inhibitor is selected from those disclosed in WO 2022061348, WO 2022060583, WO 2022058344, WO 2022028506, WO 2022026465, WO 2022017339, WO 2022017519, WO 2021259972, WO 2021249519, WO 2021249475, WO 2021228028, WO 2021225982, WO 2021203768, WO 2021173524, WO 2021130731, WO 2021127429, WO 2021105960, WO 2021074227, WO 2020173935, WO 2020146470, WO 2019201848, WO 2019/122129, WO 2018172250, WO 2018115380, CN 113912608, CN 113801114, CN 113200981, US 20210338694, and US 8,232,283, or a pharmaceutically acceptable salt, solvate, isomer (e.g., stereoisomer), prodrug, or tautomer thereof, each of which is incorporated herein by reference as if set forth in its entirety.

In some embodiments, the SOS1 inhibitor is a compound as disclosed in WO 2020/180768, which is incorporated by reference in its entirety, or a pharmaceutically acceptable salt, solvate, isomer, stereoisomer, prodrug, or tautomer thereof. In some embodiments, the SOS1 inhibitor is a compound described by any of Formula (I), Formula (I-a), Formula (II), Formula (II-a), Formula (III), and Formula (III-a) in WO 2020/180768, or a pharmaceutically acceptable salt, solvate, isomer, stereoisomer, prodrug, or tautomer thereof. In some embodiments, the SOS1 inhibitor is selected from a compound of Table A of WO 2020/180768, or a pharmaceutically acceptable salt, solvate, isomer, stereoisomer, prodrug, or tautomer thereof. In some embodiments, the SOS1 inhibitor is selected from a compound of Collection 1, Collection 2, Collection 3, or Collection 4 of WO 2020/180768, or a pharmaceutically acceptable salt, solvate, isomer, stereoisomer, prodrug, or tautomer thereof.

In some embodiments, the SOS1 inhibitor is a compound having the structure of Formula (41-I),

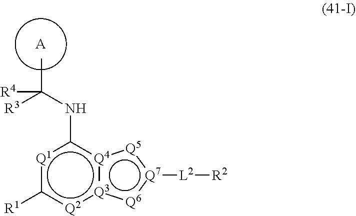

or a pharmaceutically acceptable salt, solvate, isomer, stereoisomer, prodrug, or tautomer thereof, wherein:
Q¹ and Q² are independently CH or N;
Q³, Q⁴, and Q⁷ are independently C or N, wherein at least one of Q³ and Q⁴ is C and wherein Q³, Q⁴, and Q⁷ are not all N;
Q⁵ is CH, N, NH, O, or S;
Q⁶ is CH, N, NH, N—C₁₋₆ alkyl, N—C₁₋₆ heteroalkyl, N-(3-7 membered cycloalkyl), N-(3-7 membered heterocyclyl), O, or S;
wherein at least one of Q¹, Q², Q³, Q⁴, Q⁵, Q⁶, and Q⁷ is N, NH, O, or S;
R¹ is selected from the group consisting of H, C₁₋₆ alkyl, halogen, —NHR^{1a}, —OR^{1a}, cyclopropyl, and —CN; wherein C₁₋₆ alkyl is optionally substituted with halogen, —NHR^{1a}, or —OR^{1a}, wherein R^{1a} is H, C₁₋₆ alkyl, 3-6 membered heterocyclyl, or C₁₋₆ haloalkyl;
L² is selected from the group consisting of a bond, —C(O)—, —C(O)O—, —C(O)NH(CH₂)ₒ—, —S(O)₂—,

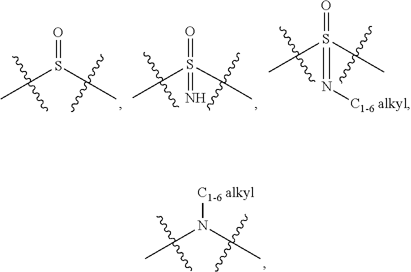

—C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, C₂₋₆ alkenyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, C₂₋₆ alkenyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, —OH, —OR^{2a}, oxo, halogen, —C(O)R^{2a}, —C(O)OR^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl;
   wherein R^{2a} is H, C₁₋₆ alkyl, C₁₋₆ haloalkyl, 3-7 membered heterocyclyl, or —(CH₂)ᵣOCH₃, wherein r is 1, 2, or 3;
   wherein R^{2b} is H or C₁₋₆ alkyl;
   wherein R^{2c} is H or C₁₋₆ alkyl;
R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and

A is an optionally substituted 6-membered aryl or an optionally substituted 5-6 membered heteroaryl.

In some embodiments, the SOS1 inhibitor is a compound as disclosed in WO 2020/180770, which is incorporated by reference in its entirety, or a pharmaceutically acceptable salt, solvate, isomer, stereoisomer, prodrug, or tautomer thereof. In some embodiments, the SOS1 inhibitor is a compound described by any of Formula (I), Formula (I-a), Formula (II), Formula (III), Formula (IV), Formula (V), Formula (V-a), Formula (VI), (VI-a), Formula (VII-a), Formula (VII-b), Formula (VII-c), and Formula (VII-d), in WO 2020/180770, or a pharmaceutically acceptable salt, solvate, isomer, stereoisomer, prodrug, or tautomer thereof. In some embodiments, the SOS1 inhibitor is selected from a compound of Table A of WO 2020/180770, or a pharmaceutically acceptable salt, solvate, isomer, stereoisomer, prodrug, or tautomer thereof. In some embodiments, the SOS1 inhibitor is selected from a compound of Collection 1, Collection 2, or Collection 3 of WO 2020/180770, or a pharmaceutically acceptable salt, solvate, isomer, stereoisomer, prodrug, or tautomer thereof.

In some embodiments, the SOS1 inhibitor is a compound having the structure of Formula (42-I),

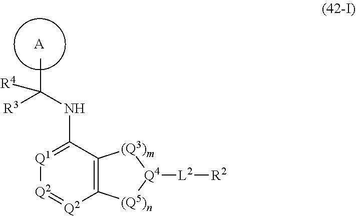

or a pharmaceutically acceptable salt, solvate, isomer, stereoisomer, prodrug, or tautomer thereof, wherein:
Q¹ is CH or N;
Q⁴ is CH, C, or N;
each Q² is independently C—R¹ or N, wherein one Q² is N and the other Q² is C—R¹;
each Q³ and Q⁵ are independently C(R^{QC})₂, NR^{QN}, CO, O, S, or SO₂, wherein each R^{QC} is independently H, F, Cl, Br, or 6-10 membered aryl, and wherein each R^{QN} is independently H, C₁₋₆ alkyl, or 6-10 membered aryl;
wherein at least one of Q¹, Q², Q³, Q⁴, and Q⁵ is N, NR^{QN}, O, or SO₂;
m is 0, 1, 2, or 3;
n is 0, 1, 2, or 3;
wherein when m is 0, then n is not 0;
R¹ is selected from the group consisting of H, C₁₋₆ alkyl, halogen, —CONHR^{1a}, —NHR^{1a}, —OR^{1a}, cyclopropyl, azetidinyl, and —CN; wherein each C₁₋₆ alkyl and azetidinyl is optionally substituted with halogen, R^{1a}, —NHR^{1a}, or —OR^{1a}, wherein R^{1a} is H, C₁₋₆ alkyl, cyclopropyl, 3-6 membered heterocyclyl, or C₁₋₆ haloalkyl;
L² is selected from the group consisting of a bond, —C(O)—, —C(O)O—, —C(O)NH(CH₂)ₒ—, —S(O)₂—,

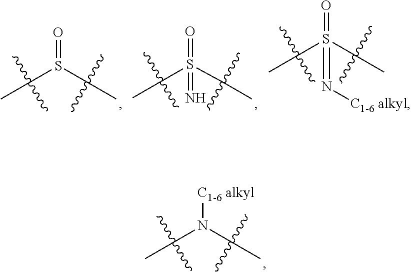

—C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, C₁₋₆ hydroxyalkyl, C₁₋₆ methoxyalkyl, —OH, —OR^{2a}, oxo, ═N, halogen, C(O)R^{2a}, —C(O)OR^{2a}, —C(O)NR^{2b}R^{2c}, —SO₂R^{2a}, —CN, —NR^{2b}R^{2c}, 3-6 membered cycloalkyl, 3-7 membered heterocyclyl, 6-10 membered aryl, or 5-10 membered heteroaryl;
   wherein R^{2a} is H, C₁₋₆ alkyl, C₁₋₆ haloalkyl, 3-7 membered heterocyclyl, or —(CH₂)ᵣOCH₃, wherein r is 1, 2, or 3;
   wherein R^{2b} is H or C₁₋₆ alkyl;
   wherein R^{2c} is H or C₁₋₆ alkyl;
R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and
A is an optionally substituted 6-membered aryl or an optionally substituted 5-6 membered heteroaryl;
with the proviso that when

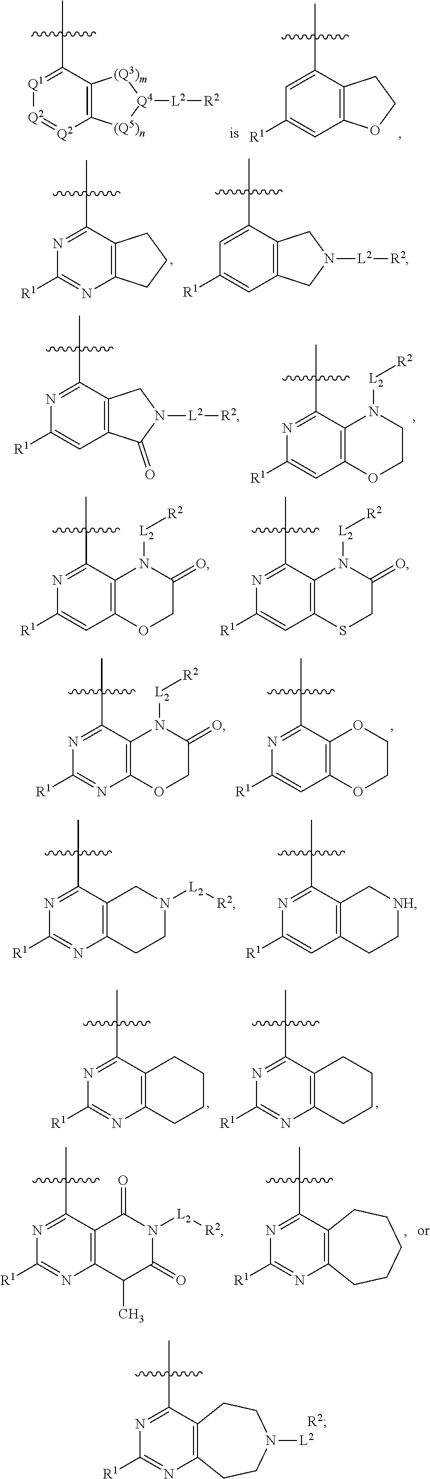

then R¹ is not H.

In some embodiments, the SOS1 inhibitor is a compound as disclosed in WO 2021/092115, which is incorporated by reference in its entirety, or a pharmaceutically acceptable salt, solvate, isomer, stereoisomer, prodrug, or tautomer thereof. In some embodiments, the SOS1 inhibitor is a compound described by any of Formula (I), Formula (II), Formula (III), Formula (IV-a), Formula (IV-b), and Formula (IV-c), in WO 2021/092115, or a pharmaceutically acceptable salt, solvate, isomer, stereoisomer, prodrug, or tautomer thereof. In some embodiments, the SOS1 inhibitor is selected from a compound of Table A of WO 2021/092115, or a pharmaceutically acceptable salt, solvate, isomer, stereoisomer, prodrug, or tautomer thereof. In some embodiments, the SOS1 inhibitor is selected from a compound of Collection 1, Collection 2, Collection 3, Collection 4, or Collection 5 of WO 2021/092115, or a pharmaceutically acceptable salt, solvate, isomer, stereoisomer, prodrug, or tautomer thereof.

In some embodiments, the SOS1 inhibitor is a compound having the structure of Formula (48-I),

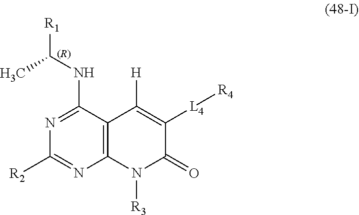

or a pharmaceutically acceptable salt, solvate, isomer, stereoisomer, prodrug, or tautomer thereof, wherein:
R¹ is selected from the group consisting of optionally substituted 3-6 membered cycloalkyl, optionally substituted 3-6 membered heterocyclyl, optionally substituted 6-membered aryl, and optionally substituted 5-6 membered heteroaryl;
R² is selected from the group consisting of H, C₁₋₆ alkyl, halogen, —NHR₂ₐ, —OR₂ₐ, cyclopropyl, and —CN; wherein C₁₋₆ alkyl is optionally substituted with halogen, —NHR₂ₐ, —OR₂ₐ, or 5-6 membered heterocyclyl, and further wherein R^{2a} is selected from the group consisting of H, C₁₋₆ alkyl, 3-6 membered heterocyclyl, and C₁₋₆ haloalkyl;
R³ is selected from the group consisting of H, C₁₋₃ alkyl, OR₃ₐ, cyclopropyl, and 3-6 membered heterocyclyl, wherein each of C₁₋₃ alkyl, cyclopropyl, and 3-6 membered heterocyclyl is optionally substituted with R₃ₐ, and further wherein R₃ₐ is selected from the group consisting of C₁₋₃ alkyl, halogen, —OH, or —CN;
L⁴ is selected from the group consisting of bond, —C(O)—, —C(O)O—, —C(O)NH(CH₂)ₒ—, —NH—, —S, —S(O)₂—,

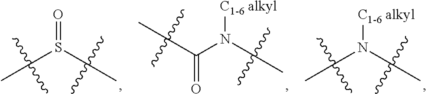

—(CH₂)ₚ—, and —O—; wherein o is 0, 1, or 2; and wherein p is a number from 1 to 6; and
R⁴ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —R^{4a}, —OR^{4a}, —O—C₁₋₆ alkyl-R₄ₐ, ═O, halogen, —C(O)R₄ₐ, —C(OO)R₄ₐ, —C(O)NR_{4b}R_{4c}, —NR_{4b}C(O)R_{4c}, —CN, ═NR₄ₐ, —NR_{4b}R_{4c}, —SO₂R₄ₐ, 3-6 membered cycloalkyl optionally substituted with R₄ₐ, 3-7 membered heterocyclyl optionally substituted with R₄ₐ, 6-10 membered aryl optionally substituted with R₄ₐ, or 5-10 membered heteroaryl optionally substituted with R₄ₐ;
   wherein R₄ₐ is H, C₁₋₆ alkyl, C₁₋₆ haloalkyl, —C(O)R_{4b}, C(O)NR₄₆R_{4c}, ═O, 3-6 membered cycloalkyl, 6-10 membered aryl optionally substituted with —OR_{4b}, —CN, ═N-3-6 membered cycloalkyl, 3-7 membered heterocyclyl, —(CH₂)ᵣOCH₃, or —(CH₂)ᵣOH, wherein r is 1, 2, or 3;
   wherein each R_{4b} is independently H, C₁₋₆ alkyl; and
   wherein each R^{4c} is independently H or C₁₋₆ alkyl.

In some embodiments of compounds of Formula (48-I), R¹ is the optionally substituted 6-membered aryl. In some embodiments, the 6-membered aryl has the following structure:

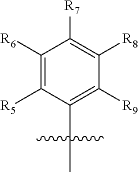

wherein R₅, R₆, R₇, R₈, and Ry are as defined below in connection with Formula (48-II)-(48-IV).

In some embodiments of compounds of Formula (48-I), R¹ is the optionally substituted 5-6 membered heteroaryl. In some embodiments, R¹ is a 6-membered heteroaryl having any of the following structures:

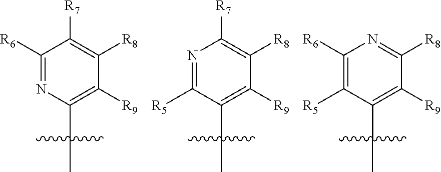

wherein R₅, R₆, R₇, R₈, and R₉ are as defined below in connection with Formula (48-II)-(48-IV).

In some embodiments of compounds of Formula (I), R₁ is the optionally substituted 5-6 membered heteroaryl. In some embodiments, R₁ is a 5-membered heteroaryl having the following structure:

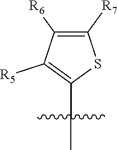

wherein R₅, R₆, and R₇ are as defined below in connection with Formula (48-II)-(48-IV).

In some embodiments, the SOS1 inhibitor is a compound having the structure of Formula (48-II),

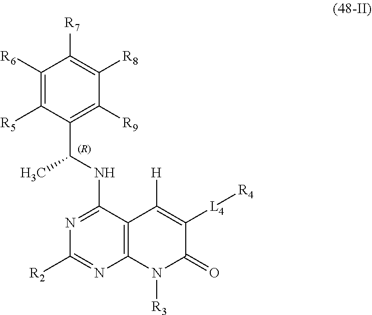

or a pharmaceutically acceptable salt, solvate, isomer, stereoisomer, prodrug, or tautomer thereof, wherein R₂, R₃, L₄, and R₄ are as defined in Formula (48-I);
R₅, R₆, R₇, R₈, and Ry are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, —C(O)R₁₀, —CO₂R₁₀, 6-10 membered aryl, and 5-10 membered heteroaryl, wherein each C₁₋₆ alkyl, C₂₋₆ alkenyl, 4-8 membered cycloalkenyl, C₂₋₆ alkynyl, 3-8 membered cycloalkyl, 3-14 membered heterocyclyl, 6-10 membered aryl, and 5-10 membered heteroaryl is optionally substituted with —OH, C₁₋₆ alkyl, halogen, —NO₂, oxo, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, 3-8 membered cycloalkyl, 3-14 membered heterocyclyl, 6-10 membered aryl, or 5-10 membered heteroaryl, or any two adjacent R₅, R₆, R₇, R₈, and R₉ forms an optionally substituted 3-14 membered fused ring;
R₁₀, R₁₁, and R₁₂ are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR₁₃, —SR₁₃, halogen, —NR₁₃R₁₄, —NO₂, and —CN; and
R₁₃ and R₁₄ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments, the SOS1 inhibitor is a compound having the structure of Formula (48-II),

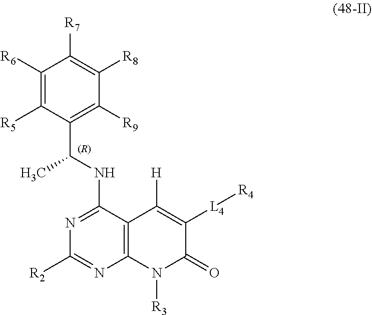

or a pharmaceutically acceptable salt or a stereoisomer thereof, wherein:
R² is H;
R³ is selected from the group consisting of H and C₁₋₃ alkyl;
L⁴ is a bond;
R⁴ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —R₄ₐ, —OR^{4a}, —O—C₁₋₆ alkyl-R₄ₐ, —O, halogen, —C(O)R₄ₐ, —C(OO)R₄ₐ, —C(O)NR_{4b}R_{4c}, —NR_{4b}C(O)R_{4c}, —CN, ═NR₄ₐ, —NR_{4b}R_{4c}, —SO₂R₄ₐ, 3-6 membered cycloalkyl optionally substituted with R₄ₐ, 3-7 membered heterocyclyl optionally substituted with R₄ₐ, 6-10 membered aryl optionally substituted with R₄ₐ, or 5-10 membered heteroaryl optionally substituted with R₄ₐ;
   wherein R₄ₐ is H, C₁₋₆ alkyl, C₁₋₆ haloalkyl, —C(O)R_{4b}, —C(O)NR_{4b}R_{4c}, ═O, 3-6 membered cycloalkyl, 6-10 membered aryl optionally substituted with —OR_{4b}, —CN, ═N-3-6 membered cycloalkyl, 3-7 membered heterocyclyl, —(CH₂)ᵣOCH₃, or —(CH₂)ᵣOH wherein r is 1, 2, or 3;
   wherein each Rob is independently H, C₁₋₆ alkyl;
   wherein each R_{4c} is independently H or C₁₋₆ alkyl;
R₅, R₆, R₇, R₈, and Ry are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, —C(O)R₁₀, —CO₂R₁₀, 6-10 membered aryl, and 5-10 membered heteroaryl, wherein each C₁₋₆ alkyl, C₂₋₆ alkenyl, 4-8 membered cycloalkenyl, C₂₋₆ alkynyl, 3-8 membered cycloalkyl, 3-14 membered heterocyclyl, 6-10 membered aryl, and 5-10 membered heteroaryl is optionally substituted with —OH, C₁₋₆ alkyl optionally substituted with —R₁₀, halogen, —NO₂, ═O, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, 3-8 membered cycloalkyl, 3-14 membered heterocyclyl optionally substituted with R₁₀, 6-10 membered aryl, or 5-10 membered heteroaryl;
R₁₀, R₁₁, and R₁₂ are at each occurrence independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR₁₃, —SR₁₃, halogen, —NR₁₃R₁₄, —NO₂, and —CN; and
R₁₃ and R₁₄ are at each occurrence independently selected from the group consisting of H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments, the present disclosure relates to compounds having the structure of Formula (48-III),

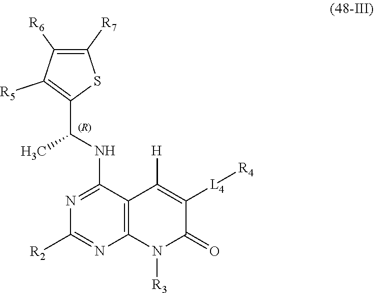

or a pharmaceutically acceptable salt, solvate, isomer, stereoisomer, prodrug, or tautomer thereof, wherein R₂, R₃, L₄, and R₄ are as defined Formula (48-I);
R₅, R₆, R₇, R₈, and Ry are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, —C(O)R₁₀, —CO₂R₁₀, 6-10 membered aryl, and 5-10 membered heteroaryl, wherein each C₁₋₆ alkyl, C₂₋₆ alkenyl, 4-8 membered cycloalkenyl, C₂₋₆ alkynyl, 3-8 membered cycloalkyl, 3-14 membered heterocyclyl, 6-10 membered aryl, and 5-10 membered heteroaryl is optionally substituted with —OH, C₁₋₆ alkyl optionally substituted with —R₁₀, halogen, —NO₂, oxo, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, 3-8 membered cycloalkyl, 3-14 membered heterocyclyl optionally substituted with R₁₀, 6-10 membered aryl, or 5-10 membered heteroaryl, or any two adjacent R₅, R₆, R₇, R₈, and R₉ forms an optionally substituted 3-14 membered fused ring;
R₁₀, R₁₁, and R₁₂ are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR₁₃, —SR₁₃, halogen, —NR₁₃R₁₄, —NO₂, and —CN; and
R₁₃ and R₁₄ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments, the present disclosure relates to compounds having the structure of Formula (48-IV-a), (48-IV-b), or (48-IV-c),

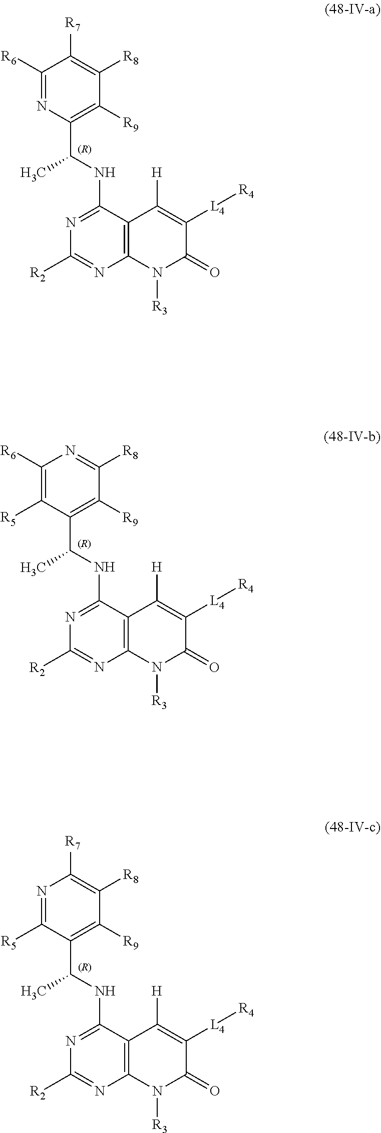

or a pharmaceutically acceptable salt, solvate, isomer, stereoisomer, prodrug, or tautomer thereof, wherein R², R³, L⁴, and R⁴ are as defined Formula (48-I);
R₅, R₆, R₇, R₈, and Ry are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, —C(O)R₁₀, —CO₂R₁₀, 6-10 membered aryl, and 5-10 membered heteroaryl, wherein each C₁₋₆ alkyl, C₂₋₆ alkenyl, 4-8 membered cycloalkenyl, C₂₋₆ alkynyl, 3-8 membered cycloalkyl, 3-14 membered heterocyclyl, 6-10 membered aryl, and 5-10 membered heteroaryl is optionally substituted with —OH, C₁₋₆ alkyl optionally substituted with —R₁₀, halogen, —NO₂, oxo, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, 3-8 membered cycloalkyl, 3-14 membered heterocyclyl optionally substituted with R₁₀, 6-10 membered aryl, or 5-10 membered heteroaryl, or any two adjacent R₅, R₆, R₇, R₈, and R₉ forms an optionally substituted 3-14 membered fused ring;
R₁₀, R₁₁, and R₁₂ are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR₁₃, —SR₁₃, halogen, —NR₁₃R₁₄, —NO₂, and —CN; and
R₁₃ and R₁₄ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments of compounds of Formula (48-II)-(48-IV), one to three of R₅, R₆, R₇, R₈, and R₉ is C₁₋₆ alkyl, wherein the alkyl is optionally substituted with halogen.

In some embodiments of compounds of Formula (48-II)-(48-IV), one to three of R₅, R₆, R₇, R₈, and R₉ is C₁₋₆ alkyl, wherein the alkyl is optionally substituted with halogen or —OH.

In some embodiments of compounds of Formula (48-II)-(48-IV), one to three of R₅, R₆, R₇, R₈, and R₉ is C₁₋₆ alkyl, and one to three of R₅, R₆, R₇, R₈, and R₉ is C₁₋₆ alkyl optionally substituted with halogen.

In some embodiments of compounds of Formula (48-II)-(48-IV), one to three of R₅, R₆, R₇, R₈, and R₉ is halogen, and one to three of R₅, R₆, R₇, R₈, and R₉ is C₁₋₆ alkyl optionally substituted with halogen.

In some embodiments of compounds of Formula (48-II)-(48-IV), one to three of R₅, R₆, R₇, R₈, and R₉ is —NH₂.

In some embodiments of compounds of Formula (48-II)-(48-IV), one of R₅, R₆, R₇, R₈, and R₉ is —NH₂; and one of R₅, R₆, R₇, R₈, and R₉ is C₁₋₆ alkyl optionally substituted with halogen.

In some embodiments of compounds of Formula (48-II)-(48-IV), any two adjacent R⁵, R⁶, R⁷, R⁸, and R⁹ forms a 3-14 membered fused ring. In some embodiments of compounds of Formula (48-II)-(48-IV), any two adjacent R⁵, R⁶, R⁷, R⁸, and R⁹ forms a 3-8 membered fused ring. In some embodiments of compounds of Formula (48-II)-(48-IV), any two adjacent R⁵, R⁶, R⁷, R⁸, and R⁹ forms a 4-8 membered fused ring. In some embodiments of compounds of Formula (48-II)-(48-IV), any two adjacent R⁵, R⁶, R⁷, R⁸, and R⁹ forms a 4-membered fused ring or a 5-membered fused ring. In some embodiments, the fused ring is a 3-8 membered heterocyclyl or a 3-8 membered cycloalkyl. In some embodiments, the fused ring is a 4-8 membered heterocyclyl or a 4-8 membered cycloalkyl. In some embodiments, the fused ring is a 4-membered heterocyclyl or a 5-membered heterocyclyl. In some embodiments, the fused ring is a 4-membered cycloalkyl or a 5-membered cycloalkyl. In some embodiments, the fused ring is optionally substituted with —OH, C₁₋₆ alkyl, halogen, —NO₂, oxo, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, 3-8 membered cycloalkyl, 3-14 membered heterocyclyl, 6-10 membered aryl, or 5-10 membered heteroaryl. In some embodiments, the fused ring is optionally substituted with halogen.

In some embodiments of compounds of Formula (48-II)-(48-IV), one or more of R₅, R₆, R₇, R₈, and Ry is selected from among-CF₃, —NH₂, —F, and —CF₂CH₂OH. In some embodiments of compounds of Formula (II), one of R₅, R₆, R₇, R₈, and R₉ is —CF₃ and one of R₅, R₆, R₇, R₈, and R₉ is —NH₂. In some embodiments of compounds of Formula (48-II), one of R₅, R₆, R₇, R₈, and R₉ is —F and one of R₅, R₆, R₇, R₈, and R₉ is —CF₂CH₂OH.

In some embodiments of compounds of Formula (48-I), R₁ is selected from among:

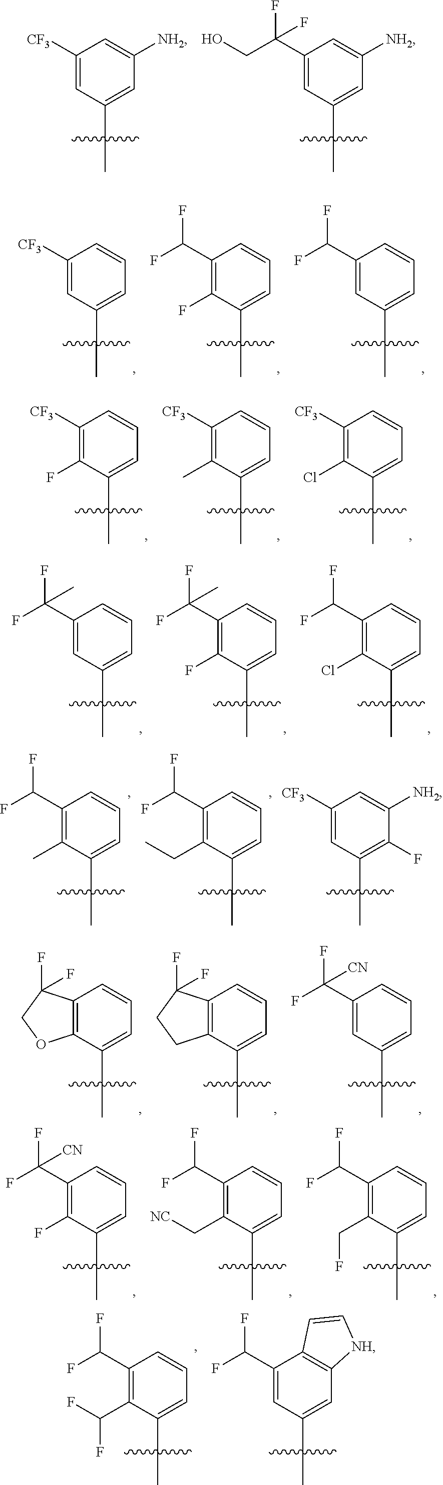

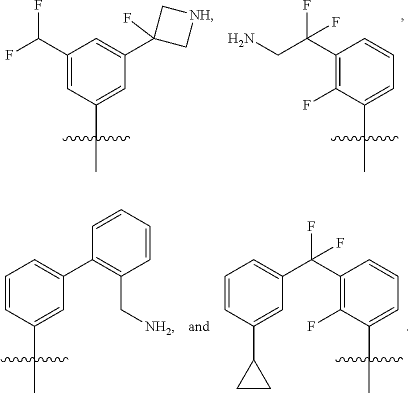

In some embodiments of compounds of Formula (48-I), R₁ is selected from among:

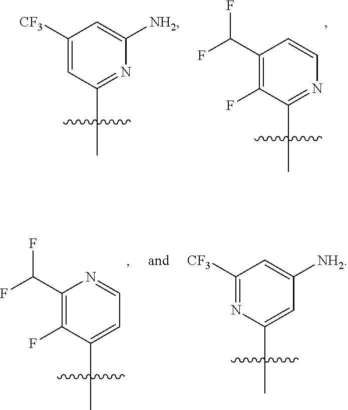 [SMILES: *c1cc(C(F)(F)F)cc(N)n1 ; *c1nccc(C(F)F)c1F ; *c1ccnc(C(F)F)c1F ; *c1cc(N)cc(C(F)(F)F)n1]

In some embodiments of compounds of Formula (48-I), R₁ is selected from among:

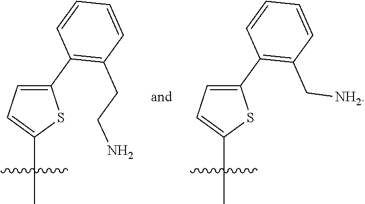 [SMILES: *c1ccc(-c2ccccc2CCN)s1 ; *c1ccc(-c2ccccc2CN)s1]

In some embodiments of compounds of Formula (48-I), R₁ is selected from among:

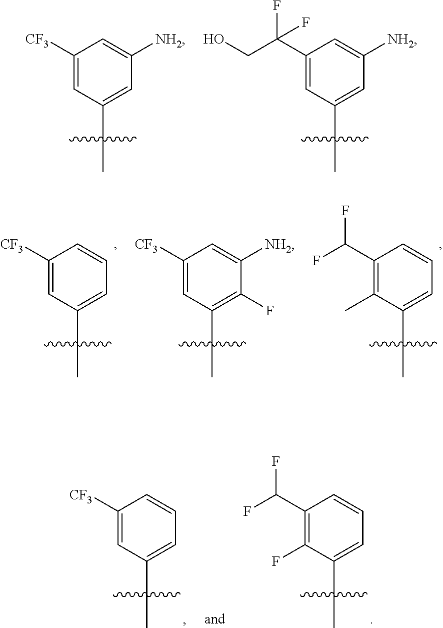

In some embodiments of compounds of Formula (48-I)-(48-IV), R₂ is H.

In some embodiments of compounds of Formula (48-I)-(48-IV), R₂ is C₁₋₆ alkyl. In some embodiments of compounds of Formula (I)—(IV), R₂ is —CH₃.

In some embodiments of compounds of Formula (48-I)-(48-IV), R₂ is C₁₋₆ alkyl substituted with 5-6 membered heterocycloalkyl. In some embodiments of compounds of Formula (48-I)-(48-IV), R₂ is

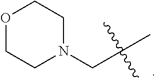 [SMILES: *CN1CCOCC1]

In some embodiments of compounds of Formula (48-I)-(48-IV), R₂ is C₁₋₆ alkyl substituted with —NHR₂ₐ, wherein R₂ₐ is C₁₋₆ alkyl or 3-6 membered heterocyclyl. In some embodiments of compounds of Formula (48-I)-(48-IV), R₂ is selected from among

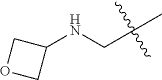 [SMILES: *CNC1COC1]

and —CH₂NHCH₃.

In some embodiments of compounds of Formula (48-I)-(48-IV), R² is C₁₋₆ alkyl substituted with —OR₂ₐ, wherein R^{2a} is H or C₁₋₆ alkyl. In some embodiments of compounds of Formula (48-I)-(48-IV), R² is —CH₂OH.

In some embodiments of compounds of Formula (48-I)-(48-IV), R² is —NHR₂ₐ, wherein R₂ₐ is C₁₋₆ alkyl. In some embodiments of compounds of Formula (48-I)-(48-IV), R₂ is —NHCH₃.

In some embodiments of compounds of Formula (48-I)-(48-IV), R₂ is —OR₂ₐ; wherein R₂ₐ is C₁₋₆ alkyl. In some embodiments of compounds of Formula (48-I)-(48-IV), R₂ is —OCH₃.

In some embodiments of compounds of Formula (48-I)-(48-IV), R₃ is C₁₋₃ alkyl. In some embodiments of compounds of Formula (48-I)-(48-IV), R₃ is —CH₃. In some embodiments of compounds of Formula (48-1)-(48-IV), R³ is —CD₃.

In some embodiments of compounds of Formula (48-1)-(48-IV), R₃ is C₁₋₃ alkyl substituted with —OH. In some embodiments of compounds of Formula (I)—(IV), R₃ is —CH₂CH₂OH.

In some embodiments of compounds of Formula (48-I)-(48-IV), R₃ is H.

In some embodiments of compounds of Formula (48-I)-(48-IV), R₃ is —OR₃ₐ. In some embodiments of compounds of Formula (I)—(IV), R₃ is —OCH₃.

In some embodiments of compounds of Formula (48-1)-(48-IV), R₃ is cyclopropyl.

In some embodiments of compounds of Formula (48-I)-(48-IV), R₃ is 3-6 membered heterocyclyl. In some embodiments of compounds of Formula (48-I)-(48-IV), R₃ is

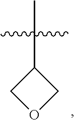 [SMILES: *C1COC1]

In some embodiments of compounds of Formula (48-I)-(48-IV), L₄ is selected from the group consisting of bond, —C(O)—, —C(O)O—, —C(O)NH(CH₂)ₒ—, —NH—, —S—, —S(O)₂—,

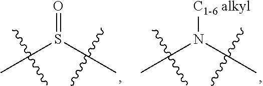

—(CH₂)ₚ—, and —O—; wherein o is 0, 1, or 2; and wherein p is a number from 1 to 6.

In some embodiments of compounds of Formula (48-I)-(48-IV), L₄ is a bond.

In some embodiments of compounds of Formula (48-1)-(48-IV), L₄ is —C(O)—.

In some embodiments of compounds of Formula (48-I)-(48-IV), L₄ is —(CH₂)ₚ—. In some embodiments of compounds of Formula (I), L⁴ is —(CH₂)—.

In some embodiments of compounds of Formula (48-I)-(48-IV), R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —R₄ₐ, —OR₄ₐ, —O—C₁₋₆ alkyl-R₄ₐ, ═O, halogen, —C(O)R₄ₐ, —C(OO)R₄ₐ, —C(O)NR_{4b}R_{4c}, —NR_{4b}C(O)R_{4c}, —CN, ═NR₄ₐ, —NR_{4b}R_{4c}, —SO₂R₄ₐ, 3-6 membered cycloalkyl, 3-7 membered heterocyclyl, 6-10 membered aryl, or 5-10 membered heteroaryl.

In some embodiments of compounds of Formula (48-I)-(48-IV), R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl-OR₄ₐ, ═O, halogen, —C(O)R₄ₐ, —C(OO)R₄ₐ, —C(O)NR_{4b}R_{4c}, —CN, —NR_{4b}R_{4c}, 3-6 membered cycloalkyl, 3-7 membered heterocyclyl, 6-10 membered aryl, or 5-10 membered heteroaryl.

In some embodiments of compounds of Formula (48-I)-(48-IV), R₄ₐ is H, C₁₋₆ alkyl, C₁₋₆ haloalkyl, —C(O)R_{4b}, C(O)NR_{4b}R_{4c}, ═O, 3-6 membered cycloalkyl, 6-10 membered aryl optionally substituted with —OR_{4b}, CN, ═N-3-6 membered cycloalkyl, 3-7 membered heterocyclyl, —(CH₂)ᵣOCH₃, or —(CH₂)ᵣOH, wherein r is 1, 2, or 3; wherein each R_{4b} is independently H, C₁₋₆ alkyl; and wherein each R_{4c} is independently H or C₁₋₆ alkyl.

In some embodiments of compounds of Formula (48-I)-(48-IV), R₄ₐ is H, C₁₋₆ alkyl, C₁₋₆ haloalkyl, —C(O)R_{4b}, C(O)NR_{4b}R_{4c}, 3-6 membered cycloalkyl, 6-10 membered aryl optionally substituted with —OR_{4b}, —CN, 3-7 membered heterocyclyl, —(CH₂)ᵣOCH₃, or —(CH₂)ᵣOH, wherein r is 1, 2, or 3; wherein each Rib is independently H, C₁₋₆ alkyl; and wherein each R_{4c} is independently H or C₁₋₆ alkyl.

In some embodiments of compounds of Formula (48-I)-(48-IV), R₄ is 3-14 membered heterocyclyl. In some embodiments of compounds of Formula (48-I)-(48-IV), R₄ is substituted 3-14 membered heterocyclyl.

In some embodiments of compounds of Formula (48-I)-(48-IV), R₄ is 3-14 membered heterocyclyl substituted with 3-6 membered heterocyclyl. In some embodiments, the heterocyclyl substituent is oxetanyl.

In some embodiments of compounds of Formula (48-I)-(48-IV), R₄ is 3-14 membered heterocyclyl substituted with C₁₋₆ alkyl. In some embodiments of compounds of Formula (48-I)-(48-IV), R₄ is 3-14 membered heterocyclyl substituted with —CH₃. In some embodiments of compounds of Formula (48-I)-(48-IV), R₄ is 3-14 membered heterocyclyl substituted with —CH₂—, i.e., the substituent is a methylene bridge bridging 2 carbon atoms in the heterocyclyl ring.

In some embodiments of compounds of Formula (48-I)-(48-IV), R₄ is 3-14 membered heterocyclyl substituted with 3-6 membered cycloalkyl. In some embodiments, the cycloalkyl substituent is cyclopropyl.

In some embodiments of compounds of Formula (48-I)-(48-IV), R₄ is 3-14 membered heterocyclyl substituted with ═O.

In some embodiments, the R₄ is a heterocyclyl selected from among:

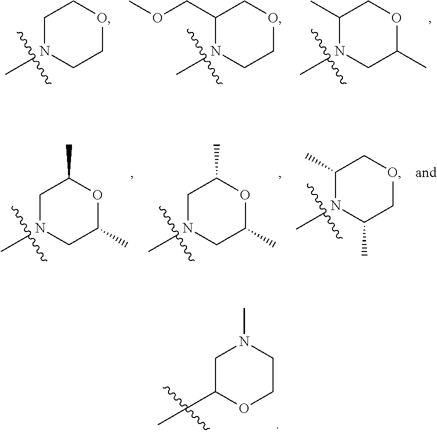

In some embodiments, the R₄ is a heterocyclyl selected from among:

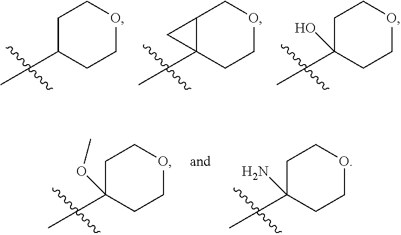

In some embodiments, the R₄ is a heterocyclyl selected from among:

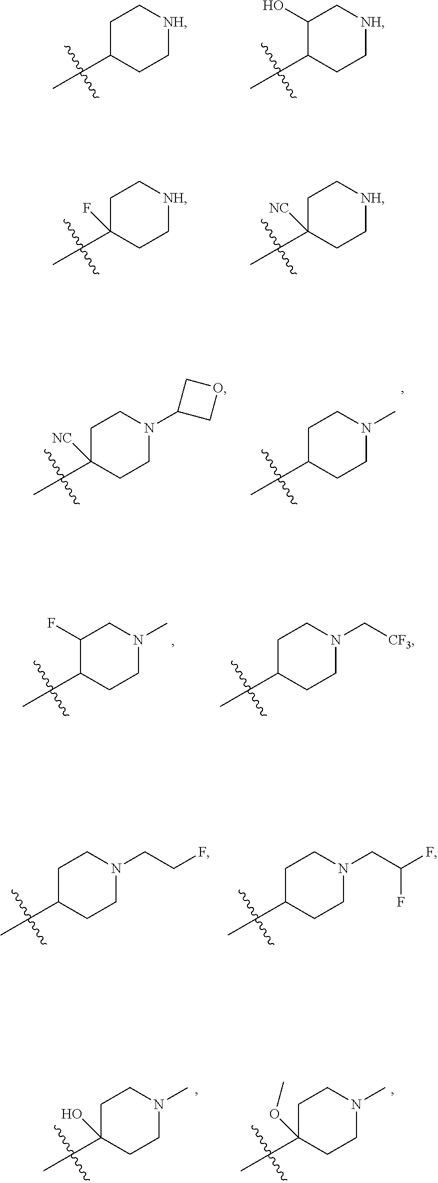

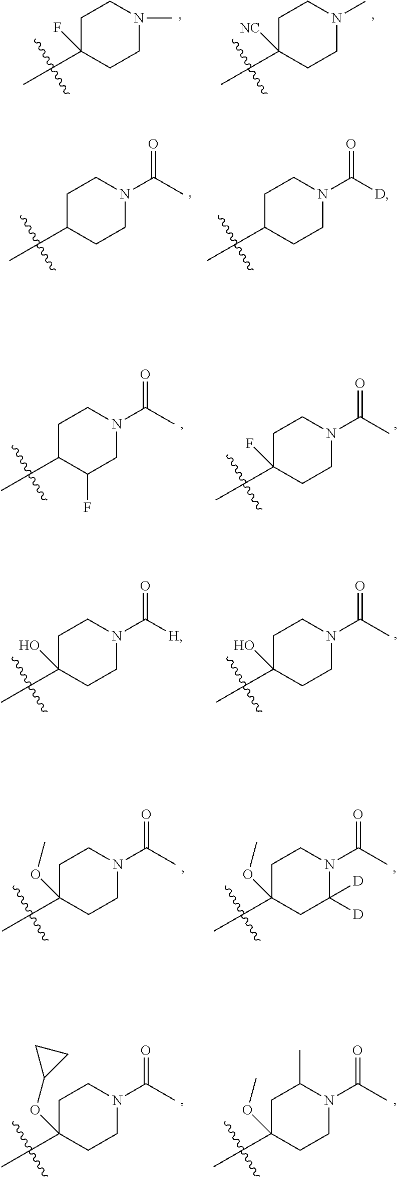

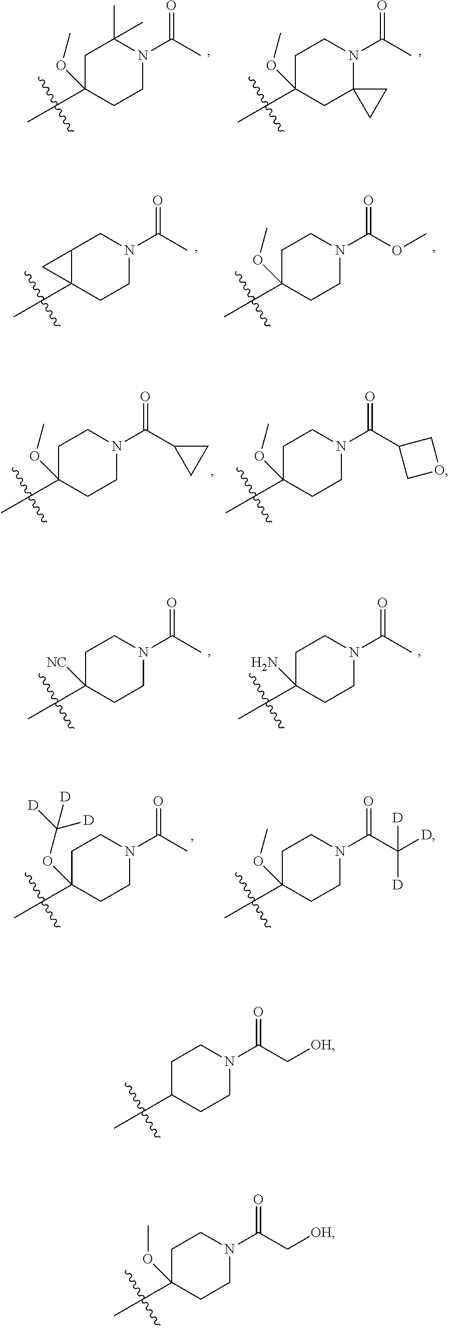

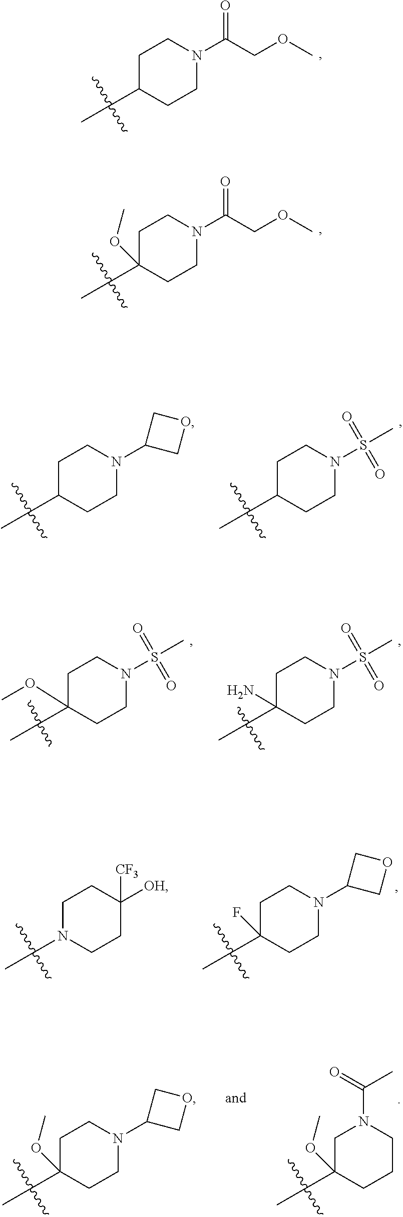

In some embodiments, the R₄ is a heterocyclyl selected from among:

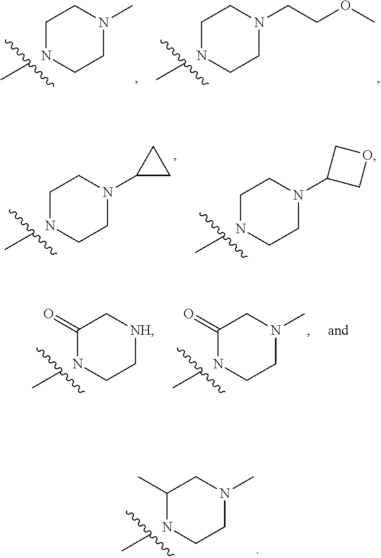

In some embodiments, the R₄ is a heterocyclyl selected from among:

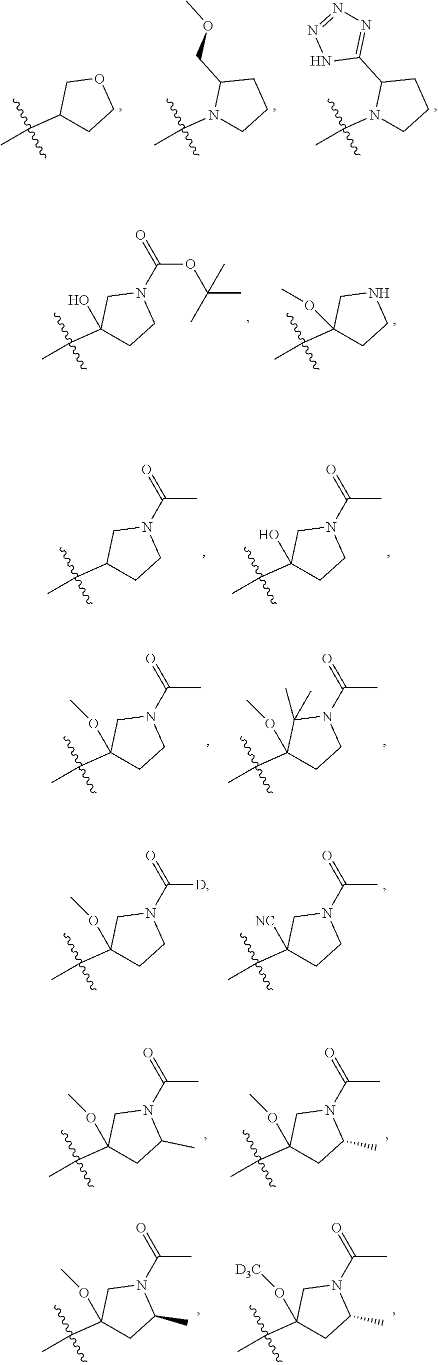

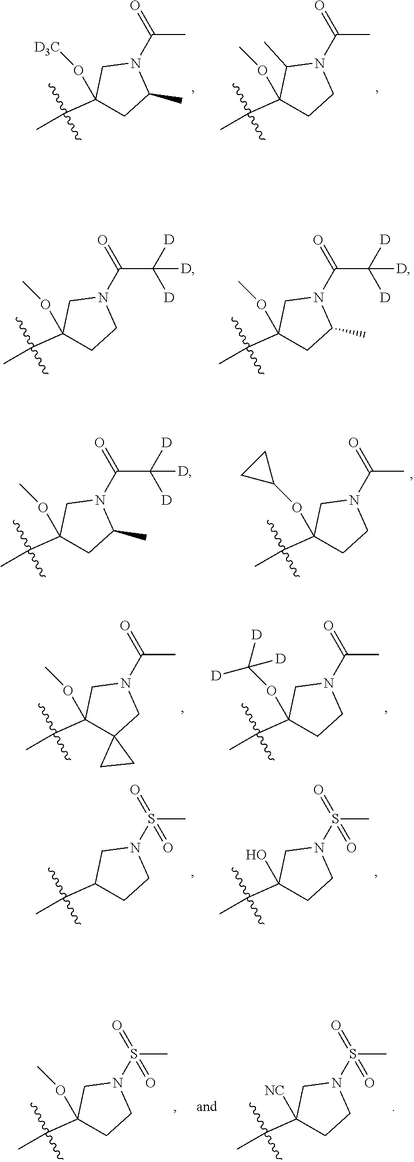

In some embodiments, the R₄ is a heterocyclyl selected from among:

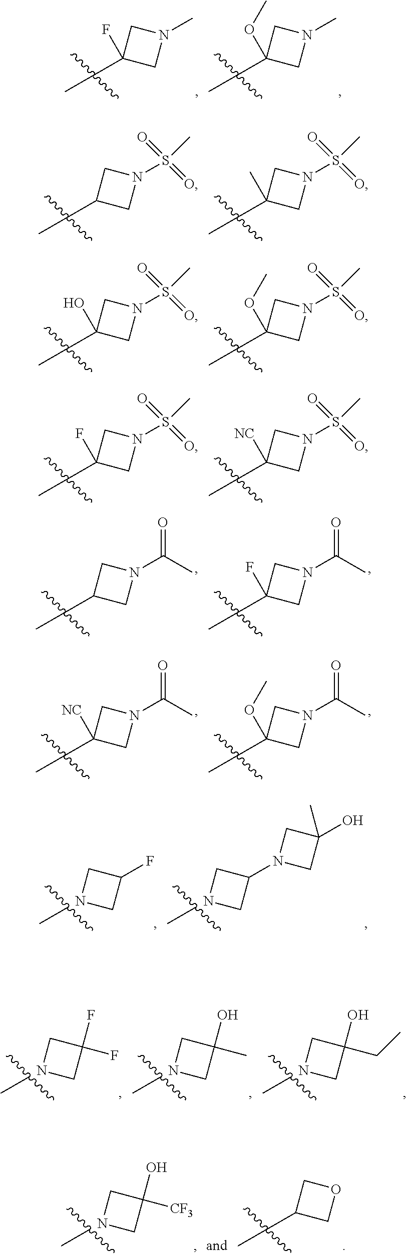

In some embodiments, the R₄ is a heterocyclyl selected from among:

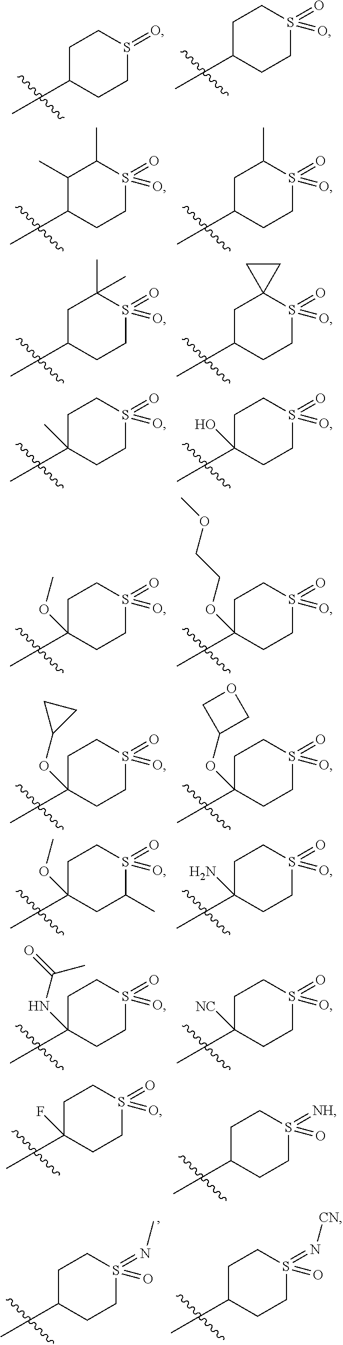

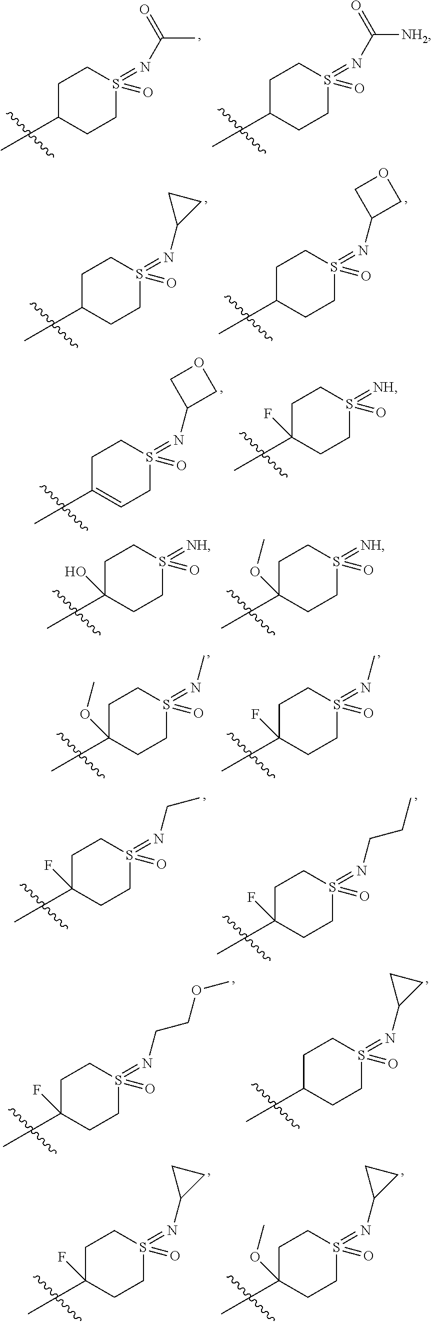

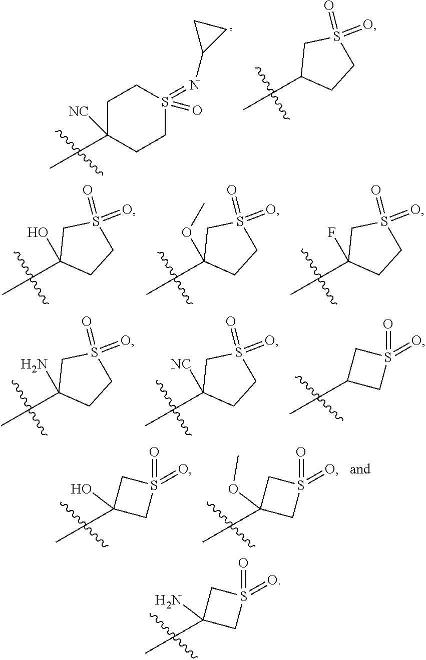

In some embodiments, the R₄ is a heterocyclyl selected from among:

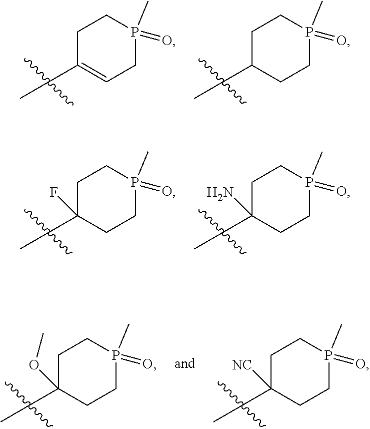

In some embodiments, the R₄ is a heterocyclyl selected from among:

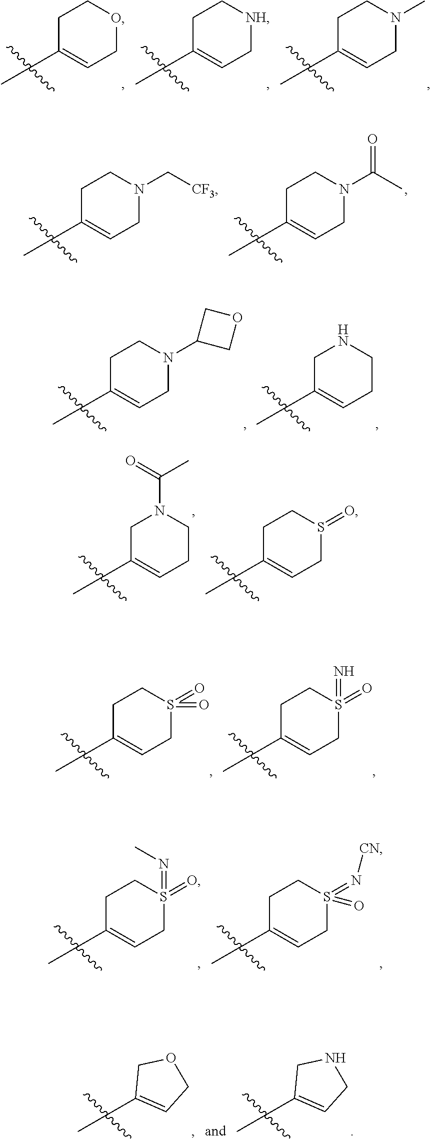

In some embodiments, the R₄ is a heterocyclyl selected from among:

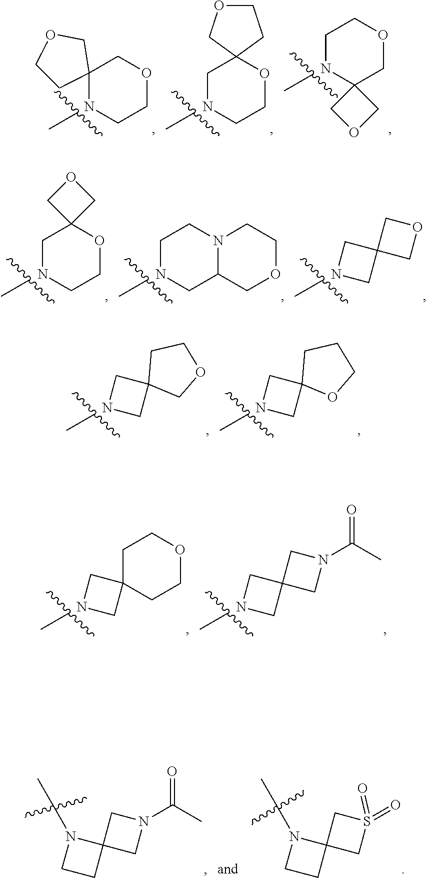

In some embodiments, the R₄ is a heterocyclyl selected from among:

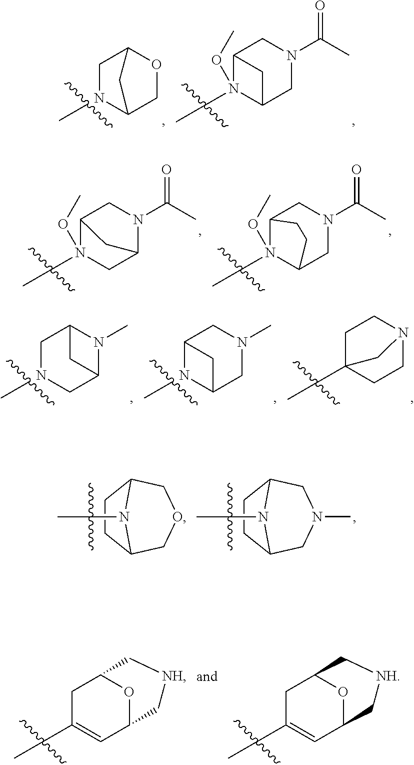

In some embodiments, the R₄ is a heterocyclyl selected from among:

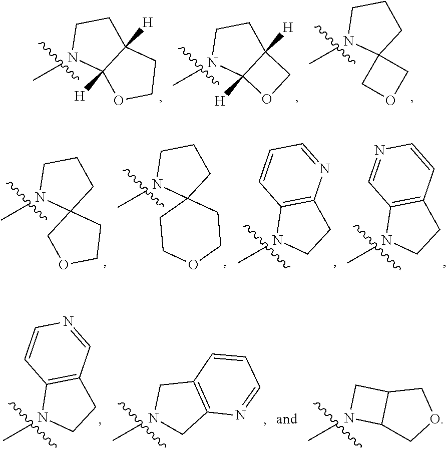

In some embodiments, the R₄ is a heterocyclyl selected from among:

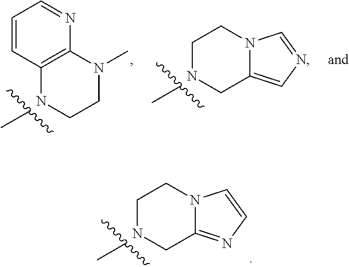

In some embodiments, the R₄ is a heterocyclyl selected from among:

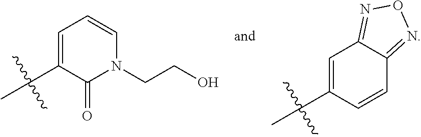 [SMILES: *c1cccn(CCO)c1=O ; *c1ccc2nonc2c1]

In some embodiments, the R₄ is a heterocyclyl selected from among:

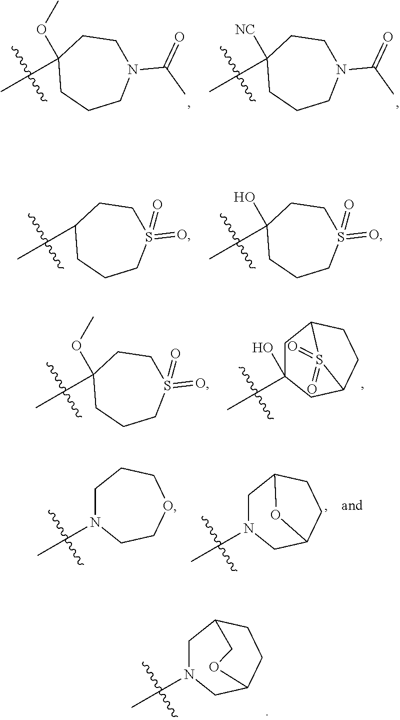

In some embodiments, R₄ is selected from among:

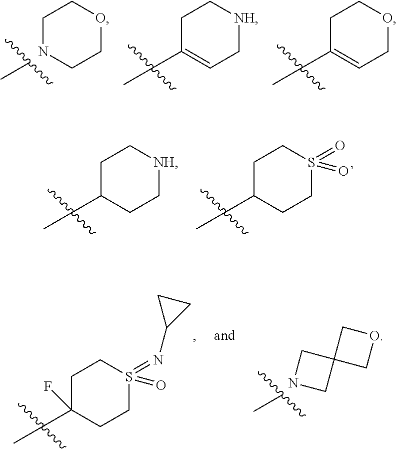

In some embodiments, R₄ is 3-14 membered cycloalkyl. In some embodiments, R₄ is substituted 3-14 membered cycloalkyl.

In some embodiments, R₄ is selected from among:

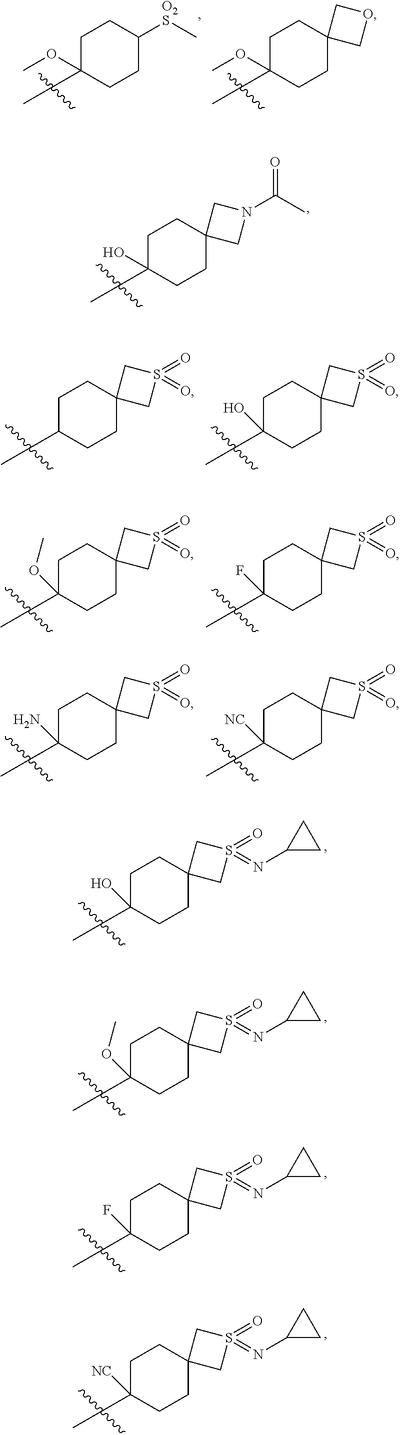

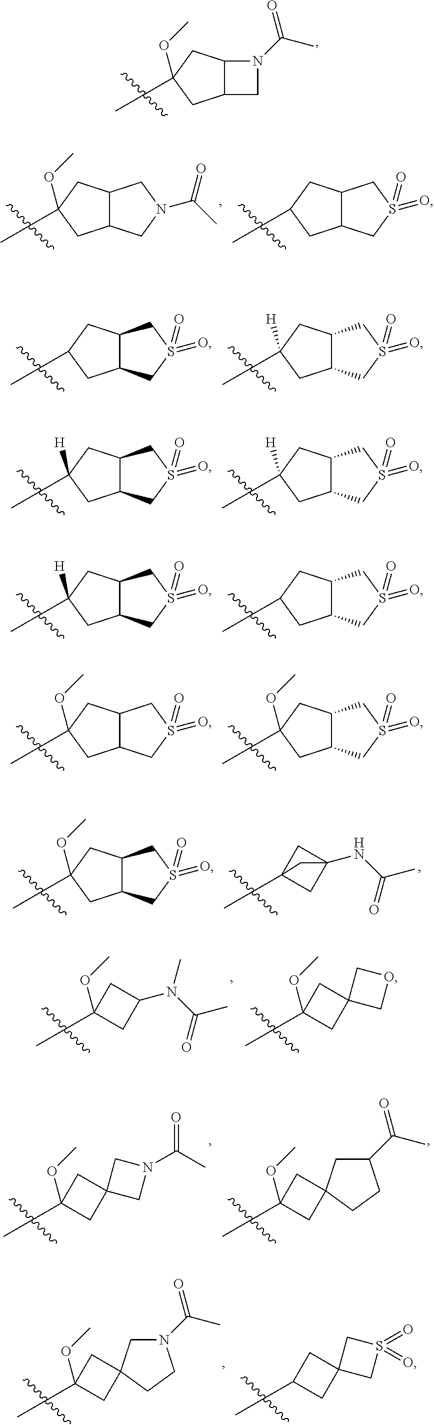

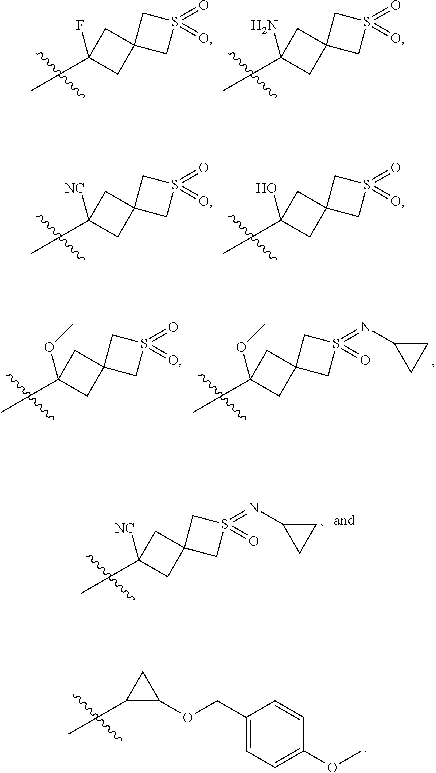

In some embodiments, R₄ is 6-10 membered aryl. In some embodiments, R₄ is substituted 6-10 membered aryl. In some embodiments, R₄ is phenyl. In some embodiments, R₄ is phenyl substituted with one or two group selected from among-OCH₃ and —CN.

In some embodiments, R₄ is 5-10 membered heteroaryl. In some embodiments, R₄ is substituted 5-10 membered heteroaryl. In some embodiments, R₄ is selected from among 1H-pyrrole, thiazole, pyridine, pyridazine, pyrimidine, each of which is optionally substituted with a group selected from among-F, —OCH₃, and —OCH₂CH₂OH.

In some embodiments, R₄ is selected from among

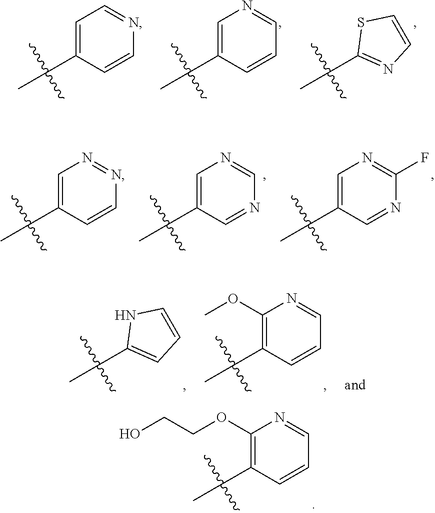

In some embodiments, the SOS1 inhibitor is a compound selected from the group consisting of the compounds in the following table, or a pharmaceutically acceptable salt, solvate, isomer, stereoisomer, prodrug, or tautomer thereof:

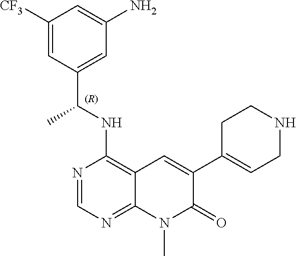

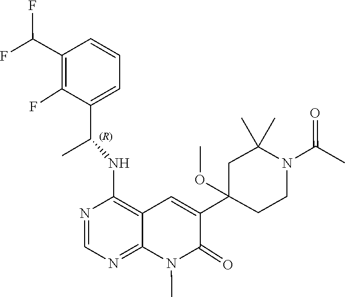

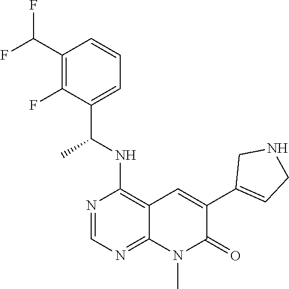 [SMILES: C[C@@H](Nc1ncnc2c1cc(C1=CCNC1)c(=O)n2C)c1cccc(C(F)F)c1F]

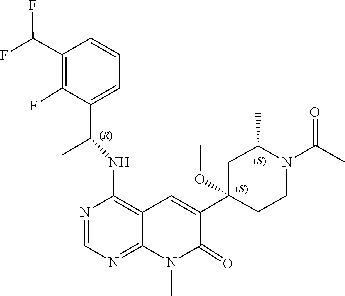

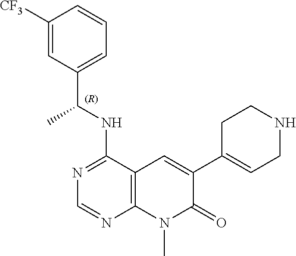

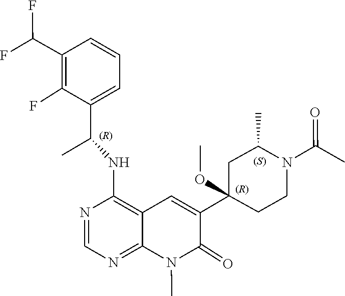

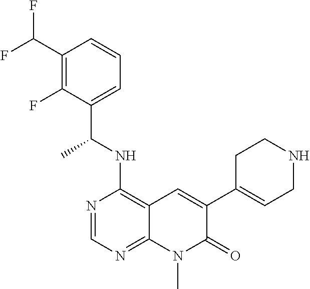 [SMILES: C[C@@H](Nc1ncnc2c1cc(C1=CCNCC1)c(=O)n2C)c1cccc(C(F)F)c1F]

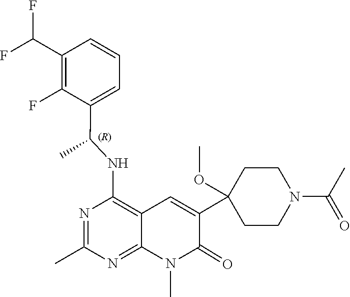

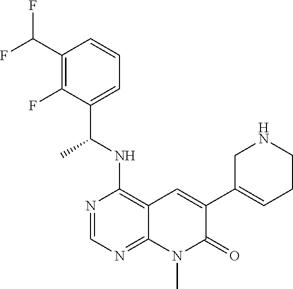 [SMILES: C[C@@H](Nc1ncnc2c1cc(C1=CCCNC1)c(=O)n2C)c1cccc(C(F)F)c1F]

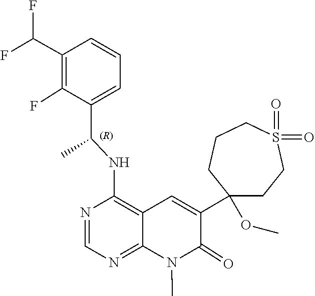

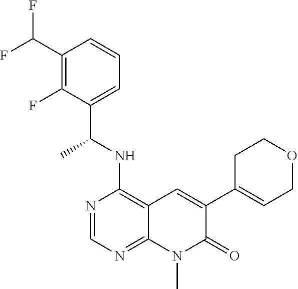 [SMILES: C[C@@H](Nc1ncnc2c1cc(C1=CCOCC1)c(=O)n2C)c1cccc(C(F)F)c1F]

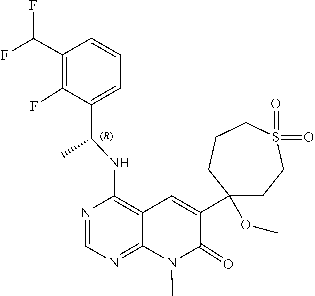

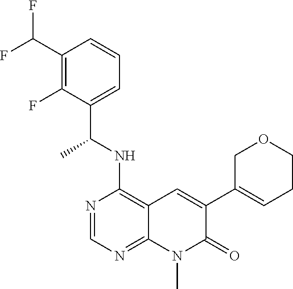 [SMILES: C[C@@H](Nc1ncnc2c1cc(C1=CCCOC1)c(=O)n2C)c1cccc(C(F)F)c1F]

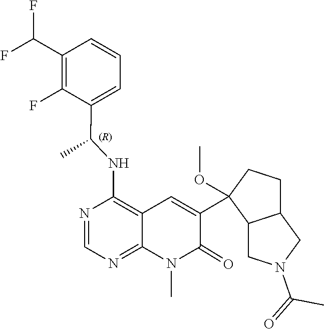

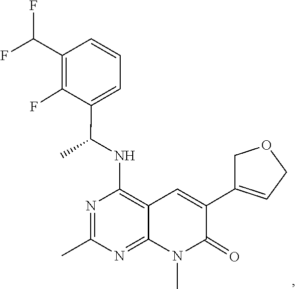 [SMILES: Cc1nc(N[C@H](C)c2cccc(C(F)F)c2F)c2cc(C3=CCOC3)c(=O)n(C)c2n1]

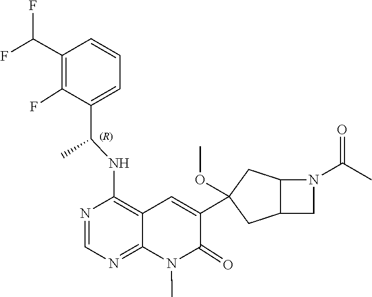

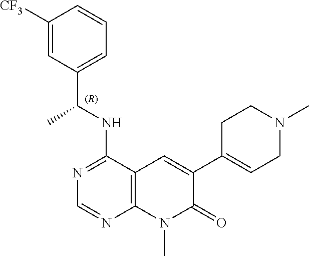

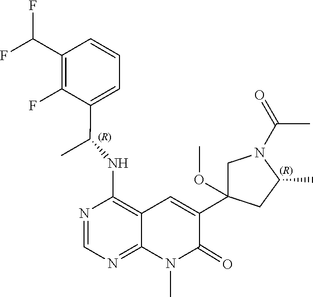

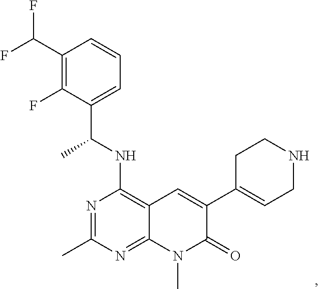 [SMILES: Cc1nc(N[C@H](C)c2cccc(C(F)F)c2F)c2cc(C3=CCNCC3)c(=O)n(C)c2n1]

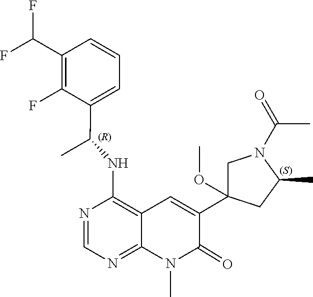

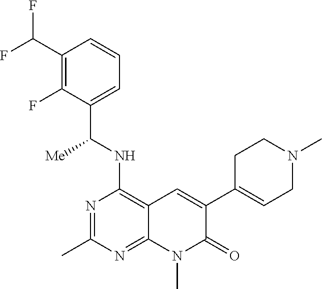 [SMILES: Cc1nc(N[C@H](C)c2cccc(C(F)F)c2F)c2cc(C3=CCN(C)CC3)c(=O)n(C)c2n1]

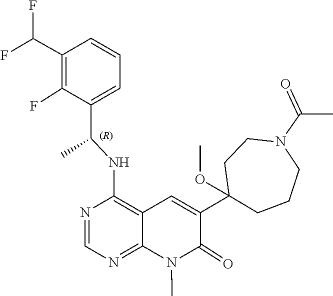

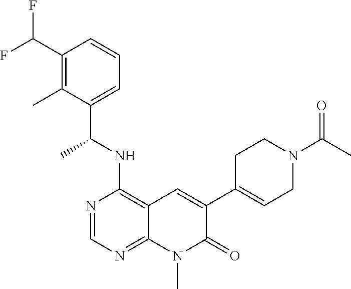 [SMILES: CC(=O)N1CC=C(c2cc3c(N[C@H](C)c4cccc(C(F)F)c4C)ncnc3n(C)c2=O)CC1]

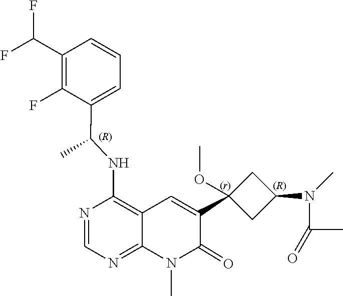

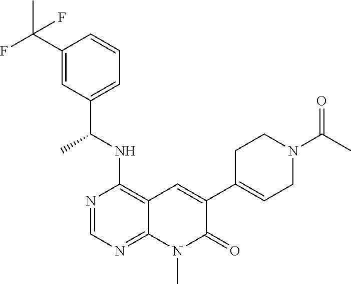 [SMILES: CC(=O)N1CC=C(c2cc3c(N[C@H](C)c4cccc(C(C)(F)F)c4)ncnc3n(C)c2=O)CC1]

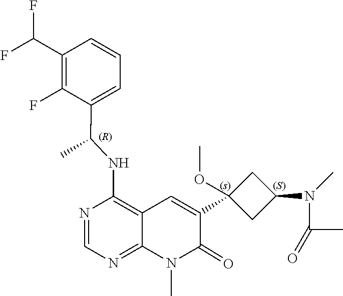

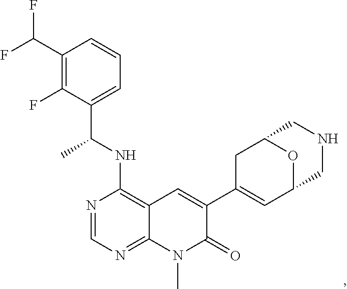 [SMILES: C[C@@H](Nc1ncnc2c1cc(C1=C[C@H]3CNC[C@@H](C1)O3)c(=O)n2C)c1cccc(C(F)F)c1F]

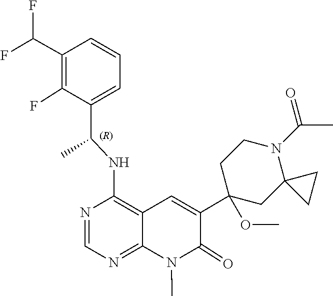

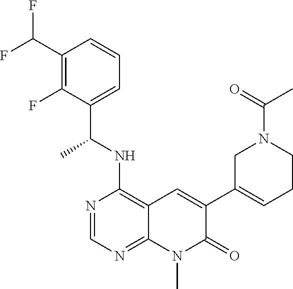 [SMILES: CC(=O)N1CCC=C(c2cc3c(N[C@H](C)c4cccc(C(F)F)c4F)ncnc3n(C)c2=O)C1]

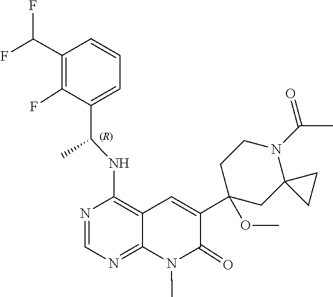

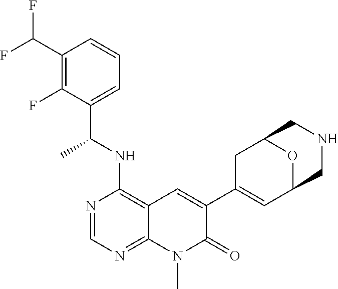 [SMILES: C[C@@H](Nc1ncnc2c1cc(C1=C[C@@H]3CNC[C@H](C1)O3)c(=O)n2C)c1cccc(C(F)F)c1F]

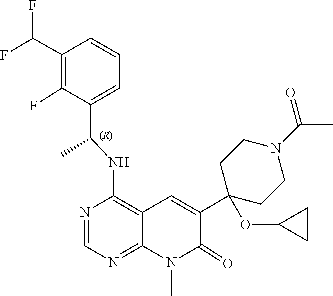

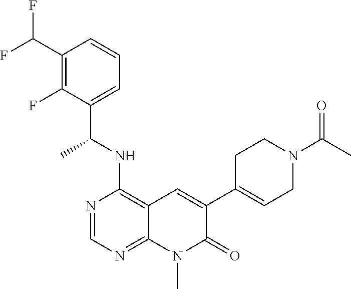 [SMILES: CC(=O)N1CC=C(c2cc3c(N[C@H](C)c4cccc(C(F)F)c4F)ncnc3n(C)c2=O)CC1]

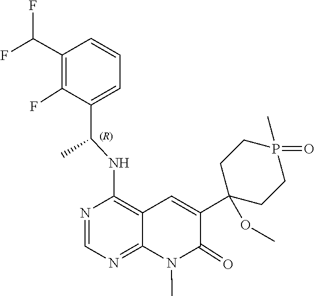

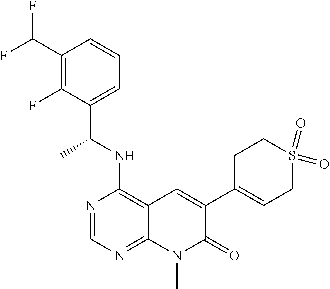 [SMILES: C[C@@H](Nc1ncnc2c1cc(C1=CCS(=O)(=O)CC1)c(=O)n2C)c1cccc(C(F)F)c1F]

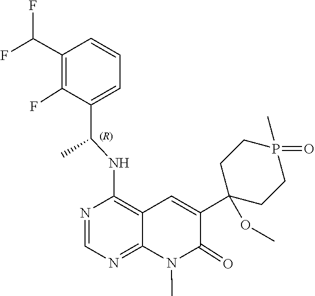

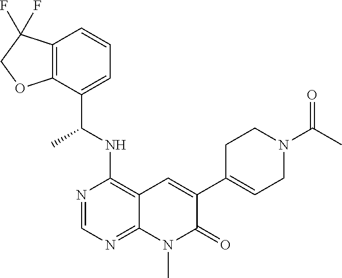 [SMILES: CC(=O)N1CC=C(c2cc3c(N[C@H](C)c4cccc5c4OCC5(F)F)ncnc3n(C)c2=O)CC1]

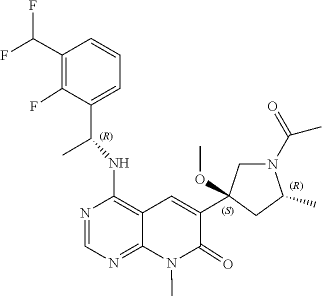

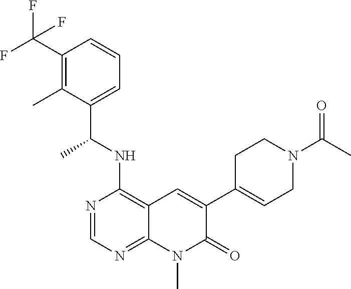 [SMILES: CC(=O)N1CC=C(c2cc3c(N[C@H](C)c4cccc(C(F)(F)F)c4C)ncnc3n(C)c2=O)CC1]

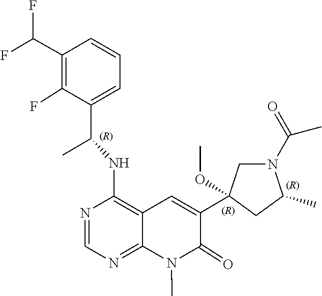

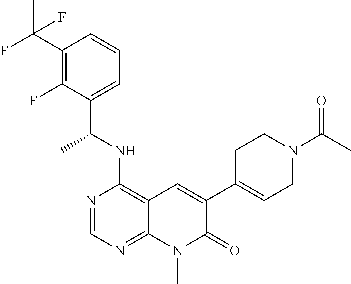 [SMILES: CC(=O)N1CC=C(c2cc3c(N[C@H](C)c4cccc(C(C)(F)F)c4F)ncnc3n(C)c2=O)CC1]

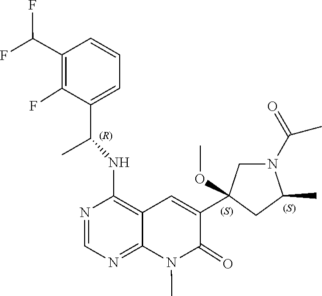

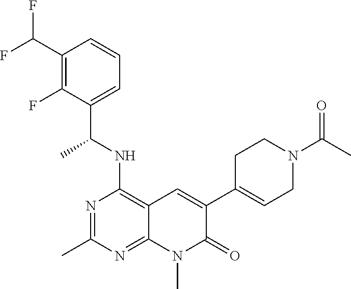 [SMILES: CC(=O)N1CC=C(c2cc3c(N[C@H](C)c4cccc(C(F)F)c4F)nc(C)nc3n(C)c2=O)CC1]

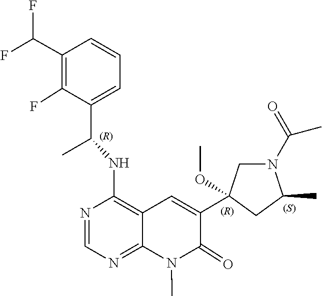

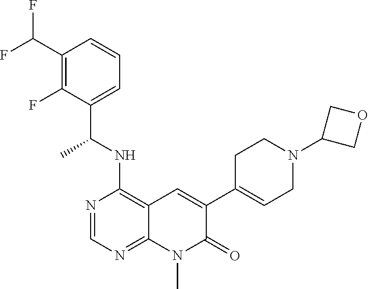 [SMILES: C[C@@H](Nc1ncnc2c1cc(C1=CCN(C3COC3)CC1)c(=O)n2C)c1cccc(C(F)F)c1F]

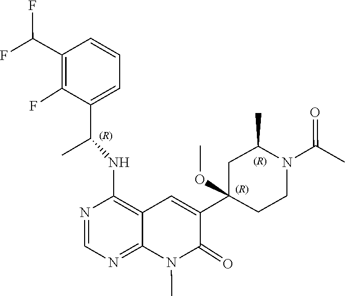

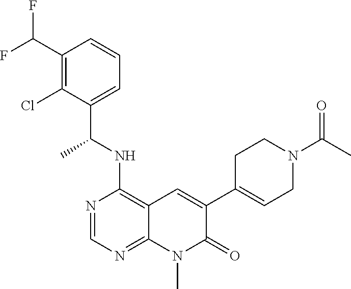 [SMILES: CC(=O)N1CC=C(c2cc3c(N[C@H](C)c4cccc(C(F)F)c4Cl)ncnc3n(C)c2=O)CC1]

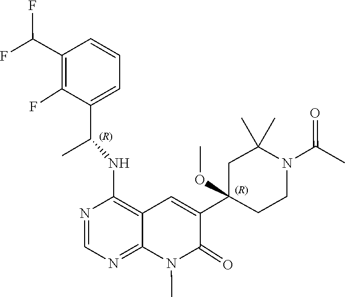

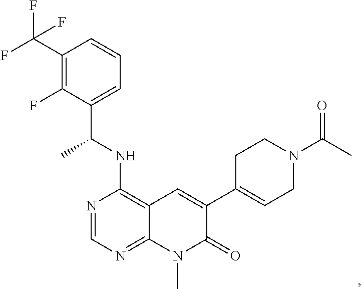 [SMILES: CC(=O)N1CC=C(c2cc3c(N[C@H](C)c4cccc(C(F)(F)F)c4F)ncnc3n(C)c2=O)CC1]

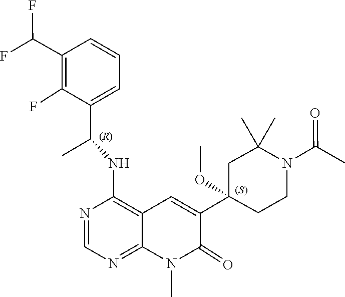

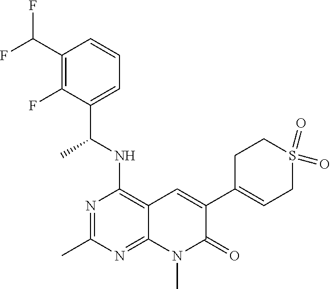 [SMILES: Cc1nc(N[C@H](C)c2cccc(C(F)F)c2F)c2cc(C3=CCS(=O)(=O)CC3)c(=O)n(C)c2n1]

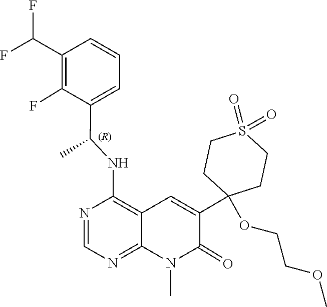

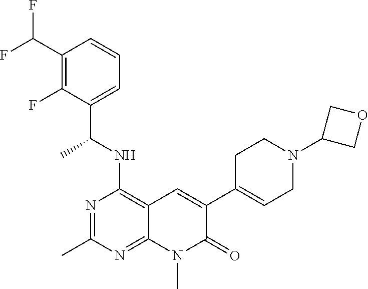 [SMILES: Cc1nc(N[C@H](C)c2cccc(C(F)F)c2F)c2cc(C3=CCN(C4COC4)CC3)c(=O)n(C)c2n1]

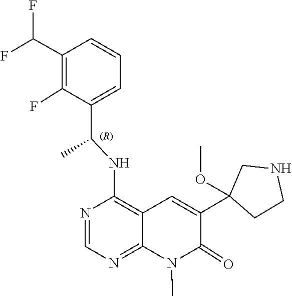

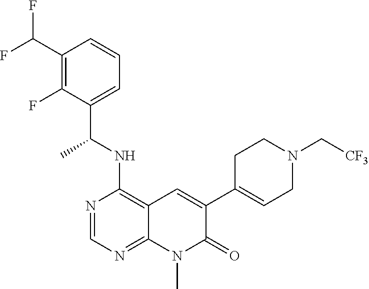 [SMILES: C[C@@H](Nc1ncnc2c1cc(C1=CCN(CC(F)(F)F)CC1)c(=O)n2C)c1cccc(C(F)F)c1F]

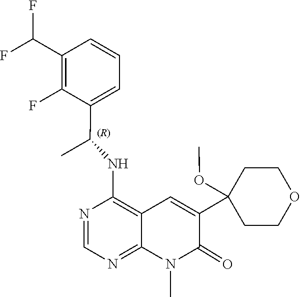

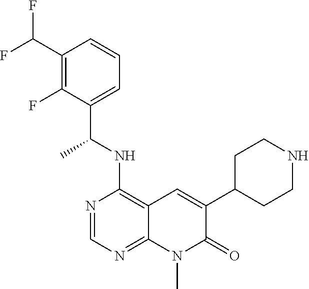 [SMILES: C[C@@H](Nc1ncnc2c1cc(C1CCNCC1)c(=O)n2C)c1cccc(C(F)F)c1F]

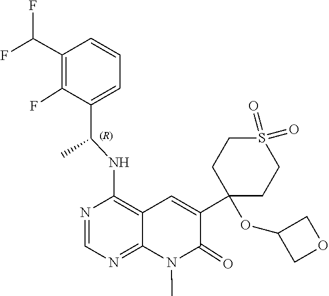

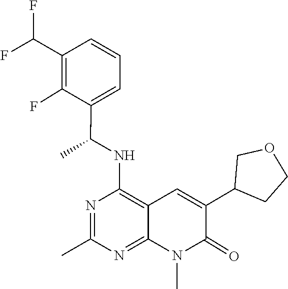 [SMILES: Cc1nc(N[C@H](C)c2cccc(C(F)F)c2F)c2cc(C3CCOC3)c(=O)n(C)c2n1]

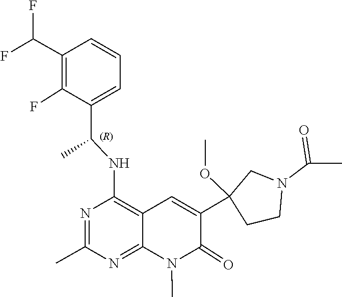

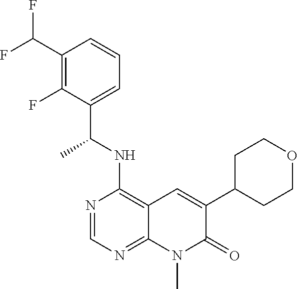 [SMILES: C[C@@H](Nc1ncnc2c1cc(C1CCOCC1)c(=O)n2C)c1cccc(C(F)F)c1F]

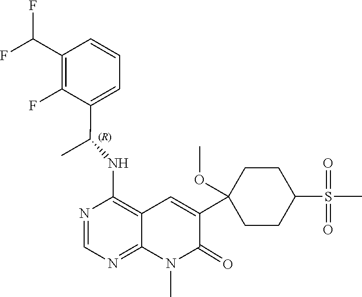

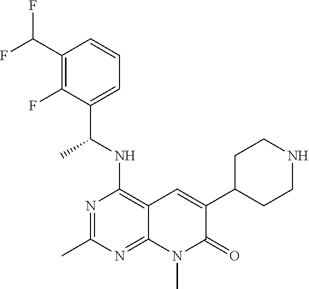 [SMILES: Cc1nc(N[C@H](C)c2cccc(C(F)F)c2F)c2cc(C3CCNCC3)c(=O)n(C)c2n1]

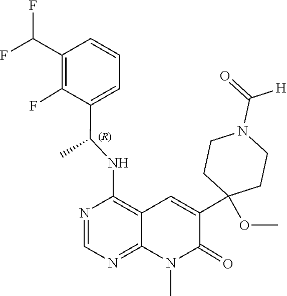

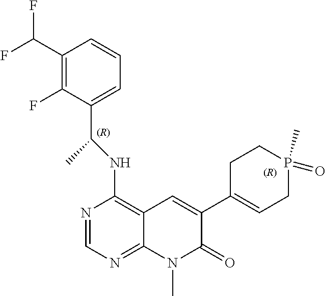

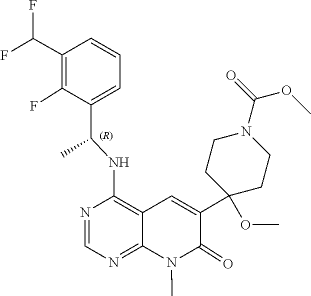

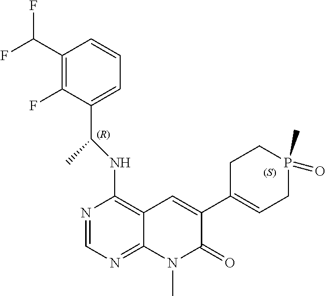

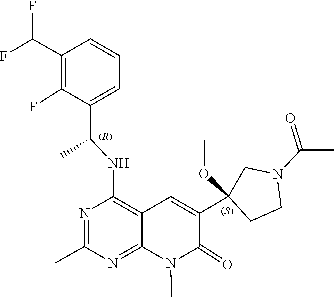

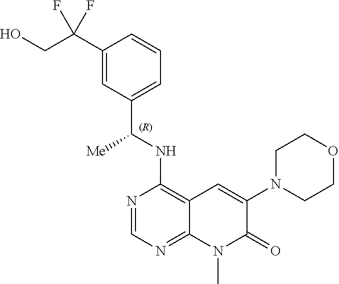

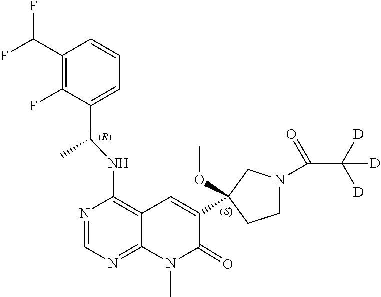

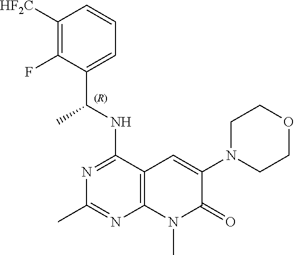

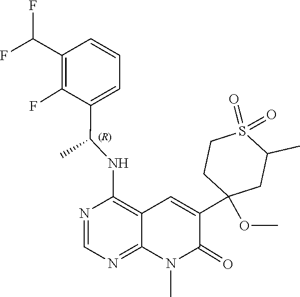

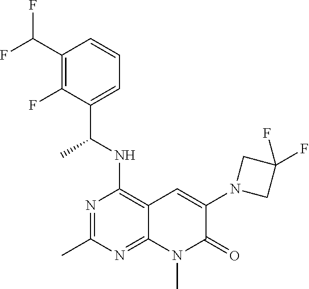 [SMILES: Cc1nc(N[C@H](C)c2cccc(C(F)F)c2F)c2cc(N3CC(F)(F)C3)c(=O)n(C)c2n1]

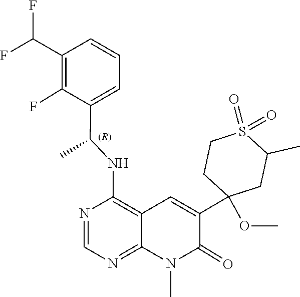

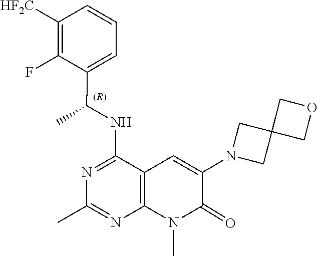

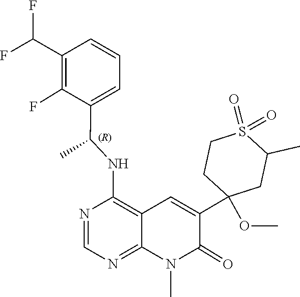

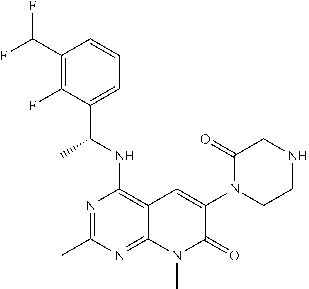 [SMILES: Cc1nc(N[C@H](C)c2cccc(C(F)F)c2F)c2cc(N3CCNCC3=O)c(=O)n(C)c2n1]

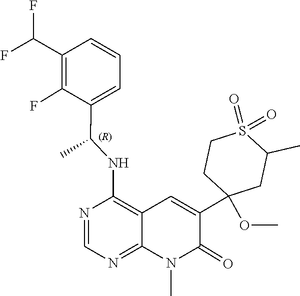

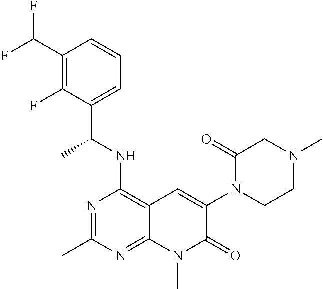 [SMILES: Cc1nc(N[C@H](C)c2cccc(C(F)F)c2F)c2cc(N3CCN(C)CC3=O)c(=O)n(C)c2n1]

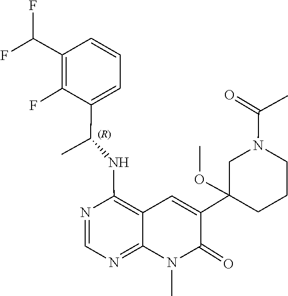

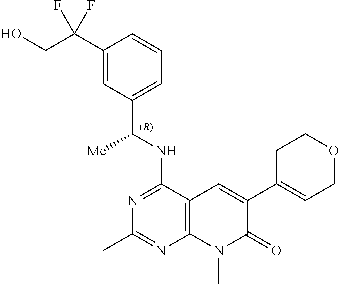

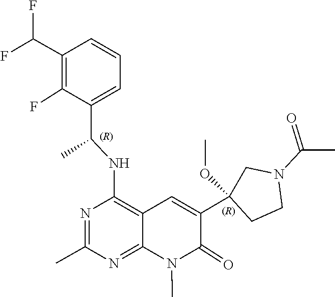

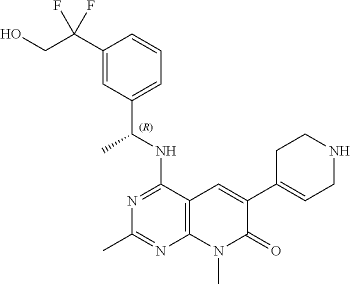

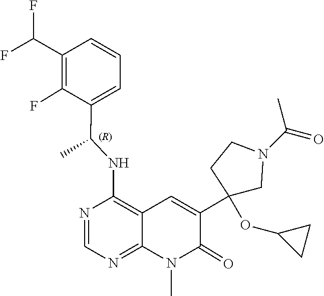

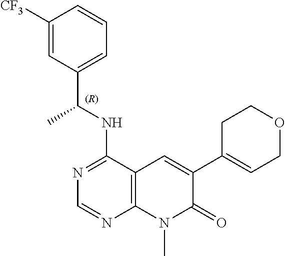

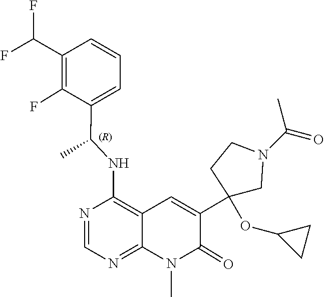

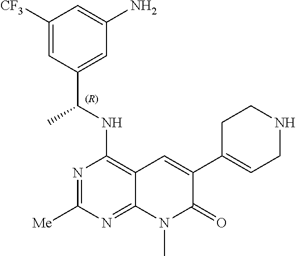

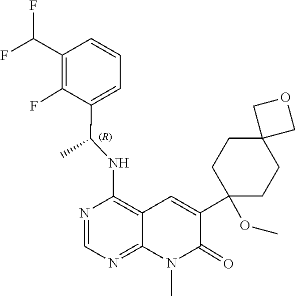

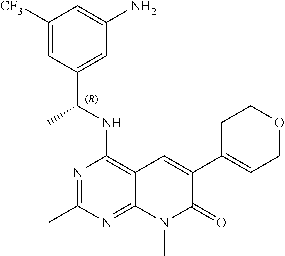

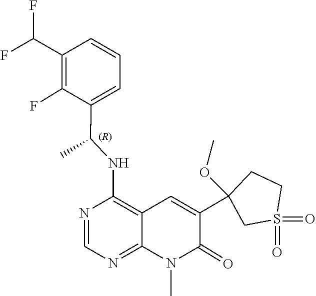

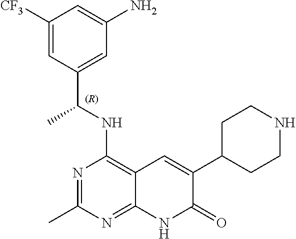

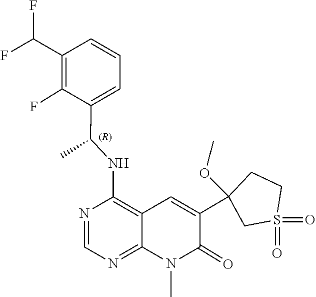

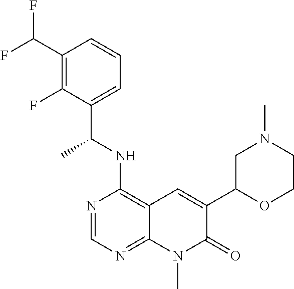 [SMILES: C[C@@H](Nc1ncnc2c1cc(C1CN(C)CCO1)c(=O)n2C)c1cccc(C(F)F)c1F]

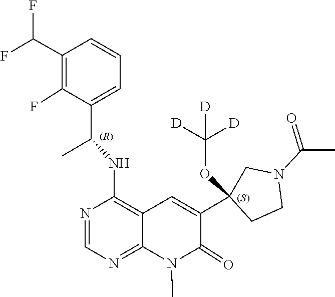

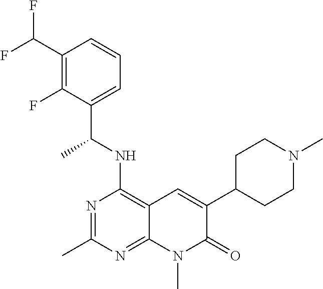 [SMILES: Cc1nc(N[C@H](C)c2cccc(C(F)F)c2F)c2cc(C3CCN(C)CC3)c(=O)n(C)c2n1]

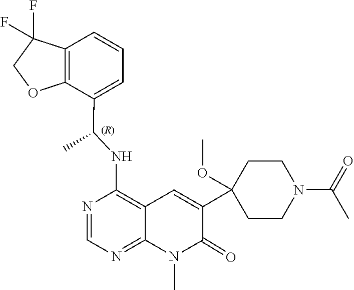

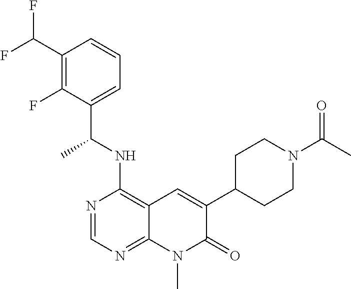 [SMILES: CC(=O)N1CCC(c2cc3c(N[C@H](C)c4cccc(C(F)F)c4F)ncnc3n(C)c2=O)CC1]

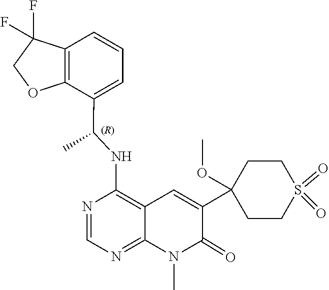

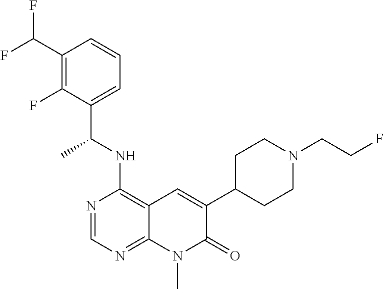 [SMILES: C[C@@H](Nc1ncnc2c1cc(C1CCN(CCF)CC1)c(=O)n2C)c1cccc(C(F)F)c1F]

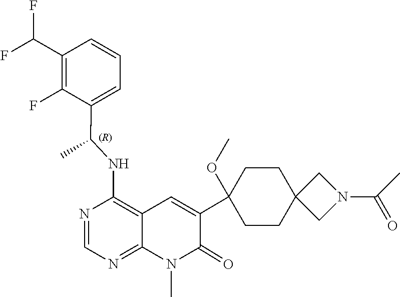

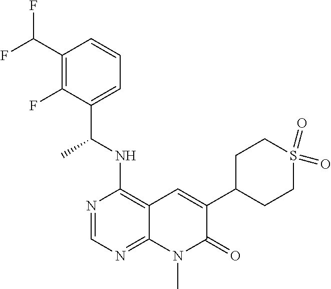 [SMILES: C[C@@H](Nc1ncnc2c1cc(C1CCS(=O)(=O)CC1)c(=O)n2C)c1cccc(C(F)F)c1F]

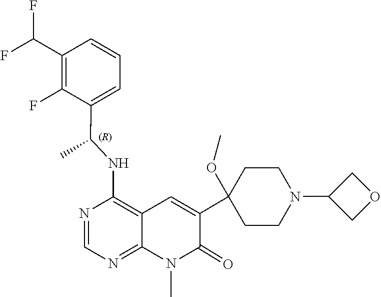

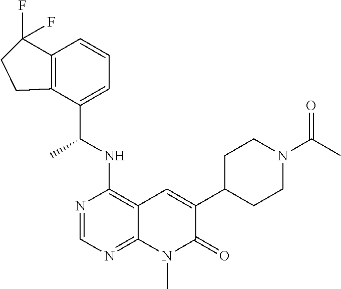 [SMILES: CC(=O)N1CCC(c2cc3c(N[C@H](C)c4cccc5c4CCC5(F)F)ncnc3n(C)c2=O)CC1]

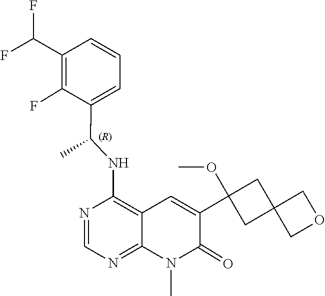

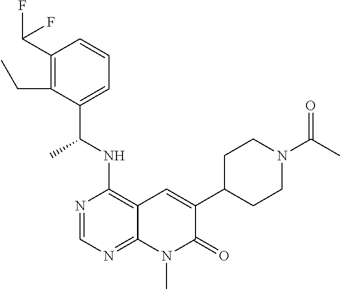 [SMILES: CCc1c(C(F)F)cccc1[C@@H](C)Nc1ncnc2c1cc(C1CCN(C(C)=O)CC1)c(=O)n2C]

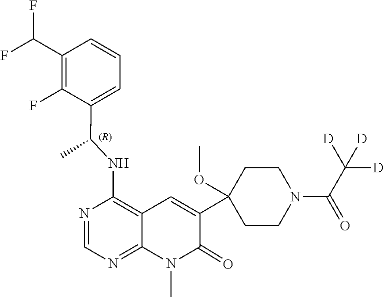

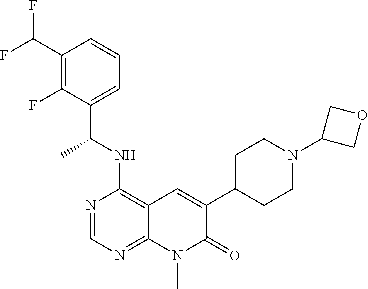 [SMILES: C[C@@H](Nc1ncnc2c1cc(C1CCN(C3COC3)CC1)c(=O)n2C)c1cccc(C(F)F)c1F]

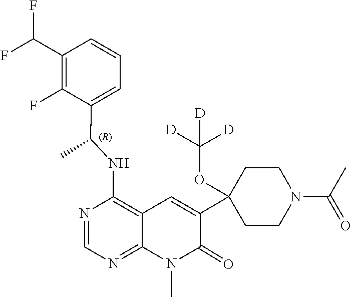

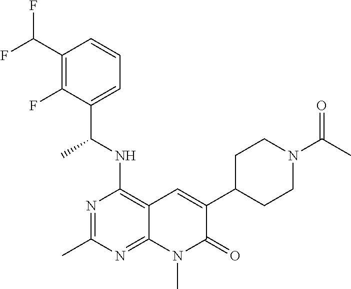 [SMILES: CC(=O)N1CCC(c2cc3c(N[C@H](C)c4cccc(C(F)F)c4F)nc(C)nc3n(C)c2=O)CC1]

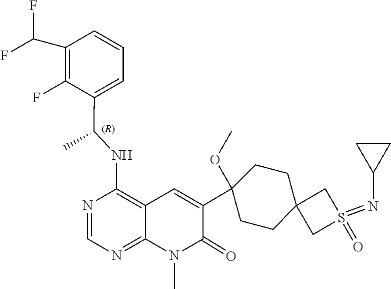

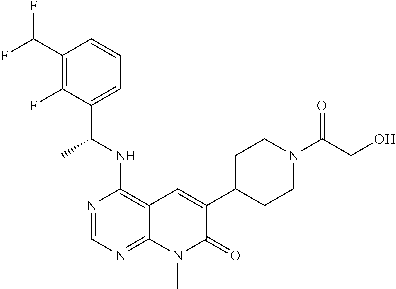 [SMILES: C[C@@H](Nc1ncnc2c1cc(C1CCN(C(=O)CO)CC1)c(=O)n2C)c1cccc(C(F)F)c1F]

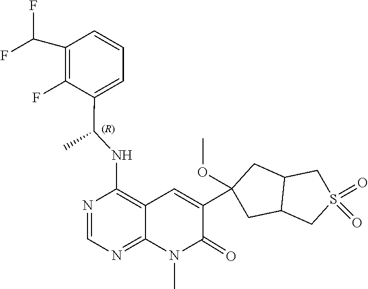

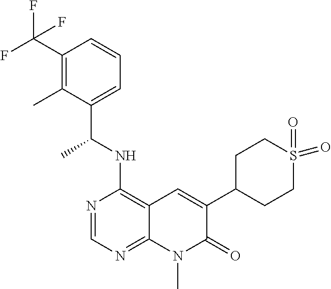 [SMILES: Cc1c([C@@H](C)Nc2ncnc3c2cc(C2CCS(=O)(=O)CC2)c(=O)n3C)cccc1C(F)(F)F]

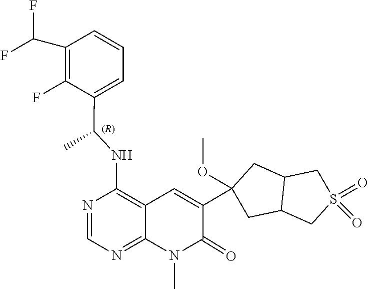

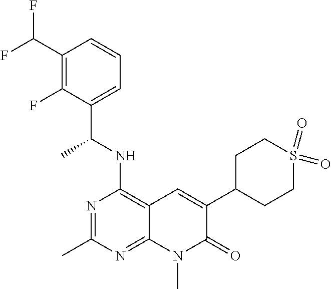 [SMILES: Cc1nc(N[C@H](C)c2cccc(C(F)F)c2F)c2cc(C3CCS(=O)(=O)CC3)c(=O)n(C)c2n1]

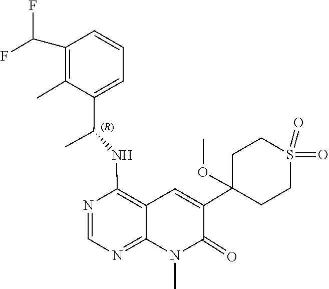

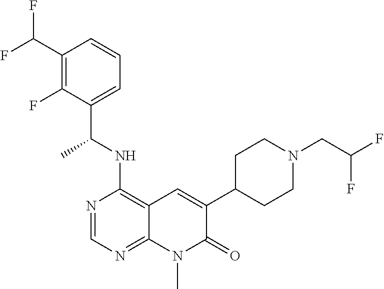 [SMILES: C[C@@H](Nc1ncnc2c1cc(C1CCN(CC(F)F)CC1)c(=O)n2C)c1cccc(C(F)F)c1F]

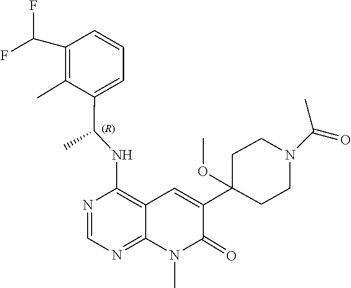

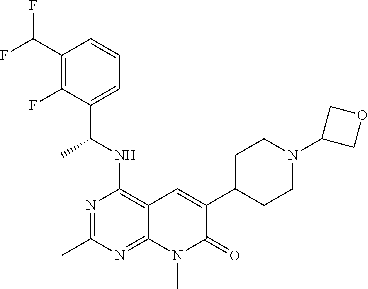 [SMILES: Cc1nc(N[C@H](C)c2cccc(C(F)F)c2F)c2cc(C3CCN(C4COC4)CC3)c(=O)n(C)c2n1]

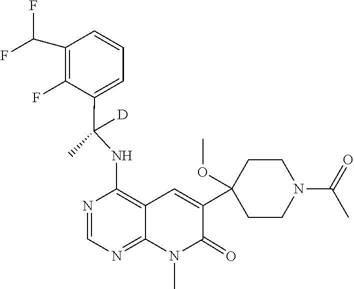 [SMILES: [2H][C@@](C)(Nc1ncnc2c1cc(C1(OC)CCN(C(C)=O)CC1)c(=O)n2C)c1cccc(C(F)F)c1F]

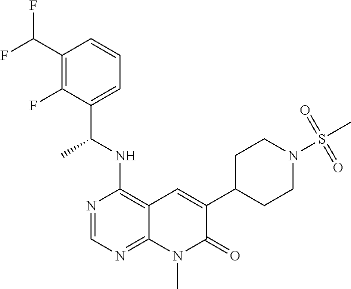 [SMILES: C[C@@H](Nc1ncnc2c1cc(C1CCN(S(C)(=O)=O)CC1)c(=O)n2C)c1cccc(C(F)F)c1F]

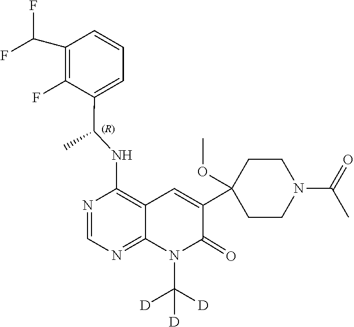

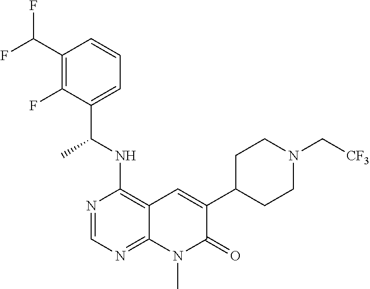 [SMILES: C[C@@H](Nc1ncnc2c1cc(C1CCN(CC(F)(F)F)CC1)c(=O)n2C)c1cccc(C(F)F)c1F]

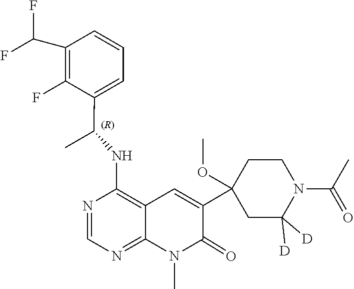

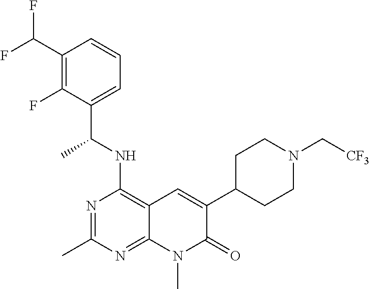 [SMILES: Cc1nc(N[C@H](C)c2cccc(C(F)F)c2F)c2cc(C3CCN(CC(F)(F)F)CC3)c(=O)n(C)c2n1]

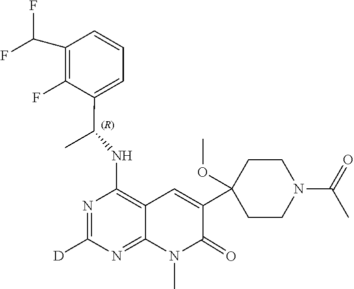

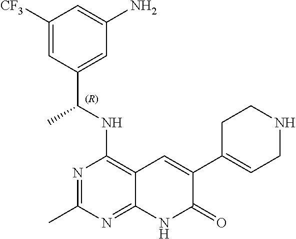

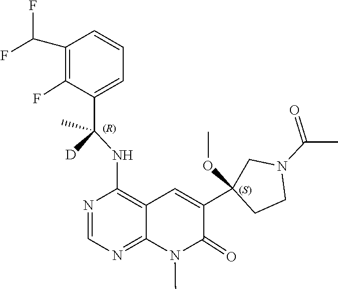

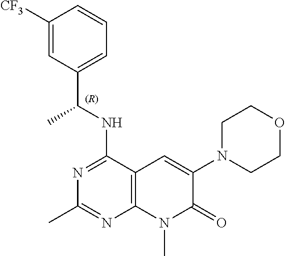

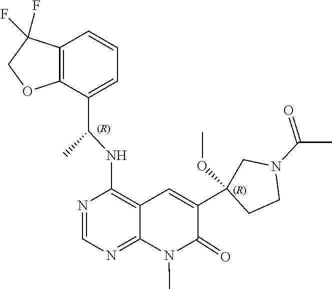

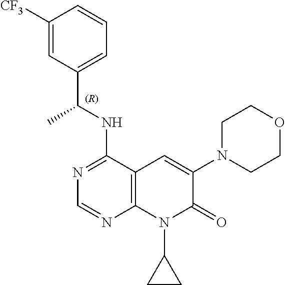

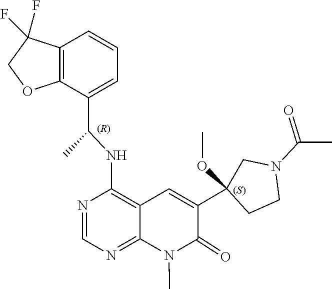

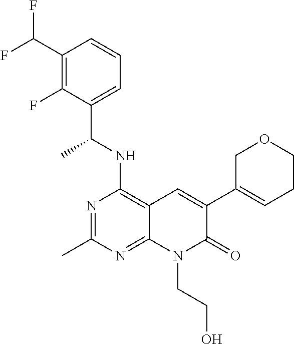 [SMILES: Cc1nc(N[C@H](C)c2cccc(C(F)F)c2F)c2cc(C3=CCCOC3)c(=O)n(CCO)c2n1]

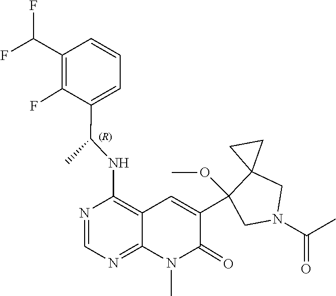

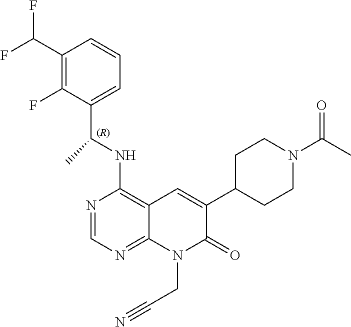

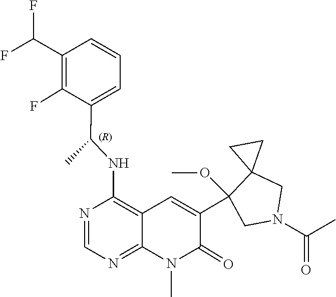

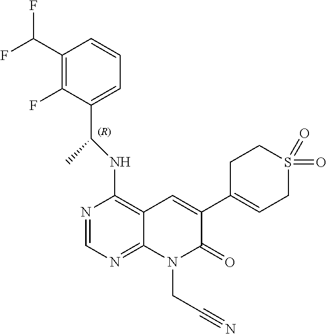

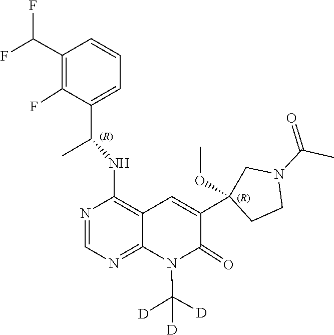

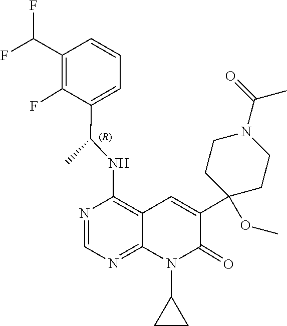

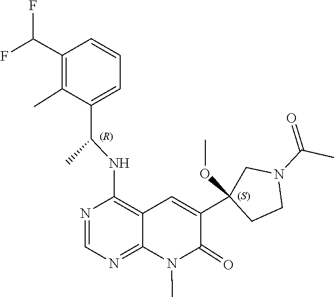

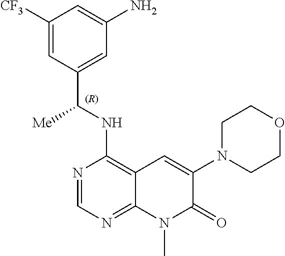

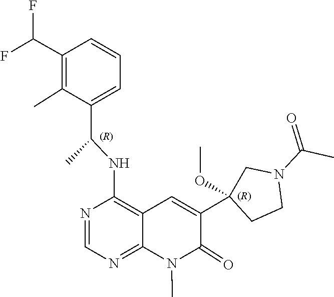

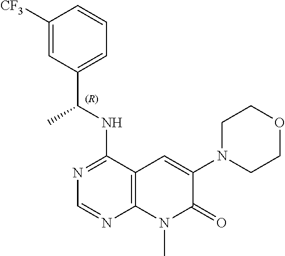

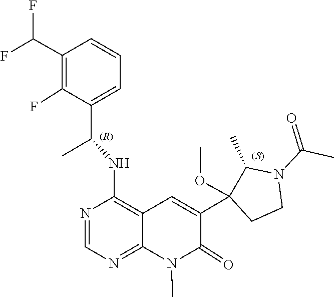

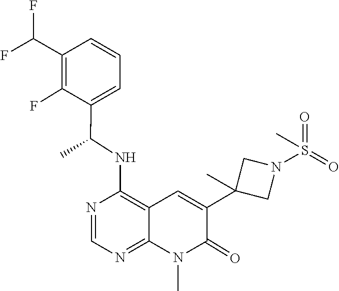 [SMILES: C[C@@H](Nc1ncnc2c1cc(C1(C)CN(S(C)(=O)=O)C1)c(=O)n2C)c1cccc(C(F)F)c1F]

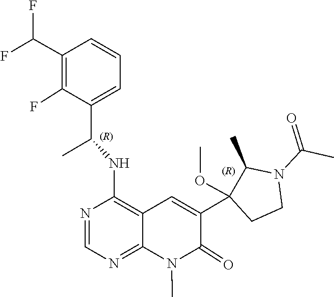

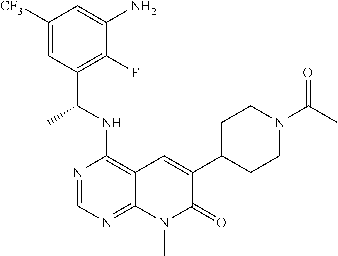 [SMILES: CC(=O)N1CCC(c2cc3c(N[C@H](C)c4cc(C(F)(F)F)cc(N)c4F)ncnc3n(C)c2=O)CC1]

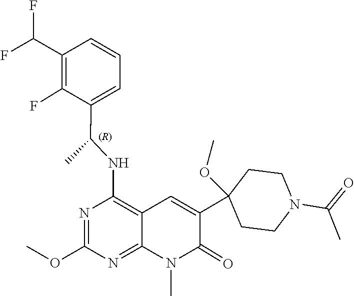

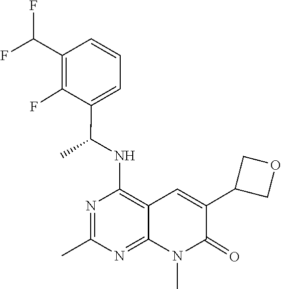 [SMILES: Cc1nc(N[C@H](C)c2cccc(C(F)F)c2F)c2cc(C3COC3)c(=O)n(C)c2n1]

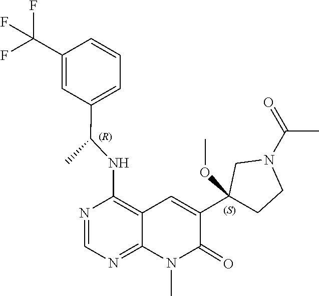

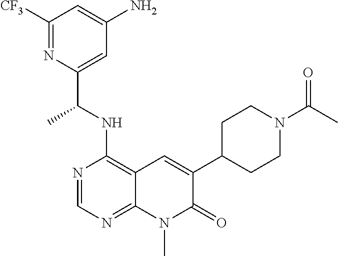 [SMILES: CC(=O)N1CCC(c2cc3c(N[C@H](C)c4cc(N)cc(C(F)(F)F)n4)ncnc3n(C)c2=O)CC1]

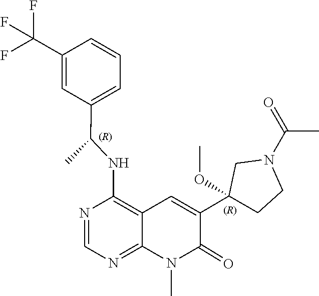

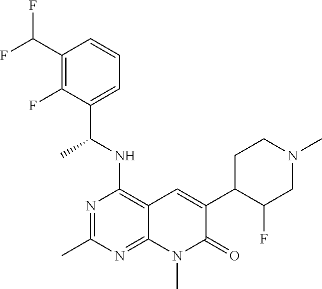 [SMILES: Cc1nc(N[C@H](C)c2cccc(C(F)F)c2F)c2cc(C3CCN(C)CC3F)c(=O)n(C)c2n1]

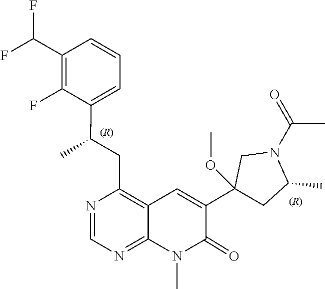

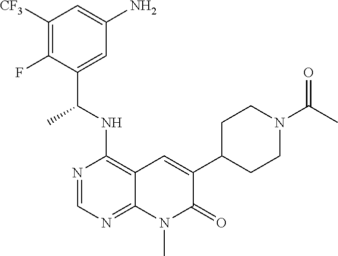 [SMILES: CC(=O)N1CCC(c2cc3c(N[C@H](C)c4cc(N)cc(C(F)(F)F)c4F)ncnc3n(C)c2=O)CC1]

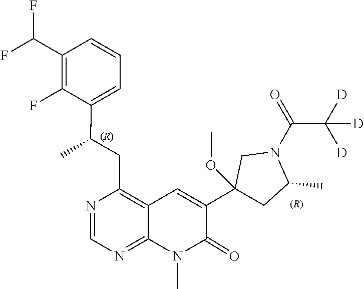

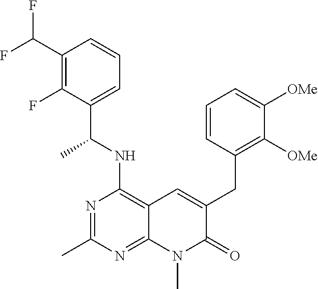 [SMILES: COc1cccc(Cc2cc3c(N[C@H](C)c4cccc(C(F)F)c4F)nc(C)nc3n(C)c2=O)c1OC]

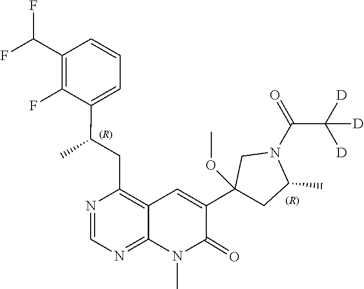

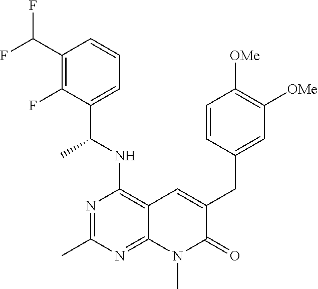 [SMILES: COc1ccc(Cc2cc3c(N[C@H](C)c4cccc(C(F)F)c4F)nc(C)nc3n(C)c2=O)cc1OC]

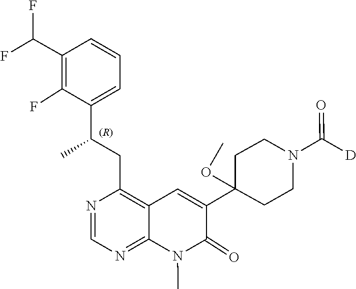

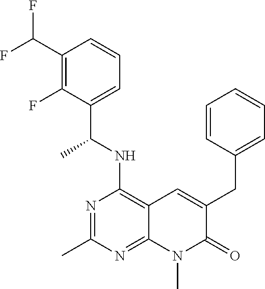 [SMILES: Cc1nc(N[C@H](C)c2cccc(C(F)F)c2F)c2cc(Cc3ccccc3)c(=O)n(C)c2n1]

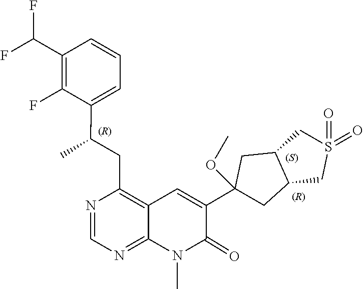

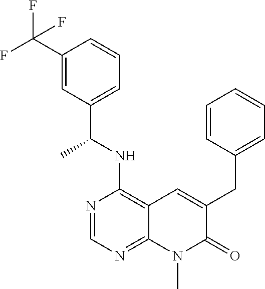 [SMILES: C[C@@H](Nc1ncnc2c1cc(Cc1ccccc1)c(=O)n2C)c1cccc(C(F)(F)F)c1]

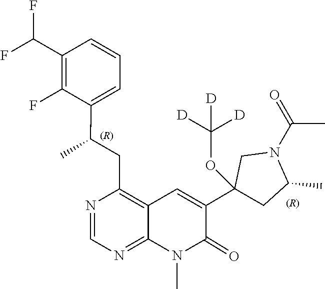

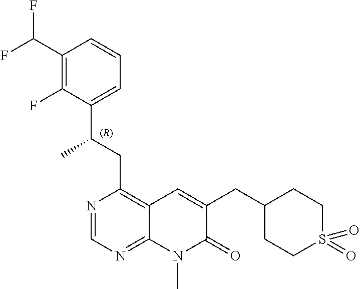

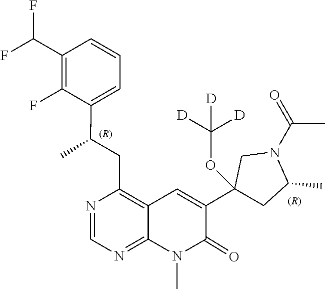

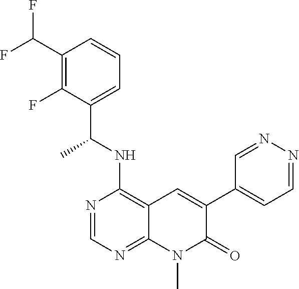 [SMILES: C[C@@H](Nc1ncnc2c1cc(-c1ccnnc1)c(=O)n2C)c1cccc(C(F)F)c1F]

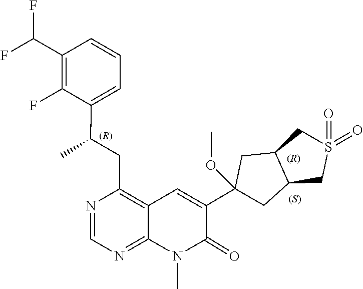

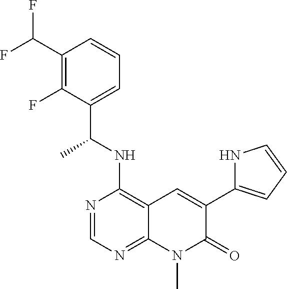 [SMILES: C[C@@H](Nc1ncnc2c1cc(-c1ccc[nH]1)c(=O)n2C)c1cccc(C(F)F)c1F]

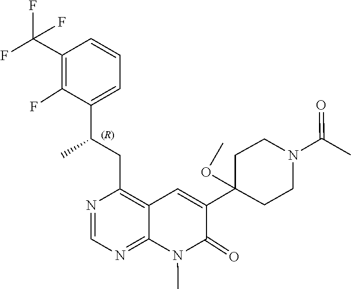

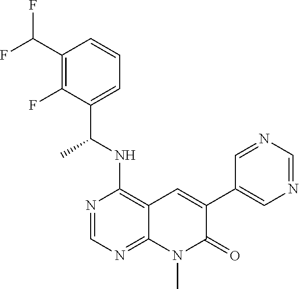 [SMILES: C[C@@H](Nc1ncnc2c1cc(-c1cncnc1)c(=O)n2C)c1cccc(C(F)F)c1F]

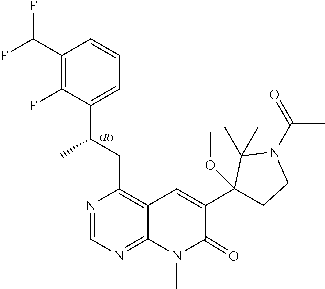

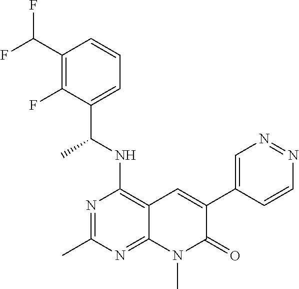 [SMILES: Cc1nc(N[C@H](C)c2cccc(C(F)F)c2F)c2cc(-c3ccnnc3)c(=O)n(C)c2n1]

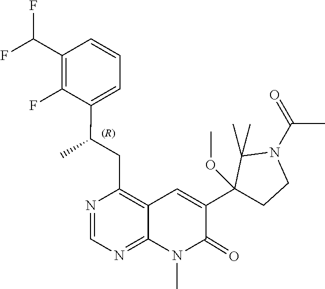

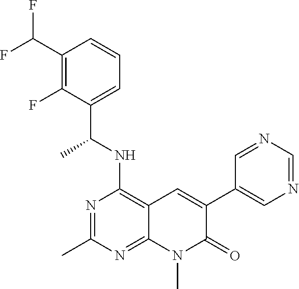 [SMILES: Cc1nc(N[C@H](C)c2cccc(C(F)F)c2F)c2cc(-c3cncnc3)c(=O)n(C)c2n1]

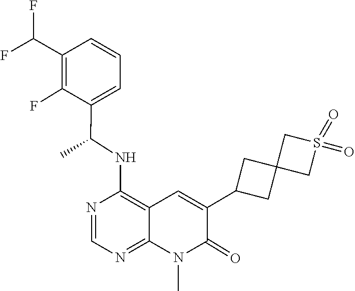 [SMILES: C[C@@H](Nc1ncnc2c1cc(C1CC3(C1)CS(=O)(=O)C3)c(=O)n2C)c1cccc(C(F)F)c1F]

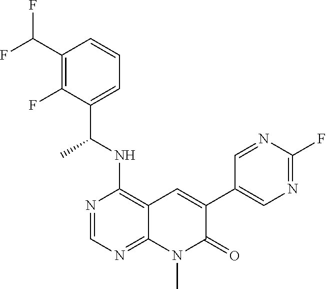 [SMILES: C[C@@H](Nc1ncnc2c1cc(-c1cnc(F)nc1)c(=O)n2C)c1cccc(C(F)F)c1F]

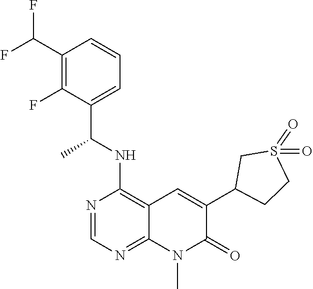 [SMILES: C[C@@H](Nc1ncnc2c1cc(C1CCS(=O)(=O)C1)c(=O)n2C)c1cccc(C(F)F)c1F]

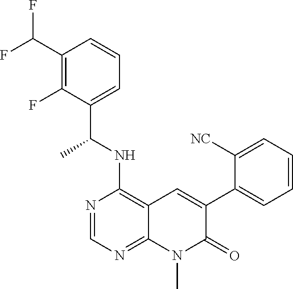 [SMILES: C[C@@H](Nc1ncnc2c1cc(-c1ccccc1C#N)c(=O)n2C)c1cccc(C(F)F)c1F]

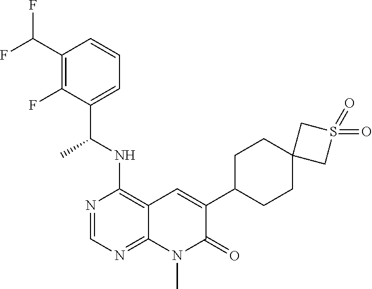 [SMILES: C[C@@H](Nc1ncnc2c1cc(C1CCC3(CC1)CS(=O)(=O)C3)c(=O)n2C)c1cccc(C(F)F)c1F]

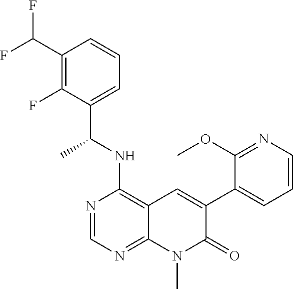 [SMILES: COc1ncccc1-c1cc2c(N[C@H](C)c3cccc(C(F)F)c3F)ncnc2n(C)c1=O]

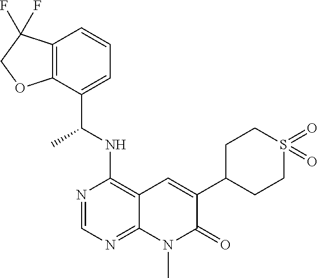 [SMILES: C[C@@H](Nc1ncnc2c1cc(C1CCS(=O)(=O)CC1)c(=O)n2C)c1cccc2c1OCC2(F)F]

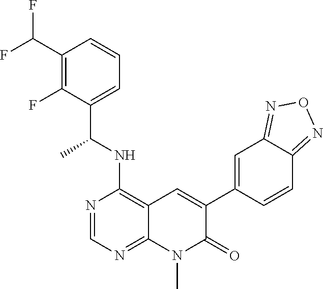 [SMILES: C[C@@H](Nc1ncnc2c1cc(-c1ccc3nonc3c1)c(=O)n2C)c1cccc(C(F)F)c1F]

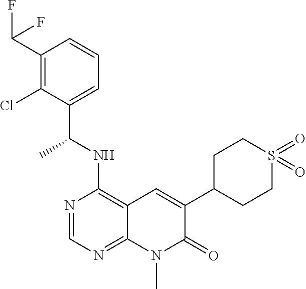 [SMILES: C[C@@H](Nc1ncnc2c1cc(C1CCS(=O)(=O)CC1)c(=O)n2C)c1cccc(C(F)F)c1Cl]

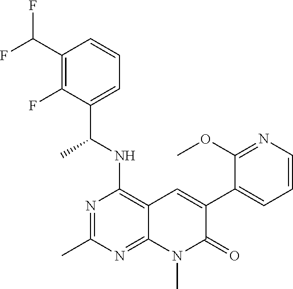 [SMILES: COc1ncccc1-c1cc2c(N[C@H](C)c3cccc(C(F)F)c3F)nc(C)nc2n(C)c1=O]

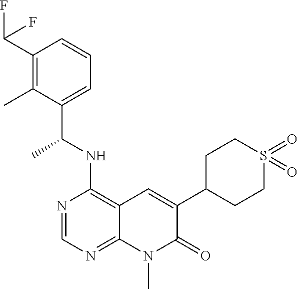 [SMILES: Cc1c(C(F)F)cccc1[C@@H](C)Nc1ncnc2c1cc(C1CCS(=O)(=O)CC1)c(=O)n2C]

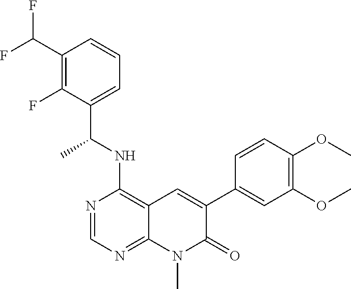 [SMILES: COc1ccc(-c2cc3c(N[C@H](C)c4cccc(C(F)F)c4F)ncnc3n(C)c2=O)cc1OC]

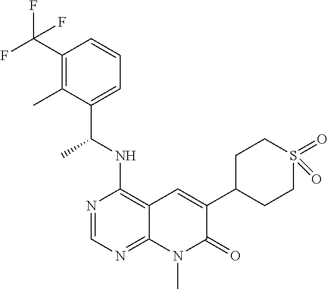 [SMILES: Cc1c([C@@H](C)Nc2ncnc3c2cc(C2CCS(=O)(=O)CC2)c(=O)n3C)cccc1C(F)(F)F]

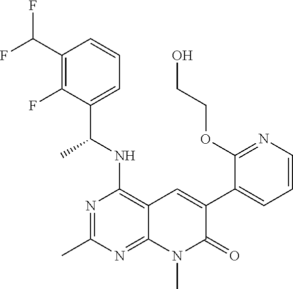 [SMILES: Cc1nc(N[C@H](C)c2cccc(C(F)F)c2F)c2cc(-c3cccnc3OCCO)c(=O)n(C)c2n1]

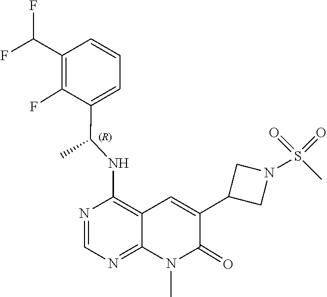

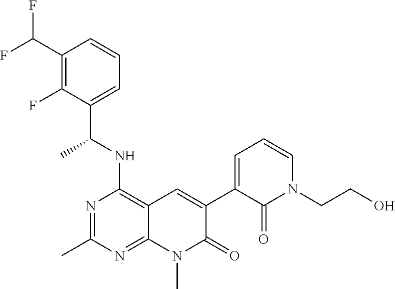 [SMILES: Cc1nc(N[C@H](C)c2cccc(C(F)F)c2F)c2cc(-c3cccn(CCO)c3=O)c(=O)n(C)c2n1]

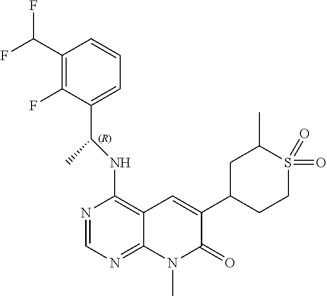

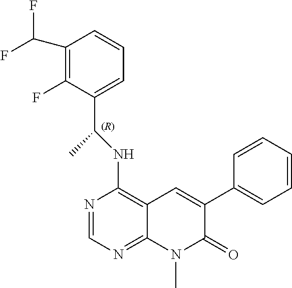

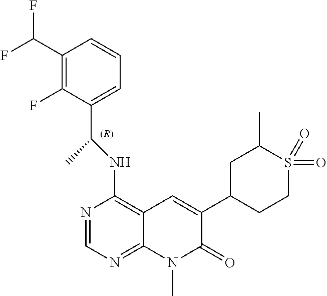

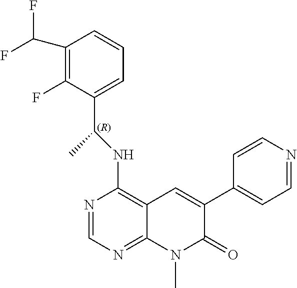

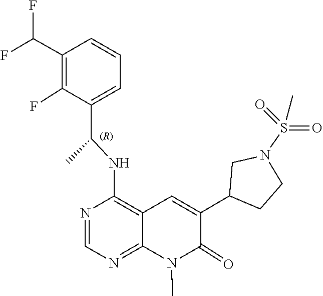

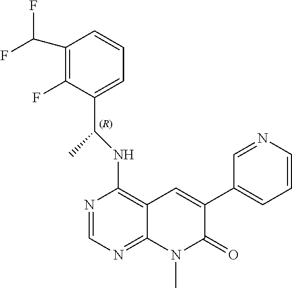

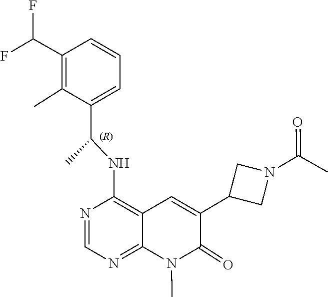

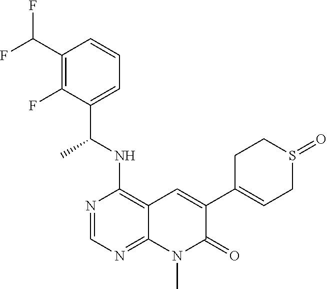 [SMILES: C[C@@H](Nc1ncnc2c1cc(C1=CCS(=O)CC1)c(=O)n2C)c1cccc(C(F)F)c1F]

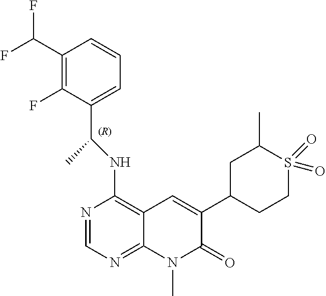

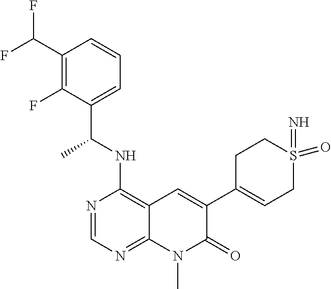 [SMILES: C[C@@H](Nc1ncnc2c1cc(C1=CCS(=N)(=O)CC1)c(=O)n2C)c1cccc(C(F)F)c1F]

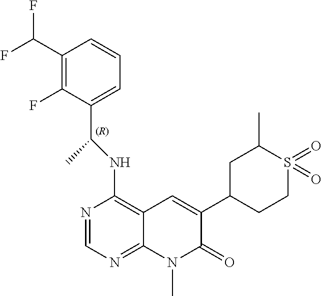

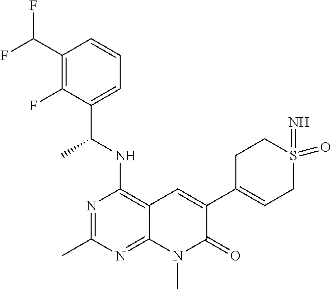 [SMILES: Cc1nc(N[C@H](C)c2cccc(C(F)F)c2F)c2cc(C3=CCS(=N)(=O)CC3)c(=O)n(C)c2n1]

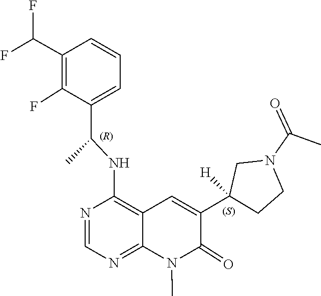

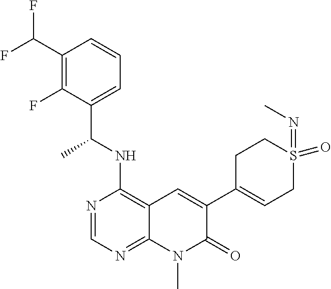 [SMILES: CN=S1(=O)CC=C(c2cc3c(N[C@H](C)c4cccc(C(F)F)c4F)ncnc3n(C)c2=O)CC1]

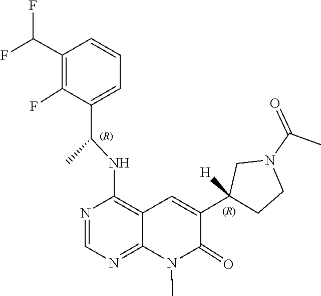

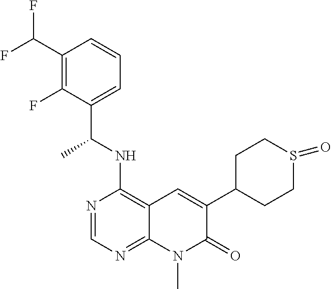 [SMILES: C[C@@H](Nc1ncnc2c1cc(C1CCS(=O)CC1)c(=O)n2C)c1cccc(C(F)F)c1F]

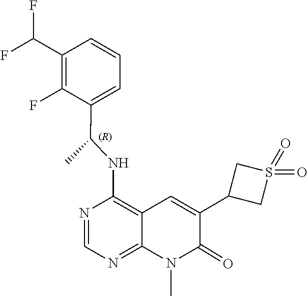

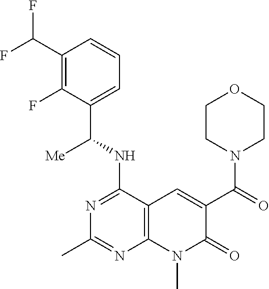 [SMILES: Cc1nc(N[C@H](C)c2cccc(C(F)F)c2F)c2cc(C(=O)N3CCOCC3)c(=O)n(C)c2n1]

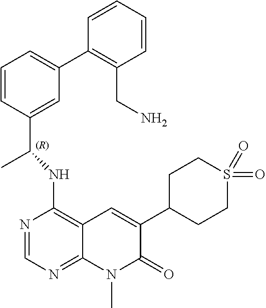

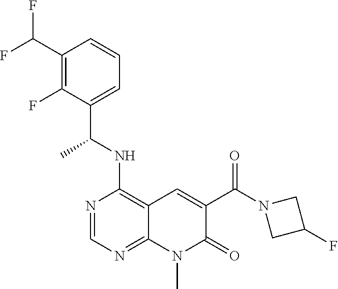 [SMILES: C[C@@H](Nc1ncnc2c1cc(C(=O)N1CC(F)C1)c(=O)n2C)c1cccc(C(F)F)c1F]

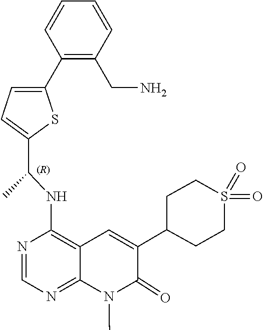

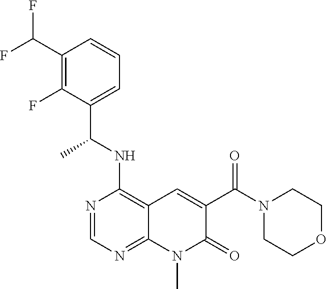 [SMILES: C[C@@H](Nc1ncnc2c1cc(C(=O)N1CCOCC1)c(=O)n2C)c1cccc(C(F)F)c1F]

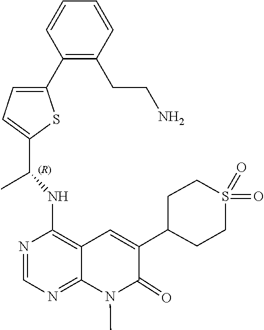

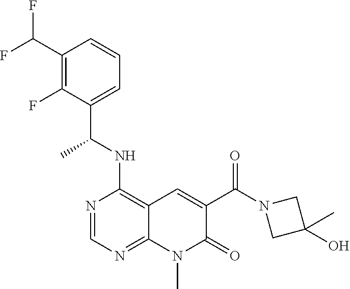 [SMILES: C[C@@H](Nc1ncnc2c1cc(C(=O)N1CC(C)(O)C1)c(=O)n2C)c1cccc(C(F)F)c1F]

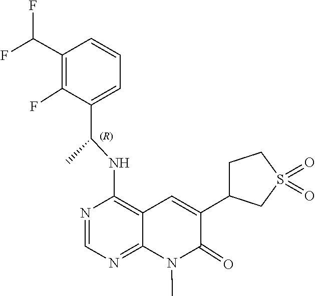

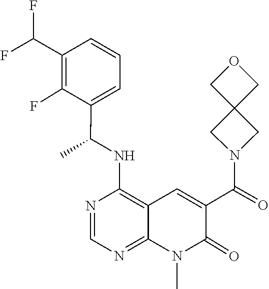 [SMILES: C[C@@H](Nc1ncnc2c1cc(C(=O)N1CC3(COC3)C1)c(=O)n2C)c1cccc(C(F)F)c1F]

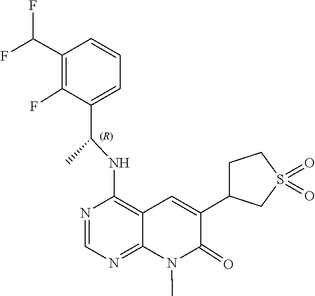

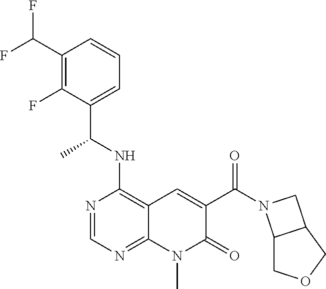 [SMILES: C[C@@H](Nc1ncnc2c1cc(C(=O)N1CC3COCC31)c(=O)n2C)c1cccc(C(F)F)c1F]

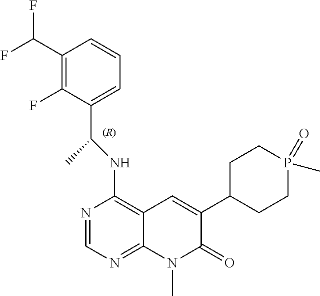

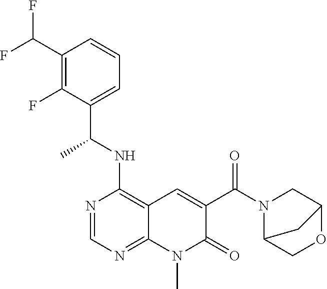 [SMILES: C[C@@H](Nc1ncnc2c1cc(C(=O)N1CC3CC1CO3)c(=O)n2C)c1cccc(C(F)F)c1F]

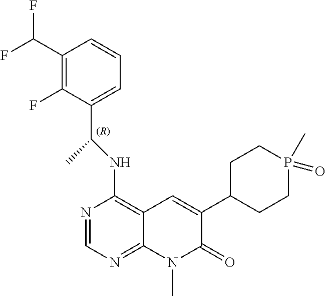

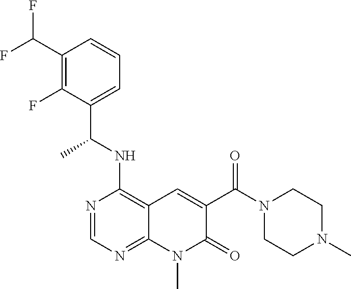 [SMILES: C[C@@H](Nc1ncnc2c1cc(C(=O)N1CCN(C)CC1)c(=O)n2C)c1cccc(C(F)F)c1F]

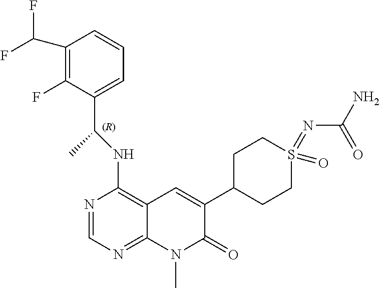

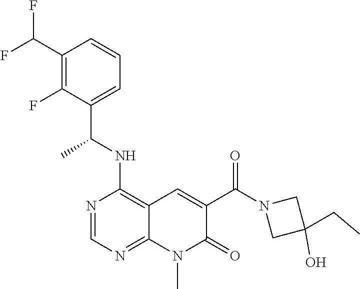 [SMILES: CCC1(O)CN(C(=O)c2cc3c(N[C@H](C)c4cccc(C(F)F)c4F)ncnc3n(C)c2=O)C1]

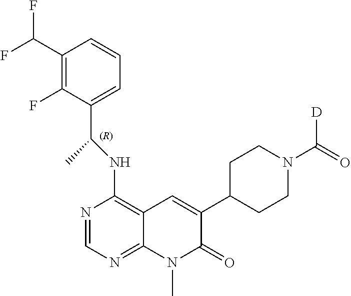

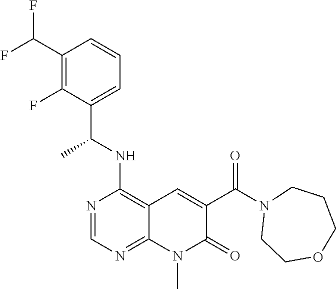 [SMILES: C[C@@H](Nc1ncnc2c1cc(C(=O)N1CCCOCC1)c(=O)n2C)c1cccc(C(F)F)c1F]

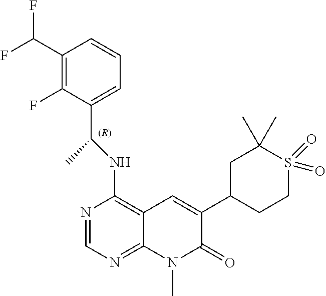

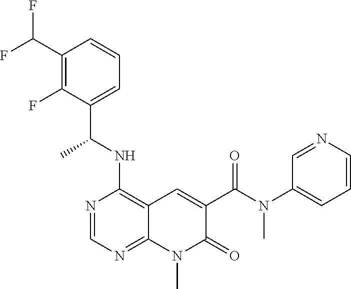 [SMILES: C[C@@H](Nc1ncnc2c1cc(C(=O)N(C)c1cccnc1)c(=O)n2C)c1cccc(C(F)F)c1F]

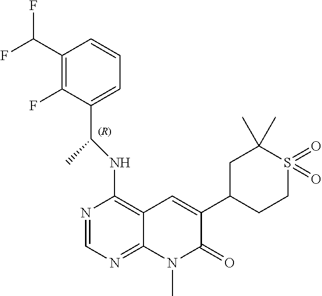

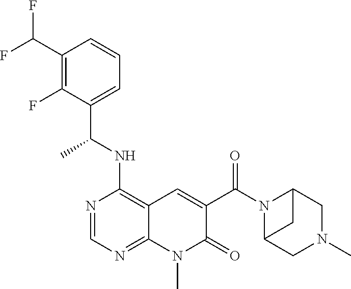 [SMILES: C[C@@H](Nc1ncnc2c1cc(C(=O)N1C3CC1CN(C)C3)c(=O)n2C)c1cccc(C(F)F)c1F]

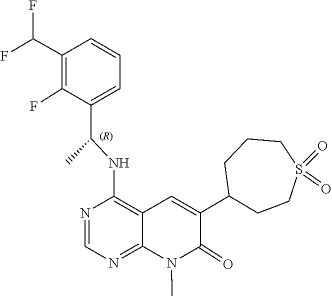

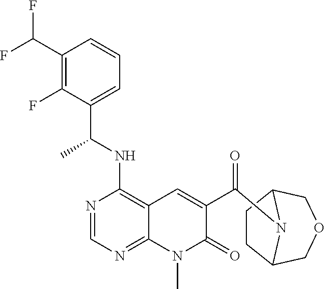 [SMILES: C[C@@H](Nc1ncnc2c1cc(C(=O)N1C3CCC1COC3)c(=O)n2C)c1cccc(C(F)F)c1F]

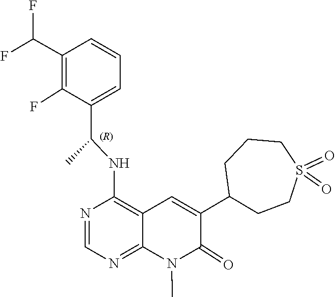

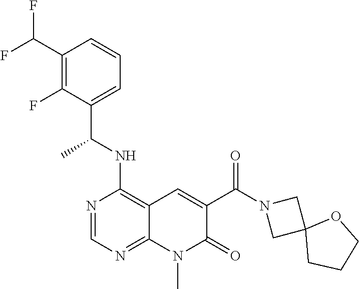 [SMILES: C[C@@H](Nc1ncnc2c1cc(C(=O)N1CC3(CCCO3)C1)c(=O)n2C)c1cccc(C(F)F)c1F]

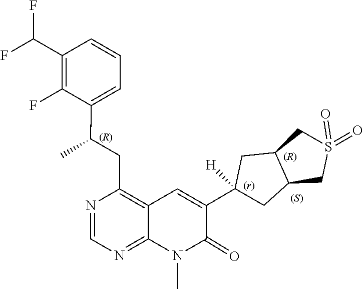

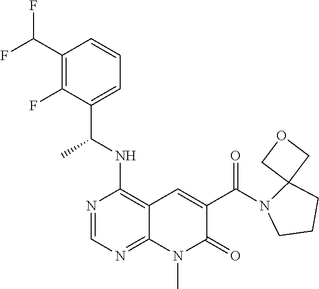 [SMILES: C[C@@H](Nc1ncnc2c1cc(C(=O)N1CCCC13COC3)c(=O)n2C)c1cccc(C(F)F)c1F]

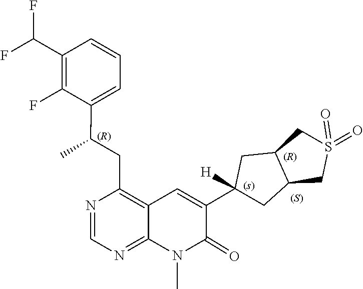

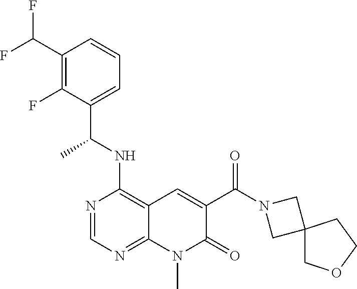 [SMILES: C[C@@H](Nc1ncnc2c1cc(C(=O)N1CC3(CCOC3)C1)c(=O)n2C)c1cccc(C(F)F)c1F]

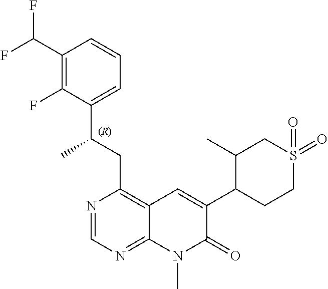

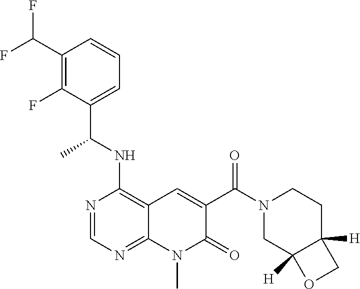 [SMILES: C[C@@H](Nc1ncnc2c1cc(C(=O)N1CC[C@@H]3CO[C@@H]3C1)c(=O)n2C)c1cccc(C(F)F)c1F]

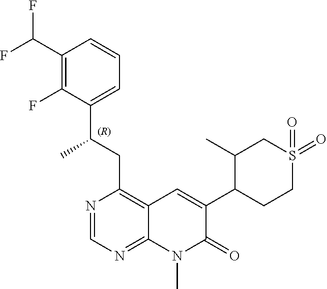

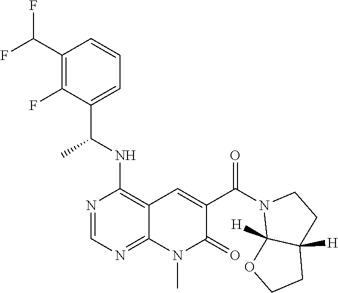 [SMILES: C[C@@H](Nc1ncnc2c1cc(C(=O)N1CC[C@@H]3CCO[C@@H]31)c(=O)n2C)c1cccc(C(F)F)c1F]

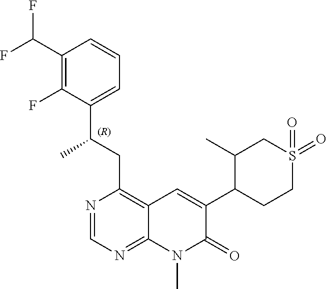

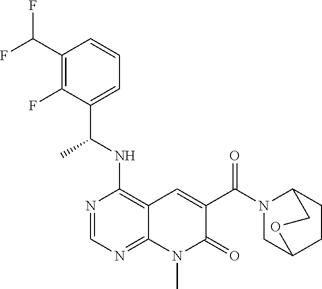 [SMILES: C[C@@H](Nc1ncnc2c1cc(C(=O)N1CC3CCC1CO3)c(=O)n2C)c1cccc(C(F)F)c1F]

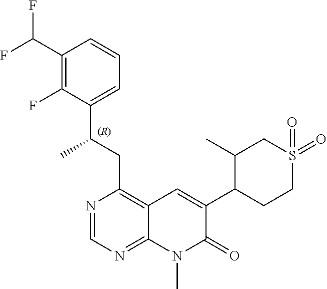

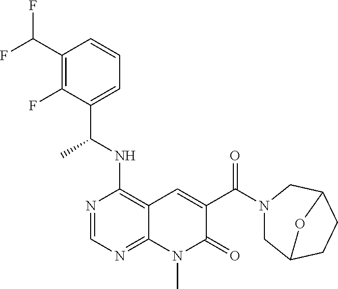 [SMILES: C[C@@H](Nc1ncnc2c1cc(C(=O)N1CC3CCC(C1)O3)c(=O)n2C)c1cccc(C(F)F)c1F]

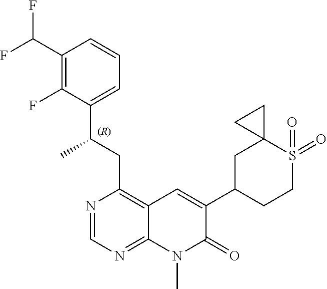

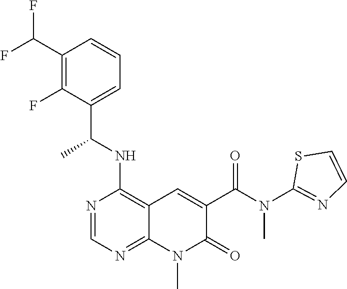 [SMILES: C[C@@H](Nc1ncnc2c1cc(C(=O)N(C)c1nccs1)c(=O)n2C)c1cccc(C(F)F)c1F]

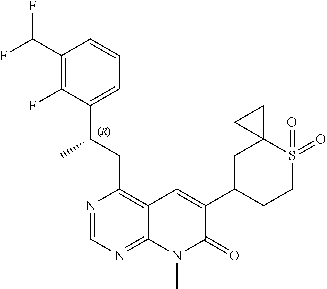

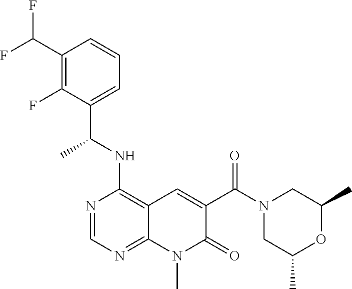 [SMILES: C[C@@H]1CN(C(=O)c2cc3c(N[C@H](C)c4cccc(C(F)F)c4F)ncnc3n(C)c2=O)C[C@@H](C)O1]

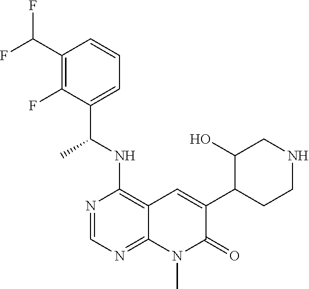 [SMILES: C[C@@H](Nc1ncnc2c1cc(C1CCNCC1O)c(=O)n2C)c1cccc(C(F)F)c1F]

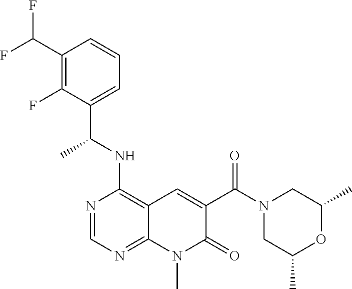 [SMILES: C[C@@H]1CN(C(=O)c2cc3c(N[C@H](C)c4cccc(C(F)F)c4F)ncnc3n(C)c2=O)C[C@H](C)O1]

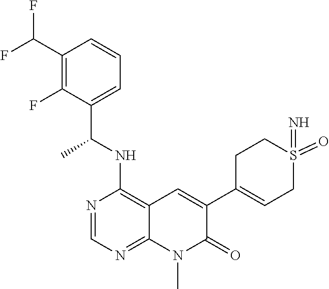 [SMILES: C[C@@H](Nc1ncnc2c1cc(C1=CCS(=N)(=O)CC1)c(=O)n2C)c1cccc(C(F)F)c1F]

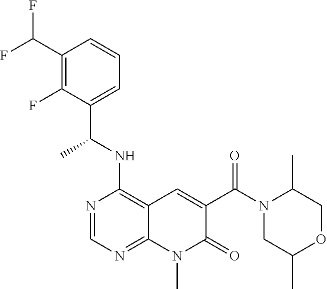 [SMILES: CC1CN(C(=O)c2cc3c(N[C@H](C)c4cccc(C(F)F)c4F)ncnc3n(C)c2=O)C(C)CO1]

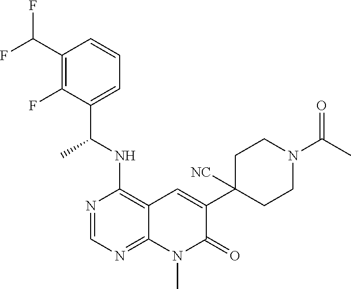 [SMILES: CC(=O)N1CCC(C#N)(c2cc3c(N[C@H](C)c4cccc(C(F)F)c4F)ncnc3n(C)c2=O)CC1]

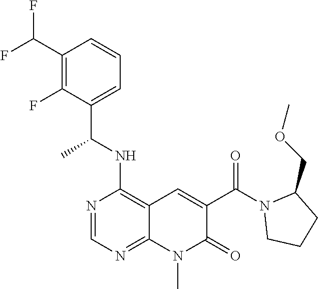 [SMILES: COC[C@H]1CCCN1C(=O)c1cc2c(N[C@H](C)c3cccc(C(F)F)c3F)ncnc2n(C)c1=O]

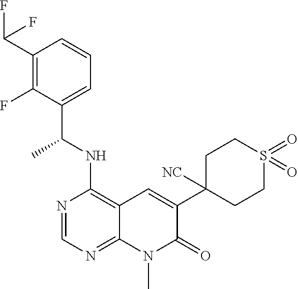 [SMILES: C[C@@H](Nc1ncnc2c1cc(C1(C#N)CCS(=O)(=O)CC1)c(=O)n2C)c1cccc(C(F)F)c1F]

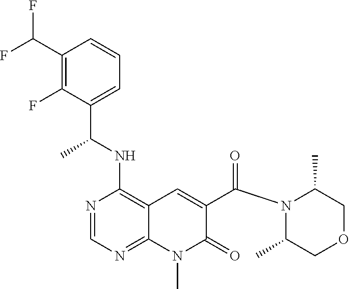 [SMILES: C[C@@H]1COC[C@H](C)N1C(=O)c1cc2c(N[C@H](C)c3cccc(C(F)F)c3F)ncnc2n(C)c1=O]

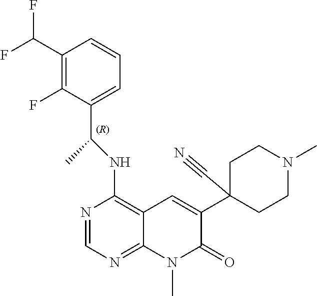

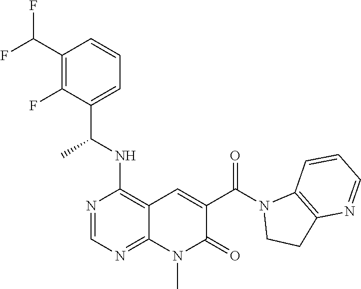 [SMILES: C[C@@H](Nc1ncnc2c1cc(C(=O)N1CCc3ncccc31)c(=O)n2C)c1cccc(C(F)F)c1F]

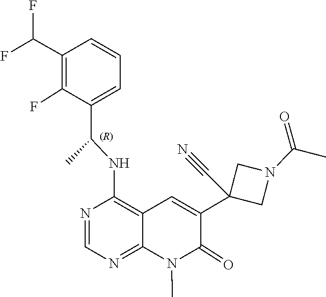

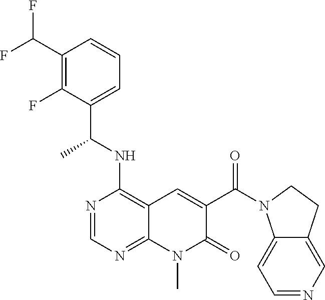 [SMILES: C[C@@H](Nc1ncnc2c1cc(C(=O)N1CCc3cnccc31)c(=O)n2C)c1cccc(C(F)F)c1F]

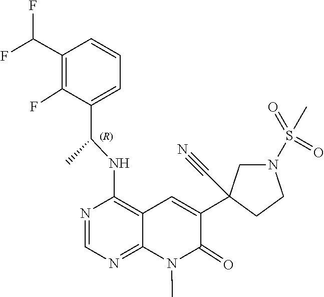

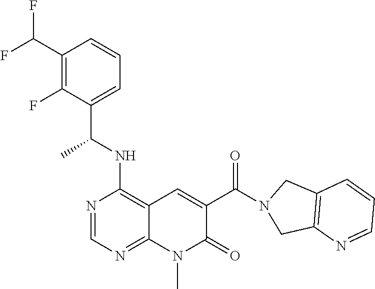 [SMILES: C[C@@H](Nc1ncnc2c1cc(C(=O)N1Cc3cccnc3C1)c(=O)n2C)c1cccc(C(F)F)c1F]

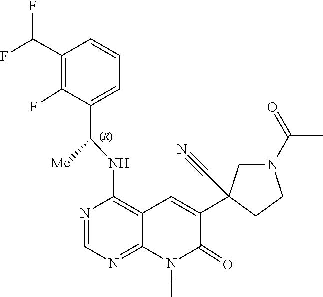

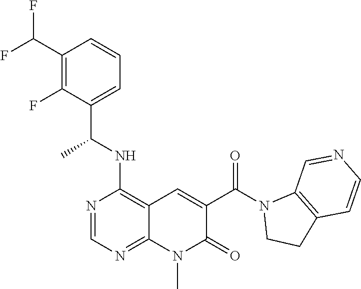 [SMILES: C[C@@H](Nc1ncnc2c1cc(C(=O)N1CCc3ccncc31)c(=O)n2C)c1cccc(C(F)F)c1F]

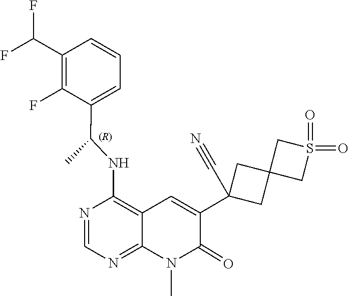

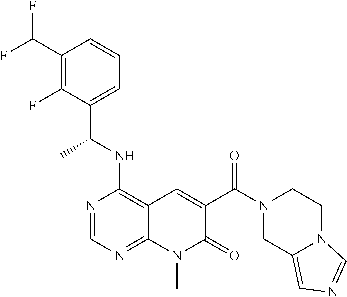 [SMILES: C[C@@H](Nc1ncnc2c1cc(C(=O)N1CCn3cncc3C1)c(=O)n2C)c1cccc(C(F)F)c1F]

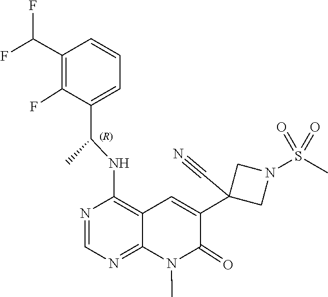

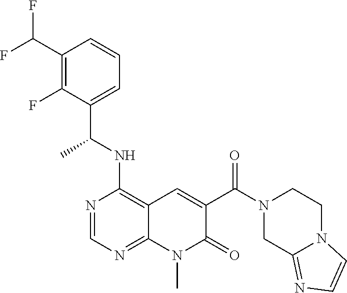 [SMILES: C[C@@H](Nc1ncnc2c1cc(C(=O)N1CCn3ccnc3C1)c(=O)n2C)c1cccc(C(F)F)c1F]

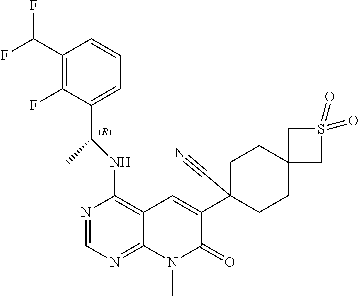

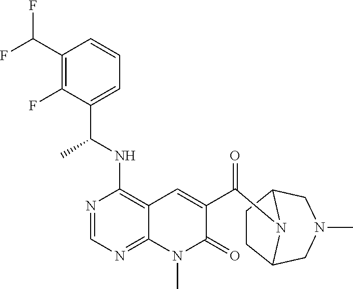 [SMILES: C[C@@H](Nc1ncnc2c1cc(C(=O)N1C3CCC1CN(C)C3)c(=O)n2C)c1cccc(C(F)F)c1F]

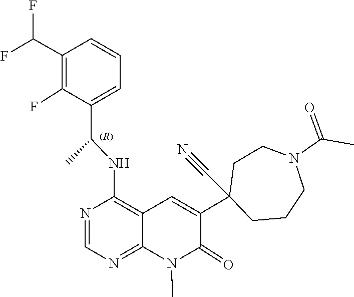

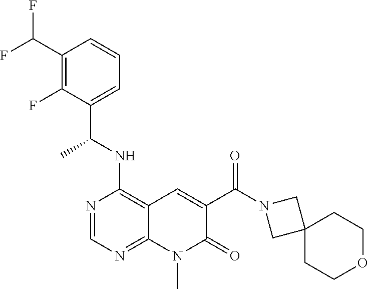 [SMILES: C[C@@H](Nc1ncnc2c1cc(C(=O)N1CC3(CCOCC3)C1)c(=O)n2C)c1cccc(C(F)F)c1F]

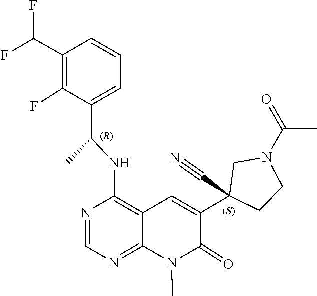

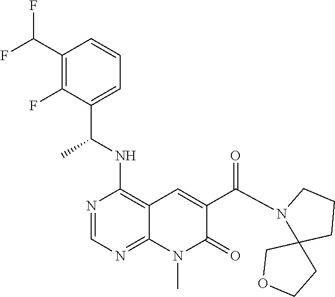 [SMILES: C[C@@H](Nc1ncnc2c1cc(C(=O)N1CCCC13CCOC3)c(=O)n2C)c1cccc(C(F)F)c1F]

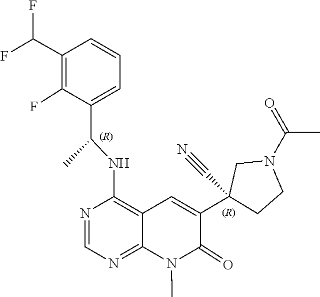

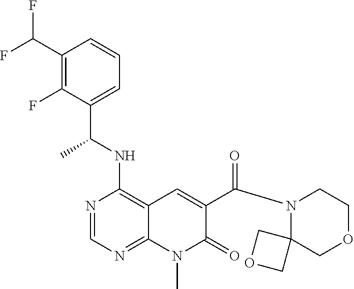 [SMILES: C[C@@H](Nc1ncnc2c1cc(C(=O)N1CCOCC13COC3)c(=O)n2C)c1cccc(C(F)F)c1F]

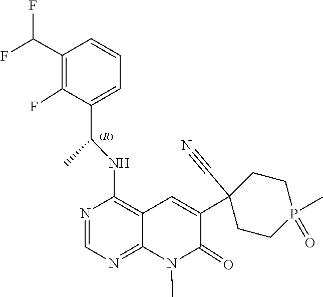

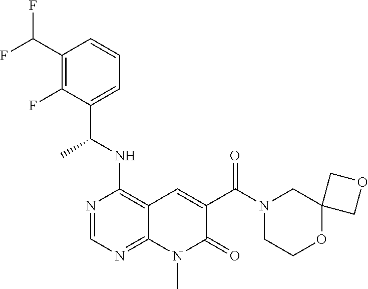 [SMILES: C[C@@H](Nc1ncnc2c1cc(C(=O)N1CCOC3(COC3)C1)c(=O)n2C)c1cccc(C(F)F)c1F]

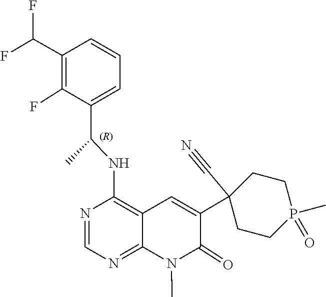

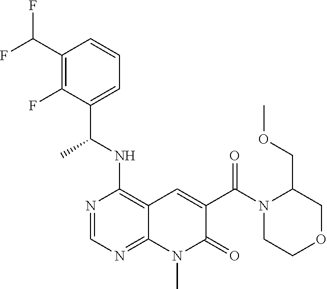 [SMILES: COCC1COCCN1C(=O)c1cc2c(N[C@H](C)c3cccc(C(F)F)c3F)ncnc2n(C)c1=O]

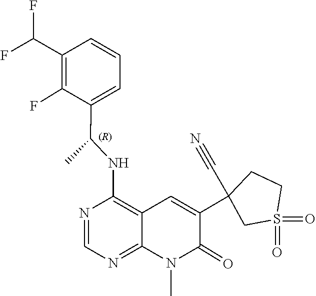

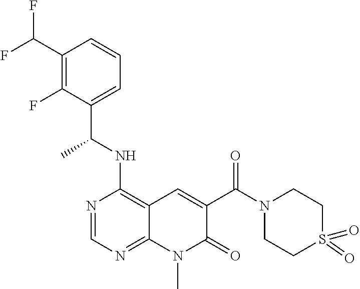 [SMILES: C[C@@H](Nc1ncnc2c1cc(C(=O)N1CCS(=O)(=O)CC1)c(=O)n2C)c1cccc(C(F)F)c1F]

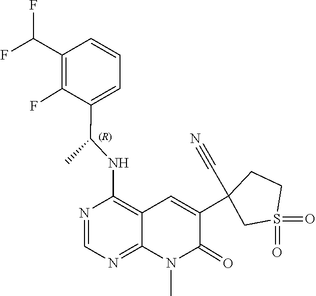

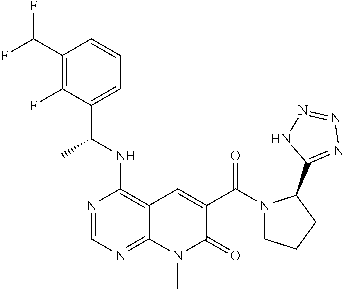 [SMILES: C[C@@H](Nc1ncnc2c1cc(C(=O)N1CCC[C@@H]1c1nnn[nH]1)c(=O)n2C)c1cccc(C(F)F)c1F]

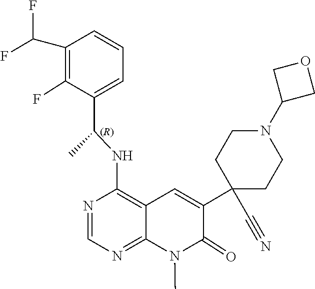

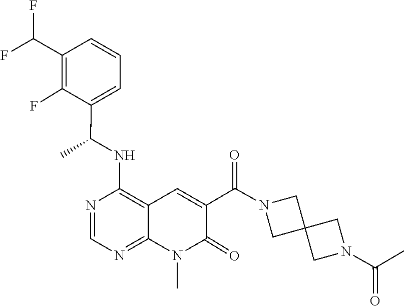 [SMILES: CC(=O)N1CC2(C1)CN(C(=O)c1cc3c(N[C@H](C)c4cccc(C(F)F)c4F)ncnc3n(C)c1=O)C2]

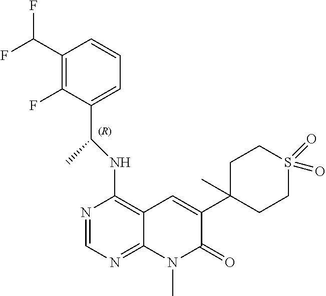

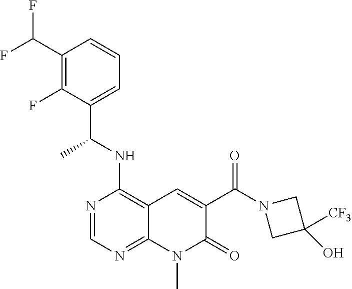 [SMILES: C[C@@H](Nc1ncnc2c1cc(C(=O)N1CC(O)(C(F)(F)F)C1)c(=O)n2C)c1cccc(C(F)F)c1F]

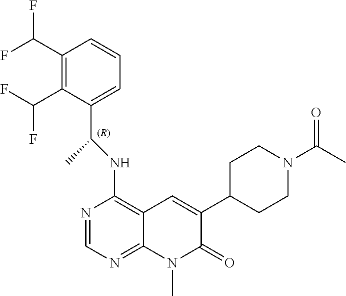

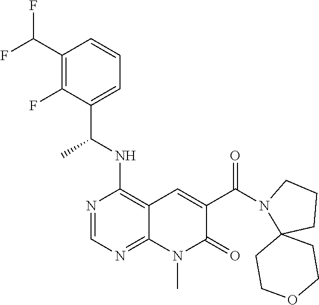 [SMILES: C[C@@H](Nc1ncnc2c1cc(C(=O)N1CCCC13CCOCC3)c(=O)n2C)c1cccc(C(F)F)c1F]

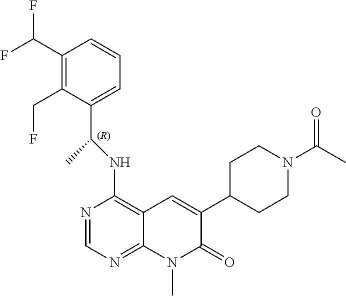

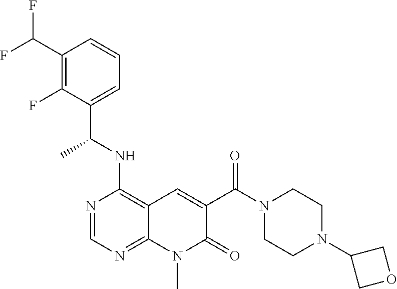 [SMILES: C[C@@H](Nc1ncnc2c1cc(C(=O)N1CCN(C3COC3)CC1)c(=O)n2C)c1cccc(C(F)F)c1F]

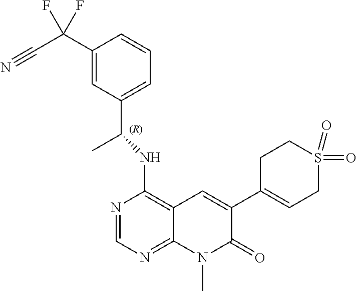

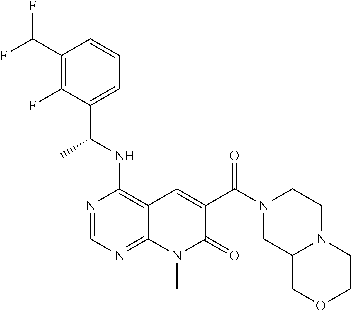 [SMILES: C[C@@H](Nc1ncnc2c1cc(C(=O)N1CCN3CCOCC3C1)c(=O)n2C)c1cccc(C(F)F)c1F]

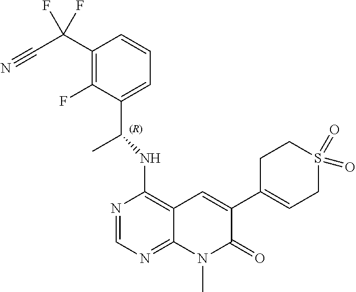

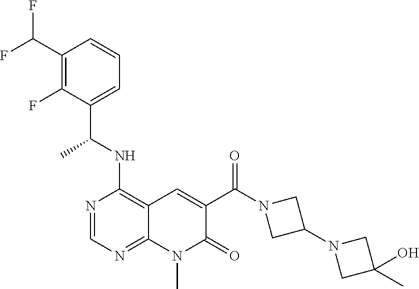 [SMILES: C[C@@H](Nc1ncnc2c1cc(C(=O)N1CC(N3CC(C)(O)C3)C1)c(=O)n2C)c1cccc(C(F)F)c1F]

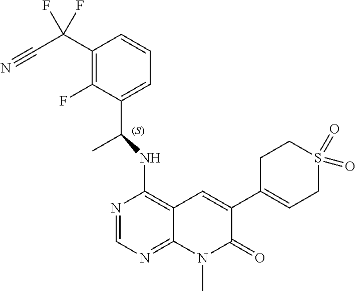

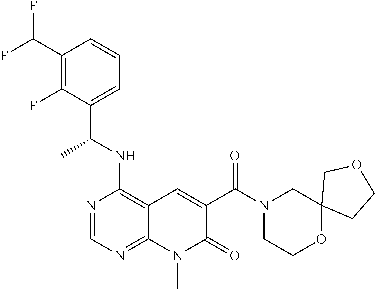 [SMILES: C[C@@H](Nc1ncnc2c1cc(C(=O)N1CCOC3(CCOC3)C1)c(=O)n2C)c1cccc(C(F)F)c1F]

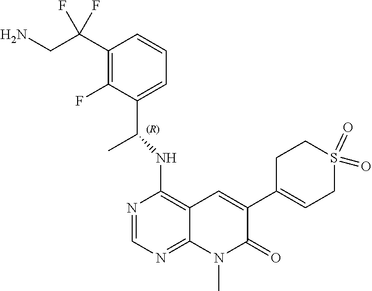

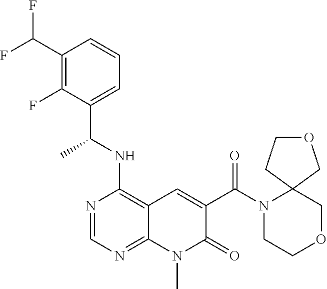 [SMILES: C[C@@H](Nc1ncnc2c1cc(C(=O)N1CCOCC13CCOC3)c(=O)n2C)c1cccc(C(F)F)c1F]

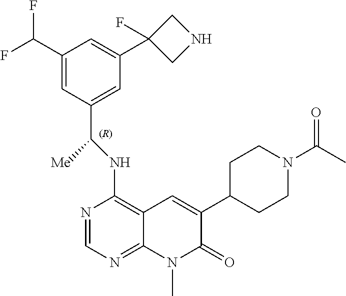

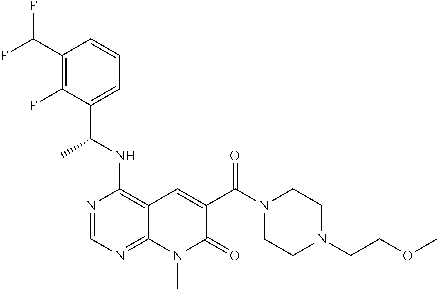 [SMILES: COCCN1CCN(C(=O)c2cc3c(N[C@H](C)c4cccc(C(F)F)c4F)ncnc3n(C)c2=O)CC1]

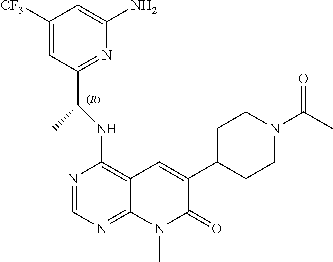

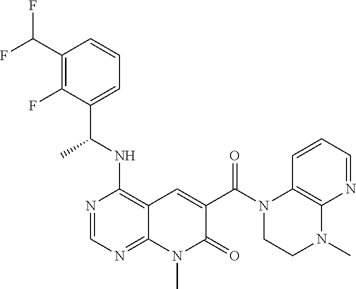 [SMILES: C[C@@H](Nc1ncnc2c1cc(C(=O)N1CCN(C)c3ncccc31)c(=O)n2C)c1cccc(C(F)F)c1F]

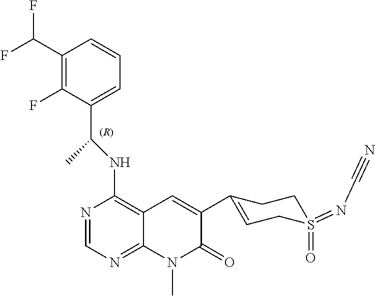

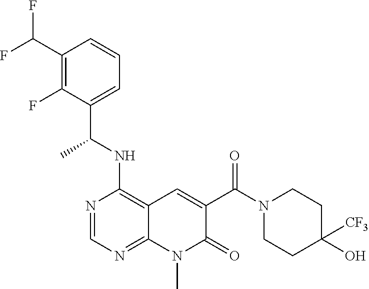 [SMILES: C[C@@H](Nc1ncnc2c1cc(C(=O)N1CCC(O)(C(F)(F)F)CC1)c(=O)n2C)c1cccc(C(F)F)c1F]

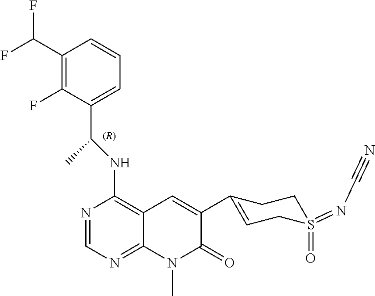

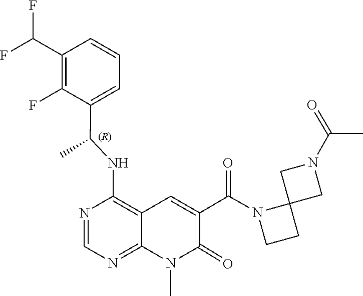

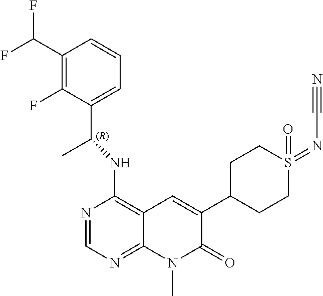

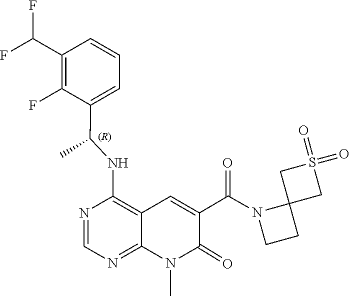

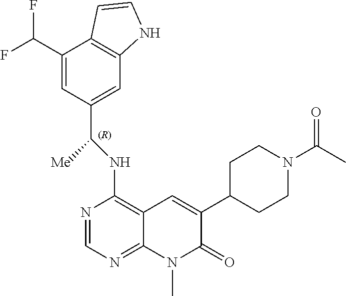

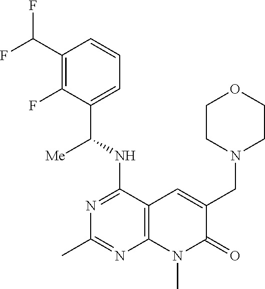 [SMILES: Cc1nc(N[C@H](C)c2cccc(C(F)F)c2F)c2cc(CN3CCOCC3)c(=O)n(C)c2n1]

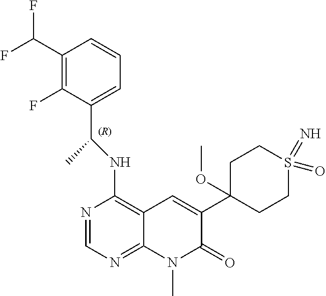

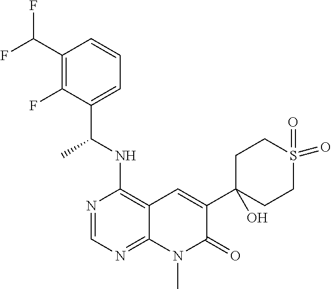 [SMILES: C[C@@H](Nc1ncnc2c1cc(C1(O)CCS(=O)(=O)CC1)c(=O)n2C)c1cccc(C(F)F)c1F]

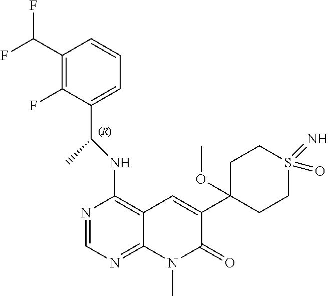

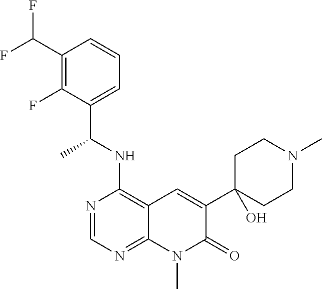 [SMILES: C[C@@H](Nc1ncnc2c1cc(C1(O)CCN(C)CC1)c(=O)n2C)c1cccc(C(F)F)c1F]

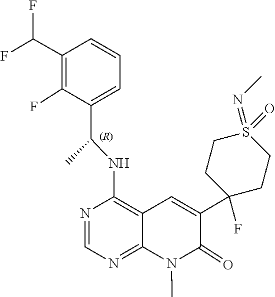

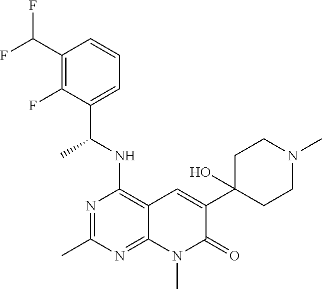 [SMILES: Cc1nc(N[C@H](C)c2cccc(C(F)F)c2F)c2cc(C3(O)CCN(C)CC3)c(=O)n(C)c2n1]

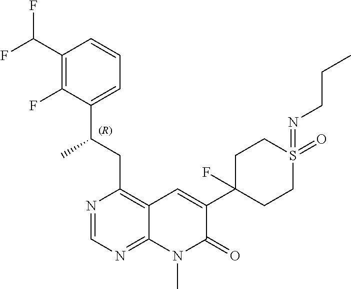

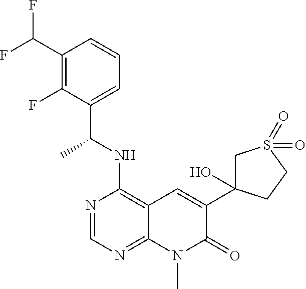 [SMILES: C[C@@H](Nc1ncnc2c1cc(C1(O)CCS(=O)(=O)C1)c(=O)n2C)c1cccc(C(F)F)c1F]

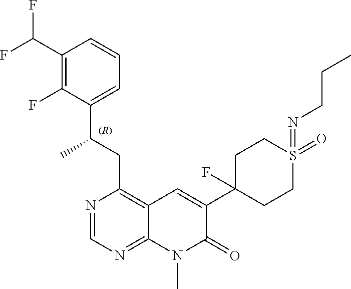

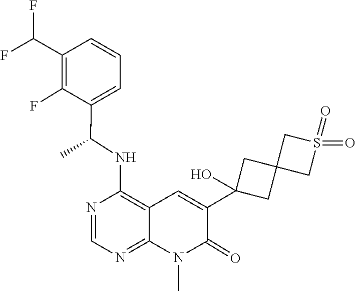 [SMILES: C[C@@H](Nc1ncnc2c1cc(C1(O)CC3(C1)CS(=O)(=O)C3)c(=O)n2C)c1cccc(C(F)F)c1F]

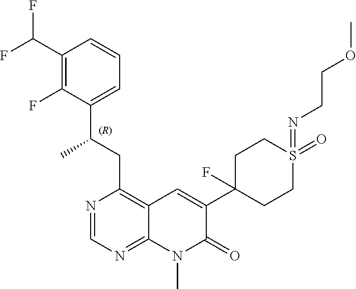

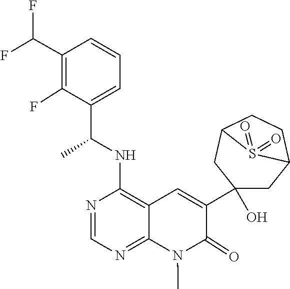 [SMILES: C[C@@H](Nc1ncnc2c1cc(C1(O)CC3CCC(C1)S3(=O)=O)c(=O)n2C)c1cccc(C(F)F)c1F]

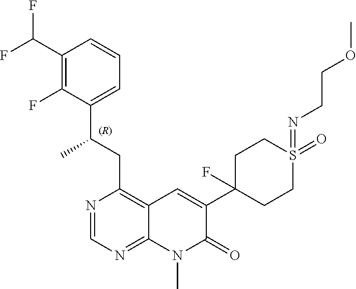

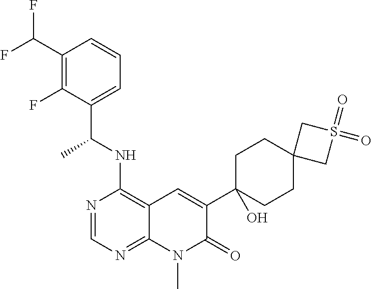 [SMILES: C[C@@H](Nc1ncnc2c1cc(C1(O)CCC3(CC1)CS(=O)(=O)C3)c(=O)n2C)c1cccc(C(F)F)c1F]

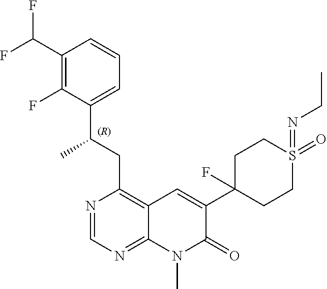

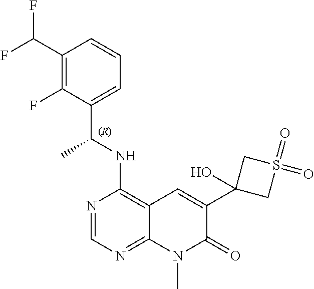

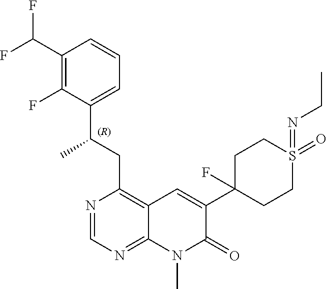

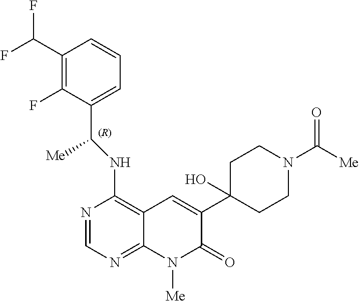

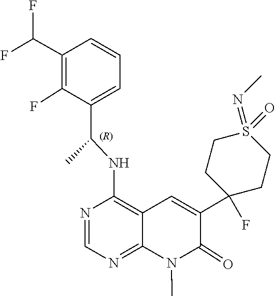

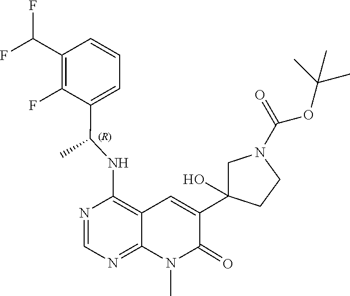

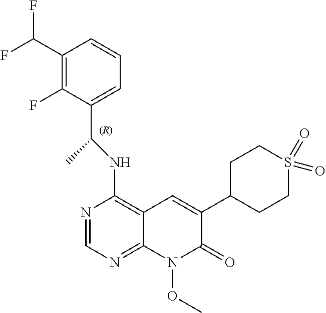

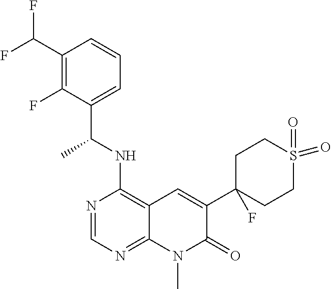 [SMILES: C[C@@H](Nc1ncnc2c1cc(C1(F)CCS(=O)(=O)CC1)c(=O)n2C)c1cccc(C(F)F)c1F]

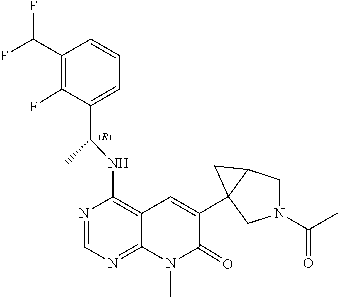

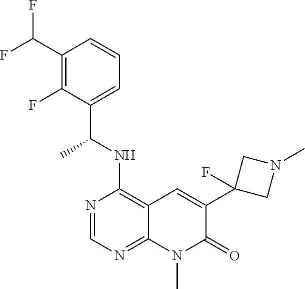 [SMILES: C[C@@H](Nc1ncnc2c1cc(C1(F)CN(C)C1)c(=O)n2C)c1cccc(C(F)F)c1F]

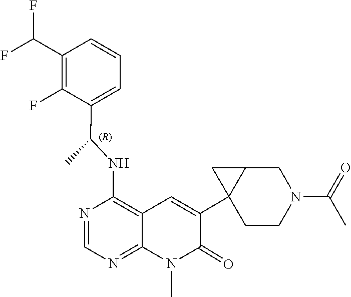

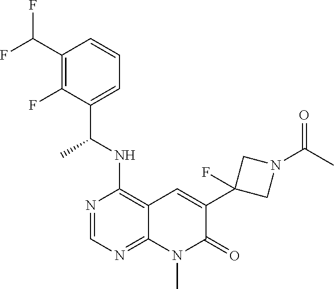 [SMILES: CC(=O)N1CC(F)(c2cc3c(N[C@H](C)c4cccc(C(F)F)c4F)ncnc3n(C)c2=O)C1]

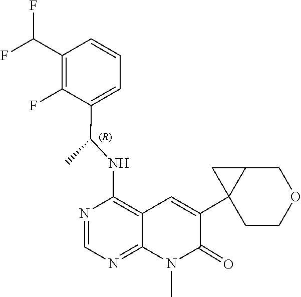

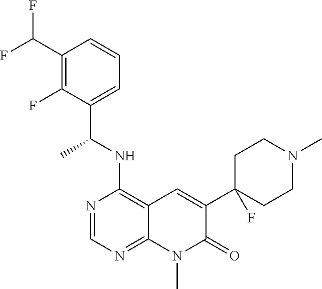 [SMILES: C[C@@H](Nc1ncnc2c1cc(C1(F)CCN(C)CC1)c(=O)n2C)c1cccc(C(F)F)c1F]

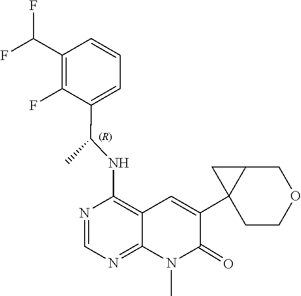

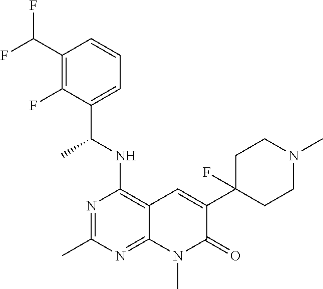 [SMILES: Cc1nc(N[C@H](C)c2cccc(C(F)F)c2F)c2cc(C3(F)CCN(C)CC3)c(=O)n(C)c2n1]

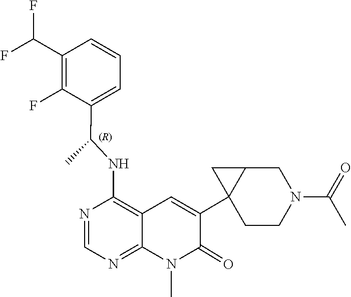

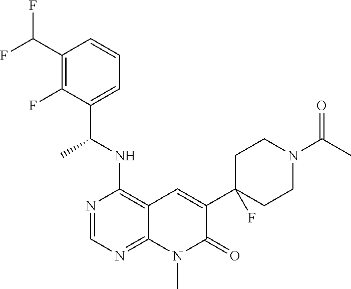 [SMILES: CC(=O)N1CCC(F)(c2cc3c(N[C@H](C)c4cccc(C(F)F)c4F)ncnc3n(C)c2=O)CC1]

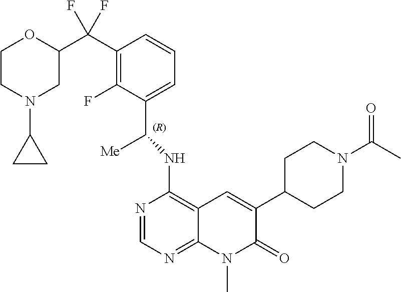

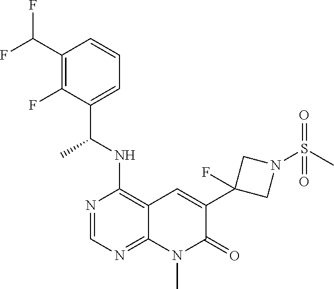 [SMILES: C[C@@H](Nc1ncnc2c1cc(C1(F)CN(S(C)(=O)=O)C1)c(=O)n2C)c1cccc(C(F)F)c1F]

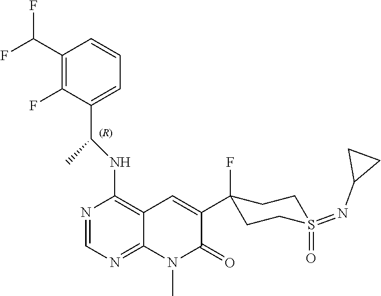

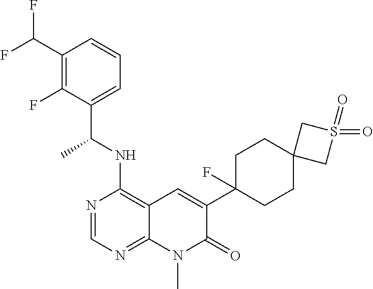 [SMILES: C[C@@H](Nc1ncnc2c1cc(C1(F)CCC3(CC1)CS(=O)(=O)C3)c(=O)n2C)c1cccc(C(F)F)c1F]

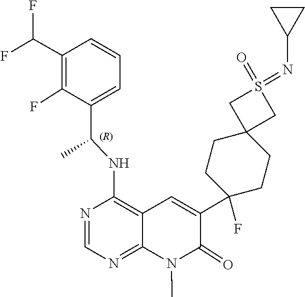

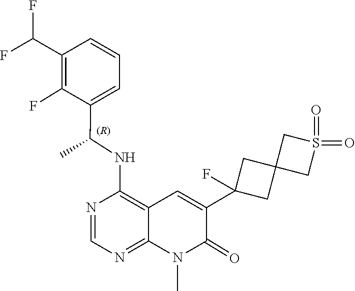

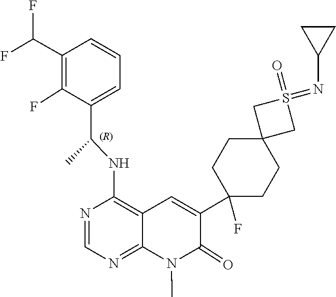

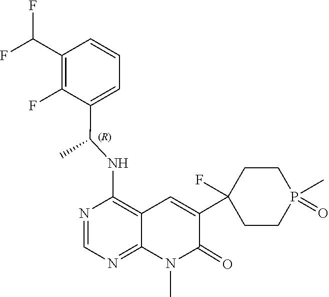

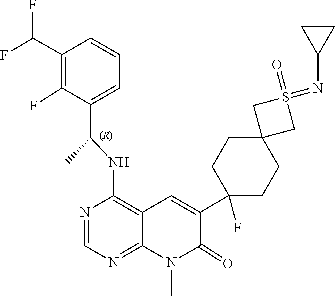

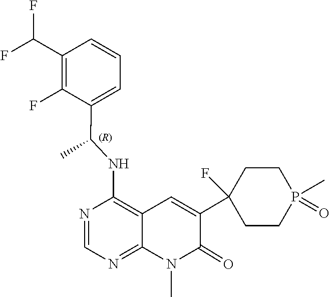

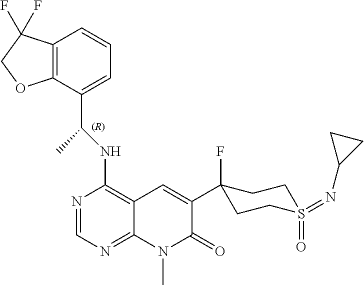

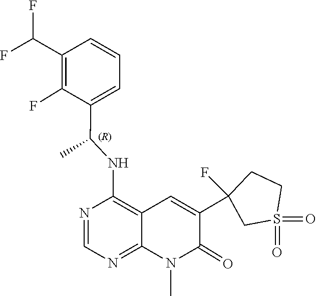

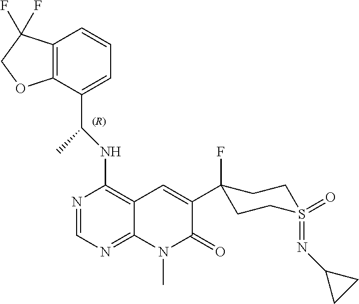

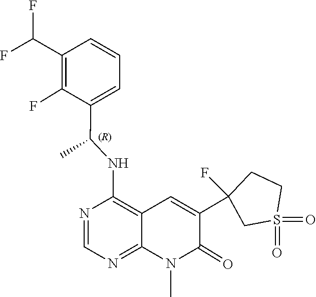

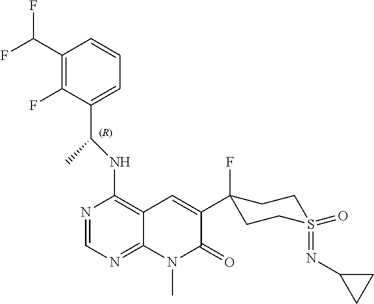

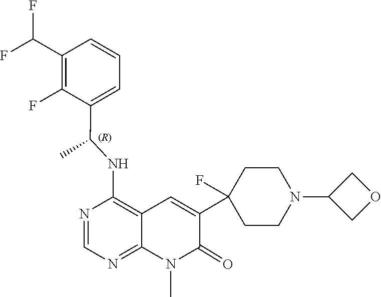

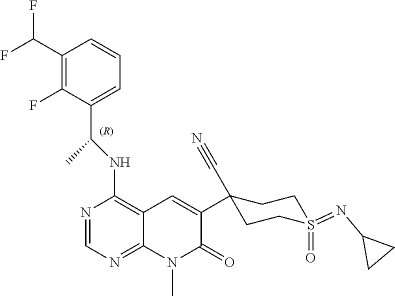

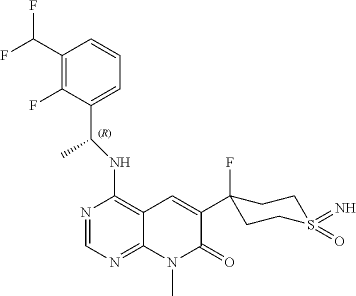

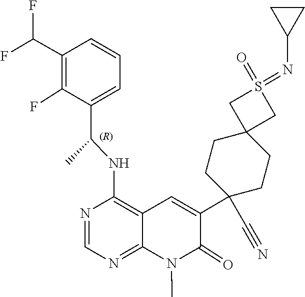

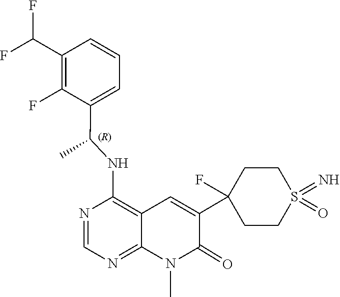

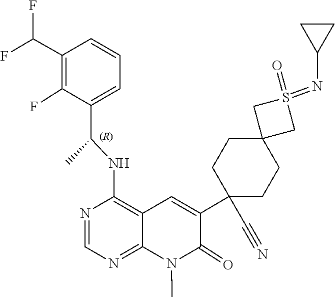

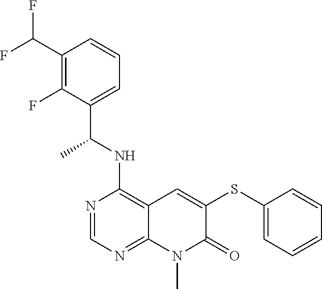 [SMILES: C[C@@H](Nc1ncnc2c1cc(Sc1ccccc1)c(=O)n2C)c1cccc(C(F)F)c1F]

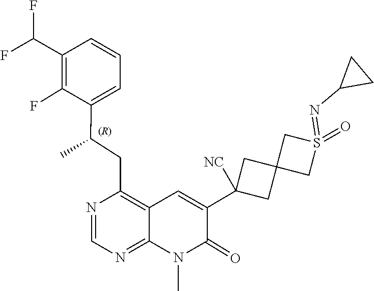

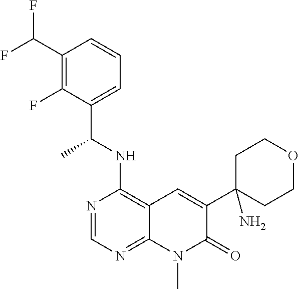 [SMILES: C[C@@H](Nc1ncnc2c1cc(C1(N)CCOCC1)c(=O)n2C)c1cccc(C(F)F)c1F]

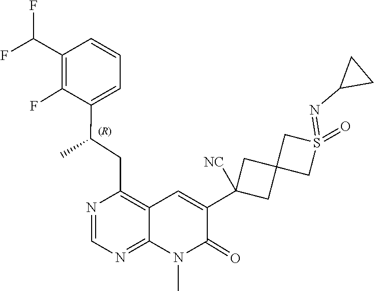

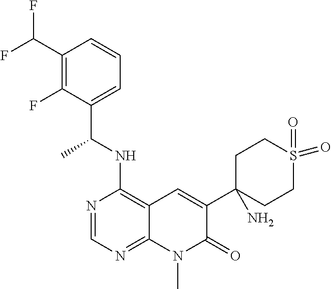 [SMILES: C[C@@H](Nc1ncnc2c1cc(C1(N)CCS(=O)(=O)CC1)c(=O)n2C)c1cccc(C(F)F)c1F]

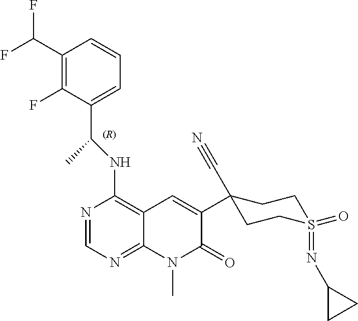

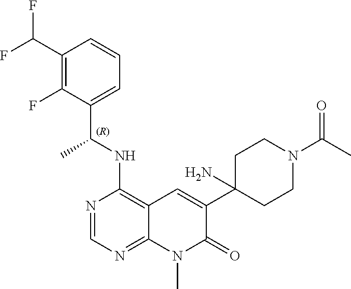

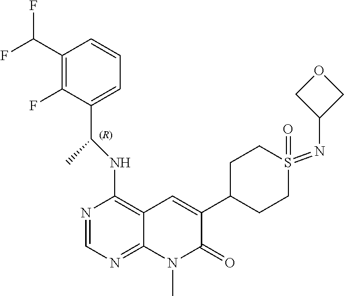

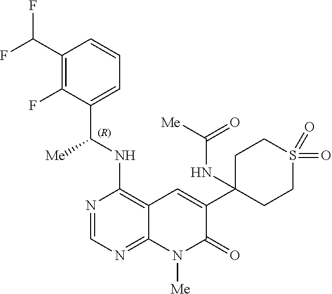

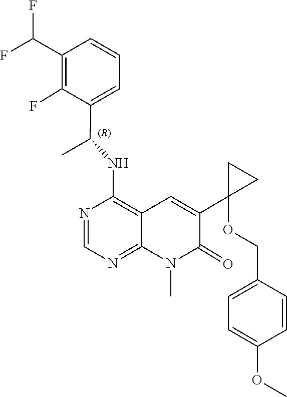

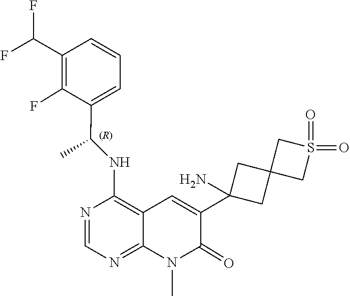

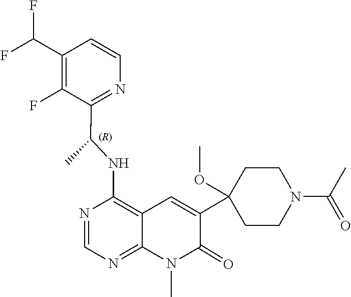

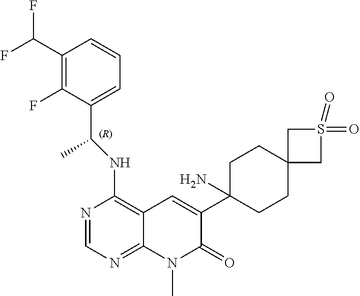

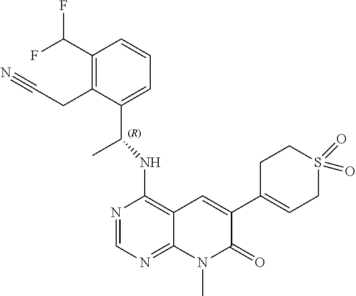

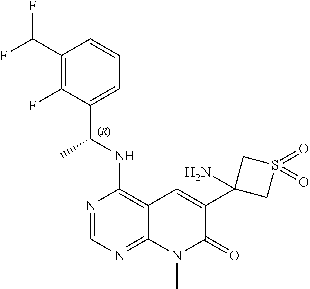

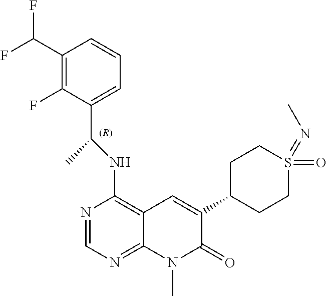

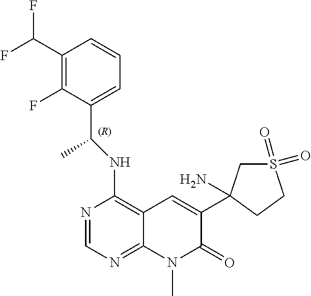

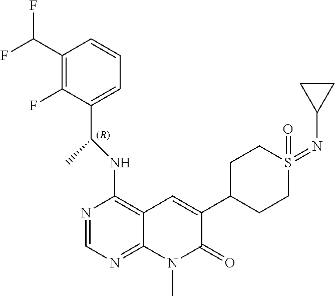

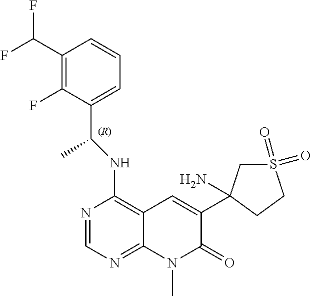

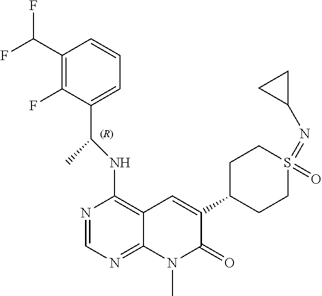

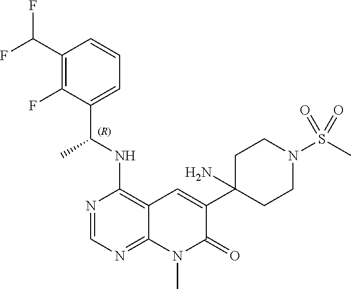

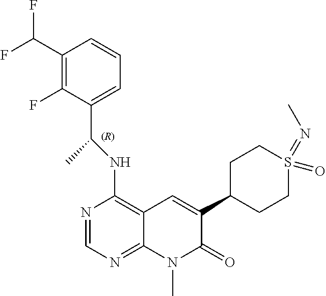

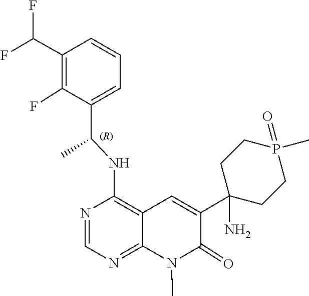

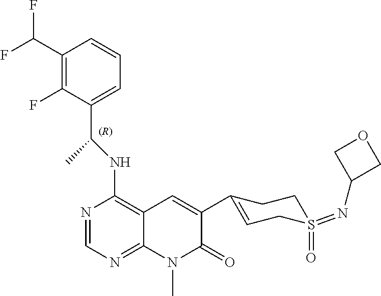

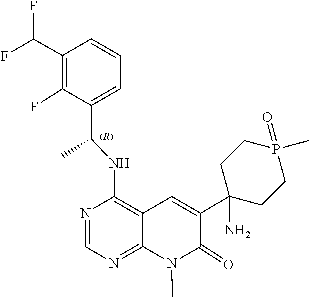

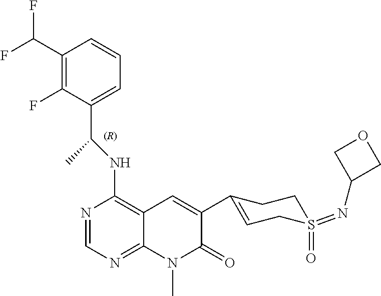

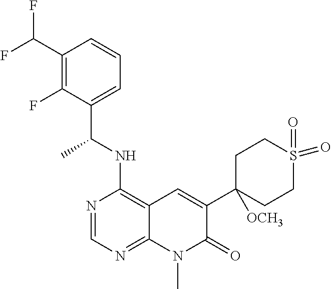 [SMILES: COC1(c2cc3c(N[C@H](C)c4cccc(C(F)F)c4F)ncnc3n(C)c2=O)CCS(=O)(=O)CC1]

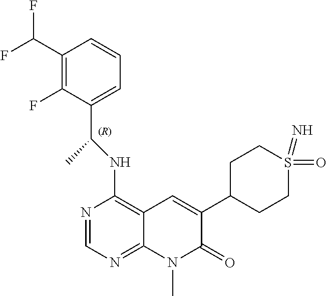

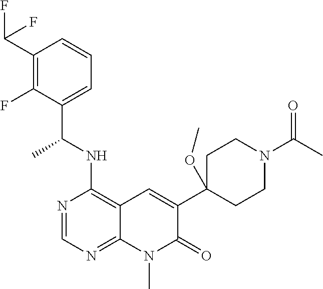 [SMILES: COC1(c2cc3c(N[C@H](C)c4cccc(C(F)F)c4F)ncnc3n(C)c2=O)CCN(C(C)=O)CC1]

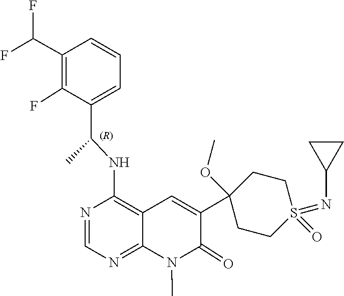

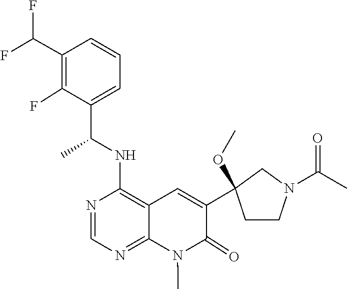 [SMILES: CO[C@]1(c2cc3c(N[C@H](C)c4cccc(C(F)F)c4F)ncnc3n(C)c2=O)CCN(C(C)=O)C1]

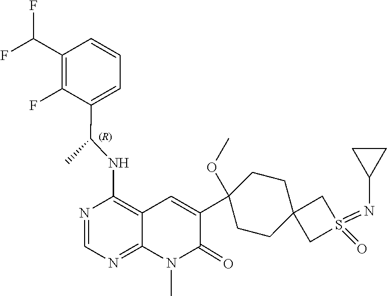

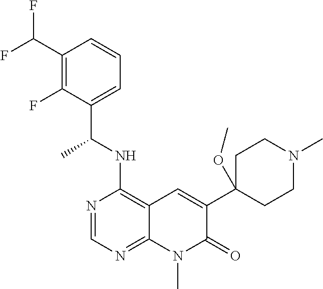 [SMILES: COC1(c2cc3c(N[C@H](C)c4cccc(C(F)F)c4F)ncnc3n(C)c2=O)CCN(C)CC1]

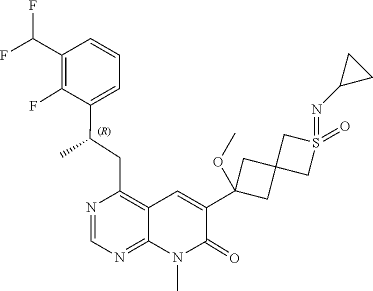

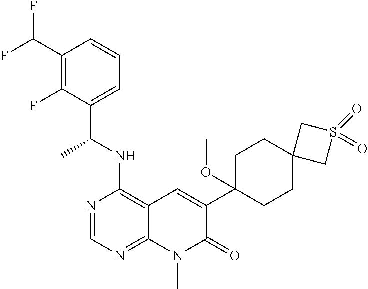 [SMILES: COC1(c2cc3c(N[C@H](C)c4cccc(C(F)F)c4F)ncnc3n(C)c2=O)CCC2(CC1)CS(=O)(=O)C2]

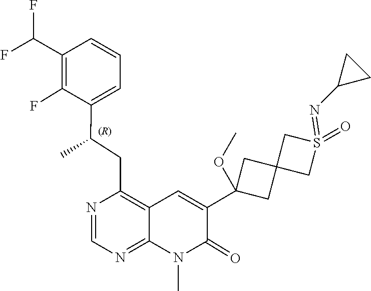

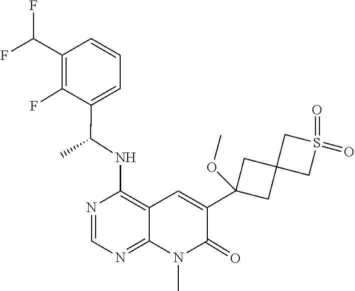 [SMILES: COC1(c2cc3c(N[C@H](C)c4cccc(C(F)F)c4F)ncnc3n(C)c2=O)CC2(C1)CS(=O)(=O)C2]

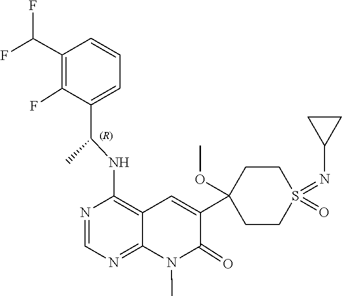

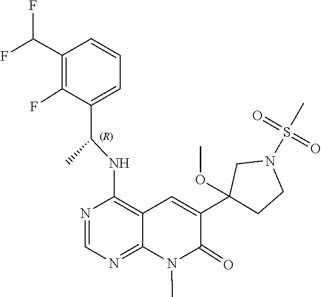

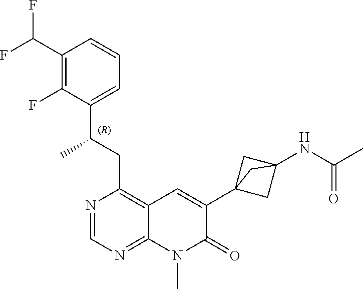

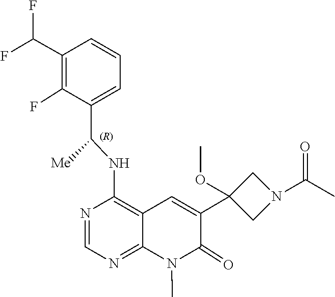

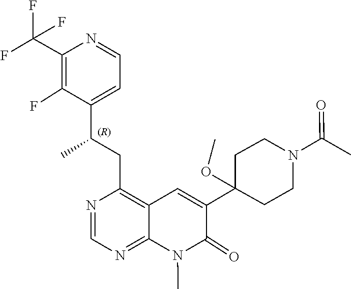

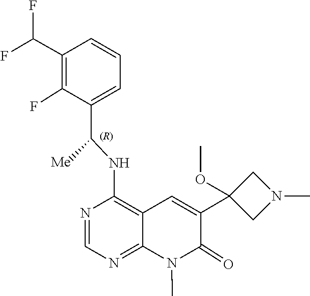

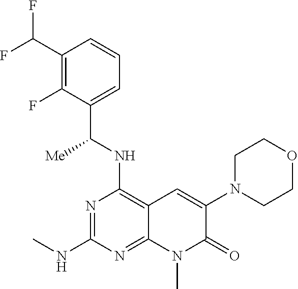 [SMILES: CNc1nc(N[C@H](C)c2cccc(C(F)F)c2F)c2cc(N3CCOCC3)c(=O)n(C)c2n1]

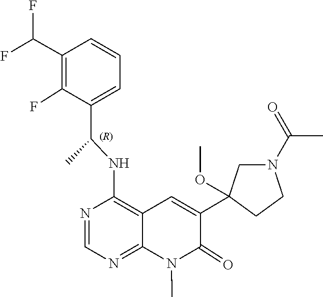

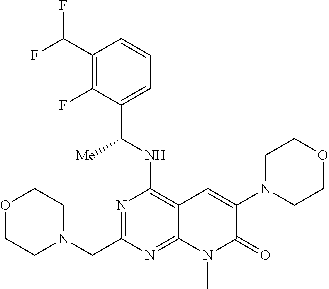 [SMILES: C[C@@H](Nc1nc(CN2CCOCC2)nc2c1cc(N1CCOCC1)c(=O)n2C)c1cccc(C(F)F)c1F]

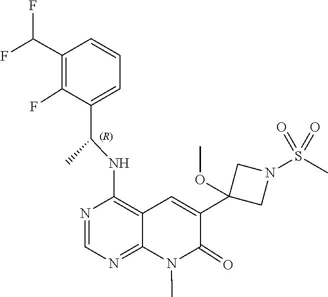

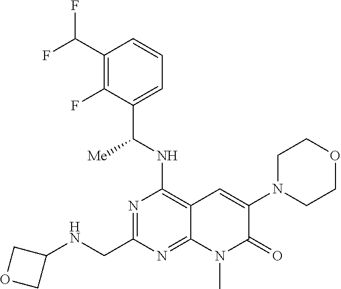 [SMILES: C[C@@H](Nc1nc(CNC2COC2)nc2c1cc(N1CCOCC1)c(=O)n2C)c1cccc(C(F)F)c1F]

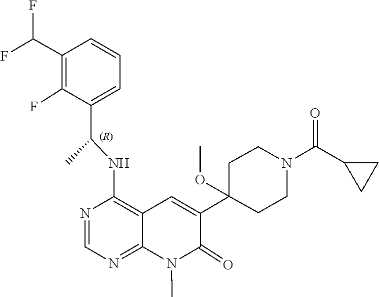

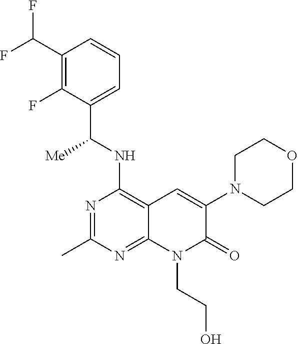 [SMILES: Cc1nc(N[C@H](C)c2cccc(C(F)F)c2F)c2cc(N3CCOCC3)c(=O)n(CCO)c2n1]

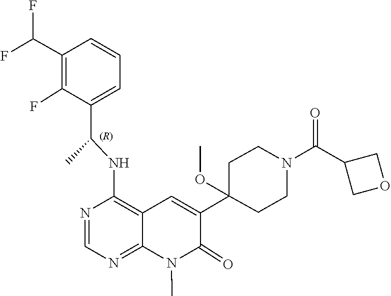

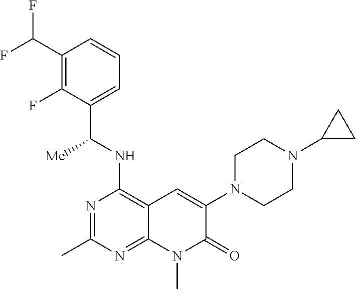 [SMILES: Cc1nc(N[C@H](C)c2cccc(C(F)F)c2F)c2cc(N3CCN(C4CC4)CC3)c(=O)n(C)c2n1]

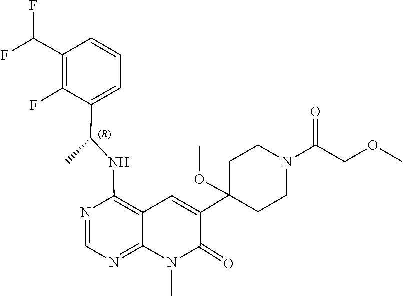

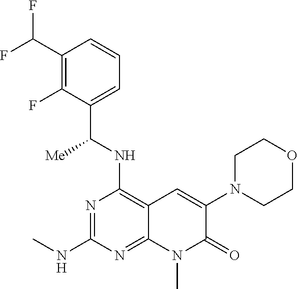 [SMILES: CNc1nc(N[C@H](C)c2cccc(C(F)F)c2F)c2cc(N3CCOCC3)c(=O)n(C)c2n1]

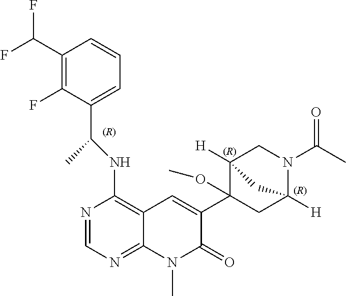

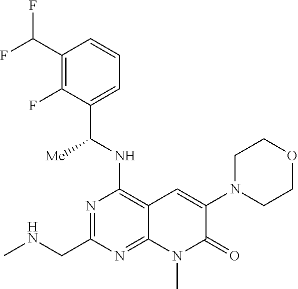 [SMILES: CNCc1nc(N[C@H](C)c2cccc(C(F)F)c2F)c2cc(N3CCOCC3)c(=O)n(C)c2n1]

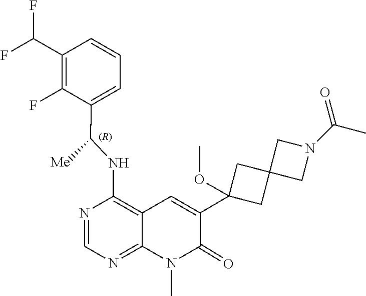

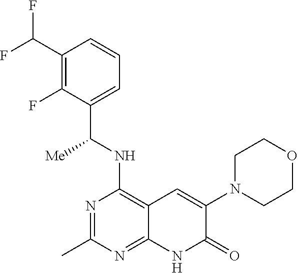 [SMILES: Cc1nc(N[C@H](C)c2cccc(C(F)F)c2F)c2cc(N3CCOCC3)c(=O)[nH]c2n1]

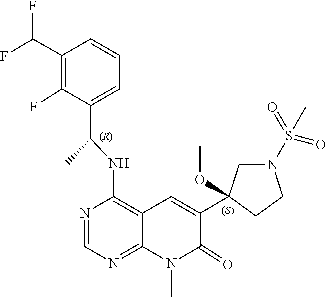

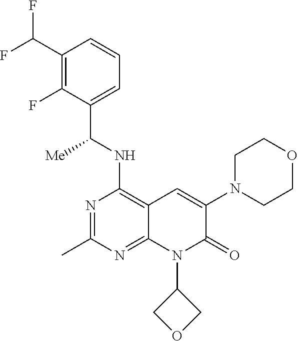 [SMILES: Cc1nc(N[C@H](C)c2cccc(C(F)F)c2F)c2cc(N3CCOCC3)c(=O)n(C3COC3)c2n1]

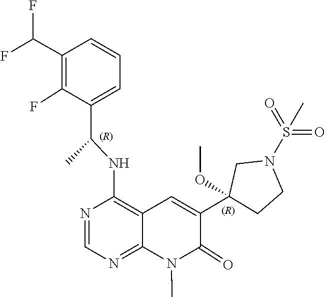

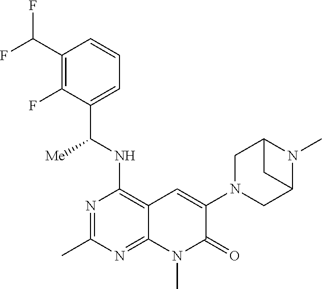 [SMILES: Cc1nc(N[C@H](C)c2cccc(C(F)F)c2F)c2cc(N3CC4CC(C3)N4C)c(=O)n(C)c2n1]

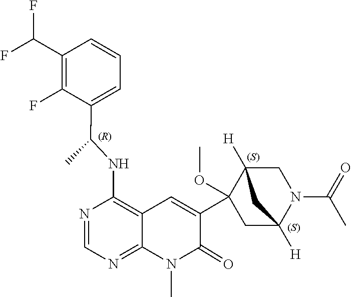

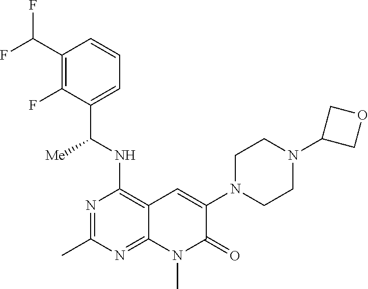 [SMILES: Cc1nc(N[C@H](C)c2cccc(C(F)F)c2F)c2cc(N3CCN(C4COC4)CC3)c(=O)n(C)c2n1]

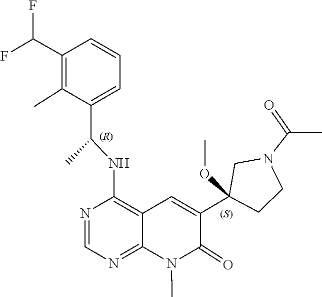

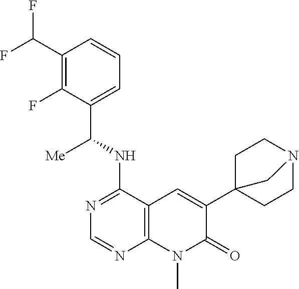 [SMILES: C[C@@H](Nc1ncnc2c1cc(C13CCN(CC1)C3)c(=O)n2C)c1cccc(C(F)F)c1F]

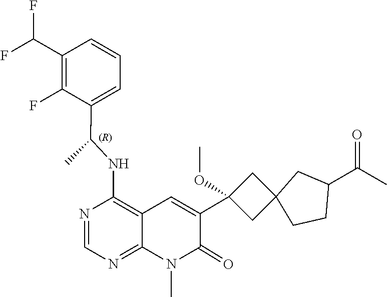

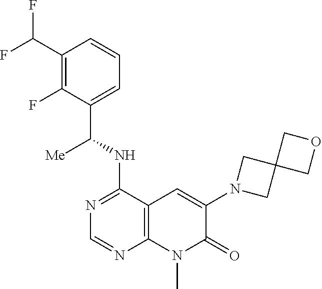 [SMILES: C[C@@H](Nc1ncnc2c1cc(N1CC3(COC3)C1)c(=O)n2C)c1cccc(C(F)F)c1F]

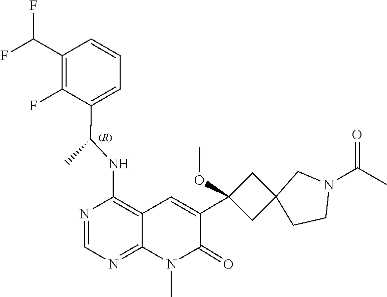

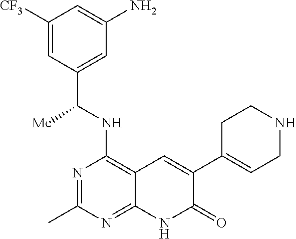 [SMILES: Cc1nc(N[C@H](C)c2cc(N)cc(C(F)(F)F)c2)c2cc(C3=CCNCC3)c(=O)[nH]c2n1]

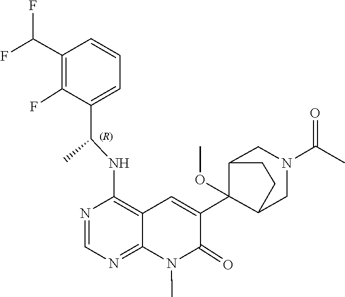

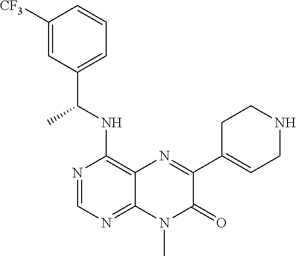 [SMILES: C[C@@H](Nc1ncnc2c1nc(C1=CCNCC1)c(=O)n2C)c1cccc(C(F)(F)F)c1]

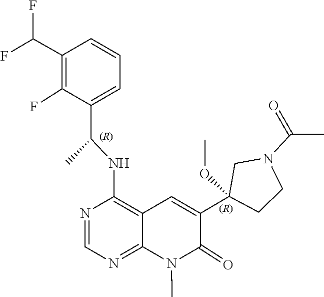

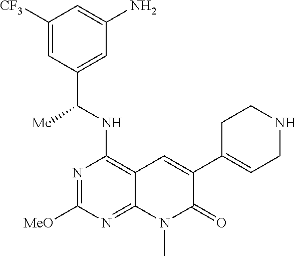 [SMILES: COc1nc(N[C@H](C)c2cc(N)cc(C(F)(F)F)c2)c2cc(C3=CCNCC3)c(=O)n(C)c2n1]

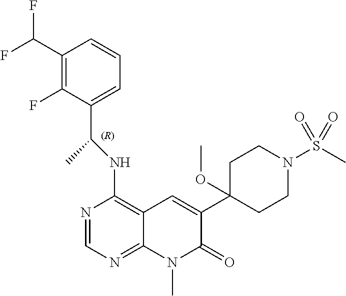

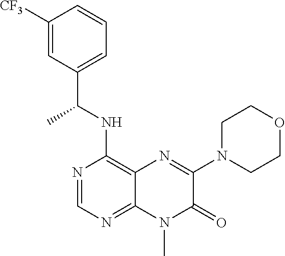 [SMILES: C[C@@H](Nc1ncnc2c1nc(N1CCOCC1)c(=O)n2C)c1cccc(C(F)(F)F)c1]

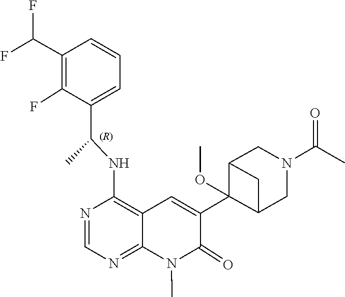

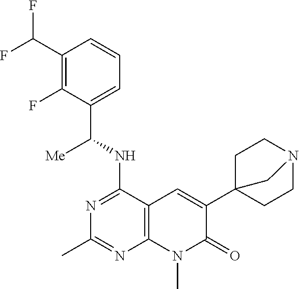 [SMILES: Cc1nc(N[C@H](C)c2cccc(C(F)F)c2F)c2cc(C34CCN(CC3)C4)c(=O)n(C)c2n1]

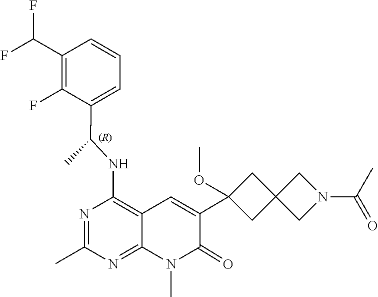

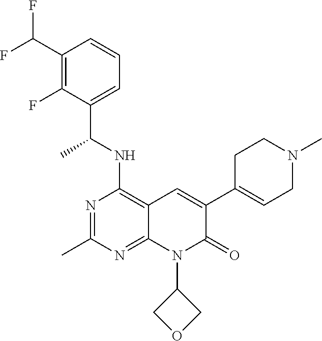 [SMILES: Cc1nc(N[C@H](C)c2cccc(C(F)F)c2F)c2cc(C3=CCN(C)CC3)c(=O)n(C3COC3)c2n1]

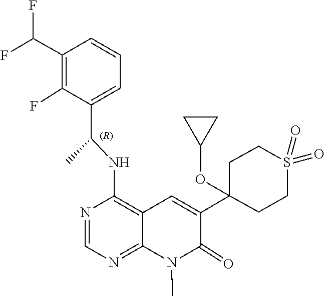

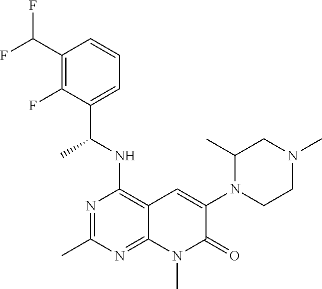 [SMILES: Cc1nc(N[C@H](C)c2cccc(C(F)F)c2F)c2cc(N3CCN(C)CC3C)c(=O)n(C)c2n1]

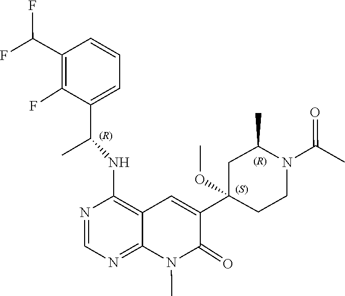

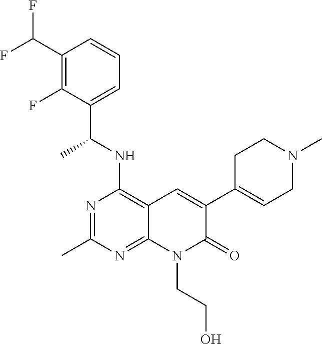 [SMILES: Cc1nc(N[C@H](C)c2cccc(C(F)F)c2F)c2cc(C3=CCN(C)CC3)c(=O)n(CCO)c2n1]

Note:
any compound shown in the foregoing table may be a diastereomer, and the absolute stereochemistry of such diastereomer may not be known.

In some embodiments, the SOS1 inhibitor is selected from the group consisting of:
(R)-4-((1-(3-amino-5-(trifluoromethyl)phenyl)ethyl)amino)-8-methyl-6-(1,2,3,6-tetrahydropyridin-4-yl)pyrido[2,3-d]pyrimidin-7 (8H)-one;
(R)-4-((1-(3-(1,1-difluoro-2-hydroxyethyl)phenyl)ethyl)amino)-8-methyl-6-morpholinopyrido[2,3-d]pyrimidin-7 (8H)-one;
(R)-6-(3,6-dihydro-2H-pyran-4-yl)-8-methyl-4-(1-(3-(trifluoromethyl)phenyl)ethyl)amino)pyrido[2,3-d]pyrimidin-7 (8H)-one;
4-{[(1R)-1-[3-amino-5-(trifluoromethyl)phenyl]ethyl]amino}-8-methyl-6-(morpholin-4-yl)-7H,8H-pyrido[2,3-d]pyrimidin-7-one;
8-methyl-6-(morpholin-4-yl)-4-{[(1R)-1-[3-(trifluoromethyl)phenyl]-ethyl]amino}-7H,8H-pyrido[2,3-d]pyrimidin-7-one;
4-{[(1R)-1-[3-(difluoromethyl)-2-fluorophenyl]ethyl]amino}-6-(1-methanesulfonyl-3-methylazetidin-3-yl)-8-methyl-7H,8H-pyrido[2,3-d]pyrimidin-7-one;
6-(1-acetyl-4-piperidyl)-4-[[(1R)-1-[3-amino-2-fluoro-5-(trifluoromethyl)phenyl]ethyl]amino]-8-methyl-pyrido[2,3-d]pyrimidin-7-one;
6-(1-acetyl-4-piperidyl)-4-[[(1R)-1-[5-amino-2-fluoro-3-(trifluoromethyl)phenyl]ethyl]amino]-8-methyl-pyrido[2,3-d]pyrimidin-7-one;
(R)-4-((1-(3-(difluoromethyl)-2-fluorophenyl)ethyl)amino)-8-methyl-6-(pyridazin-4-yl)pyrido[2,3-d]pyrimidin-7 (8H)-one;
4-(((R)-1-(3-(difluoromethyl)-2-fluorophenyl)ethyl)amino)-8-methyl-6-(1-oxido-3,6-dihydro-2H-thiopyran-4-yl)pyrido[2,3-d]pyrimidin-7 (8H)-one;
4-(((R)-1-(3-(difluoromethyl)-2-fluorophenyl)ethyl)amino)-6-(1-imino-1-oxido-1,2,3,6-tetrahydro-116-thiopyran-4-yl)-8-methylpyrido[2,3-d]pyrimidin-7 (8H)-one;
4-(((R)-1-(3-(difluoromethyl)-2-fluorophenyl)ethyl)amino)-8-methyl-6-(1-(methylimino)-1-oxido-1,2,3,6-tetrahydro-1λ⁶-thiopyran-4-yl)pyrido[2,3-d]pyrimidin-7 (8H)-one;
(R)-4-((1-(3-(difluoromethyl)-2-fluorophenyl)ethyl)amino)-8-methyl-6-(1-oxidotetrahydro-2H-thiopyran-4-yl)pyrido[2,3-d]pyrimidin-7 (8H)-one;
4-(4-{[(1R)-1-[3-(difluoromethyl)-2-fluorophenyl]-ethyl]amino}-8-methyl-7-oxo-7H, 8H-pyrido[2,3-d]pyrimidin-6-yl)-4-hydroxy-1λ⁶-thiane-1,1-dione;
4-(4-{[(1R)-1-[3-(difluoromethyl)-2-fluorophenyl]ethyl]amino}-8-methyl-7-oxo-7H,8H-pyrido[2,3-d]-pyrimidin-6-yl)-4-fluoro-1λ⁶-thiane-1,1-dione;
4-{[(1R)-1-[3-(difluoromethyl)-2-fluorophenyl]ethyl]amino}-8-methyl-6-(phenylsulfanyl)-7H,8H-pyrido[2,3-d]pyrimidin-7-one;
6-(4-aminooxan-4-yl)-4-{[(1R)-1-[3-(difluoromethyl)-2-fluorophenyl]ethyl]amino}-8-methyl-7H,8H-pyrido-[2,3-d]pyrimidin-7-one;
4-(4-{[(1R)-1-[3-(difluoro-methyl)-2-fluorophenyl]ethyl]amino}-8-methyl-7-oxo-7H,8H-pyrido[2,3-d]pyrimidin-6-yl)-4-methoxy-1λ⁶-thiane-1,1-dione;
6-(4-{[(1R)-1-[3-(difluoromethyl)-2-fluorophenyl]ethyl]amino}-8-methyl-7-oxo-7H, 8H-pyrido[2,3-d]pyrimi-din-6-yl)-2λ⁶-thiaspiro[3.3]heptane-2,2-dione;
4-(((R)-1-(3-(difluoromethyl)-2-fluorophenyl)ethyl)amino)-6-(3-hydroxypiperidin-4-yl)-8-methylpyrido[2,3-d]pyrimidin-7 (8H)-one;
(R)-4-((1-(3-(difluoromethyl)-2-fluorophenyl)ethyl)amino)-6-(1-imino-1-oxidohexahydro-1λ⁶-thiopyran-4-yl)-8-methylpyrido[2,3-d]pyrimidin-7 (8H)-one;
1-acetyl-4-(4-{[(1R)-1-[3-(difluoromethyl)-2-fluorophenyl]ethyl]amino}-8-methyl-7-oxo-7H,8H-pyrido[2,3-d]pyrimidin-6-yl) piperidine-4-carbonitrile;
4-(4-{[(1R)-1-[3-(difluoromethyl)-2-fluorophenyl]ethyl]amino}-8-methyl-7-oxo-7H, 8H-pyrido[2,3-d]pyrimidin-6-yl)-4-methyl-1λ⁶-thiane-1,1-dione;
6-(1-acetylpiperidin-4-yl)-4-{[(1R)-1-[2,3-bis(difluoromethyl)phenyl]ethyl]amino}-8-methyl-7H,8H-pyrido[2,3-d]pyrimidin-7-one;
6-(1-acetylpiperidin-4-yl)-4-{[(1R)-1-[3-(difluoromethyl)-2-(fluoromethyl)phenyl]ethyl]amino}-8-methyl-7H,8H-pyrido[2,3-d]pyrimidin-7-one;
2-{3-[(1R)-1-{[6-(1,1-dioxo-3,6-dihydro-2H-1λ⁶-thiopyran-4-yl)-8-methyl-7-oxo-7H,8H-pyrido[2,3-d]pyrimidin-4-yl]amino}ethyl]phenyl}-2,2-difluoroacetonitrile;
2-{3-[(1R)-1-{[6-(1,1-dioxo-3,6-dihydro-2H-1λ⁶-thiopyran-4-yl)-8-methyl-7-oxo-7H,8H-pyrido[2,3-d]pyrimidin-4-yl]amino}ethyl]-2-fluorophenyl}-2,2-difluoroacetonitrile;
4-(4-{[(1R)-1-[3-(2-amino-1,1-difluoroethyl)-2-fluorophenyl]ethyl]amino}-8-methyl-7-oxo-7H,8H-pyrido[2,3-d]pyrimidin-6-yl)-3,6-dihydro-2H-1λ⁶-thiopyran-1,1-dione;
6-(1-acetyl-4-piperidyl)-4-[[(1R)-1-[3-(difluoromethyl)-5-(3-fluoroazetidin-3-yl)phenyl]ethyl]amino]-8-methyl-pyrido[2,3-d]pyrimidin-7-one;
[4-[4-[[(1R)-1-[3-(difluoromethyl)-2-fluoro-phenyl]ethyl]amino]-8-methyl-7-oxo-pyrido[2,3-d]pyrimidin-6-yl]-1-oxo-3,6-dihydro-2H-thiopyran-1-ylidene]cyanamide;
[4-[4-[[(1R)-1-[3-(difluoromethyl)-2-fluoro-phenyl]ethyl]amino]-8-methyl-7-oxo-pyrido[2,3-d]pyrimidin-6-yl]-1-oxo-thian-1-ylidene]cyanamide;
4-(((R)-1-(3-(difluoromethyl)-2-fluorophenyl)ethyl)amino)-6-((1S,4s)-1-imino-4-methoxy-1-oxidohexahydro-1λ⁶-thiopyran-4-yl)-8-methylpyrido[2,3-d]pyrimidin-7 (8H)-one;
4-(((R)-1-(3-(difluoromethyl)-2-fluorophenyl)ethyl)amino)-6-((1R,4r)-1-imino-4-methoxy-1-oxidohexahydro-1λ⁶-thiopyran-4-yl)-8-methylpyrido[2,3-d]pyrimidin-7 (8H)-one;
4-(((R)-1-(3-(difluoromethyl)-2-fluorophenyl)ethyl)amino)-6-((1S,4s)-4-fluoro-1-(methylimino)-1-oxidohexahydro-1λ⁶-thiopyran-4-yl)-8-methylpyrido[2,3-d]pyrimidin-7 (8H)-one;
4-(((R)-1-(3-(difluoromethyl)-2-fluorophenyl)ethyl)amino)-6-((1R,4r)-4-fluoro-1-(methylimino)-1-oxidohexahydro-116-thiopyran-4-yl)-8-methylpyrido[2,3-d]pyrimidin-7 (8H)-one;
6-{3-acetyl-3-azabicyclo[3.1.0]hexan-1-yl}-4-{[(1R)-1-[3-(difluoromethyl)-2-fluorophenyl]ethyl]amino}-8-methyl-7H,8H-pyrido[2,3-d]pyrimidin-7-one;
6-(1-acetyl-4-piperidyl)-4-[[(1R)-1-[3-[(4-cyclopropylmorpholin-2-yl)-difluoro-methyl]-2-fluoro-phenyl]ethyl]amino]-8-methyl-pyrido[2,3-d]pyrimidin-7-one;
6-((1R,4r)-1-(cyclopropylimino)-4-fluoro-1-oxidohexahydro-1λ⁶-thiopyran-4-yl)-4-(((R)-1-(3-(difluoromethyl)-2-fluorophenyl)ethyl)amino)-8-methylpyrido[2,3-d]pyrimidin-7 (8H)-one;
6-((1S,4s)-1-(cyclopropylimino)-4-fluoro-1-oxidohexahydro-116-thiopyran-4-yl)-4-(((R)-1-(3-(difluoromethyl)-2-fluorophenyl)ethyl)amino)-8-methylpyrido[2,3-d]pyrimidin-7 (8H)-one;
(1R,4r)-1-(cyclopropylimino)-4-(4-(((R)-1-(3-(difluoromethyl)-2-fluorophenyl)ethyl)amino)-8-methyl-7-oxo-7,8-dihydropyrido[2,3-d]pyrimidin-6-yl) hexahydro-1λ⁶-thiopyran-4-carbonitrile 1-oxide;
(1S,4s)-1-(cyclopropylimino)-4-(4-(((R)-1-(3-(difluoromethyl)-2-fluorophenyl)ethyl)amino)-8-methyl-7-oxo-7,8-dihydropyrido[2,3-d]pyrimidin-6-yl) hexahydro-116-thiopyran-4-carbonitrile 1-oxide;
4-[[(1R)-1-[3-(difluoromethyl)-2-fluoro-phenyl]ethyl]amino]-8-methyl-6-[1-(oxetan-3-ylimino)-1-oxo-thian-4-yl]pyrido[2,3-d]pyrimidin-7-one;
4-[[(1R)-1-[3-(difluoromethyl)-2-fluoro-phenyl]ethyl]amino]-6-[1-[(4-methoxyphenyl) methoxy]cyclopropyl]-8-methyl-pyrido[2,3-d]pyrimidin-7-one;
2-[2-(difluoromethyl)-6-[(1R)-1-[[6-(1,1-dioxo-3,6-dihydro-2H-thiopyran-4-yl)-8-methyl-7-oxo-pyrido[2,3-d]pyrimidin-4-yl]amino]ethyl]phenyl]acetonitrile;
4-(((R)-1-(3-(difluoromethyl)-2-fluorophenyl)ethyl)amino)-8-methyl-6-((1R,4r)-1-(methylimino)-1-oxidohexahydro-1λ⁶-thiopyran-4-yl)pyrido[2,3-d]pyrimidin-7 (8H)-one;
4-(((R)-1-(3-(difluoromethyl)-2-fluorophenyl)ethyl)amino)-8-methyl-6-((S)-1-(oxetan-3-ylimino)-1-oxido-1,2,3,6-tetrahydro-1λ⁶-thiopyran-4-yl)pyrido[2,3-d]pyrimidin-7 (8H)-one;
4-(((R)-1-(3-(difluoromethyl)-2-fluorophenyl)ethyl)amino)-8-methyl-6-((R)-1-(oxetan-3-ylimino)-1-oxido-1,2,3,6-tetrahydro-1λ⁶-thiopyran-4-yl)pyrido[2,3-d]pyrimidin-7 (8H)-one;
4-[[(1R)-1-[3-(difluoromethyl)-2-fluoro-phenyl]ethyl]amino]-6-(1-imino-1-oxo-thian-4-yl)-8-methyl-pyrido[2,3-d]pyrimidin-7-one;
6-((1R,4r)-1-(cyclopropylimino)-4-methoxy-1-oxidohexahydro-116-thiopyran-4-yl)-4-(((R)-1-(3-(difluoromethyl)-2-fluorophenyl)ethyl)amino)-8-methylpyrido[2,3-d]pyrimidin-7 (8H)-one;
6-((1S,4s)-1-(cyclopropylimino)-4-methoxy-1-oxidohexahydro-1λ⁶-thiopyran-4-yl)-4-(((R)-1-(3-(difluoromethyl)-2-fluorophenyl)ethyl)amino)-8-methylpyrido[2,3-d]pyrimidin-7(8H)-one; and
N-[3-(4-{[(1R)-1-[3-(difluoromethyl)-2-fluorophenyl]ethyl]amino}-8-methyl-7-oxo-7H,8H-pyrido[2,3-d]pyrimidin-6-yl) bicyclo[1.1.1]pentan-1-yl]acetamide;

or a pharmaceutically acceptable salt or a stereoisomer thereof.

In some embodiments, the SOS1 inhibitor is selected from among the compounds in the following table, or a pharmaceutically acceptable salt or a stereoisomer thereof:

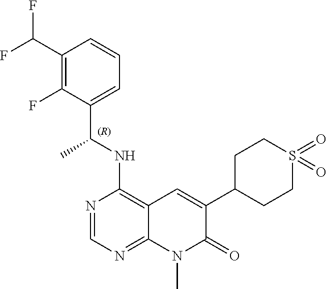

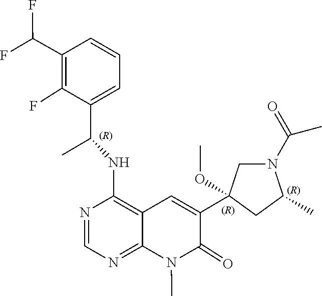

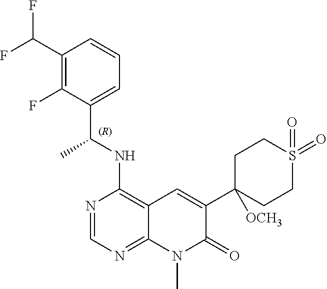

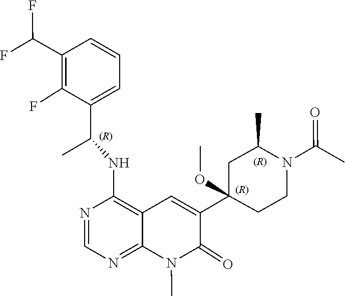

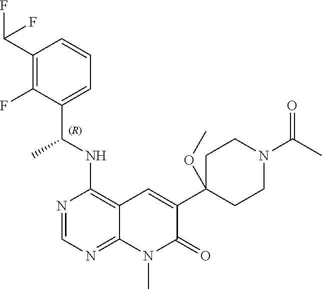

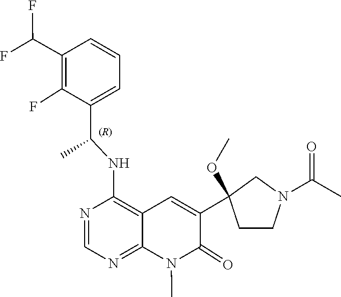

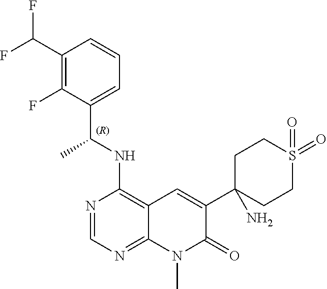

In some embodiments, the SOS1 inhibitor is a compound as disclosed in International Application No. PCT/US2021/063685, which is incorporated by reference in its entirety, or a pharmaceutically acceptable salt, solvate, isomer, stereoisomer, prodrug, or tautomer thereof. In some embodiments, the SOS1 inhibitor is a compound described by any of Formula (I), Formula (I-a), Formula (I-b), Formula (Ic-1), Formula (Ic-2), Formula (Ic-3), Formula (II), Formula (II-a), Formula (II-b), Formula (IIc-1), Formula (IIc-2), Formula (IIc-3), Formula (III), Formula (III-a), Formula (III-b), Formula (IIIc-1), Formula (IIIc-2), and Formula (IIIc-3) in International Application No. PCT/US2021/063685, or a pharmaceutically acceptable salt, solvate, isomer, stereoisomer, prodrug, or tautomer thereof. In some embodiments, the SOS1 inhibitor is selected from a compound of Table A of International Application No. PCT/US2021/063685, or a pharmaceutically acceptable salt, solvate, isomer, stereoisomer, prodrug, or tautomer thereof.

In some embodiments, the SOS1 inhibitor is a compound having the structure of Formula (53-I), (53-II), or (53-III):

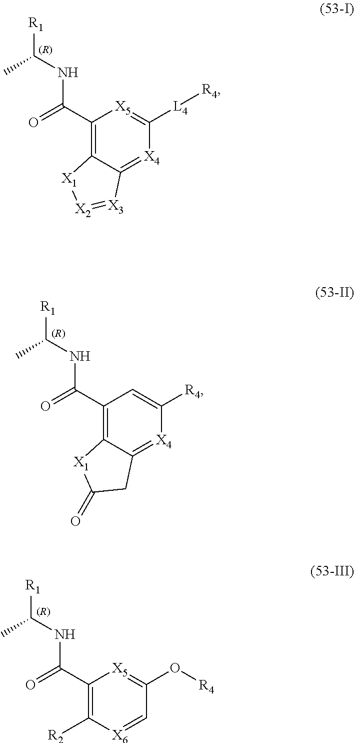

or a pharmaceutically acceptable salt, solvate, isomer, stereoisomer, prodrug, or tautomer thereof, wherein:
X₁ is NH or S;
X₂ is CH or N;
X₃ is CH or N;
X₄ is CR₃ or N;
X₅ is CH or N;
X₆ is CH or N;
R¹ is selected from the group consisting of optionally substituted 3-6 membered cycloalkyl, optionally substituted 3-6 membered heterocyclyl, optionally substituted 6-membered aryl, and optionally substituted 5-6 membered heteroaryl;
R² is selected from the group consisting of H, —NH—C₁₋₆ alkyl, and —NH₂;
R³ is selected from the group consisting of H, —O—C₁₋₆ alkyl, and —O—C₁₋₆ heteroalkyl;
L⁴ is a bond or O; and
R⁴ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with one or more C₁₋₆ alkyl, —R₄ₐ, —OR^{4a}, —O—C₁₋₆ alkyl-R₄ₐ, ═O, halogen, —C(O)R₄ₐ, C(O)OR₄ₐ, —C(O)NR_{4b}R_{4c}, —NR_{4b}C(O)R_{4c}, —CN, ═NR₄ₐ, —NR_{4b}R_{4c}, —SO₂R₄ₐ, 3-6 membered cycloalkyl optionally substituted with R₄ₐ, 3-7 membered heterocyclyl optionally substituted with R₄ₐ, 6-10 membered aryl optionally substituted with R₄ₐ, or 5-10 membered heteroaryl optionally substituted with R₄ₐ;
wherein R₄ₐ is H, C₁₋₆ alkyl, C₁₋₆ haloalkyl, —C(O)R_{4b}, C(O)NR_{4b}R_{4c}, ═O, 3-6 membered cycloalkyl, 6-10 membered aryl optionally substituted with —OR_{4b}, —CN, ═N-3-6 membered cycloalkyl, 3-7 membered heterocyclyl, —(CH₂)ᵣOCH₃, or —(CH₂)ᵣOH, wherein r is 1, 2, or 3;
wherein each R_{4b} is independently H, C₁₋₆ alkyl; and
   wherein each R_{4c} is independently H or C₁₋₆ alkyl.

In some embodiments, the present invention is directed to a method of treating a subject having a disease or disorder characterized by SHP2-mediated activation of a RAS protein comprising administering to a subject in need of such treatment a therapeutically effective amount of a SOS1 inhibitor having the structure of Formula (41-I), (42-I), (48-I), (53-I), (53-II), (53-III), or a pharmaceutically acceptable salt, solvate, isomer, stereoisomer, prodrug, or tautomer thereof. In some embodiments, the present invention is directed to a method of treating a subject having a disease or disorder characterized by SHP2-mediated activation of a RAS protein comprising administering to a subject in need of such treatment a therapeutically effective amount of a SOS1 inhibitor having the structure of any one of:
(A) Formula (I), Formula (I-a), Formula (II), Formula (II-a), Formula (III), or Formula (III-a) in WO 2020/180768, or a pharmaceutically acceptable salt, solvate, isomer, stereoisomer, prodrug, or tautomer thereof;
(B) Formula (I), Formula (I-a), Formula (II), Formula (III), Formula (IV), Formula (V), Formula (V-a), Formula (VI), (VI-a), Formula (VII-a), Formula (VII-b), Formula (VII-c), and Formula (VII-d), in WO 2020/180770, or a pharmaceutically acceptable salt, solvate, isomer, stereoisomer, prodrug, or tautomer thereof;
(C) Formula (I), Formula (II), Formula (III), Formula (IV-a), Formula (IV-b), and Formula (IV-c), in WO 2021/092115, or a pharmaceutically acceptable salt, solvate, isomer, stereoisomer, prodrug, or tautomer thereof; or
(D) Formula (I), Formula (I-a), Formula (I-b), Formula (Ic-1), Formula (Ic-2), Formula (Ic-3), Formula (II), Formula (II-a), Formula (II-b), Formula (IIc-1), Formula (IIc-2), Formula (IIc-3), Formula (III), Formula (III-a), Formula (III-b), Formula (IIIc-1), Formula (IIIc-2), and Formula (IIIc-3) in International Application No. PCT/US2021/063685, or a pharmaceutically acceptable salt, solvate, isomer, stereoisomer, prodrug, or tautomer thereof.

In some embodiments, the method of inhibiting SOS1 in a subject comprises administering to the subject in need of such treatment a therapeutically effective amount of a SOS1 inhibitor having the structure of Formula (41-I), (42-I), (48-I), (53-I), (53-II), (53-III), or a pharmaceutically acceptable salt, solvate, isomer, stereoisomer, prodrug, or tautomer thereof. In some embodiments, the method of inhibiting SOS1 in a subject comprises administering to the subject in need of such treatment a therapeutically effective amount of a SOS1 inhibitor having the structure of any one of:
(A) Formula (I), Formula (I-a), Formula (II), Formula (II-a), Formula (III), or Formula (III-a) in WO 2020/180768, or a pharmaceutically acceptable salt, solvate, isomer, stereoisomer, prodrug, or tautomer thereof;
(B) Formula (I), Formula (I-a), Formula (II), Formula (III), Formula (IV), Formula (V), Formula (V-a), Formula (VI), (VI-a), Formula (VII-a), Formula (VII-b), Formula (VII-c), and Formula (VII-d), in WO 2020/180770, or a pharmaceutically acceptable salt, solvate, isomer, stereoisomer, prodrug, or tautomer thereof;
(C) Formula (I), Formula (II), Formula (III), Formula (IV-a), Formula (IV-b), and Formula (IV-c), in WO 2021/092115, or a pharmaceutically acceptable salt, solvate, isomer, stereoisomer, prodrug, or tautomer thereof; or
(D) Formula (I), Formula (I-a), Formula (I-b), Formula (Ic-1), Formula (Ic-2), Formula (Ic-3), Formula (II), Formula (II-a), Formula (II-b), Formula (IIc-1), Formula (IIc-2), Formula (IIc-3), Formula (III), Formula (III-a), Formula (III-b), Formula (IIIc-1), Formula (IIIc-2), and Formula (IIIc-3) in International Application No. PCT/US2021/063685, or a pharmaceutically acceptable salt, solvate, isomer, stereoisomer, prodrug, or tautomer thereof.

In some embodiments, the present invention is directed to a method of inhibiting the interaction of SOS1 and a RAS-family protein in a cell or inhibiting the interaction of SOS1 and RAC1 in a cell, comprising administering to the cell a SOS1 inhibitor having the structure of Formula (41-I), (42-I), (48-I), (53-I), (53-II), (53-III), or a pharmaceutically acceptable salt, solvate, isomer, stereoisomer, prodrug, or tautomer thereof. In some embodiments, the present invention is directed to a method of inhibiting the interaction of SOS1 and a RAS-family protein in a cell or inhibiting the interaction of SOS1 and RAC1 in a cell, comprising administering to the cell a SOS1 inhibitor having the structure of any one of:
(A) Formula (I), Formula (I-a), Formula (II), Formula (II-a), Formula (III), or Formula (III-a) in WO 2020/180768, or a pharmaceutically acceptable salt, solvate, isomer, stereoisomer, prodrug, or tautomer thereof;
(B) Formula (I), Formula (I-a), Formula (II), Formula (III), Formula (IV), Formula (V), Formula (V-a), Formula (VI), (VI-a), Formula (VII-a), Formula (VII-b), Formula (VII-c), and Formula (VII-d), in WO 2020/180770, or a pharmaceutically acceptable salt, solvate, isomer, stereoisomer, prodrug, or tautomer thereof;
(C) Formula (I), Formula (II), Formula (III), Formula (IV-a), Formula (IV-b), and Formula (IV-c), in WO 2021/092115, or a pharmaceutically acceptable salt, solvate, isomer, stereoisomer, prodrug, or tautomer thereof; or
(D) Formula (I), Formula (I-a), Formula (I-b), Formula (Ic-1), Formula (Ic-2), Formula (Ic-3), Formula (II), Formula (II-a), Formula (II-b), Formula (IIc-1), Formula (IIc-2), Formula (IIc-3), Formula (III), Formula (III-a), Formula (III-b), Formula (IIIc-1), Formula (IIIc-2), and Formula (IIIc-3) in International Application No. PCT/US2021/063685, or a pharmaceutically acceptable salt, solvate, isomer, stereoisomer, prodrug, or tautomer thereof.

In some embodiments, the present invention is directed to a method of treating or preventing a disease, wherein treating or preventing the disease is characterized by inhibition of the interaction of SOS1 and a RAS-family protein or by inhibition of the interaction of SOS1 and RAC1, the method comprising administering to a subject in need thereof a therapeutically effective amount of a SOS1 inhibitor having the structure of Formula (41-I), (42-I), (48-I), (53-1), (53-II), (53-III), or a pharmaceutically acceptable salt, solvate, isomer, stereoisomer, prodrug, or tautomer thereof. In some embodiments, the present invention is directed to a method of treating or preventing a disease, wherein treating or preventing the disease is characterized by inhibition of the interaction of SOS1 and a RAS-family protein or by inhibition of the interaction of SOS1 and RAC1, the method comprising administering to a subject in need thereof a therapeutically effective amount of a SOS1 inhibitor having the structure of any one of:
(A) Formula (I), Formula (I-a), Formula (II), Formula (II-a), Formula (III), or Formula (III-a) in WO 2020/180768, or a pharmaceutically acceptable salt, solvate, isomer, stereoisomer, prodrug, or tautomer thereof;
(B) Formula (I), Formula (I-a), Formula (II), Formula (III), Formula (IV), Formula (V), Formula (V-a), Formula (VI), (VI-a), Formula (VII-a), Formula (VII-b), Formula (VII-c), and Formula (VII-d), in WO 2020/180770, or a pharmaceutically acceptable salt, solvate, isomer, stereoisomer, prodrug, or tautomer thereof;
(C) Formula (I), Formula (II), Formula (III), Formula (IV-a), Formula (IV-b), and Formula (IV-c), in WO 2021/092115, or a pharmaceutically acceptable salt, solvate, isomer, stereoisomer, prodrug, or tautomer thereof; or
(D) Formula (I), Formula (I-a), Formula (I-b), Formula (Ic-1), Formula (Ic-2), Formula (Ic-3), Formula (II), Formula (II-a), Formula (II-b), Formula (IIc-1), Formula (IIc-2), Formula (IIc-3), Formula (III), Formula (III-a), Formula (III-b), Formula (IIIc-1), Formula (IIIc-2), and Formula (IIIc-3) in International Application No. PCT/US2021/063685, or a pharmaceutically acceptable salt, solvate, isomer, stereoisomer, prodrug, or tautomer thereof.

In some embodiments, the present invention is directed to a method of treating or preventing cancer in a subject in need thereof, comprising administering to the subject a therapeutically effective amount of a SOS1 inhibitor having the structure of Formula (41-I), (42-I), (48-I), (53-I), (53-II), (53-III), or a pharmaceutically acceptable salt, solvate, isomer, stereoisomer, prodrug, or tautomer thereof. In some embodiments, the present invention is directed to a method of treating or preventing cancer in a subject in need thereof, comprising administering to the subject a therapeutically effective amount of a SOS1 inhibitor having the structure of any one of:
(A) Formula (I), Formula (I-a), Formula (II), Formula (II-a), Formula (III), or Formula (III-a) in WO 2020/180768, or a pharmaceutically acceptable salt, solvate, isomer, stereoisomer, prodrug, or tautomer thereof;
(B) Formula (I), Formula (I-a), Formula (II), Formula (III), Formula (IV), Formula (V), Formula (V-a), Formula (VI), (VI-a), Formula (VII-a), Formula (VII-b), Formula (VII-c), and Formula (VII-d), in WO 2020/180770, or a pharmaceutically acceptable salt, solvate, isomer, stereoisomer, prodrug, or tautomer thereof;
(C) Formula (I), Formula (II), Formula (III), Formula (IV-a), Formula (IV-b), and Formula (IV-c), in WO 2021/092115, or a pharmaceutically acceptable salt, solvate, isomer, stereoisomer, prodrug, or tautomer thereof; or
(D) Formula (I), Formula (I-a), Formula (I-b), Formula (Ic-1), Formula (Ic-2), Formula (Ic-3), Formula (II), Formula (II-a), Formula (II-b), Formula (IIc-1), Formula (IIc-2), Formula (IIc-3), Formula (III), Formula (III-a), Formula (III-b), Formula (IIIc-1), Formula (IIIc-2), and Formula (IIIc-3) in International Application No. PCT/US2021/063685, or a pharmaceutically acceptable salt, solvate, isomer, stereoisomer, prodrug, or tautomer thereof.

In some embodiments, the SOS1 inhibitor is BI-3406, having the structure:

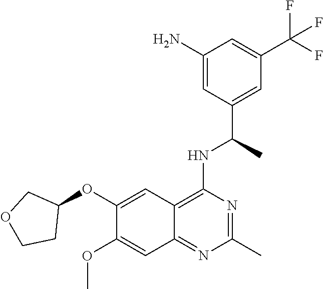 [SMILES: COc1cc2nc(C)nc(N[C@H](C)c3cc(N)cc(C(F)(F)F)c3)c2cc1O[C@H]1CCOC1]

or a pharmaceutically acceptable salt, solvate, isomer, stereoisomer, prodrug, or tautomer thereof.

In some embodiments, the SOS1 inhibitor is BI-1701963 or a pharmaceutically acceptable salt, solvate, isomer, stereoisomer, prodrug, or tautomer thereof.

In some embodiments, the SOS1 inhibitor is BAY-293, having the structure:

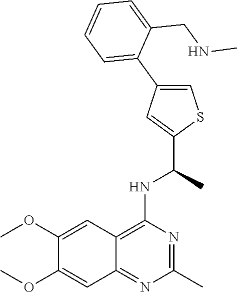

or a pharmaceutically acceptable salt, solvate, isomer, stereoisomer, prodrug, or tautomer thereof.

In some embodiments, the SOS1 inhibitor is SDR5 or MRTX0902 or a pharmaceutically acceptable salt, solvate, isomer, stereoisomer, prodrug, or tautomer thereof.

In some embodiments, the SOS1 inhibitor is Compound SOS1-(A) (also called RMC-0331), having the structure:

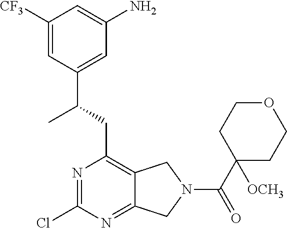 [SMILES: COC1(C(=O)N2Cc3nc(Cl)nc(C[C@H](C)c4cc(N)cc(C(F)(F)F)c4)c3C2)CCOCC1]

or a pharmaceutically acceptable salt, solvate, isomer, stereoisomer, prodrug, or tautomer thereof.

In some embodiments, the SOS1 inhibitor is Compound SOS1-(B), or a pharmaceutically acceptable salt, solvate, isomer, stereoisomer, prodrug, or tautomer thereof.

In some embodiments, the SOS1 inhibitor is Compound SOS1-(C), or a pharmaceutically acceptable salt, solvate, isomer, stereoisomer, prodrug, or tautomer thereof.

The SOS1 inhibitor dose may range from a dose sufficient to elicit a response to the maximum tolerated dose. Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses, such as from 100 mg to 1300 mg, from 200 mg to 1300 mg, from 600 mg to 1300 mg, from 700 mg to 1200 mg, or from 800 mg to 1000 mg. In one embodiment, the compositions are in the form of a tablet that can be scored. The SOS1 inhibitor can be dosed once per day, twice per day, three times per day, or four times per day. In some aspects, the SOS1 inhibitor is dosed once per day. In some aspects, the SOS1 inhibitor is dosed twice per day. Dosing may be done with or without food. The dosing schedule may suitably be every day of a 28-day schedule, or 21 or more days of a 28-day schedule.

mTOR Inhibitors

According to some embodiments of the present disclosure, the method comprises treating a subject having a disease or disorder, the method comprising administering to a subject in need of such treatment (a) a therapeutically effective amount of a SOS1 inhibitor or a pharmaceutically acceptable salt, solvate, isomer, stereoisomer, prodrug, or tautomer thereof; and (b) a therapeutically effective amount of a bi-steric mTOR inhibitor, or a pharmaceutically acceptable salt, solvate, isomer, stereoisomer, prodrug, or tautomer thereof.

According to some embodiments, the bi-steric mTOR inhibitor is selected from those disclosed in WO 2016/040806, or a pharmaceutically acceptable salt, solvate, isomer, stereoisomer, prodrug, or tautomer thereof, the disclosure of which is hereby incorporated by reference as if set forth in its entirety.

According to some embodiments, the bi-steric mTOR inhibitor is selected from those disclosed in WO 2018/204416, or a pharmaceutically acceptable salt, solvate, isomer, stereoisomer, prodrug, or tautomer thereof, the disclosure of which is hereby incorporated by reference as if set forth in its entirety.

According to some embodiments, the bi-steric mTOR inhibitor is selected from those disclosed in WO 2019/212990, or a pharmaceutically acceptable salt, solvate, isomer, stereoisomer, prodrug, or tautomer thereof, the disclosure of which is hereby incorporated by reference as if set forth in its entirety.

According to some embodiments, the bi-steric mTOR inhibitor is selected from those disclosed in WO 2019/212991, or a pharmaceutically acceptable salt, solvate, isomer, stereoisomer, prodrug, or tautomer thereof, the disclosure of which is hereby incorporated by reference as if set forth in its entirety.

In some embodiments, the mTOR inhibitor is a compound as disclosed in WO 2019/212990, or a pharmaceutically acceptable salt, solvate, isomer, stereoisomer, prodrug, or tautomer thereof, which is incorporated by reference in its entirety. In some embodiments, the mTOR inhibitor is a compound described by any of Formula (I), Formula (Ia), Formula (Ib), Formula (Ic), Formula (II), Formula (IIb), in WO 2019/212990, or a pharmaceutically acceptable salt, solvate, isomer, stereoisomer, prodrug, or tautomer thereof. In some embodiments, the mTOR inhibitor is selected from a compound of the Tables of WO 2019/212990, or a pharmaceutically acceptable salt, solvate, isomer, stereoisomer, prodrug, or tautomer thereof.

According to some embodiments, the bi-steric mTOR inhibitor is RM-006 (also known as RMC-6272) having the following structure:

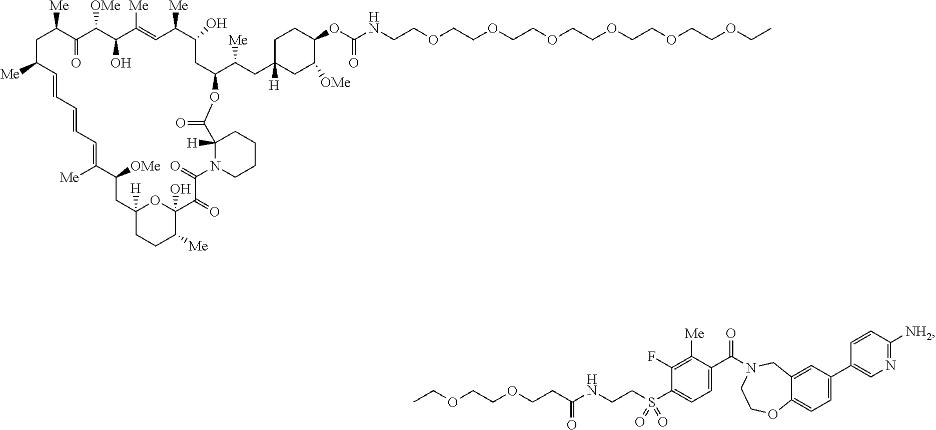

or a pharmaceutically acceptable salt, solvate, isomer, stereoisomer, prodrug, or tautomer thereof.

According to some embodiments, the bi-steric mTOR inhibitor is RMC-5552 having the following structure:

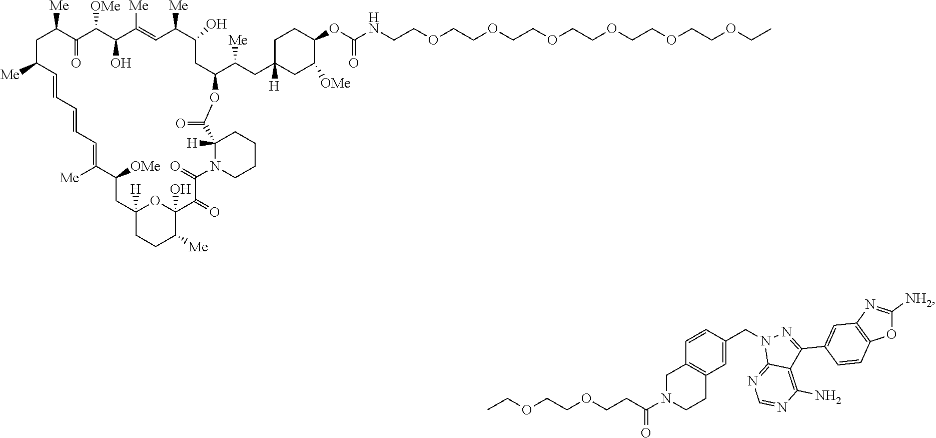

or a pharmaceutically acceptable salt, solvate, isomer, stereoisomer, prodrug, or tautomer thereof. In some embodiments, the bi-steric mTOR inhibitor is RMC-5552 having the following structure:

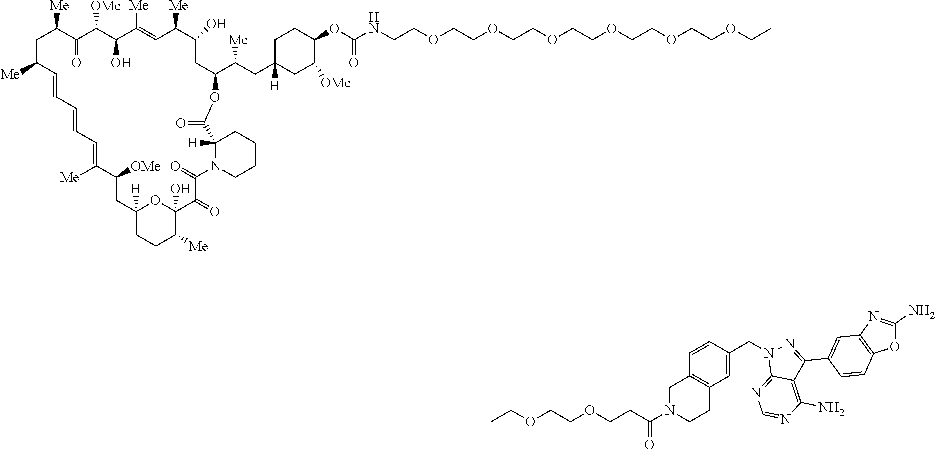

or a stereoisomer, tautomer or oxepane isomer thereof.

The bi-steric mTOR inhibitor dose may range from a dose sufficient to elicit a response to the maximum tolerated dose. Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses, such as from 100 mg to 1300 mg, from 200 mg to 1300 mg, from 600 mg to 1300 mg, from 700 mg to 1200 mg, or from 800 mg to 1000 mg. In one embodiment, the compositions are in the form of a tablet that can be scored. The mTOR inhibitor can be dosed once per day, twice per day, three times per day, or four times per day. In some aspects, the bi-steric mTOR inhibitor is dosed once per day. In some aspects, the bi-steric mTOR inhibitor is dosed twice per day. Dosing may be done with or without food. The dosing schedule may suitably be every day of a 28-day schedule, or 21 or more days of a 28-day schedule.

Exemplary Combinations of mTOR Inhibitors and SOS1 Inhibitors

In some embodiments, the method comprises administering a combination of a bi-steric mTOR inhibitor and a SOS1 inhibitor. Exemplary, non-limiting combinations of such inhibitors include the following.

In some embodiments, the SOS1 inhibitor is Compound SOS1-(A) having the structure:

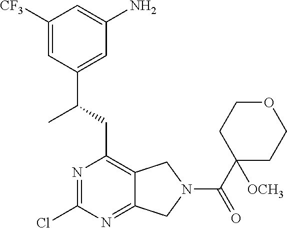 [SMILES: COC1(C(=O)N2Cc3nc(Cl)nc(C[C@H](C)c4cc(N)cc(C(F)(F)F)c4)c3C2)CCOCC1]

or a pharmaceutically acceptable salt, solvate, isomer, stereoisomer, prodrug, or tautomer thereof; and
the bi-steric mTOR inhibitor is RMC-5552 having the following structure:

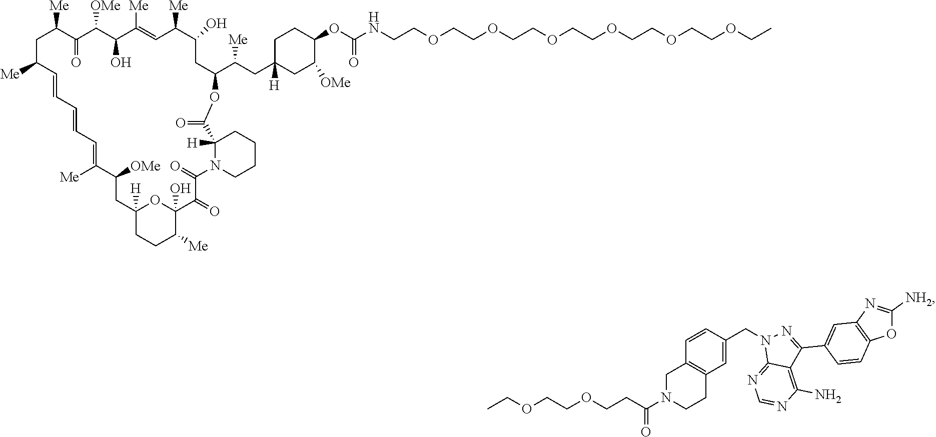

or a pharmaceutically acceptable salt, solvate, isomer, stereoisomer, prodrug, or tautomer thereof. In some embodiments, the bi-steric mTOR inhibitor is RMC-5552 having the following structure:

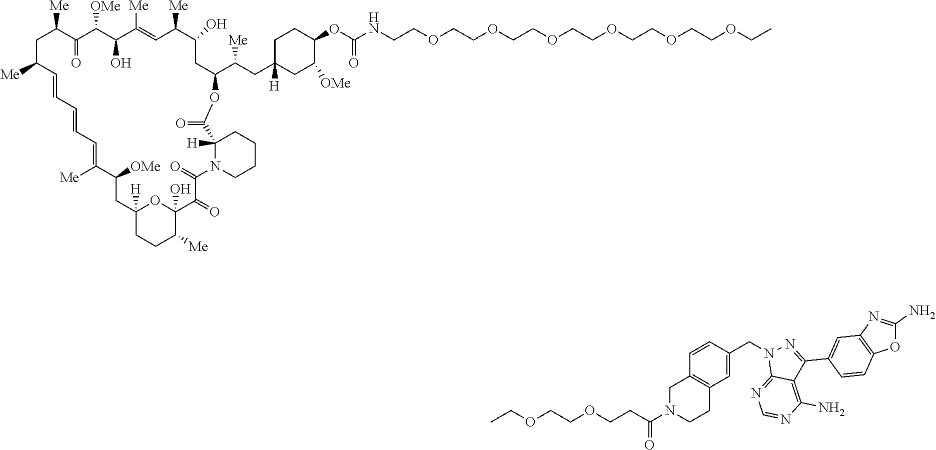

or a stereoisomer, tautomer or oxepane isomer thereof.

In some embodiments, the SOS1 inhibitor is Compound SOS1-(B) or a pharmaceutically acceptable salt, solvate, isomer, stereoisomer, prodrug, or tautomer thereof; and

the bi-steric mTOR inhibitor is RMC-5552 having the following structure:

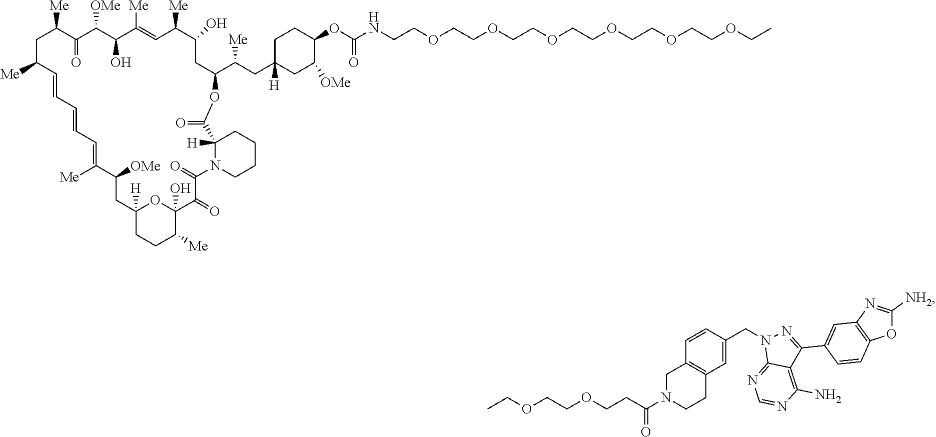

or a pharmaceutically acceptable salt, solvate, isomer, stereoisomer, prodrug, or tautomer thereof. In some embodiments, the bi-steric mTOR inhibitor is RMC-5552 having the following structure:

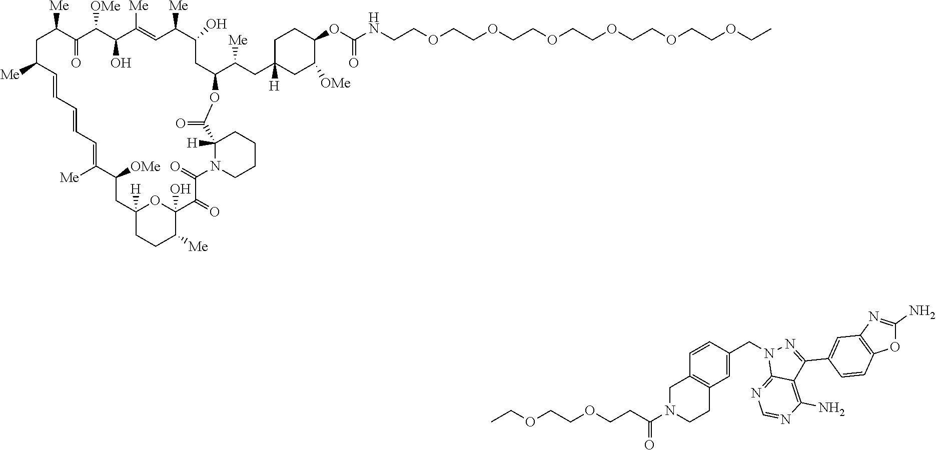

or a stereoisomer, tautomer or oxepane isomer thereof.

In some embodiments, the SOS1 inhibitor is Compound SOS1-(C) or a pharmaceutically acceptable salt, solvate, isomer, stereoisomer, prodrug, or tautomer thereof; and

the bi-steric mTOR inhibitor is RMC-5552 having the following structure:

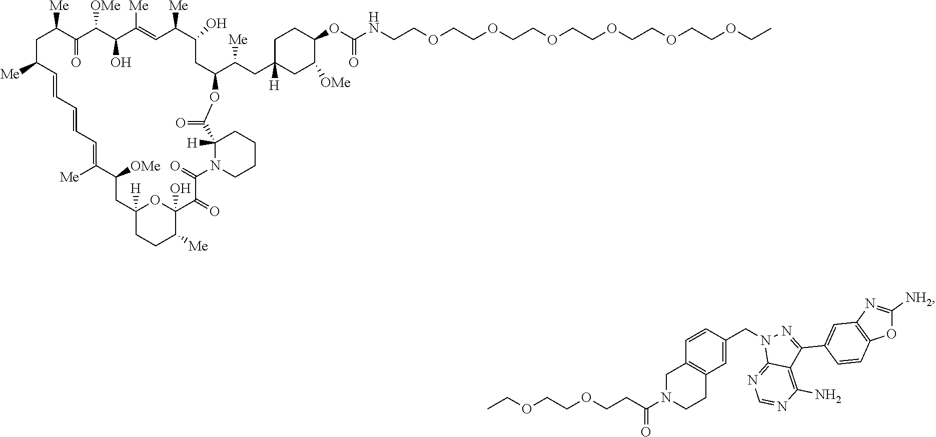

or a pharmaceutically acceptable salt, solvate, isomer, stereoisomer, prodrug, or tautomer thereof. In some embodiments, the bi-steric mTOR inhibitor is RMC-5552 having the following structure:

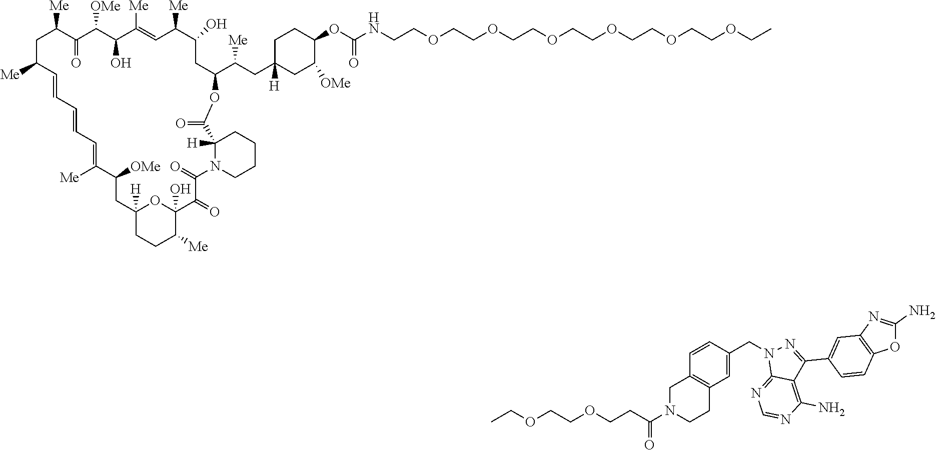

or a stereoisomer, tautomer or oxepane isomer thereof.

In some embodiments, the SOS1 inhibitor is Compound SOS1-(B) or a pharmaceutically acceptable salt, solvate, isomer, stereoisomer, prodrug, or tautomer thereof; and

the bi-steric mTOR inhibitor is RM-006 (also known as RMC-6272) having the following structure:

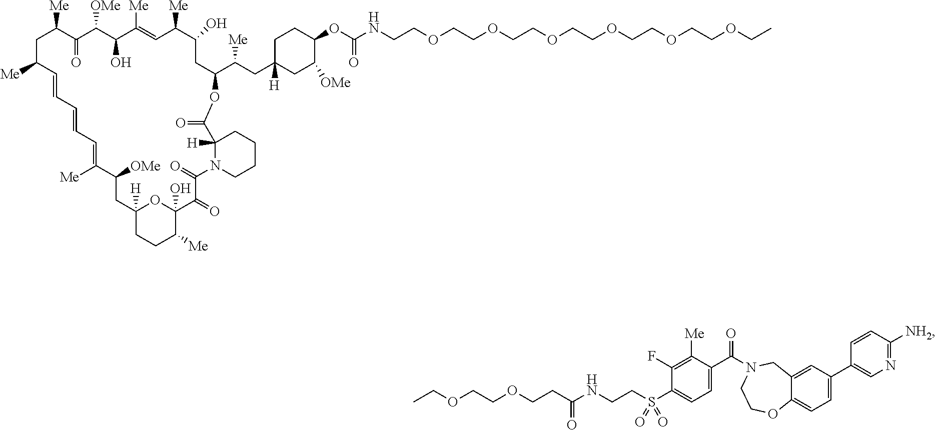

or a pharmaceutically acceptable salt, solvate, isomer, stereoisomer, prodrug, or tautomer thereof. In some embodiments, the bi-steric mTOR inhibitor is RMC-5552 having the following structure:

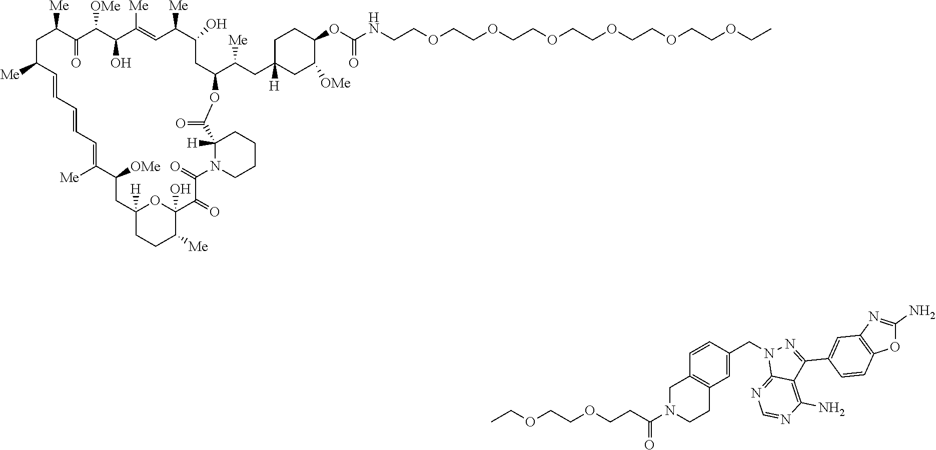

or a stereoisomer, tautomer or oxepane isomer thereof.

### Therapeutic Use

Due to their biological properties the compounds of the present disclosure, their tautomers, racemates, enantiomers, diastereomers, mixtures thereof and the salts of all the above-mentioned forms may be suitable for treating diseases characterized by excessive or abnormal cell proliferation such as cancer.

For example, the following cancers, tumors and other proliferative diseases may be treated with compounds of the present disclosure, without being restricted thereto:
cancers/tumors/carcinomas of the head and neck: e.g., tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands); intraocular cancers (e.g., uveal melanoma), and orbital and adnexal cancers;
cancers/tumors/carcinomas of the lung: e.g., non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);
neoplasms of the mediastinum: e.g., neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma), astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g., acoustic), spinal axis tumors;
cancers/tumors/carcinomas of the gastrointestinal (GI) tract: e.g., tumors/carcinomas/cancers of the esophagus, stomach (gastric cancer), pancreas, liver and biliary tree (including hepatocellular carcinoma (HCC), e.g., childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), genitourinary system (including kidney, e.g., renal pelvis, renal cell carcinoma (RCC), nephroblastoma (Wilms tumor), hypernephroma, Grawitz tumor; ureter; urinary bladder, e.g., urachal cancer, urothelial cancer; urethra, e.g., distal, bulbomembranous, prostatic; prostate (androgen dependent, androgen independent, castration resistant, hormone independent, hormone refractory), penis);
cancers/tumors/carcinomas of the testis: e.g., seminomas, non-seminomas;
gynecologic cancers/tumors/carcinomas: e.g., tumors/carcinomas/cancers of the ovary, fallopian tube, peritoneum, cervix, vulva, vagina, uterine body (including endometrium, fundus);
cancers/tumors/carcinomas of the breast: e.g., mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), HER2 positive breast cancer, triple negative breast cancer, Paget's disease of the breast;
cancers/tumors/carcinomas of the endocrine system: e.g., tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; non-functional PET, PPoma, gastrinoma, insulinoma, VIPoma, glucagonoma, somatostatinoma, GRFoma, ACTHoma), carcinoid tumors;
sarcomas of the soft tissues: e.g., fibrosarcoma, fibrous histiocytoma, liposarcoma, leiomyosarcoma, rhabdomyosarcoma, angiosarcoma, lymphangiosarcoma, Kaposi s sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewings sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor;
sarcomas of the bone: e.g., myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;
mesothelioma: e.g., pleural mesothelioma, peritoneal mesothelioma;
cancers of the skin: e.g., basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer;
neoplasms of the peripheral and central nervous system and brain: e.g., astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g., acoustic), spinal axis tumors, neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);
lymphomas and leukemias: e.g., B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), Burkitt leukemia, T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (B-CLL), chronic T-cell lymphocytic leukemia (T-CLL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML), JMML (juvenile myelomonocytic leukemia), acute leukemia of ambiguous lineage, myeloproliferative neoplasms, blastic plasmacytoid dendritic cell neoplasm, early T-cell precursor leukemia, natural killer cell leukemia/lymphoma, myeloid/lymphoid neoplasms with eosinophilia, myeloid sarcoma, transient abnormal myelopoiesis; and
cancers of unknown primary site (CUP).

All cancers/tumors/carcinomas mentioned above which are characterized by their specific location/origin in the body are meant to include both the primary tumors and the metastatic tumors derived therefrom.

All cancers/tumors/carcinomas mentioned above may be further differentiated by their histopathological classification:
epithelial cancers, e.g., squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma; and
nonepithilial and mesenchymal cancers, e.g., sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas.

A disease or disorder may be a hematologic cancer, for example: blood (myeloid leukemia (acute and chronic), acute lymphoblastic leukemia, chronic lymphocytic leukemia, myeloproliferative diseases (e.g., myelofibrosis and myeloproliferative neoplasms), multiple myeloma, myelodysplastic syndrome), Hodgkin's disease, or non-Hodgkin's lymphoma (malignant lymphoma).

In some embodiments, a disease or disorder is selected from the group consisting of pancreatic cancer, colorectal cancer, non-small cell lung carcinoma and glioblastoma. In some embodiments, the disease or disorder is selected from endometrial cancer, uterine cancer, breast cancer, small-cell lung cancer, non-small cell lung carcinoma (e.g., harboring a KRAS mutation (e.g., KRASG12C), a STK11 mutation or a KEAP1 mutation, or a combination thereof), squamous cell lung carcinoma, cervical cancer, glioblastoma, colorectal cancer, melanoma, head and neck cancer (e.g., squamous cell cancer of the head and neck (HNSCC)), ovarian cancer, prostate cancer, FGFR mutant cancer, hepatocellular carcinoma, hepatoblastoma, glioma, rhabdomyosarcoma, bladder cancer, hematological cancer, pancreatic cancer, renal cancer, neuro-endocrine cancer, thyroid gland adenocarcinoma, a cancer characterized by increased mTORC1 activity, a cancer with an mTORC1 pathway mutation (e.g., STK11, PIK3CA, PTEN, mTOR, KEAP1, TSC1, or TSC2), a cancer having a MYC amplification (e.g., MYC, MYCL, or MYCN), a cancer characterized by decreased 4EBP1 activity, and a cancer with a PTEN mutation.

The compounds of the present disclosure may be used in therapeutic regimens in the context of first line, second line, or any further line treatments.

The compounds of the invention may be used for the prevention, short-term or long-term treatment of the above-mentioned diseases, optionally also in combination with radiotherapy and/or surgery and/or other compounds.

Of course, the above also includes the use of the compounds of the present disclosure in various methods of treating the above diseases by administering a therapeutically effective dose to a patient in need thereof, as well as the use of these compounds for the manufacture of medicaments for the treatment of such diseases, as well as pharmaceutical compositions including such compounds of the invention, as well as the preparation and/or manufacture of medicaments including such compounds of the invention, and the like.

### Additional Combination Therapies

In some embodiments, the subject is co-administered a therapeutically effective amount of an additional therapeutic agent.

For example, a therapeutic agent may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PDL-1. In some embodiments, the checkpoint inhibitor is an inhibitor or antagonist (e.g., an inhibitory antibody or Fc fusion or small molecule inhibitor) of PDL-2 (e.g., a PDL-2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MED14736, MPDL3280A, MSB0010718C, BMS986016, IMP321, lirilumab, IPH2101, 1-7F9, and KW-6002.

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18: 233a (1999), and Douillard et al., Lancet 355 (9209): 1041-1047 (2000).

Other non-limiting examples of anti-cancer agents include Gleevec® (Imatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antibiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4 (5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., a CDK 4/6 inhibitor such as ribociclib, abemaciclib, or palbociclib), seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTOR inhibitors (e.g., vistusertib, temsirolimus, everolimus, ridaforolimus, and sirolimus), KSP(Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CSI (e.g., elotuzumab), HSP90 inhibitors (e.g., 17 AAG and KOS 953), P13K/Akt inhibitors (e.g., perifosine), Akt inhibitors (e.g., GSK-2141795), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, the anti-cancer agent is a colony-stimulating factor 1 receptor (CSF1R) inhibitor. See, e.g., Cannarile et al., J ImmunoTherapy Cancer 5:53 (2017) and Xun et al., Curr Med Chem 27:3944 (2020).

In some embodiments, an anti-cancer agent is an anti-CD40 antibody, such as APX005M.

A therapeutic agent may be an anti-TIGIT antibody, such as MBSA43, BMS-986207, MK-7684, COM902, AB154, MTIG7192A or OMP-313M32 (etigilimab).

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, an anti-cancer agent is an ALK inhibitor. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., ERAS-601, SHP099, SH3809, PF-07284892, TNO155, RMC-4550, RMC-4630, JAB-3068, JAB-3312, BBP-398, RLY-1971), a Raf inhibitor, a MEK inhibitor, an ERK inhibitor, a PI3K inhibitor, a PTEN inhibitor, an AKT inhibitor, or an mTOR inhibitor (e.g., mTORC1 inhibitor or mTORC2 inhibitor).

In some embodiments, an anti-cancer agent is a Ras inhibitor (e.g., AMG 510, MRTX1257, MRTX849, JNJ-74699157 (ARS-3248), LY3537982, ARS-853, ARS-1620, GDC-6036, BPI-421286, JDQ443, JAB-21000, JAB-22000, JAB-23000, JAB-21822, GH-35, ICP-915, IBI351, ERAS-3490, D-1553, RMC-6291, RMC-6236, RMC-9805 or RMC-8839, or a Ras vaccine, or another therapeutic modality designed to directly or indirectly decrease the oncogenic activity of Ras.

In some embodiments, an anti-cancer agent is a RAS(ON) inhibitor. As used herein, the term “RAS(ON) inhibitor” refers to an inhibitor that targets, that is, selectively binds to or inhibits, the GTP-bound, active state of RAS (e.g., selective over the GDP-bound, inactive state of RAS). Inhibition of the GTP-bound, active state of RAS includes, for example, the inhibition of oncogenic signaling from the GTP-bound, active state of RAS. In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS).

In some embodiments, an anti-cancer agent is a RAS(OFF) inhibitor. As used herein, the term “RAS(OFF) inhibitor” refers to an inhibitor that targets, that is, selectively binds to or inhibits the GDP-bound, inactive state of RAS (e.g., selective over the GTP-bound, active state of RAS). Inhibition of the GDP-bound, inactive state of RAS includes, for example, sequestering the inactive state by inhibiting the exchange of GDP for GTP, thereby inhibiting RAS from adopting the active conformation. In certain embodiments, RAS(OFF) inhibitors may also bind to or inhibit the GTP-bound, active state of RAS (e.g., with a lower affinity or inhibition constant than for the GDP-bound, inactive state of RAS).

In some embodiments, a therapeutic agent is an inhibitor of the MAP kinase (MAPK) pathway (or “MAPK inhibitor”). MAPK inhibitors include, but are not limited to, one or more MAPK inhibitor described in Cancers (Basel) 2015 September; 7 (3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLOS One. 2014 Nov. 25; 9 (11)); and GSK1120212 (or JTP-74057, described in Clin Cancer Res. 2011 Mar. 1; 17 (5): 989-1000).

In some embodiments, an anti-cancer agent is a disrupter or inhibitor of the RAS-RAF-ERK or PI3K-AKT-TOR or PI3K-AKT signaling pathways. The PI3K/AKT inhibitor may include, but is not limited to, one or more PI3K/AKT inhibitor described in Cancers (Basel) 2015 September; 7 (3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-L1 antagonist.

In some embodiments, additional therapeutic agents include EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies.

IGF-1R inhibitors include linsitinib, or a pharmaceutically acceptable salt thereof.

EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59 (8): 1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), erlotinib (Tarceva®), and lapatinib (TykerBR). See, e.g., Yan et al., Pharmacogenetics and Pharmacogenomics In Oncology Therapeutic Antibody Development, BioTechniques 2005, 39 (4): 565-8; and Paez et al., EGFR Mutations In Lung Cancer Correlation With Clinical Response To Gefitinib Therapy, Science 2004, 304 (5676): 1497-500. Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8 (12): 1599-1625. In some embodiments, an EGFR inhibitor is osimertinib.

MEK inhibitors include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from AE51-Q58; AF53-Q58; E203K; L177M; C121S; F53L; K57E; Q56P; and K57N.

PI3K inhibitors include, but are not limited to, wortmannin; 17-hydroxywortmannin analogs described in WO06/044453; 4-[2-(1H-Indazol-4-yl)-6-[[4-(methylsulfonyl) piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-1-(4-((2-(2-aminopyrimidin-5-yl)-7-methyl-4-morpholinothieno[3,2-d]pyrimidin-6-yl)methyl) piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′: 4,5]furo[3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c] quinazolin-5-yl)-nicotinamide (available from Axon Medchem); AS-252424 (5-[1-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a] pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits Aktl) (Barnett et al., Biochem. J. 2005, 385 (Pt. 2): 399-408); Akt-1-1,2 (inhibits Akl and 2) (Barnett et al., Biochem. J. 2005, 385 (Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134 (12 Suppl): 3493S-3498S); perifosine (e.g., interferes with Akt membrane localization; Dasmahapatra et al. Clin. Cancer Res. 2004, 10 (15): 5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

BRAF inhibitors that may be used in combination with compounds of the invention include, for example, vemurafenib, dabrafenib, and encorafenib. A BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N581I; D594N; D594G; D594A; D594H; F595L; G596D; G596R and A762E.

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PDL-1, anti-CTLA4, anti-LAG1, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110 (1): 186-192; Thompson et al., Clin. Cancer Res. 2007, 13 (6): 1757-1761; and WO06/121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the invention is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloprotienase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix® (panitumumab), erlotinib (Tarceva®), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and Medlmmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProlX, USA); METASTATIN, (EntreMed, USA); troponin I, (Harvard University, USA); SU 6668, (SUGEN, USA); OXI 4503, (OXIGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (Proteom Tech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists (ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with compounds of the invention is an autophagy inhibitor. Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

Another example of a therapeutic agent that may be used in combination with compounds of the invention is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the invention include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

In some embodiments, an additional compound is selected from the group consisting of ABT-737, AT-7519, carfilzomib, cobimetinib, danusertib, dasatinib, doxorubicin, GSK-343, JQ1, MLN-7243, NVP-ADW742, paclitaxel, palbociclib and volasertib. In some embodiments, an additional compound is selected from the group consisting of neratinib, acetinib and reversine.

MCL-1 inhibitors include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In some embodiments, effective combination therapy may be achieved with a single composition or pharmaceutical formulation that includes, for example, two agents, or with two distinct compositions or formulations, administered at the same time, wherein one composition or formulation includes a SOS1 inhibitor as described herein and one composition or formulation includes a bi-steric mTOR inhibitor as described herein. Alternatively, administration of one agent may precede or follow the other agent administration by intervals ranging from minutes to months.

Various combinations of therapeutic agents described herein may be employed, such as when a SOS1 inhibitor as described herein is “A” and “B” represents a bi-steric mTOR inhibitor as described herein, non-limiting examples of which are described below:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of agents in combination will take into account the toxicity, if any, of any of the agents being administered. It is expected that treatment cycles would be repeated as necessary.

### Embodiments

Embodiment 1 is a method of treating a subject having a disease or disorder, the method comprising administering to a subject in need of such treatment:
(a) a therapeutically effective amount of a SOS1 inhibitor, or a pharmaceutically acceptable salt, solvate, isomer, stereoisomer, prodrug, or tautomer thereof; and
(b) a therapeutically effective amount of a bi-steric mTOR inhibitor, or a pharmaceutically acceptable salt, solvate, isomer, stereoisomer, prodrug, or tautomer thereof.

Embodiment 2 is a method of Embodiment 1, wherein the SOS1 inhibitor or a pharmaceutically acceptable salt, solvate, isomer, stereoisomer, prodrug, or tautomer thereof is selected from those disclosed in WO 2018/115380, WO 2018/172250, WO 2019/122129, WO 2019/201848, and WO 2021/074227.

Embodiment 3 is a method of Embodiment 1, wherein the SOS1 inhibitor is a compound having the structure of Formula (41-I),

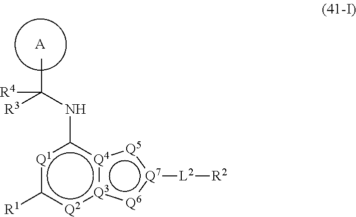

or a pharmaceutically acceptable salt, solvate, isomer, stereoisomer, prodrug, or tautomer thereof, wherein:
Q¹ and Q² are independently CH or N;
Q³, Q⁴, and Q⁷ are independently C or N, wherein at least one of Q³ and Q⁴ is C and wherein Q³, Q⁴, and Q⁷ are not all N;
Q⁵ is CH, N, NH, O, or S;
Q⁶ is CH, N, NH, N—C₁₋₆ alkyl, N—C₁₋₆ heteroalkyl, N-(3-7 membered cycloalkyl), N-(3-7 membered heterocyclyl), O, or S;
wherein at least one of Q¹, Q², Q³, Q⁴, Q⁵, Q⁶, and Q⁷ is N, NH, O, or S;
R¹ is selected from the group consisting of H, C₁₋₆ alkyl, halogen, —NHR^{1a}, —OR^{1a}, cyclopropyl, and —CN; wherein C₁₋₆ alkyl is optionally substituted with halogen, —NHR^{1a}, or —OR^{1a}, wherein R^{1a} is H, C₁₋₆ alkyl, 3-6 membered heterocyclyl, or C₁₋₆ haloalkyl;
L² is selected from the group consisting of a bond, —C(O)—, —C(O)O—, —C(O)NH(CH₂)ₒ—, —S(O)₂—,

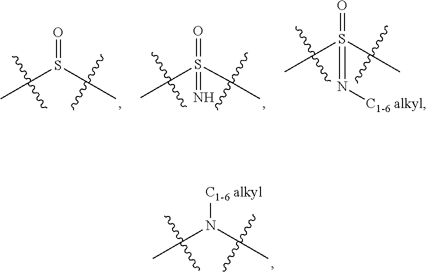

C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, C₂₋₆ alkenyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, C₂₋₆ alkenyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, —OH, —OR^{2a}, oxo, halogen, —C(O)R^{2a}, —C(O)OR^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl;
   wherein R^{2a} is H, C₁₋₆ alkyl, C₁₋₆ haloalkyl, 3-7 membered heterocyclyl, or —(CH₂)ᵣOCH₃, wherein r is 1, 2, or 3;
   wherein R^{2b} is H or C₁₋₆ alkyl;
   wherein R^{2c} is H or C₁₋₆ alkyl;
R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and
A is an optionally substituted 6-membered aryl or an optionally substituted 5-6 membered heteroaryl.

Embodiment 4 is a method of Embodiment 1, wherein the SOS1 inhibitor is a compound having the structure of Formula (42-I),

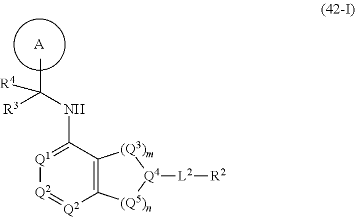

or a pharmaceutically acceptable salt, solvate, isomer, stereoisomer, prodrug, or tautomer thereof, wherein:
Q¹ is CH or N;
Q⁴ is CH, C, or N;
each Q² is independently C—R¹ or N, wherein one Q² is N and the other Q² is C—R¹;
each Q³ and Q⁵ are independently C(R^{QC})₂, NR^{QN}, CO, O, S, or SO₂, wherein each R^{QC} is independently H, F, Cl, Br, or 6-10 membered aryl, and wherein each RON is independently H, C₁₋₆ alkyl, or 6-10 membered aryl;
wherein at least one of Q¹, Q², Q³, Q⁴, and Q⁵ is N, NR^{QN}, O, or SO₂;
m is 0, 1, 2, or 3;
n is 0, 1, 2, or 3;
wherein when m is 0, then n is not 0;
R¹ is selected from the group consisting of H, C₁₋₆ alkyl, halogen, —CONHR^{1a}, —NHR^{1a}, —OR^{1a}, cyclopropyl, azetidinyl, and —CN; wherein each C₁₋₆ alkyl and azetidinyl is optionally substituted with halogen, R^{1a}, —NHR^{1a}, or —OR^{1a}, wherein R^{1a} is H, C₁₋₆ alkyl, cyclopropyl, 3-6 membered heterocyclyl, or C₁₋₆ haloalkyl;
L² is selected from the group consisting of a bond, —C(O)—, —C(O)O—, —C(O)NH(CH₂)ₒ—, —S(O)₂—,

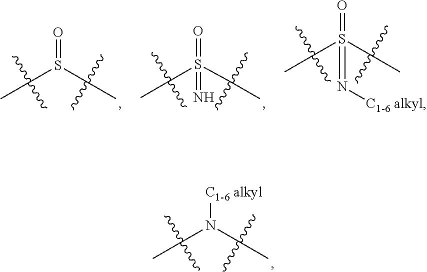

C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, —NR_{2b}R_{2c}, —OR₂ₐ, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, C₁₋₆ hydroxyalkyl, C₁₋₆ methoxyalkyl, —OH, —OR^{2a}, oxo, ═N, halogen, —C(O)R^{2a}, —C(O)OR^{2a}, —C(O)NR^{2b}R^{2c}, —SO^{2b}R^{2a}, —CN, —NR^{2b}R^{2c}, 3-6 membered cycloalkyl, 3-7 membered heterocyclyl, 6-10 membered aryl, or 5-10 membered heteroaryl;
   wherein R^{2a} is H, C₁₋₆ alkyl, C₁₋₆ haloalkyl, 3-7 membered heterocyclyl, or —(CH₂)ᵣOCH₃, wherein r is 1, 2, or 3;
   wherein R^{2b} is H or C₁₋₆ alkyl;
   wherein R^{2c} is H or C₁₋₆ alkyl;
R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and
A is an optionally substituted 6-membered aryl or an optionally substituted 5-6 membered heteroaryl;
with the proviso that when

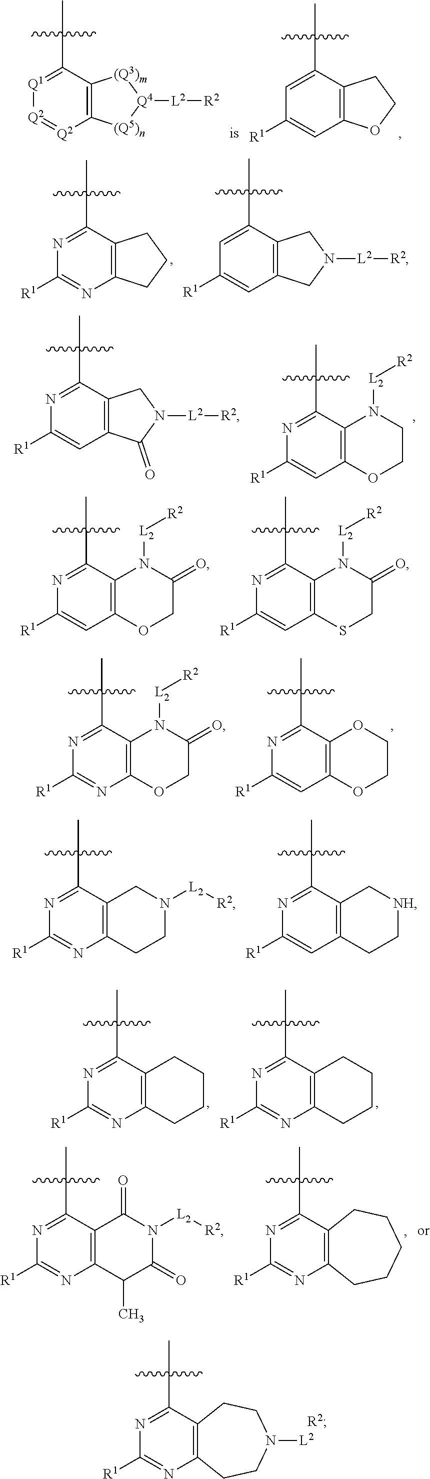

then R¹ is not H.

Embodiment 5 is a method of Embodiment 1, wherein the SOS1 inhibitor is a compound having the structure of Formula (48-I),

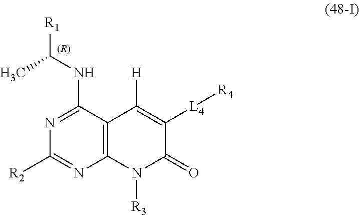

or a pharmaceutically acceptable salt, solvate, isomer, stereoisomer, prodrug, or tautomer thereof, wherein:
R₁ is selected from the group consisting of optionally substituted 3-6 membered cycloalkyl, optionally substituted 3-6 membered heterocyclyl, optionally substituted 6-membered aryl, and optionally substituted 5-6 membered heteroaryl;
R² is selected from the group consisting of H, C₁₋₆ alkyl, halogen, —NHR₂ₐ, —OR₂ₐ, cyclopropyl, and —CN; wherein C₁₋₆ alkyl is optionally substituted with halogen, —NHR₂ₐ, —OR₂ₐ, or 5-6 membered heterocyclyl, and further wherein R^{2a} is selected from the group consisting of H, C₁₋₆ alkyl, 3-6 membered heterocyclyl, and C₁₋₆ haloalkyl;
R³ is selected from the group consisting of H, C₁₋₃ alkyl, —OR₃ₐ, cyclopropyl, and 3-6 membered heterocyclyl, wherein each of C₁₋₃ alkyl, cyclopropyl, and 3-6 membered heterocyclyl is optionally substituted with R₃ₐ, and further wherein R₃ₐ is selected from the group consisting of C₁₋₃ alkyl, halogen, —OH, and —CN;
L₄ is selected from the group consisting of bond, —C(O)—, —C(O)O—, —C(O)NH(CH₂)ₒ—, —NH—, —S—, —S(O)₂—,

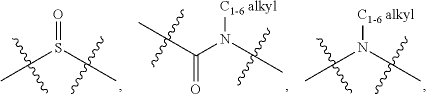

(CH₂)ₚ—, and —O—; wherein o is 0, 1, or 2; and wherein p is a number from 1 to 6; and
R⁴ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —R₄ₐ, —OR^{4a}, —O—C₁₋₆ alkyl-R₄ₐ, ═O, halogen, —C(O)R₄ₐ, —C(OO)R₄ₐ, —C(O)NR_{4b}R_{4c}, —NR_{4b}C(O)R_{4c}, —CN, ═NR₄ₐ, —NR₄₆R_{4c}, —SO₂R₄ₐ, 3-6 membered cycloalkyl, 3-7 membered heterocyclyl, 6-10 membered aryl, or 5-10 membered heteroaryl;
   wherein Ria is H, C₁₋₆ alkyl, C₁₋₆ haloalkyl, —C(O)NR_{4b}R_{4c}, 3-6 membered cycloalkyl, 6-10 membered aryl optionally substituted with —OR_{4b}, —CN, 3-7 membered heterocyclyl, —(CH₂)ᵣOCH₃, or —(CH₂)ᵣOH, wherein r is 1, 2, or 3;
   wherein each R_{4b} is independently H, C₁₋₆ alkyl; and
   wherein each R_{4c} is independently H or C₁₋₆ alkyl.

Embodiment 6 is a method of Embodiment 1, wherein the SOS1 inhibitor is a compound having the structure of Formula (53-I), (53-II), or (53-III),

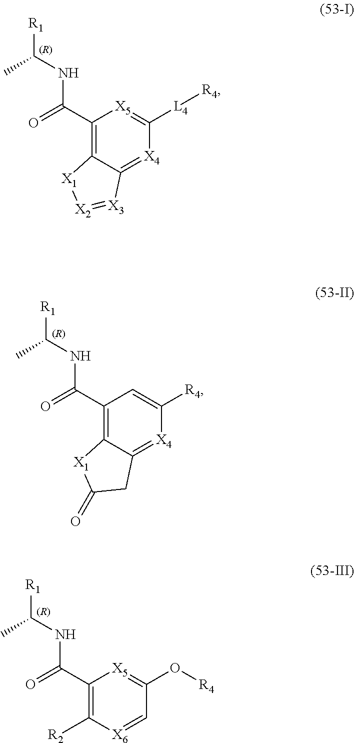

or a pharmaceutically acceptable salt, solvate, isomer, stereoisomer, prodrug, or tautomer thereof, wherein:
X₁ is NH or S;
X₂ is CH or N;
X₃ is CH or N;
X₄ is CR₃ or N;
X₅ is CH or N;
X₆ is CH or N;
R₁ is selected from the group consisting of optionally substituted 3-6 membered cycloalkyl, optionally substituted 3-6 membered heterocyclyl, optionally substituted 6-membered aryl, and optionally substituted 5-6 membered heteroaryl;
R₂ is selected from the group consisting of H, —NH—C₁₋₆ alkyl, and —NH₂;
R₃ is selected from the group consisting of H, —O—C₁₋₆ alkyl, and —O—C₁₋₆ heteroalkyl;
L₄ is a bond or O; and
R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with one or more C₁₋₆ alkyl, —R₄ₐ, —OR₄ₐ, —O—C₁₋₆ alkyl-R₄ₐ, ═O, halogen, —C(O)R₄ₐ, C(O)OR^{4a}, —C(O)NR₄₆R_{4c}, —NR_{4b}C(O)R_{4c}, —CN, ═NR₄ₐ, —NR_{4b}R_{4c}, —SO₂R₄ₐ, 3-6 membered cycloalkyl optionally substituted with R₄ₐ, 3-7 membered heterocyclyl optionally substituted with R₄ₐ, 6-10 membered aryl optionally substituted with R₄ₐ, or 5-10 membered heteroaryl optionally substituted with R₄ₐ;
wherein R₄ₐ is H, C₁₋₆ alkyl, C₁₋₆ haloalkyl, —C(O)R_{4b}, —C(O)NR_{4b}R_{4c}, ═O, 3-6 membered cycloalkyl, 6-10 membered aryl optionally substituted with —OR_{4b}, —CN, ═N-3-6 membered cycloalkyl, 3-7 membered heterocyclyl, —(CH₂)ᵣOCH₃, or —(CH₂)ᵣOH, wherein r is 1, 2, or 3;
wherein each R_{4b} is independently H, C₁₋₆ alkyl; and
   wherein each R_{4c} is independently H or C₁₋₆ alkyl.

Embodiment 7 is a method of Embodiment 1, wherein the SOS1 inhibitor is Compound SOS1-(A) (also called RMC-0331), having the structure:

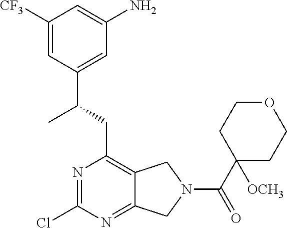 [SMILES: COC1(C(=O)N2Cc3nc(Cl)nc(C[C@H](C)c4cc(N)cc(C(F)(F)F)c4)c3C2)CCOCC1]

or a pharmaceutically acceptable salt, solvate, isomer, stereoisomer, prodrug, or tautomer thereof.

Embodiment 8 is a method of Embodiment 1, wherein the SOS1 inhibitor is Compound SOS1-(B) or a pharmaceutically acceptable salt, solvate, isomer, stereoisomer, prodrug, or tautomer thereof.

Embodiment 9 is a method of Embodiment 1, wherein the SOS1 inhibitor is Compound SOS1-(C) or a pharmaceutically acceptable salt, solvate, isomer, stereoisomer, prodrug, or tautomer thereof.

Embodiment 10 is a method of Embodiment 1, wherein the SOS1 inhibitor is BI-3406, having the structure:

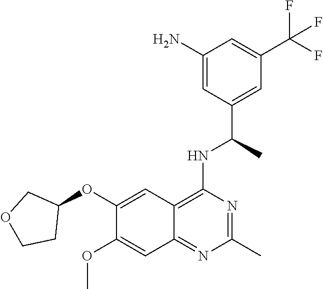 [SMILES: COc1cc2nc(C)nc(N[C@H](C)c3cc(N)cc(C(F)(F)F)c3)c2cc1O[C@H]1CCOC1]

or a pharmaceutically acceptable salt, solvate, isomer, stereoisomer, prodrug, or tautomer thereof.

Embodiment 11 is a method of Embodiment 1, wherein the SOS1 inhibitor is BI-1701963, or a pharmaceutically acceptable salt, solvate, isomer, stereoisomer, prodrug, or tautomer thereof.

Embodiment 12 is a method of Embodiment 1, wherein the SOS1 inhibitor is BAY-293, having the structure:

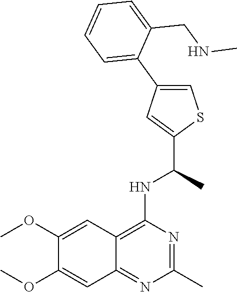

or a pharmaceutically acceptable salt, solvate, isomer, stereoisomer, prodrug, or tautomer thereof.

Embodiment 13 is a method of Embodiment 1, wherein the SOS1 inhibitor is SDR5 or MRTX0902, or a pharmaceutically acceptable salt, solvate, isomer, stereoisomer, prodrug, or tautomer thereof.

Embodiment 14 is a method of any of Embodiments 1 through 13, wherein the bi-steric mTOR inhibitor is selected from those disclosed in WO 2016/040806, WO 2018/204416, WO 2019/212990, and WO 2019/212991.

Embodiment 15 is a method of any of Embodiments 1 through 13, wherein the bi-steric mTOR inhibitor is RM-006 (also called RMC-6272) having the following structure:

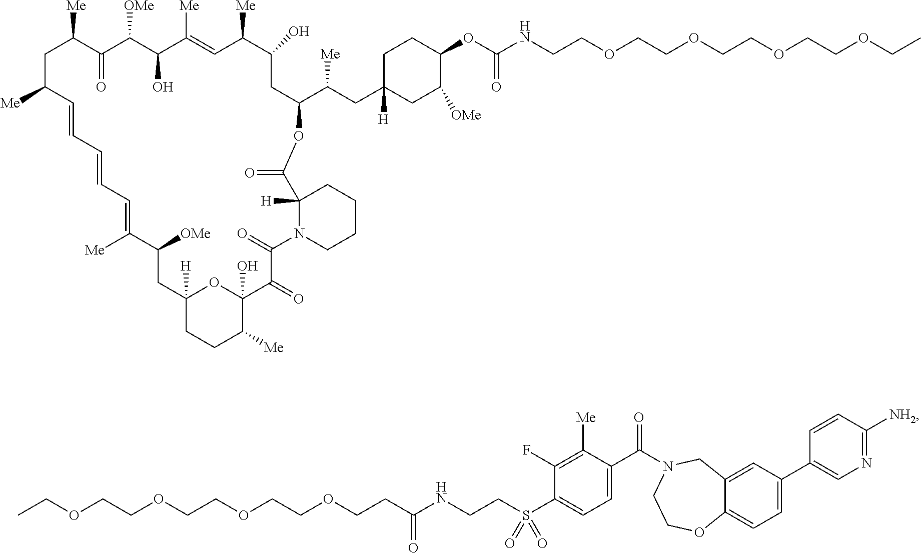

or a pharmaceutically acceptable salt, solvate, isomer, stereoisomer, prodrug, or tautomer thereof. Alternatively, Embodiment 15 is a method of any of Embodiments 1 through 13, wherein the bi-steric mTOR inhibitor is RMC-5552 having the following structure:

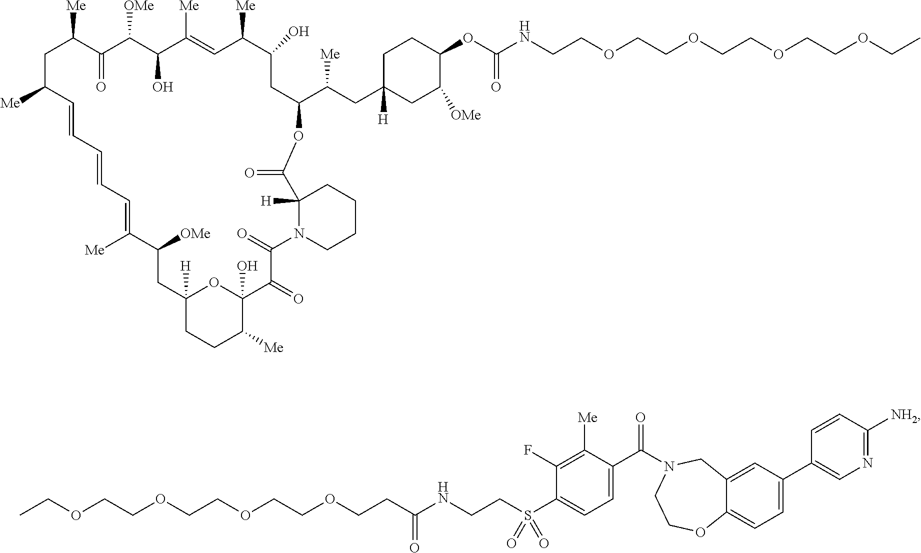

or a stereoisomer, tautomer or oxepane isomer thereof.

Embodiment 16 is a method of any of Embodiments 1 through 13, wherein the bi-steric mTOR inhibitor is RMC-5552 having the following structure:

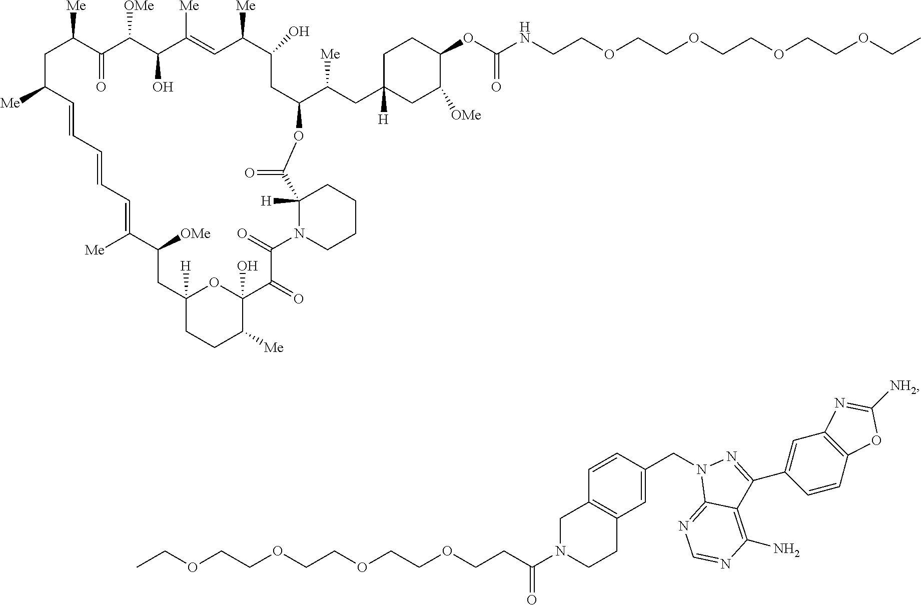

or a pharmaceutically acceptable salt, solvate, isomer, stereoisomer, prodrug, or tautomer thereof. Alternatively, Embodiment 16 is a method of any of Embodiments 1 through 13, wherein the bi-steric mTOR inhibitor is RMC-5552 having the following structure:

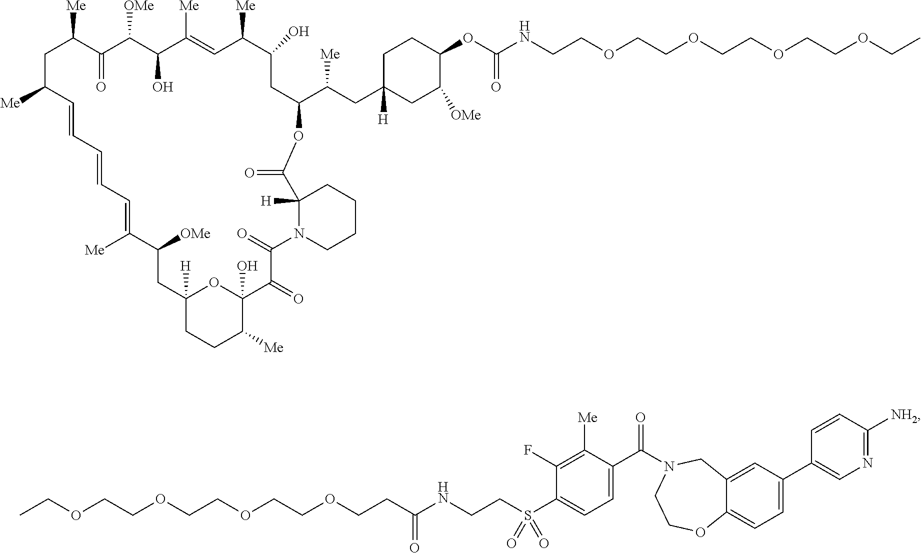

or a stereoisomer, tautomer or oxepane isomer thereof.

Embodiment 17 is a method of Embodiment 1, wherein:
(a) the SOS1 inhibitor is Compound SOS1-(A) having the structure:

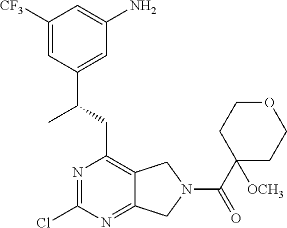 [SMILES: COC1(C(=O)N2Cc3nc(Cl)nc(C[C@H](C)c4cc(N)cc(C(F)(F)F)c4)c3C2)CCOCC1]

or a pharmaceutically acceptable salt, solvate, isomer, stereoisomer, prodrug, or tautomer thereof; and
(b) the bi-steric mTOR inhibitor is RMC-5552 having the following structure:

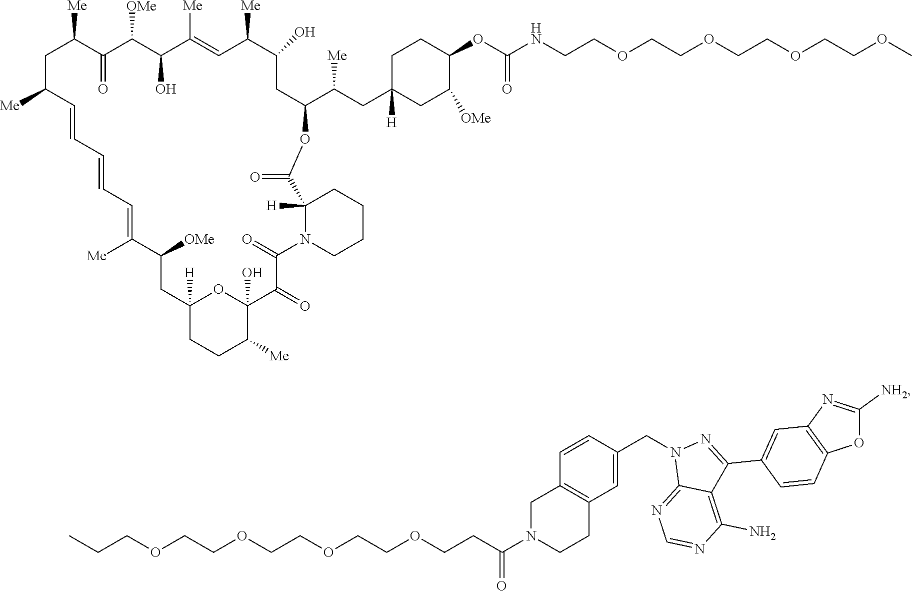

or a stereoisomer, tautomer or oxepane isomer thereof. Alternatively, Embodiment 17 is a method of Embodiment 1, wherein the bi-steric mTOR inhibitor is RMC-5552 having the following structure:

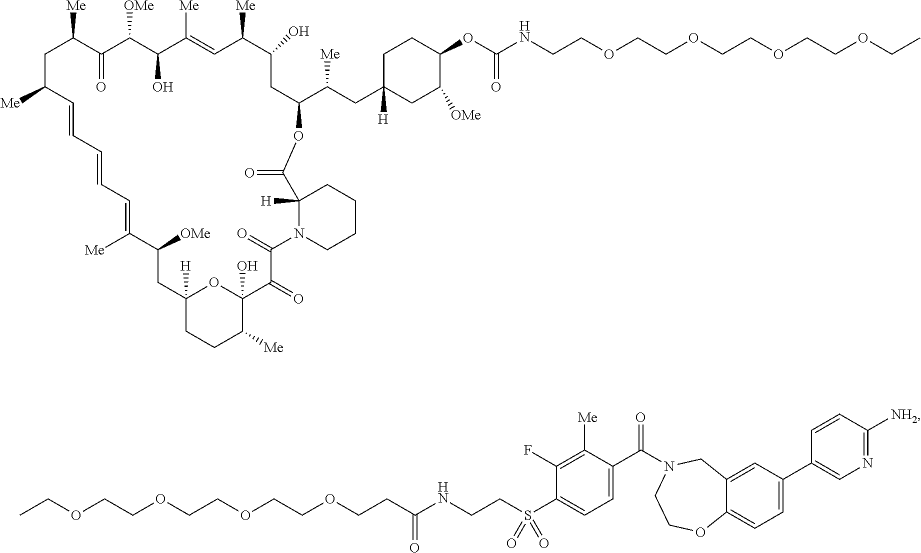

or a stereoisomer, tautomer or oxepane isomer thereof.

Embodiment 18 is a method of Embodiment 1, wherein:
(a) the SOS1 inhibitor is Compound SOS1-(C) or a pharmaceutically salt, solvate, isomer, stereoisomer, prodrug, or tautomer thereof; and
(b) the bi-steric mTOR inhibitor is RMC-5552 having the following structure:

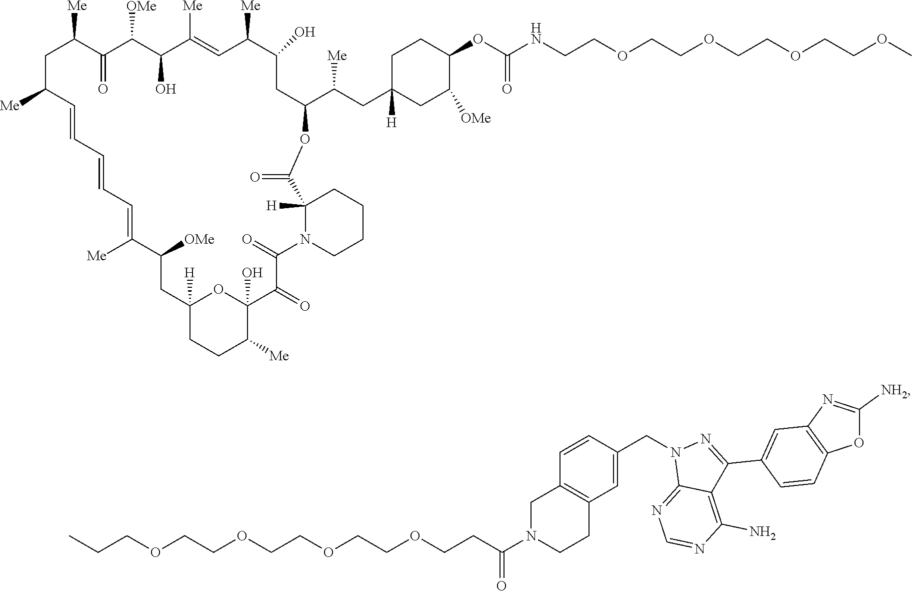

or a stereoisomer, tautomer or oxepane isomer thereof.

Embodiment 19 is a method of Embodiments 1 through 18, wherein the disease or disorder is selected from the group consisting of tumors of hematopoietic and lymphoid system; a myeloproliferative syndrome; a myelodysplastic syndromes; leukemia; acute myeloid leukemia; juvenile myelomonocytic leukemia; esophageal cancer; breast cancer; lung cancer; colon cancer; gastric cancer; neuroblastoma; bladder cancer; prostate cancer; glioblastoma; urothelial carcinoma; uterine carcinoma; adenoid and ovarian serous cystadenocarcinoma; paraganglioma; pheochromocytoma; pancreatic cancer; adrenocortical carcinoma; stomach adenocarcinoma; sarcoma; rhabdomyosarcoma; lymphoma; head and neck cancer; skin cancer; peritoneum cancer; intestinal cancer (e.g., small and/or large intestinal cancer); thyroid cancer; endometrial cancer; cancer of the biliary tract; soft tissue cancer; ovarian cancer; central nervous system cancer (e.g., primary CNS lymphoma); stomach cancer; pituitary cancer; genital tract cancer; urinary tract cancer; salivary gland cancer; cervical cancer; liver cancer; eye cancer; cancer of the adrenal gland; cancer of autonomic ganglia; cancer of the upper aerodigestive tract; bone cancer; testicular cancer; pleura cancer; kidney cancer; penis cancer; parathyroid cancer; cancer of the meninges; vulvar cancer; and melanoma. Alternatively, Embodiment 19 may be a method of Embodiments 1 through 18, wherein the disease or disorder is selected from pancreatic cancer, colorectal cancer, non-small cell lung carcinoma, head and neck cancer and glioblastoma. Alternatively, Embodiment 19 may be a method of Embodiments 1 through 18 wherein the disease or disorder is selected from endometrial cancer, uterine cancer, breast cancer, small-cell lung cancer, non-small cell lung carcinoma (e.g., harboring a KRAS mutation (e.g., KRASG12C), a STK11 mutation or a KEAP1 mutation, or a combination thereof), squamous cell lung carcinoma, cervical cancer, glioblastoma, colorectal cancer, melanoma, head and neck cancer (e.g., squamous cell cancer of the head and neck (HNSCC)), ovarian cancer, prostate cancer, FGFR mutant cancer, hepatocellular carcinoma, hepatoblastoma, glioma, rhabdomyosarcoma, bladder cancer, hematological cancer, pancreatic cancer, renal cancer, neuro-endocrine cancer, thyroid gland adenocarcinoma, a cancer characterized by increased mTORC1 activity, a cancer with an mTORC1 pathway mutation (e.g., STK11, PIK3CA, PTEN, mTOR, KEAP1, TSC1, or TSC2), a cancer having a MYC amplification (e.g., MYC, MYCL, or MYCN), a cancer characterized by decreased 4EBP1 activity, and a cancer with a PTEN mutation.

Embodiment 20 is a method of any one of Embodiments 1 through 18, wherein the disease or disorder is a RASopathy.

Embodiment 21 is a method of Embodiment 20, wherein the RASopathy is selected from the group consisting of Neurofibromatosis type 1 (NF1), Noonan Syndrome (NS), Noonan Syndrome with Multiple Lentigines (NSML), Capillary Malformation-Arteriovenous Malformation Syndrome (CM-AVM), Costello Syndrome (CS), Cardio-Facio-Cutaneous Syndrome (CFC), Legius Syndrome, and Hereditary gingival fibromatosis.

Embodiment 22 is a method of Embodiment 21, wherein the disease or disorder is Noonan Syndrome or Leopard Syndrome.

Embodiment 23 is a method of any one of Embodiments 1 through 18, wherein the disease or disorder is associated with cells having a SHP2 mutation.

Embodiment 24 is a method of Embodiment 23, wherein the SHP2 mutation induces an activated form of SHP2.

Embodiment 25 is a method of Embodiment 23, wherein the subject expressed the SHP2 mutation after prior treatment with a SHP2 inhibitor or a pharmaceutically acceptable salt, solvate, isomer, stereoisomer, prodrug, or tautomer thereof.

Embodiment 26 is a method of Embodiment 23, wherein the subject expressed the SHP2 mutation after prior treatment with an allosteric SHP2 inhibitor or a pharmaceutically acceptable salt, solvate, isomer, stereoisomer, prodrug, or tautomer thereof.

Embodiment 27 is a method of Embodiment 25 or Embodiment 26, wherein the SHP2 inhibitor is selected from those disclosed in WO 2022063190, WO 2022043685, WO 2022042331, WO 2022033430, WO 2022033430, WO 2022017444, WO 2022007869, WO 2021259077, WO 2021249449, WO 2021249057, WO 2021244659, WO 2021218755, WO 2021281752, WO 2021197542, WO 2021176072, WO 2021149817, WO 2021148010, WO 2021147879, WO 2021143823, WO 2021143701, WO 2021143680, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, US 20210085677, US 10858359, US 10934302, US 10954243, US 10988466, US 11001561, US 11033547, US 11034705, US 11044675, CN 114213417, CN 114163457, CN 113896710, CN 113248521, CN 113248449, CN 113135924, CN 113024508, CN 112920131, CN 112823796, CN 112402385, CN 111848599, CN 111704611, CN 111265529, and CN 108113848, or a pharmaceutically acceptable salt, solvate, isomer (e.g., stereoisomer), prodrug, or tautomer thereof, each of which is incorporated herein by reference.

Embodiment 28 is a method of Embodiment 25 or Embodiment 26, wherein the SHP2 inhibitor or a pharmaceutically acceptable salt, solvate, isomer, stereoisomer, prodrug, or tautomer thereof is selected from the group consisting of ERAS-601, BBP-398, RLY-1971, JAB-3068, JAB-3312, TNO155, SHP099, SH3809, PF-07284892, RMC-4550, and RMC-4630.

Embodiment 29 is a method of Embodiment 23, wherein the SHP2 mutation confers resistance to a SHP2 inhibitor or a pharmaceutically acceptable salt, solvate, isomer, stereoisomer, prodrug, or tautomer thereof or an allosteric SHP2 inhibitor or a pharmaceutically acceptable salt, solvate, isomer, stereoisomer, prodrug, or tautomer thereof.

Embodiment 30 is a method of Embodiment 23, wherein SHP2 mutation is selected from the group consisting of G60V, D61G, D61V, E69K, A72S, A72T, A72V, T73I, E76A, E76G, E76K, E76Q, S189A, L262R, F285S, N308D, T468M, P491S, G503V, Q506P, T507K, S502P, T253M/Q257L, and any combination thereof.

Embodiment 31 is a method of Embodiment 30, wherein SHP2 mutation is G503V.

Embodiment 32 is a method of Embodiment 1, wherein the disease or disorder is a RAS protein-related disease or disorder.

Embodiment 33 is a method of Embodiment 32, wherein the RAS protein-related disease or disorder is associated with a RAS protein mutation.

Embodiment 34 is a method of Embodiment 33, wherein the RAS protein mutation is at position G12, G13, Q61, A146, K117, L19, Q22, V14, A59, or a combination thereof, of a RAS protein.

Embodiment 35 is a method of Embodiment 33 or Embodiment 34, wherein the RAS protein mutation is selected from the group consisting of G12C, G12D, G12A, G12S, G12V, G13C, G13D, Q61K, and Q61L.

Embodiment 36 is a method of any one of Embodiments 32 to 35, wherein the RAS protein is KRAS.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Cell Lines Used in the Examples

The following table shows the cell lines used in the Examples.

| Cell Line | Species | Histotype | Genotype |
|---|---|---|---|
| PAN02 | Mouse | Pancreatic | SHP2^{G503V} |
| SW837 | Human | CRC | KRAS^{G12C} |
| MIAPaCa-2 | Human | Pancreatic | KRAS^{G12C} |
| AsPC-1 | Human | Pancreatic | KRAS^{G12D} |
| HCT-15 | Human | CRC | KRAS^{G13D} |
| NCI-H1355 | Human | NSCLC | KRAS^{G13C} |

### Methods Used in the Examples

### NTRC Methods

### SynergyScreen™

Assay stocks were thawed and diluted in the recommended ATCC medium, supplemented with 10% serum and 1% pen/strep (final concentration) and dispensed in a 384-well plate. Depending on the cell line used, a cell density of 100-6400 cells per well in 45 μl medium was used. The margins of the plate were filled with phosphate-buffered saline. Plated cells were incubated in a humidified atmosphere of 5% CO2 at 37° C. After 24 hours, 5 μl of reference compound dilution, containing the single dose compound at indicated concentration was added to the plates and these were further incubated for 120 hrs. At t=120 hours, 24 μl of ATPlite 1Step™ (PerkinElmer) solution was added to each well, and subsequently shaken for 2 minutes. After 5 minutes incubation in the dark, the luminescence was recorded on an Envision multilabel reader (PerkinElmer). On a parallel t=0 plate, 45 μl cells were dispensed and incubated in a humidified atmosphere of 5% CO2 at 37° C. After 24 hours 5 μl DMSO-containing Hepes buffer and 24 μl ATPlite 1 Step™ solution were mixed, and luminescence measured after 5 minutes incubation (=luminescencet=0).

### Data Analysis

Curves and IC₅₀s were calculated by non-linear regression using IDBS XLfit 5. The percentage growth after incubation until t-end (% growth) was calculated as follows: 100%×(luminescencet_{=end}/luminescence_{untreated,t=end}). This was fitted to the ¹⁰ log compound concentration (conc) by a 4 parameter logistics curve: %-growth=bottom+(top−bottom)(1+10 (logIC₅₀−conc)*hill)) where hill is the Hill-coefficient, and bottom and top the asymptotic minimum and maximum cell growth that the compound allows in that assay.

### Example 1. SOS1 Inhibitor Shows Combination Benefits with mTOR Inhibitor in Vitro

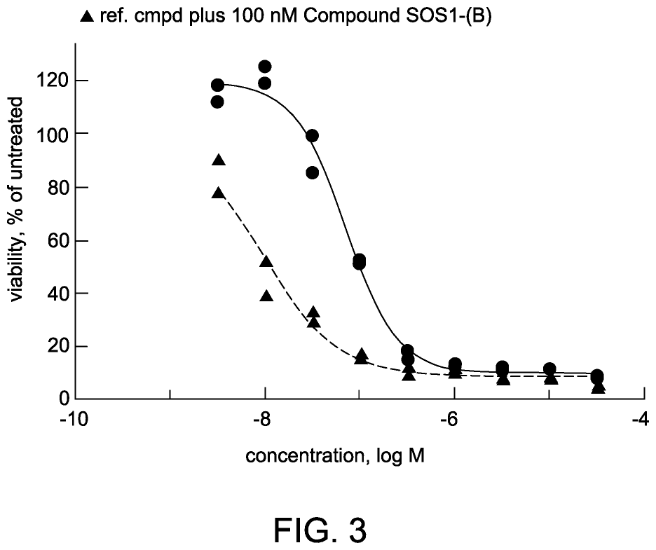

In this example, PAN02 cells (pancreatic cancer, mouse) were characterized. The PAN02 cell line contained the SHP2^{G503V} mutation. The cell lines were treated with DMSO (vehicle) and a constant concentration (100 nM) of SOS1 inhibitor, Compound SOS1-(B). The cell lines were treated with varying concentrations of mTORC1 inhibitor, Compound RM-006 (also known as RMC-6272). FIG. 3 is a graph depicting the in vitro combination effect of a SOS1 inhibitor and an mTORC1 inhibitor observed in PAN02 cells. The effect of the mTORC1 inhibitor as a single agent is shown with black circles. The effect of the mTORC1 inhibitor in combination with 100 nM of the SOS1 inhibitor is shown with grey triangles. An increase in apparent potency (left-ward shift of curve) in the combination is an indication of a positive interaction between the compounds. The data were obtained according to the NTRC methods experimental protocol.

### Example 2. SOS1 Inhibitor Shows Combination Benefits with mTOR Inhibitor in Vitro

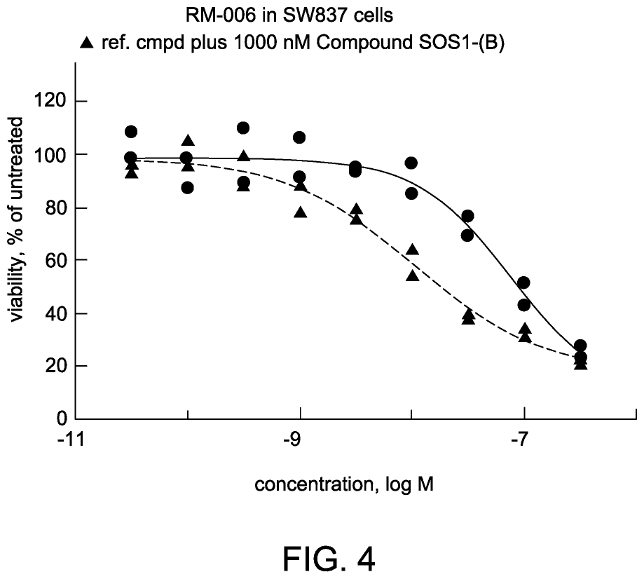

In this example, SW837 cells colorectal cancer, human) were characterized. The SW837 cell line contained the KRAS^{G12C} mutation. The cell lines were treated with DMSO (vehicle) and a constant concentration (1000 nM) of SOS1 inhibitor, Compound SOS1-(B). The cell lines were treated with varying concentrations of mTORC1 inhibitor, Compound RM-006 (also known as RMC-6272). FIG. 4 is a graph depicting the in vitro combination effect of a SOS1 inhibitor and an mTORC1 inhibitor observed in SW837 cells. The effect of the mTORC1 inhibitor as a single agent is shown with black circles. The effect of the mTORC1 inhibitor in combination with 1000 nM (1 μM) of the SOS1 inhibitor is shown with grey triangles. An increase in apparent potency (left-ward shift of curve) in the combination is an indication of a positive interaction between the compounds. The data were obtained according to the NTRC methods experimental protocol.

### Example 3. SOS1 Inhibitor Shows Combination Benefits with mTOR Inhibitor in Vitro

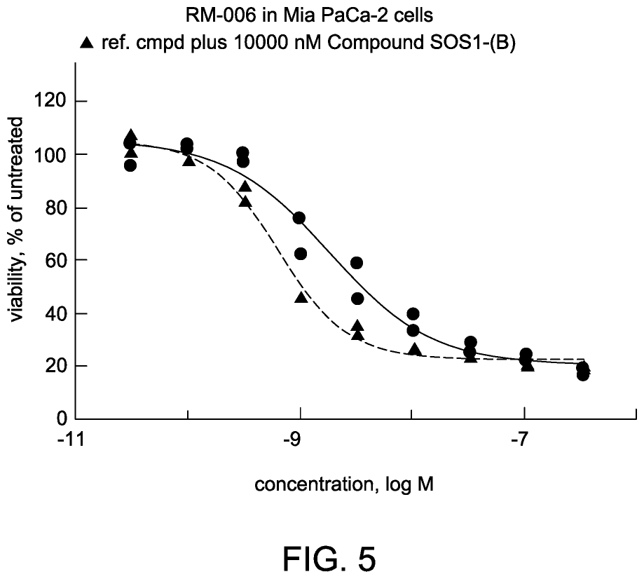

In this example, MIA PaCa-2 cells (pancreatic cancer, human) were characterized. The MIA PaCa-2 cell line contained the KRAS^{G12C} mutation. The cell lines were treated with DMSO (vehicle) and a constant concentration (10,000 nM, 10 μM) of SOS1 inhibitor, Compound SOS1-(B). The cell lines were treated with varying concentrations of mTORC1 inhibitor, Compound RM-006 (also known as RMC-6272). FIG. 5 is a graph depicting the in vitro combination effect of a SOS1 inhibitor and an mTORC1 inhibitor observed in MIA PaCa-2 cells. The effect of the mTORC1 inhibitor as a single agent is shown with black circles. The effect of the mTORC1 inhibitor in combination with 10 μM of the SOS1 inhibitor is shown with grey triangles. An increase in apparent potency (left-ward shift of curve) in the combination is an indication of a positive interaction between the compounds. The data were obtained according to the NTRC methods experimental protocol.

### Example 4. SOS1 Inhibitor Shows Combination Benefits with mTOR Inhibitor in vitro

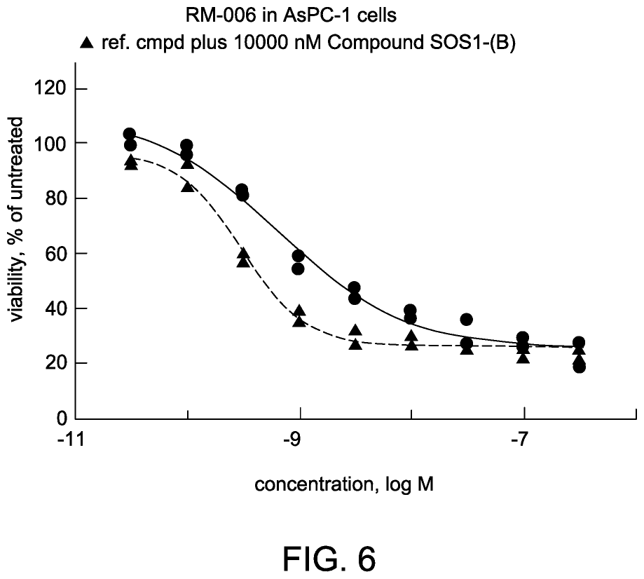

In this example, AsPC-1 cells (pancreatic cancer, human) were characterized. The AsPC-1 cell line contained the KRAS^{G12D} mutation. The cell lines were treated with DMSO (vehicle) and a constant concentration (10,000 nM, 10 μM) of SOS1 inhibitor, Compound SOS1-(B). The cell lines were treated with varying concentrations of mTORC1 inhibitor, Compound RM-006 (also known as RMC-6272). FIG. 6 is a graph depicting the in vitro combination effect of a SOS1 inhibitor and an mTORC1 inhibitor observed in AsPc-1 cells. The effect of the mTORC1 inhibitor as a single agent is shown with black circles. The effect of the mTORC1 inhibitor in combination with 10 μM of the SOS1 inhibitor is shown with grey triangles. An increase in apparent potency (left-ward shift of curve) in the combination is an indication of a positive interaction between the compounds. The data were obtained according to the NTRC methods experimental protocol.

### Example 5. SOS1 Inhibitor Shows Combination Benefits with mTOR Inhibitor in vitro

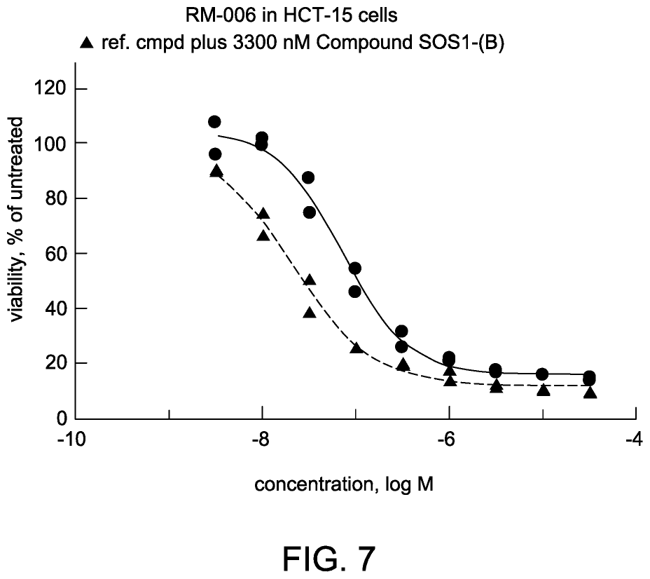

In this example, HCT-15 cells (colorectal cancer, human) were characterized. The HCT-15 cell line contained the KRAS^{G13D} mutation. The cell lines were treated with DMSO (vehicle) and a constant concentration (3300 nm, 3.3 μM) of SOS1 inhibitor, Compound SOS1-(B). The cell lines were treated with varying concentrations of mTORC1 inhibitor, Compound RM-006 (also known as RMC-6272). FIG. 7 is a graph depicting the in vitro combination effect of a SOS1 inhibitor and an mTORC1 inhibitor observed in HCT-15 cells. The effect of the mTORC1 inhibitor as a single agent is shown with black circles. The effect of the mTORC1 inhibitor in combination with 3.3 μM of the SOS1 inhibitor is shown with grey triangles. An increase in apparent potency (left-ward shift of curve) in the combination is an indication of a positive interaction between the compounds. The data were obtained according to the NTRC methods experimental protocol.

### Example 6. SOS1 Inhibitor Shows Combination Benefits with mTOR Inhibitor in vitro

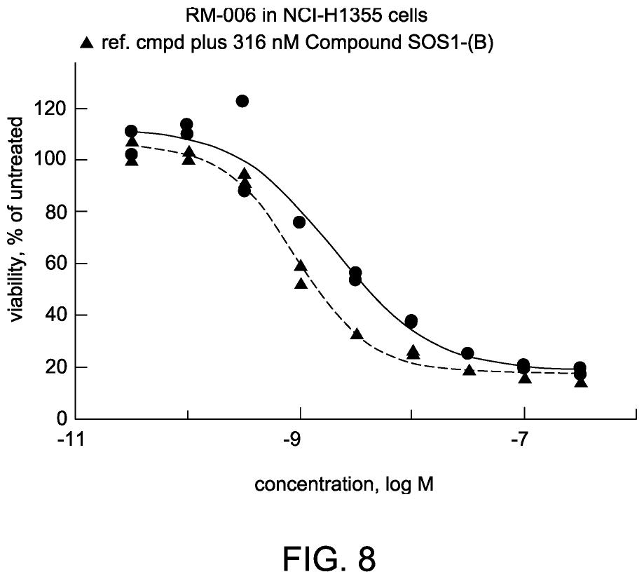

In this example, NCI-H1355 cells (NSCLC, human) were characterized. The NCI-H1355 cell line contained the KRAS^{G13C} mutation. The cell lines were treated with DMSO (vehicle) and a constant concentration (316 nM) of SOS1 inhibitor, Compound SOS1-(B). The cell lines were treated with varying concentrations of mTORC1 inhibitor, Compound RM-006 (also known as RMC-6272). FIG. 8 is a graph depicting the in vitro combination effect of a SOS1 inhibitor and an mTORC1 inhibitor observed in NCI-H1355 cells. The effect of the mTORC1 inhibitor as a single agent is shown with black circles. The effect of the mTORC1 inhibitor in combination with 316 nM of the SOS1 inhibitor is shown with grey triangles. An increase in apparent potency (left-ward shift of curve) in the combination is an indication of a positive interaction between the compounds. The data were obtained according to the NTRC methods experimental protocol.

### Example 7. SOS1 Inhibitor Shows Combination Benefits with mTOR Inhibitor in vivo

The combinatorial effects of Compound SOS1-(C) with RM-006 (also known as RMC-6272) on tumor cell growth in vivo were evaluated in the murine pancreatic ductal adenocarcinoma PAN02 PTPN11^{G503V} syngeneic model using female C57BL/6J mice (6-7 weeks old). Mice were implanted with PAN02 tumor cells in PBS (5×10⁶ cells/mouse) subcutaneously in the flank. Once tumors reached an average size of ˜145 mm³, mice were randomized to treatment groups to start the administration of test articles or control. SOS1-(C) treatment was administered daily by oral gavage and RM-006 was administered weekly by intra-peritoneal injection. Body weight and tumor volumes (using calipers) were measured twice weekly until study endpoint. Regressions (R) are defined as decrease of 10% or more from initial tumor volume.

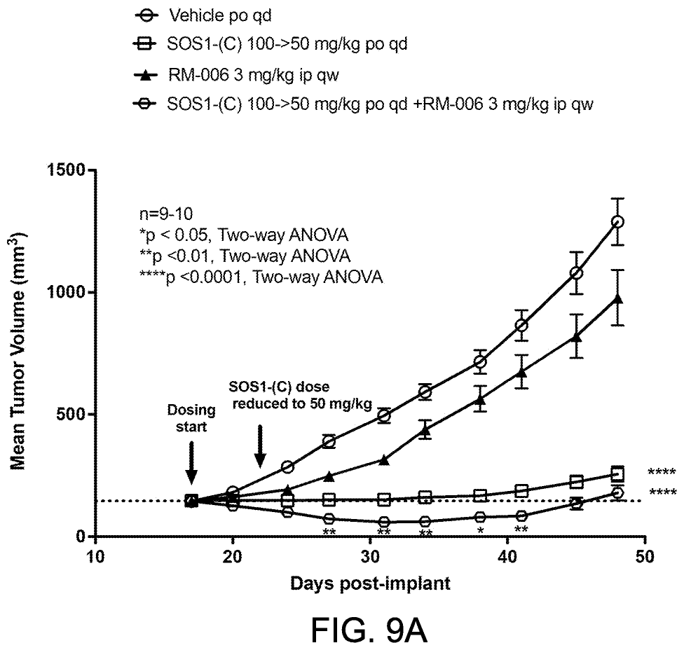

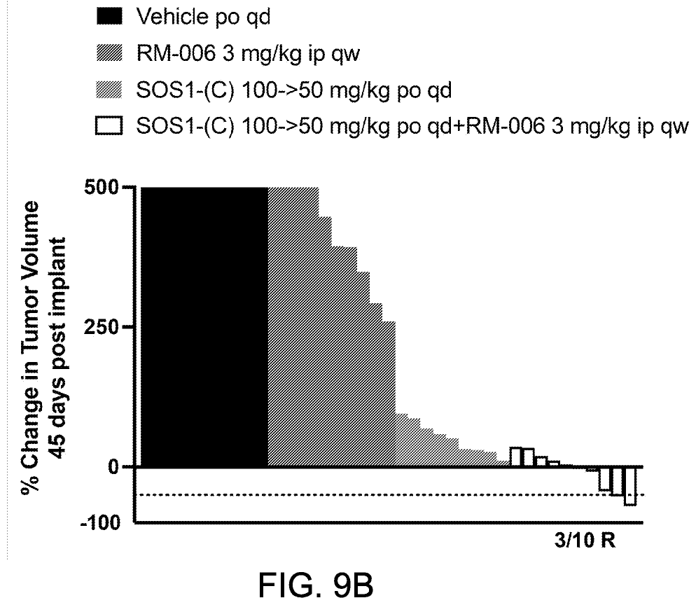

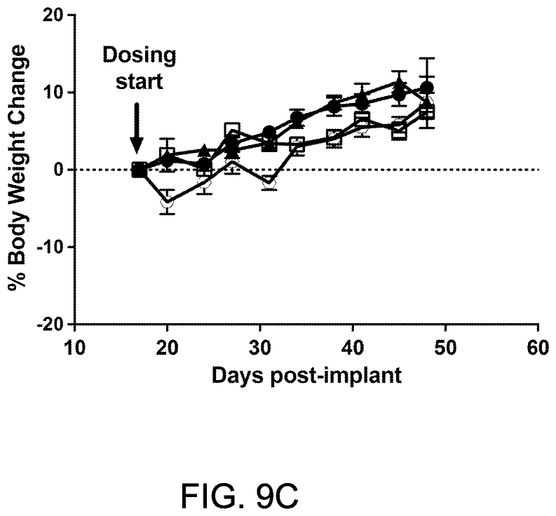

In FIG. 9A, single-agent Compound SOS1-(C) administered daily at 100 mg/kg PO for the first 5 days of treatment and then 50 mg/kg PO daily for the remainder of the study led to tumor growth inhibition (TGI) of 88%; single-agent RM-006 administered at 3 mg/kg IP weekly led to a TGI of 11%, in the PAN02 murine PDAC model with a PTPN11G5031 mutation. The combination treatment led to tumor growth inhibition of 96% at the end of study. Combination treatment was statistically significant as compared to SOS1-(C) on days 27-41 post implant (days 10-24 of treatment). SOS1-(C) and combination treatment were statistically significant from vehicle group at all timepoints starting 24 days post-implant (7 days following initiation of treatment). (*p<0.05, ** p<0.01, **** p<0.0001. assessed by two-way ANOVA of tumor volumes along with multiple comparisons via a post-hoc Tukey's test in GraphPad Prism software). The waterfall plot (FIG. 9B) shows individual tumor responses 45 days post implant (28 days of treatment), 3/10 tumors from the combination group showed regression (R), however single agent SOS1-(C) or RM-006 did not induce regressions at any timepoint in this study. All treatment was well tolerated as determined by body weight (FIG. 9C).

### Example 8. SOS1 Inhibitor Shows Combination Benefits with mTOR Inhibitor in vivo

The combinatorial effects of Compound SOS1-(C) with RM-006 (also known as RMC-6272) on tumor cell growth in a human model of PTPN11-mutant glioblastoma, LN229 PTPN11^{A72S/WT}. 6-7 week athymic nude mice were implanted with LN229 in 50% matrigel (10×10⁶ cells/mouse) subcutaneously in the flank. Once tumors reached an average size of 200 mm³, mice were randomized into treatment groups (n=10/group) to start the administration of test articles or control. SOS1-(C) treatment was administered daily by oral gavage and RM-006 was administered weekly by intra-peritoneal injection. Body weight and tumor volume (using calipers) was measured twice weekly until study endpoint.

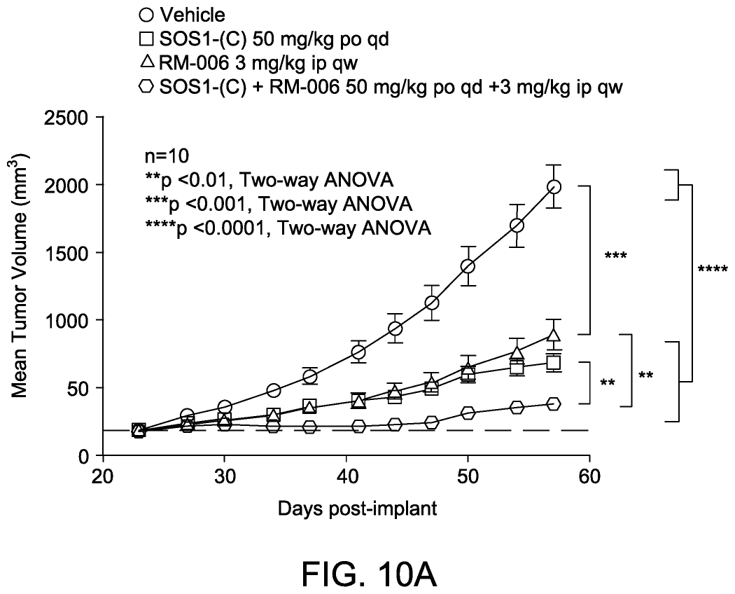

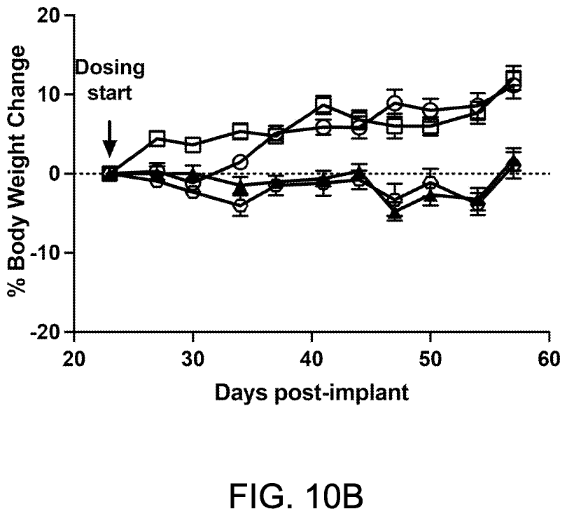

In FIG. 10A, single-agent Compound SOS1-(C) administered at 50 mg/kg PO daily led to tumor growth inhibition (TGI) of 61%; single-agent RM-006 administered at 3 mg/kg IP weekly led to a TGI of 72% in the LN229 human glioblastoma CDX model with a PTPN11^{A72S/WT} mutation. The combination treatment led to TGI of 89% for the 50 mg/kg SOS1-(C)+3 mg/kg RM-006 group. All treatment arms were statistically significant from the vehicle control group and the combination treatment was statistically significant from the monotherapy groups (** p<0.01, *** p<0.001, and **** p<0.0001, assessed by a two-way ANOVA of tumor volumes along with multiple comparisons via a post-hoc Tukey test in GraphPad Prism software). Monotherapy treatments were well tolerated as determined by body weight (FIG. 10B). In the combination group one mouse out of 10 was off treatment on days 45-55 post implant due to weight loss >15%.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1-8. (canceled)

9. A method of treating a subject having a disease or disorder, the method comprising administer to the subject in need of such treatment:
(a) a therapeutically effective amount of a SOS1 inhibitor, wherein the SOS1 inhibitor is Compound SOS1-(A) having the structure: 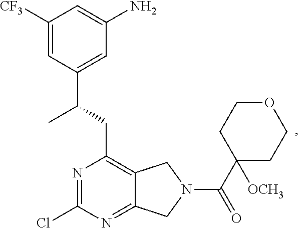 [SMILES: COC1(C(=O)N2Cc3nc(Cl)nc(C[C@H](C)c4cc(N)cc(C(F)(F)F)c4)c3C2)CCOCC1]
or a pharmaceutically acceptable salt, solvate, isomer, stereoisomer, prodrug, or tautomer thereof; and
(b) a therapeutically effective amount of a bi-steric mTOR inhibitor, wherein the bi-steric mTOR inhibitor is RMC-5552 having the following structure: 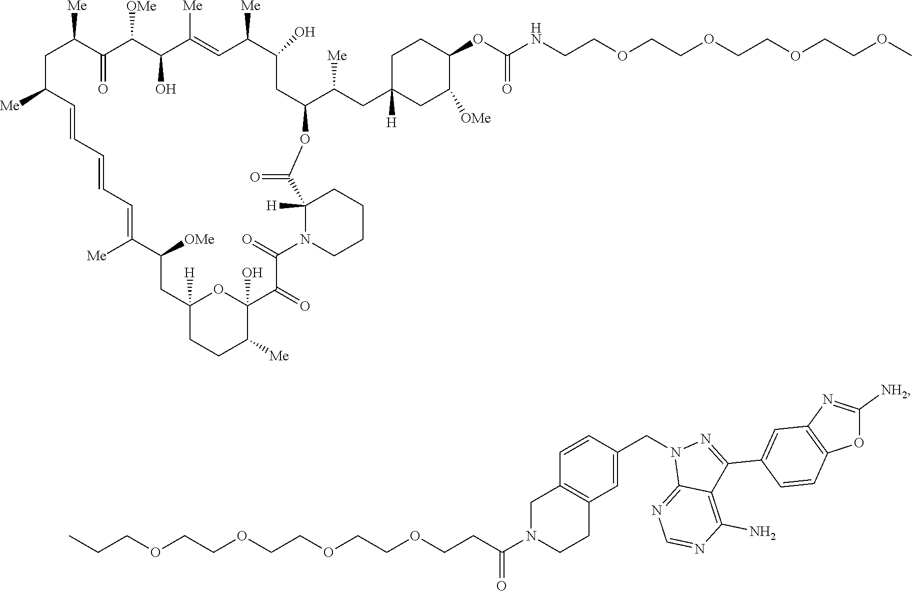
or a pharmaceutically acceptable salt, solvate, isomer, stereoisomer, prodrug, or tautomer thereof.

10. The method of claim 9, wherein the bi-steric mTOR inhibitor is RMC-5552, or a stereoisomer, tautomer or oxepane isomer thereof.

11. The method of claim 9, wherein the disease or disorder is selected from the group consisting of tumors of hematopoietic and lymphoid system; a myeloproliferative syndrome; a myelodysplastic syndromes; leukemia; acute myeloid leukemia; juvenile myelomonocytic leukemia; esophageal cancer; breast cancer; lung cancer; colon cancer; gastric cancer; neuroblastoma; bladder cancer; prostate cancer; glioblastoma; urothelial carcinoma; uterine carcinoma; adenoid and ovarian serous cystadenocarcinoma; paraganglioma; pheochromocytoma; pancreatic cancer; adrenocortical carcinoma; stomach adenocarcinoma; sarcoma; rhabdomyosarcoma; lymphoma; head and neck cancer; skin cancer; peritoneum cancer; intestinal cancer; thyroid cancer; endometrial cancer; cancer of the biliary tract; soft tissue cancer; ovarian cancer; central nervous system cancer; stomach cancer; pituitary cancer; genital tract cancer; urinary tract cancer; salivary gland cancer; cervical cancer; liver cancer; eye cancer; cancer of the adrenal gland; cancer of autonomic ganglia; cancer of the upper aerodigestive tract; bone cancer; testicular cancer; pleura cancer; kidney cancer; penis cancer; parathyroid cancer; cancer of the meninges; vulvar cancer; and melanoma.

12. The method of claim 9, wherein the disease or disorder is a RASopathy.

13. The method of claim 9, wherein the disease or disorder is associated with cells having a SHP2 mutation.

14. The method of claim 13, wherein the SHP2 mutation induces an activated form of SHP2.

15. The method of claim 13, wherein the subject expressed the SHP2 mutation after prior treatment with a SHP2 inhibitor or a pharmaceutically acceptable salt, solvate, isomer, stereoisomer, prodrug, or tautomer thereof.

16. The method of claim 13, wherein the subject expressed the SHP2 mutation after prior treatment with an allosteric SHP2 inhibitor or a pharmaceutically acceptable salt, solvate, isomer, stereoisomer, prodrug, or tautomer thereof.

17. The method of claim 13, wherein the SHP2 mutation confers resistance to a SHP2 inhibitor or a pharmaceutically acceptable salt, solvate, isomer, stereoisomer, prodrug, or tautomer thereof or an allosteric SHP2 inhibitor or a pharmaceutically acceptable salt, solvate, isomer, stereoisomer, prodrug, or tautomer thereof.

18. The method of claim 13, wherein SHP2 mutation is selected from the group consisting of G60V, D61G, D61V, E69K, A72S, A72T, A72V, T73I, E76A, E76G, E76K, E76Q, S189A, L²⁶²R, F285S, N308D, T468M, P491S, G503V, Q⁵⁰⁶P, T507K, S502P, T253M/Q²⁵⁷L, and any combination thereof.

19. The method of claim 18, wherein SHP2 mutation is G503V.

20. The method of claim 9, wherein the disease or disorder is a RAS protein-related disease or disorder.

21. The method of claim 20, wherein the RAS protein-related disease or disorder is associated with a RAS protein mutation.

22. The method of claim 21, wherein the RAS protein mutation is at position G12, G13, Q⁶¹, A146, K117, L¹⁹, Q²², V14, A59, or a combination thereof, of a RAS protein.

23. The method of claim 21, wherein the RAS protein mutation is selected from the group consisting of G12C, G12D, G12A, G12S, G12V, G13C, G13D, Q⁶¹K, and Q⁶¹L.

24. The method of claim 20, wherein the RAS protein is KRAS.
